SEQUENCE LISTING (115 sequences)

SEQ ID NO: 1 - DNA, 3719 bp, synthetic construct
  source 1..3719: mol_type = other DNA
cgggatctcgacgctctcccttatgcgactcctgcattaggaaattaatacgactcactataggggaattgtgagcggataacaattcccctgtagaaataattttgtttaactttaataaggagatataccatgggcagcagccatcaccatcatcaccacagccaggatccgaattcgagctcggcgcgcctgcaggtcgacaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggcagatctcaattggatatcggccggccacgcgatcgctgacgtcggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgacaagctgacgaccgggtctccgcaagtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcggtcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggcatgctagcgcagaaacgtcctagaagatgccaggaggatacttagcagagagacaataaggccggagcgaagccgtttttccataggctccgcccccctgacgaacatcacgaaatctgacgctcaaatcagtggtggcgaaacccgacaggactataaagataccaggcgtttccccctgatggctccctcttgcgctctcctgttcccgtcctgcggcgtccgtgttgtggtggaggctttacccaaatcaccacgtcccgttccgtgtagacagttcgctccaagctgggctgtgtgcaagaaccccccgttcagcccgactgctgcgccttatccggtaactatcatcttgagtccaacccggaaagacacgacaaaacgccactggcagcagccattggtaactgagaattagtggatttagatatcgagagtcttgaagtggtggcctaacagaggctacactgaaaggacagtatttggtatctgcgctccactaaagccagttaccaggttaagcagttccccaactgacttaaccttcgatcaaaccgcctccccaggcggttttttcgtttacagagcaggagattacgacgatcgtaaaaggatctcaagaagatcctttacggattcccgacaccatcactctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtc

SEQ ID NO: 2 - DNA, 8115 bp, synthetic construct
  source 1..8115: mol_type = other DNA
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttataagaaggagatatacatatgaacagtcagcaaaatcctgatttagctgttcatcccttggcaattcctatggaaggcttactaggagaaagtgctacaactcttgagaagaatgtaattgccacacaactctcagaggaagcccaagtaaagctagaggtaatccaaagtttactggaaccctgcgatcgcacaacttatgggcaaaagttgcgggaagcagcagagaaactaaatgtatcgttgcgaacggtacaaaggttggtgaaaaactgggaacaagatggcttagtcggcctcactcaaacaagtagggctgataaaggaaaacaccgcattggtgagttttgggaaaacttcattaccaaaacctacaaggagggtaacaagggaagtaaacgtatgacccctaaacaagttgctctcagagtcgaggctaaagcccgtgaattaaaagactctaagccgcccaattacaaaaccgtgttacgggtattagcacccattttggaaaagcaacaaaaagccaagagtatccgcagtcctggctggagaggaactacgctttcggttaaaacccgtgaaggaaaagatttatcggttgattacagtaaccatgtttggcaatgtgaccatacccgcgtggatgtgttgctggtagatcaacatggtgaaattttaagtcgtccctggctaacaacagtaattgatacttactctcgttgcattatgggtatcaacttgggctttgatgcacccagttctggggtagtagcattagcgttacgccatgcaattctaccaaagcgttacggttccgagtacaaactgcattgtgagtggggaacctatggaaaaccagaacatttttatactgatggcggtaaagactttcgctctaaccacttgagtcagattggggcgcagttgggatttgtctgtcatttacgcgatcgcccttctgaaggtggagtagtagaacgtcccttcaaaacattaaatgaccaactattttcaacgcttcctgggtacaccggatctaatgtgcaggaacgcccagaagatgcagagaaggacgcaagacttactttgcgagaactagaacagttacttgtgcgttacatcgtagatcgttacaaccaaagtattgatgcgcggatgggcgaccaaacgcgctttgagcgttgggaagcaggattgcctacagtgccagtaccaataccagaacgagatttggatatttgtttaatgaagcagtcacggcgcactgtgcaaagaggtggttgtttgcagtttcagaatttaatgtatcggggggagtatttggcaggttatgccggagaaactgtcaacttgaggtttgaccccagagacattacaacaattttggtttatcgccaggaaaacaatcaggaagtatttctgactcgcgctcacgctcaaggtttggagacagagcaactggcattagatgaggctgaggcagcaagtcgcagactccgtaccgcagggaaaactatcagtaaccaatcattattgcaagaagttgttgaccgcgatgctcttgtcgctaccaagaaaagccgtaaggagcgtcaaaaattggaacagactgttttgcgatctgctgctgttgatgaaagtaatagagaatccttgccttctcaaatagttgaaccagatgaagtggaatctacagaaacggttcactctcaatacgaggacattgaggtgtgggactatgaacaacttcgtgaagaatatgggttttaaacaatgacagaagctcaggcgatcgccaagcagttgggtggggtaaaaccggatgatgagtggttacaagctgaaattgctcgtctcaagggtaagagcattgtgcctttacagcaggtaaaaactctccatgattggttagatggcaagcgcaaggcaagaaaatcttgccgagtagttggggaatcgagaactggcaagacagttgcttgtgatgcctacagatacaggcacaaacctcagcaggaagctggacgaccgccaactgtgcctgtcgtttatattcgacctcaccaaaaatgtggccccaaggatttgtttaaaaagattactgagtacctcaagtatcgggtaacaaaagggactgtatctgattttcgagataggacgatagaagtactcaagggttgtggcgtagagatgctaattattgatgaagctgaccgtctcaagcctgaaacttttgctgatgtgcgagatattgccgaagatttaggaattgctgtggtactggtaggaacagaccgtttggatgcggtaattaagcgggatgagcaggttctcgaacgctttcgggcgcatcttcgctttggtaaattgtcgggagaggattttaagaacaccgtagaaatgtgggaacaaatggttttgaaactgccagtatcttctaatctaaagagcaaggagatgctacggattctcacgtcagcaactgaaggctacattggtcgccttgatgagattcttagggaagctgcaattcgttccttatcaagaggattgaagaagattgacaaggctgttttacaggaagtagctaaggagtacaaatgatagaagcaccagatgttaaaccttggctattcttgattaaaccctatgaaggggaaagcctgagccactttcttggcaggttcagacgtgccaaccatttatccgcaagtggattgggtactttggcaggaattggtgctatagtggcacgttgggaaagatttcattttaatcctcgccctagtcagcaagaattggaagcgatcgcatctgtagtagaagtggatgctcaaaggttagcccagatgttaccgcctgctggagtgggaatgcagcatgagccaattcgcttgtgtggggcttgttatgccgagtcgccttgtcaccgaattgaatggcagtacaagtcggtgtggaagtgcgatcgccatcaactcaagattttagcaaagtgtccaaactgtcaagcaccttttaaaatgcctgcgctgtgggaggatgggtgctgtcacagatgtaggatgccgtttgcagaaatggcaaagctacagaaggtttgatgataaaaccagaaaaaggtcttaaataaggagatataccatgagtcaaataactattcaagctcgacttatttcctttgaatcaaaccgccaacaactctggaagttgatggcagatttaaacacgccgttaattaacgaactgctttgccagttaggtcaacaccccgacttcgagaagtggcaacaaaagggtaaactcccgtctaccgttgtgagccagttatgtcaacctctcaaaactgaccctcgctttgcaggtcagcccagccgtttatatatgtcggcaattcatattgtggactacatctacaagtcctggctggctatacagaaacggcttcaacagcagctagatggaaagacgcgctggctagaaatgctcaatagcgatgctgaattagtagaacttagtggtgacactttagaggctattcgtgtcaaagctgctgaaattttggcaatagctatgccagcatctgagtcagatagcgcttcacctaaagggaaaaaaggtaaaaaggagaaaaaaccctcatcttctagccctaagcgtagtttatccaagacattatttgacgcttaccaagaaacggaagatattaagagccgtagcgccatcagctacctgttaaaaaatggctgcaaacttactgacaaagaagaagattcagaaaaatttgctaaacgtcgtcgtcaagttgaaatccaaattcaaaggcttaccgaaaagttaataagtcgcatgcctaaaggtcgagatttgaccaatgctaaatggttagagacactcttgactgctacaaccactgttgctgaagataacgcccaagccaaacgctggcaggatattctgttgactcgatcaagttctctcccattcccccttgtttttgaaaccaacgaggatatggtttggtcaaagaatcaaaagggtaggctgtgtgttcacttcaatggcttgagcgatttaatttttgaggtgtactgcggcaatcgtcaacttcactggtttcaacgcttcctagaagatcaacagactaaacgcaaaagcaaaaatcagcattctagcggcttgttcacactcagaaatggtcatctagtttggcttgaaggtgagggtaaaggggaaccttggaatcttcaccacttgaccctttactgctgtgttgacaatcgcttgtggacagaggagggaacagaaatcgttcgccaagagaaagcagatgaaattactaaattcatcacaaacatgaagaagaaaagcgatctaagcgatacacagcaggctttgattcaacgtaaacaatcaacacttactcgaataaacaattcctttgagcgtcctagccaacccctttatcaaggtcaatcacacattttggttggcgtaagcctgggactagaaaaacctgccacagtagcagtagtagatgcgatcgccaacaaagtcttggcttaccggagtattaaacaattacttggcgacaattacgaactgctaaatcgccagagacgacaacagcagtacctatctcacgaacgccacaaagcacaaaaaaacttctctcccaatcaatttggagcatctgagttagggcaacatatagacagattattagctaaagccattgtagcgttagcgagaacctacaaagctggcagtattgtcttgcccaagttaggggatatgcgggaggttgtccaaagtgaaattcaagctatagcagaacaaaaatttcccggttatattgaaggtcagcaaaaatatgccaaacagtaccgggttaatgttcatcggtggagctacggcagattaattcaaagcattcaaagtaaagcagctcaaacaggaattgtgattgaggagggaaaacaacctattcgaggtagtccccacgacaaagcaaaggaattagcactttctgcttacaatctccgcctaactaggcgaagctaacaaatatctgaaccttgataatagaacattaatagcgccgcaattcatgcttgacagctagctcagtcctaggtataatactagtatattaatagcgccgcaattcatgctgcttgcagcctctgaattttgttaaatgagggttagtttgactgtataaatacagtcttgctttctgaccctggtagctgctcaccctgatgctgctgtcaatagacaggataggtgcgctcccagcaataagggcgcggatgtactgctgtagtggctactgaatcacccccgatcaagggggaaccctccaaaaggtgggttgaaagcggtcttctcgacgagaagacacgtgaatttttttgttcagaacgctcggtcttgcacaccgggcgttttttctttgtgagtccagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttc

SEQ ID NO: 3 - DNA, 4593 bp, synthetic construct
  source 1..4593: mol_type = other DNA
ccgccgcagtctcacgcccggagcgtagcgaccgagtgagctagctatttgtttatttttctaaatacattcaaatatgtatccgctcatgagacaataaccctgataaatgcttcaataatattgaaaaaggaagagtatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgaggggccgcaagatccggccacgatgacccggtcgtcggttcagggcagggtcgttaaatagccgcttatgtctattgctggtttaccggtttattgactaccggaagcagtgtgaccgtgtgcttctcaaatgcctgaggtttcagcaaaaaacccctcaagacccgtttagaggccccaaggggttatgctagttattgctcagcggtggcagcagcctaggcatctccacaaaaggcgtagtgtacagtgacaaattatctgtcgtcggtgacagattaatgtcattgtgactatttaattgtcgtcgtgacccatcagcgttgcttaattaattgatgacaaattaaatgtcatcaatataatatgctctgcaattattatacaaagcaattaaaacaagcggataaaaggacttgctttcaacccacccctaagtttaatagttactgagactctagaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatagtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattatttacctcctgcgacagtcaatttgtcattatgaaaatacacaaaagctttttcctatcttgcaaagcgacagctaatttgtcacaatcacggacaacgacatctattttgtcactgcaaagaggttatgctaaaactgccaaagcgctataatctatactgtataaggattttactgatgacaataatttgtcacaacgacatataattagtcactgtacacgtagagacgtagcaatgctccatggtatatctccttattaaagttaaacaaaattatttctacaggggaattgttatccgctcacaattcccctatagtgagtcgtattaatttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggacatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactaacatgagaattacaacttatatcgtatggggctgacttcaggtgctacatttgaagagataaattgcactgaaatctagagcggttcagtagaaaagatcaaaggatcttcttgagatcctttttttctgcgcgtaatcttttgccctgtaaacgaaaaaaccacctggggaggtggtttgatcgaaggttaagtcagttggggaactgcttaaccgtggtaactggctttcgcagagcacagcaaccaaatctgtccttccagtgtagccggactttggcgcacacttcaagagcaaccgcgtgtttagctaaacaaatcctctgcgaactcccagttaccaatggctgctgccagtggcgttttaccgtgcttttccgggttggactcaagtgaacagttaccggataaggcgcagcagtcgggctgaacggggagttcttgcttacagcccagcttggagcgaacgacctacaccgagccgagataccagtgtgtgagctatgagaaagcgccacacttcccgtaagggagaaaggcggaacaggtatccggtaaacggcagggtcggaacaggagagcgcaagagggagcgacccgccggaaacggtggggatctttaagtcctgtcgggtttcgcccgtactgtcagattcatggttgagcctcacggctcccacagatgcaccggaaaagcgtctgtttatgtgaactctggcaggagggcggagcctatggaaaaacgccaccggcgcggccctgctgttttgcctcacatgttagtcccctgcttatccacggaatctgtgggtaactttgtatgtgtccgcagcgc

SEQ ID NO: 4 - DNA, 8115 bp, synthetic construct
  source 1..8115: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttataagaaggagatatacatatgaacagtcagcaaaatcctgatttagctgttcatcccttggcaattcctatggaaggcttactaggagaaagtgctacaactcttgagaagaatgtaattgccacacaactctcagaggaagcccaagtaaagctagaggtaatccaaagtttactggaaccctgcgatcgcacaacttatgggcaaaagttgcgggaagcagcagagaaactaaatgtatcgttgcgaacggtacaaaggttggtgaaaaactgggaacaagatggcttagtcggcctcactcaaacaagtagggctgataaaggaaaacaccgcattggtgagttttgggaaaacttcattaccaaaacctacaaggagggtaacaagggaagtaaacgtatgacccctaaacaagttgctctcagagtcgaggctaaagcccgtgaattaaaagactctaagccgcccaattacaaaaccgtgttacgggtattagcacccattttggaaaagcaacaaaaagccaagagtatccgcagtcctggctggagaggaactacgctttcggttaaaacccgtgaaggaaaagatttatcggttgattacagtaaccatgtttggcaatgtgaccatacccgcgtggatgtgttgctggtagatcaacatggtgaaattttaagtcgtccctggctaacaacagtaattgatacttactctcgttgcattatgggtatcaacttgggctttgatgcacccagttctggggtagtagcattagcgttacgccatgcaattctaccaaagcgttacggttccgagtacaaactgcattgtgagtggggaacctatggaaaaccagaacatttttatactgatggcggtaaagactttcgctctaaccacttgagtcagattggggcgcagttgggatttgtctgtcatttacgcgatcgcccttctgaaggtggagtagtagaacgtcccttcaaaacattaaatgaccaactattttcaacgcttcctgggtacaccggatctaatgtgcaggaacgcccagaagatgcagagaaggacgcaagacttactttgcgagaactagaacagttacttgtgcgttacatcgtagatcgttacaaccaaagtattgatgcgcggatgggcgaccaaacgcgctttgagcgttgggaagcaggattgcctacagtgccagtaccaataccagaacgagatttggatatttgtttaatgaagcagtcacggcgcactgtgcaaagaggtggttgtttgcagtttcagaatttaatgtatcggggggagtatttggcaggttatgccggagaaactgtcaacttgaggtttgaccccagagacattacaacaattttggtttatcgccaggaaaacaatcaggaagtatttctgactcgcgctcacgctcaaggtttggagacagagcaactggcattagatgaggctgaggcagcaagtcgcagactccgtaccgcagggaaaactatcagtaaccaatcattattgcaagaagttgttgaccgcgatgctcttgtcgctaccaagaaaagccgtaaggagcgtcaaaaattggaacagactgttttgcgatctgctgctgttgatgaaagtaatagagaatccttgccttctcaaatagttgaaccagatgaagtggaatctacagaaacggttcactctcaatacgaggacattgaggtgtgggactatgaacaacttcgtgaagaatatgggttttaaacaatgacagaagctcaggcgatcgccaagcagttgggtggggtaaaaccggatgatgagtggttacaagctgaaattgctcgtctcaagggtaagagcattgtgcctttacagcaggtaaaaactctccatgattggttagatggcaagcgcaaggcaagaaaatcttgccgagtagttggggaatcgagaactggcaagacagttgcttgtgatgcctacagatacaggcacaaacctcagcaggaagctggacgaccgccaactgtgcctgtcgtttatattcgacctcaccaaaaatgtggccccaaggatttgtttaaaaagattactgagtacctcaagtatcgggtaacaaaagggactgtatctgattttcgagataggacgatagaagtactcaagggttgtggcgtagagatgctaattattgatgaagctgaccgtctcaagcctgaaacttttgctgatgtgcgagatattgccgaagatttaggaattgctgtggtactggtaggaacagaccgtttggatgcggtaattaagcgggatgagcaggttctcgaacgctttcgggcgcatcttcgctttggtaaattgtcgggagaggattttaagaacaccgtagaaatgtgggaacaaatggttttgaaactgccagtatcttctaatctaaagagcaaggagatgctacggattctcacgtcagcaactgaaggctacattggtcgccttgatgagattcttagggaagctgcaattcgttccttatcaagaggattgaagaagattgacaaggctgttttacaggaagtagctaaggagtacaaatgatagaagcaccagatgttaaaccttggctattcttgattaaaccctatgaaggggaaagcctgagccactttcttggcaggttcagacgtgccaaccatttatccgcaagtggattgggtactttggcaggaattggtgctatagtggcacgttgggaaagatttcattttaatcctcgccctagtcagcaagaattggaagcgatcgcatctgtagtagaagtggatgctcaaaggttagcccagatgttaccgcctgctggagtgggaatgcagcatgagccaattcgcttgtgtggggcttgttatgccgagtcgccttgtcaccgaattgaatggcagtacaagtcggtgtggaagtgcgatcgccatcaactcaagattttagcaaagtgtccaaactgtcaagcaccttttaaaatgcctgcgctgtgggaggatgggtgctgtcacagatgtaggatgccgtttgcagaaatggcaaagctacagaaggtttgatgataaaaccagaaaaaggtcttaaataaggagatataccatgagtcaaataactattcaagctcgacttatttcctttgaatcaaaccgccaacaactctggaagttgatggcagatttaaacacgccgttaattaacgaactgctttgccagttaggtcaacaccccgacttcgagaagtggcaacaaaagggtaaactcccgtctaccgttgtgagccagttatgtcaacctctcaaaactgaccctcgctttgcaggtcagcccagccgtttatatatgtcggcaattcatattgtggactacatctacaagtcctggctggctatacagaaacggcttcaacagcagctagatggaaagacgcgctggctagaaatgctcaatagcgatgctgaattagtagaacttagtggtgacactttagaggctattcgtgtcaaagctgctgaaattttggcaatagctatgccagcatctgagtcagatagcgcttcacctaaagggaaaaaaggtaaaaaggagaaaaaaccctcatcttctagccctaagcgtagtttatccaagacattatttgacgcttaccaagaaacggaagatattaagagccgtagcgccatcagctacctgttaaaaaatggctgcaaacttactgacaaagaagaagattcagaaaaatttgctaaacgtcgtcgtcaagttgaaatccaaattcaaaggcttaccgaaaagttaataagtcgcatgcctaaaggtcgagatttgaccaatgctaaatggttagagacactcttgactgctacaaccactgttgctgaagataacgcccaagccaaacgctggcaggatattctgttgactcgatcaagttctctcccattcccccttgtttttgaaaccaacgaggatatggtttggtcaaagaatcaaaagggtaggctgtgtgttcacttcaatggcttgagcgatttaatttttgaggtgtactgcggcaatcgtcaacttcactggtttcaacgcttcctagaagatcaacagactaaacgcaaaagcaaaaatcagcattctagcggcttgttcacactcagaaatggtcatctagtttggcttgaaggtgagggtaaaggggaaccttggaatcttcaccacttgaccctttactgctgtgttgacaatcgcttgtggacagaggagggaacagaaatcgttcgccaagagaaagcagatgaaattactaaattcatcacaaacatgaagaagaaaagcgatctaagcgatacacagcaggctttgattcaacgtaaacaatcaacacttactcgaataaacaattcctttgagcgtcctagccaacccctttatcaaggtcaatcacacattttggttggcgtaagcctgggactagaaaaacctgccacagtagcagtagtagatgcgatcgccaacaaagtcttggcttaccggagtattaaacaattacttggcgacaattacgaactgctaaatcgccagagacgacaacagcagtacctatctcacgaacgccacaaagcacaaaaaaacttctctcccaatcaatttggagcatctgagttagggcaacatatagacagattattagctaaagccattgtagcgttagcgagaacctacaaagctggcagtattgtcttgcccaagttaggggatatgcgggaggttgtccaaagtgaaattcaagctatagcagaacaaaaatttcccggttatattgaaggtcagcaaaaatatgccaaacagtaccgggttaatgttcatcggtggagctacggcagattaattcaaagcattcaaagtaaagcagctcaaacaggaattgtgattgaggagggaaaacaacctattcgaggtagtccccacgacaaagcaaaggaattagcactttctgcttacaatctccgcctaactaggcgaagctaacaaatatctgaaccttgataatagaacattaatagcgccgcaattcatgcttgacagctagctcagtcctaggtataatactagtatattaatagcgccgcaattcatgctgcttgcagcctctgaattttgttaaatgagggttagtttgactgtataaatacagtcttgctttctgaccctggtagctgctcaccctgatgctgctgtcaatagacaggataggtgcgctcccagcaataagggcgcggatgtactgctgtagtggctactgaatcacccccgatcaagggggaaccctccaaaaggtgggttgaaagatgccgatcgcgtcacactacgtgtgaatttttttgttcagaacgctcggtcttgcacaccgggcgttttttctttgtgagtccagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 5 - DNA, 8115 bp, synthetic construct
  source 1..8115: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttataagaaggagatatacatatgaacagtcagcaaaatcctgatttagctgttcatcccttggcaattcctatggaaggcttactaggagaaagtgctacaactcttgagaagaatgtaattgccacacaactctcagaggaagcccaagtaaagctagaggtaatccaaagtttactggaaccctgcgatcgcacaacttatgggcaaaagttgcgggaagcagcagagaaactaaatgtatcgttgcgaacggtacaaaggttggtgaaaaactgggaacaagatggcttagtcggcctcactcaaacaagtagggctgataaaggaaaacaccgcattggtgagttttgggaaaacttcattaccaaaacctacaaggagggtaacaagggaagtaaacgtatgacccctaaacaagttgctctcagagtcgaggctaaagcccgtgaattaaaagactctaagccgcccaattacaaaaccgtgttacgggtattagcacccattttggaaaagcaacaaaaagccaagagtatccgcagtcctggctggagaggaactacgctttcggttaaaacccgtgaaggaaaagatttatcggttgattacagtaaccatgtttggcaatgtgaccatacccgcgtggatgtgttgctggtagatcaacatggtgaaattttaagtcgtccctggctaacaacagtaattgatacttactctcgttgcattatgggtatcaacttgggctttgatgcacccagttctggggtagtagcattagcgttacgccatgcaattctaccaaagcgttacggttccgagtacaaactgcattgtgagtggggaacctatggaaaaccagaacatttttatactgatggcggtaaagactttcgctctaaccacttgagtcagattggggcgcagttgggatttgtctgtcatttacgcgatcgcccttctgaaggtggagtagtagaacgtcccttcaaaacattaaatgaccaactattttcaacgcttcctgggtacaccggatctaatgtgcaggaacgcccagaagatgcagagaaggacgcaagacttactttgcgagaactagaacagttacttgtgcgttacatcgtagatcgttacaaccaaagtattgatgcgcggatgggcgaccaaacgcgctttgagcgttgggaagcaggattgcctacagtgccagtaccaataccagaacgagatttggatatttgtttaatgaagcagtcacggcgcactgtgcaaagaggtggttgtttgcagtttcagaatttaatgtatcggggggagtatttggcaggttatgccggagaaactgtcaacttgaggtttgaccccagagacattacaacaattttggtttatcgccaggaaaacaatcaggaagtatttctgactcgcgctcacgctcaaggtttggagacagagcaactggcattagatgaggctgaggcagcaagtcgcagactccgtaccgcagggaaaactatcagtaaccaatcattattgcaagaagttgttgaccgcgatgctcttgtcgctaccaagaaaagccgtaaggagcgtcaaaaattggaacagactgttttgcgatctgctgctgttgatgaaagtaatagagaatccttgccttctcaaatagttgaaccagatgaagtggaatctacagaaacggttcactctcaatacgaggacattgaggtgtgggactatgaacaacttcgtgaagaatatgggttttaaacaatgacagaagctcaggcgatcgccaagcagttgggtggggtaaaaccggatgatgagtggttacaagctgaaattgctcgtctcaagggtaagagcattgtgcctttacagcaggtaaaaactctccatgattggttagatggcaagcgcaaggcaagaaaatcttgccgagtagttggggaatcgagaactggcaagacagttgcttgtgatgcctacagatacaggcacaaacctcagcaggaagctggacgaccgccaactgtgcctgtcgtttatattcgacctcaccaaaaatgtggccccaaggatttgtttaaaaagattactgagtacctcaagtatcgggtaacaaaagggactgtatctgattttcgagataggacgatagaagtactcaagggttgtggcgtagagatgctaattattgatgaagctgaccgtctcaagcctgaaacttttgctgatgtgcgagatattgccgaagatttaggaattgctgtggtactggtaggaacagaccgtttggatgcggtaattaagcgggatgagcaggttctcgaacgctttcgggcgcatcttcgctttggtaaattgtcgggagaggattttaagaacaccgtagaaatgtgggaacaaatggttttgaaactgccagtatcttctaatctaaagagcaaggagatgctacggattctcacgtcagcaactgaaggctacattggtcgccttgatgagattcttagggaagctgcaattcgttccttatcaagaggattgaagaagattgacaaggctgttttacaggaagtagctaaggagtacaaatgatagaagcaccagatgttaaaccttggctattcttgattaaaccctatgaaggggaaagcctgagccactttcttggcaggttcagacgtgccaaccatttatccgcaagtggattgggtactttggcaggaattggtgctatagtggcacgttgggaaagatttcattttaatcctcgccctagtcagcaagaattggaagcgatcgcatctgtagtagaagtggatgctcaaaggttagcccagatgttaccgcctgctggagtgggaatgcagcatgagccaattcgcttgtgtggggcttgttatgccgagtcgccttgtcaccgaattgaatggcagtacaagtcggtgtggaagtgcgatcgccatcaactcaagattttagcaaagtgtccaaactgtcaagcaccttttaaaatgcctgcgctgtgggaggatgggtgctgtcacagatgtaggatgccgtttgcagaaatggcaaagctacagaaggtttgatgataaaaccagaaaaaggtcttaaataaggagatataccatgagtcaaataactattcaagctcgacttatttcctttgaatcaaaccgccaacaactctggaagttgatggcagatttaaacacgccgttaattaacgaactgctttgccagttaggtcaacaccccgacttcgagaagtggcaacaaaagggtaaactcccgtctaccgttgtgagccagttatgtcaacctctcaaaactgaccctcgctttgcaggtcagcccagccgtttatatatgtcggcaattcatattgtggactacatctacaagtcctggctggctatacagaaacggcttcaacagcagctagatggaaagacgcgctggctagaaatgctcaatagcgatgctgaattagtagaacttagtggtgacactttagaggctattcgtgtcaaagctgctgaaattttggcaatagctatgccagcatctgagtcagatagcgcttcacctaaagggaaaaaaggtaaaaaggagaaaaaaccctcatcttctagccctaagcgtagtttatccaagacattatttgacgcttaccaagaaacggaagatattaagagccgtagcgccatcagctacctgttaaaaaatggctgcaaacttactgacaaagaagaagattcagaaaaatttgctaaacgtcgtcgtcaagttgaaatccaaattcaaaggcttaccgaaaagttaataagtcgcatgcctaaaggtcgagatttgaccaatgctaaatggttagagacactcttgactgctacaaccactgttgctgaagataacgcccaagccaaacgctggcaggatattctgttgactcgatcaagttctctcccattcccccttgtttttgaaaccaacgaggatatggtttggtcaaagaatcaaaagggtaggctgtgtgttcacttcaatggcttgagcgatttaatttttgaggtgtactgcggcaatcgtcaacttcactggtttcaacgcttcctagaagatcaacagactaaacgcaaaagcaaaaatcagcattctagcggcttgttcacactcagaaatggtcatctagtttggcttgaaggtgagggtaaaggggaaccttggaatcttcaccacttgaccctttactgctgtgttgacaatcgcttgtggacagaggagggaacagaaatcgttcgccaagagaaagcagatgaaattactaaattcatcacaaacatgaagaagaaaagcgatctaagcgatacacagcaggctttgattcaacgtaaacaatcaacacttactcgaataaacaattcctttgagcgtcctagccaacccctttatcaaggtcaatcacacattttggttggcgtaagcctgggactagaaaaacctgccacagtagcagtagtagatgcgatcgccaacaaagtcttggcttaccggagtattaaacaattacttggcgacaattacgaactgctaaatcgccagagacgacaacagcagtacctatctcacgaacgccacaaagcacaaaaaaacttctctcccaatcaatttggagcatctgagttagggcaacatatagacagattattagctaaagccattgtagcgttagcgagaacctacaaagctggcagtattgtcttgcccaagttaggggatatgcgggaggttgtccaaagtgaaattcaagctatagcagaacaaaaatttcccggttatattgaaggtcagcaaaaatatgccaaacagtaccgggttaatgttcatcggtggagctacggcagattaattcaaagcattcaaagtaaagcagctcaaacaggaattgtgattgaggagggaaaacaacctattcgaggtagtccccacgacaaagcaaaggaattagcactttctgcttacaatctccgcctaactaggcgaagctaacaaatatctgaaccttgataatagaacattaatagcgccgcaattcatgcttgacagctagctcagtcctaggtataatactagtatattaatagcgccgcaattcatgctgcttgcagcctctgaattttgttaaatgagggttagtttgactgtataaatacagtcttgctttctgaccctggtagctgctcaccctgatgctgctgtcaatagacaggataggtgcgctcccagcaataagggcgcggatgtactgctgtagtggctactgaatcacccccgatcaagggggaaccctccaaaaggtgggttgaaaggccactcgctttaatgatgatttgtgaatttttttgttcagaacgctcggtcttgcacaccgggcgttttttctttgtgagtccagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 6 - DNA, 8838 bp, synthetic construct
  source 1..8838: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttataagaaggagatatacatatgaacagtcagcaaaatcctgatttagctgttcatcccttggcaattcctatggaaggcttactaggagaaagtgctacaactcttgagaagaatgtaattgccacacaactctcagaggaagcccaagtaaagctagaggtaatccaaagtttactggaaccctgcgatcgcacaacttatgggcaaaagttgcgggaagcagcagagaaactaaatgtatcgttgcgaacggtacaaaggttggtgaaaaactgggaacaagatggcttagtcggcctcactcaaacaagtagggctgataaaggaaaacaccgcattggtgagttttgggaaaacttcattaccaaaacctacaaggagggtaacaagggaagtaaacgtatgacccctaaacaagttgctctcagagtcgaggctaaagcccgtgaattaaaagactctaagccgcccaattacaaaaccgtgttacgggtattagcacccattttggaaaagcaacaaaaagccaagagtatccgcagtcctggctggagaggaactacgctttcggttaaaacccgtgaaggaaaagatttatcggttgattacagtaaccatgtttggcaatgtgaccatacccgcgtggatgtgttgctggtagatcaacatggtgaaattttaagtcgtccctggctaacaacagtaattgatacttactctcgttgcattatgggtatcaacttgggctttgatgcacccagttctggggtagtagcattagcgttacgccatgcaattctaccaaagcgttacggttccgagtacaaactgcattgtgagtggggaacctatggaaaaccagaacatttttatactgatggcggtaaagactttcgctctaaccacttgagtcagattggggcgcagttgggatttgtctgtcatttacgcgatcgcccttctgaaggtggagtagtagaacgtcccttcaaaacattaaatgaccaactattttcaacgcttcctgggtacaccggatctaatgtgcaggaacgcccagaagatgcagagaaggacgcaagacttactttgcgagaactagaacagttacttgtgcgttacatcgtagatcgttacaaccaaagtattgatgcgcggatgggcgaccaaacgcgctttgagcgttgggaagcaggattgcctacagtgccagtaccaataccagaacgagatttggatatttgtttaatgaagcagtcacggcgcactgtgcaaagaggtggttgtttgcagtttcagaatttaatgtatcggggggagtatttggcaggttatgccggagaaactgtcaacttgaggtttgaccccagagacattacaacaattttggtttatcgccaggaaaacaatcaggaagtatttctgactcgcgctcacgctcaaggtttggagacagagcaactggcattagatgaggctgaggcagcaagtcgcagactccgtaccgcagggaaaactatcagtaaccaatcattattgcaagaagttgttgaccgcgatgctcttgtcgctaccaagaaaagccgtaaggagcgtcaaaaattggaacagactgttttgcgatctgctgctgttgatgaaagtaatagagaatccttgccttctcaaatagttgaaccagatgaagtggaatctacagaaacggttcactctcaatacgaggacattgaggtgtgggactatgaacaacttcgtgaagaatatgggttttaaacaatggtgagcaagggcgaggaggataacatggcctctctcccagcgacacatgagttacacatctttggctccatcaacggtgtggactttgacatggtgggtcagggcaccggcaatccaaatgatggttatgaggagttaaacctgaagtccaccaagggtgacctccagttctccccctggattctggtccctcatatcgggtatggcttccatcagtacctgccctaccctgacgggatgtcgcctttccaggccgccatggtagatggctccggctaccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagcgctaccggagcactgcgcggaccacctacacctttgccaagccaatggcggctaactatctgaagaaccagccgatgtacgtgttccgtaagacggagctcaagcactccaagaccgagctcaacttcaaggagtggcaaaaggcctttaccgatgtgatgggcatggacgagctgtacaagggaggaagcggatcgatgacagaagctcaggcgatcgccaagcagttgggtggggtaaaaccggatgatgagtggttacaagctgaaattgctcgtctcaagggtaagagcattgtgcctttacagcaggtaaaaactctccatgattggttagatggcaagcgcaaggcaagaaaatcttgccgagtagttggggaatcgagaactggcaagacagttgcttgtgatgcctacagatacaggcacaaacctcagcaggaagctggacgaccgccaactgtgcctgtcgtttatattcgacctcaccaaaaatgtggccccaaggatttgtttaaaaagattactgagtacctcaagtatcgggtaacaaaagggactgtatctgattttcgagataggacgatagaagtactcaagggttgtggcgtagagatgctaattattgatgaagctgaccgtctcaagcctgaaacttttgctgatgtgcgagatattgccgaagatttaggaattgctgtggtactggtaggaacagaccgtttggatgcggtaattaagcgggatgagcaggttctcgaacgctttcgggcgcatcttcgctttggtaaattgtcgggagaggattttaagaacaccgtagaaatgtgggaacaaatggttttgaaactgccagtatcttctaatctaaagagcaaggagatgctacggattctcacgtcagcaactgaaggctacattggtcgccttgatgagattcttagggaagctgcaattcgttccttatcaagaggattgaagaagattgacaaggctgttttacaggaagtagctaaggagtacaaatgatagaagcaccagatgttaaaccttggctattcttgattaaaccctatgaaggggaaagcctgagccactttcttggcaggttcagacgtgccaaccatttatccgcaagtggattgggtactttggcaggaattggtgctatagtggcacgttgggaaagatttcattttaatcctcgccctagtcagcaagaattggaagcgatcgcatctgtagtagaagtggatgctcaaaggttagcccagatgttaccgcctgctggagtgggaatgcagcatgagccaattcgcttgtgtggggcttgttatgccgagtcgccttgtcaccgaattgaatggcagtacaagtcggtgtggaagtgcgatcgccatcaactcaagattttagcaaagtgtccaaactgtcaagcaccttttaaaatgcctgcgctgtgggaggatgggtgctgtcacagatgtaggatgccgtttgcagaaatggcaaagctacagaaggtttgatgataaaaccagaaaaaggtcttaaataaggagatataccatgagtcaaataactattcaagctcgacttatttcctttgaatcaaaccgccaacaactctggaagttgatggcagatttaaacacgccgttaattaacgaactgctttgccagttaggtcaacaccccgacttcgagaagtggcaacaaaagggtaaactcccgtctaccgttgtgagccagttatgtcaacctctcaaaactgaccctcgctttgcaggtcagcccagccgtttatatatgtcggcaattcatattgtggactacatctacaagtcctggctggctatacagaaacggcttcaacagcagctagatggaaagacgcgctggctagaaatgctcaatagcgatgctgaattagtagaacttagtggtgacactttagaggctattcgtgtcaaagctgctgaaattttggcaatagctatgccagcatctgagtcagatagcgcttcacctaaagggaaaaaaggtaaaaaggagaaaaaaccctcatcttctagccctaagcgtagtttatccaagacattatttgacgcttaccaagaaacggaagatattaagagccgtagcgccatcagctacctgttaaaaaatggctgcaaacttactgacaaagaagaagattcagaaaaatttgctaaacgtcgtcgtcaagttgaaatccaaattcaaaggcttaccgaaaagttaataagtcgcatgcctaaaggtcgagatttgaccaatgctaaatggttagagacactcttgactgctacaaccactgttgctgaagataacgcccaagccaaacgctggcaggatattctgttgactcgatcaagttctctcccattcccccttgtttttgaaaccaacgaggatatggtttggtcaaagaatcaaaagggtaggctgtgtgttcacttcaatggcttgagcgatttaatttttgaggtgtactgcggcaatcgtcaacttcactggtttcaacgcttcctagaagatcaacagactaaacgcaaaagcaaaaatcagcattctagcggcttgttcacactcagaaatggtcatctagtttggcttgaaggtgagggtaaaggggaaccttggaatcttcaccacttgaccctttactgctgtgttgacaatcgcttgtggacagaggagggaacagaaatcgttcgccaagagaaagcagatgaaattactaaattcatcacaaacatgaagaagaaaagcgatctaagcgatacacagcaggctttgattcaacgtaaacaatcaacacttactcgaataaacaattcctttgagcgtcctagccaacccctttatcaaggtcaatcacacattttggttggcgtaagcctgggactagaaaaacctgccacagtagcagtagtagatgcgatcgccaacaaagtcttggcttaccggagtattaaacaattacttggcgacaattacgaactgctaaatcgccagagacgacaacagcagtacctatctcacgaacgccacaaagcacaaaaaaacttctctcccaatcaatttggagcatctgagttagggcaacatatagacagattattagctaaagccattgtagcgttagcgagaacctacaaagctggcagtattgtcttgcccaagttaggggatatgcgggaggttgtccaaagtgaaattcaagctatagcagaacaaaaatttcccggttatattgaaggtcagcaaaaatatgccaaacagtaccgggttaatgttcatcggtggagctacggcagattaattcaaagcattcaaagtaaagcagctcaaacaggaattgtgattgaggagggaaaacaacctattcgaggtagtccccacgacaaagcaaaggaattagcactttctgcttacaatctccgcctaactaggcgaagctaacaaatatctgaaccttgataatagaacattaatagcgccgcaattcatgcttgacagctagctcagtcctaggtataatactagtatattaatagcgccgcaattcatgctgcttgcagcctctgaattttgttaaatgagggttagtttgactgtataaatacagtcttgctttctgaccctggtagctgctcaccctgatgctgctgtcaatagacaggataggtgcgctcccagcaataagggcgcggatgtactgctgtagtggctactgaatcacccccgatcaagggggaaccctccaaaaggtgggttgaaagatgccgatcgcgtcacactacgtgtgaatttttttgttcagaacgctcggtcttgcacaccgggcgttttttctttgtgagtccagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 7 - DNA, 2713 bp, synthetic construct
  source 1..2713: mol_type = other DNA
gatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttggttagcaagttactaccggattttgtcacacaggaaacagctatgaccatgattacgccaagcttgcatgcctgcaggtcgactctagaggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaa

SEQ ID NO: 8 - DNA, 8115 bp, synthetic construct
  source 1..8115: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttataagaaggagatatacatatgaacagtcagcaaaatcctgatttagctgttcatcccttggcaattcctatggaaggcttactaggagaaagtgctacaactcttgagaagaatgtaattgccacacaactctcagaggaagcccaagtaaagctagaggtaatccaaagtttactggaaccctgcgatcgcacaacttatgggcaaaagttgcgggaagcagcagagaaactaaatgtatcgttgcgaacggtacaaaggttggtgaaaaactgggaacaagatggcttagtcggcctcactcaaacaagtagggctgataaaggaaaacaccgcattggtgagttttgggaaaacttcattaccaaaacctacaaggagggtaacaagggaagtaaacgtatgacccctaaacaagttgctctcagagtcgaggctaaagcccgtgaattaaaagactctaagccgcccaattacaaaaccgtgttacgggtattagcacccattttggaaaagcaacaaaaagccaagagtatccgcagtcctggctggagaggaactacgctttcggttaaaacccgtgaaggaaaagatttatcggttgattacagtaaccatgtttggcaatgtgaccatacccgcgtggatgtgttgctggtagatcaacatggtgaaattttaagtcgtccctggctaacaacagtaattgatacttactctcgttgcattatgggtatcaacttgggctttgatgcacccagttctggggtagtagcattagcgttacgccatgcaattctaccaaagcgttacggttccgagtacaaactgcattgtgagtggggaacctatggaaaaccagaacatttttatactgatggcggtaaagactttcgctctaaccacttgagtcagattggggcgcagttgggatttgtctgtcatttacgcgatcgcccttctgaaggtggagtagtagaacgtcccttcaaaacattaaatgaccaactattttcaacgcttcctgggtacaccggatctaatgtgcaggaacgcccagaagatgcagagaaggacgcaagacttactttgcgagaactagaacagttacttgtgcgttacatcgtagatcgttacaaccaaagtattgatgcgcggatgggcgaccaaacgcgctttgagcgttgggaagcaggattgcctacagtgccagtaccaataccagaacgagatttggatatttgtttaatgaagcagtcacggcgcactgtgcaaagaggtggttgtttgcagtttcagaatttaatgtatcggggggagtatttggcaggttatgccggagaaactgtcaacttgaggtttgaccccagagacattacaacaattttggtttatcgccaggaaaacaatcaggaagtatttctgactcgcgctcacgctcaaggtttggagacagagcaactggcattagatgaggctgaggcagcaagtcgcagactccgtaccgcagggaaaactatcagtaaccaatcattattgcaagaagttgttgaccgcgatgctcttgtcgctaccaagaaaagccgtaaggagcgtcaaaaattggaacagactgttttgcgatctgctgctgttgatgaaagtaatagagaatccttgccttctcaaatagttgaaccagatgaagtggaatctacagaaacggttcactctcaatacgaggacattgaggtgtgggactatgaacaacttcgtgaagaatatgggttttaaacaatgacagaagctcaggcgatcgccaagcagttgggtggggtaaaaccggatgatgagtggttacaagctgaaattgctcgtctcaagggtaagagcattgtgcctttacagcaggtaaaaactctccatgattggttagatggcaagcgcaaggcaagaaaatcttgccgagtagttggggaatcgagaactggcaagacagttgcttgtgatgcctacagatacaggcacaaacctcagcaggaagctggacgaccgccaactgtgcctgtcgtttatattcgacctcaccaaaaatgtggccccaaggatttgtttaaaaagattactgagtacctcaagtatcgggtaacaaaagggactgtatctgattttcgagataggacgatagaagtactcaagggttgtggcgtagagatgctaattattgatgaagctgaccgtctcaagcctgaaacttttgctgatgtgcgagatattgccgaagatttaggaattgctgtggtactggtaggaacagaccgtttggatgcggtaattaagcgggatgagcaggttctcgaagaatttcgggcgcatcttcgctttggtaaattgtcgggagaggattttaagaacaccgtagaaatgtgggaacaaatggttttgaaactgccagtatcttctaatctaaagagcaaggagatgctacggattctcacgtcagcaactgaaggctacattggtcgccttgatgagattcttagggaagctgcaattcgttccttatcaagaggattgaagaagattgacaaggctgttttacaggaagtagctaaggagtacaaatgatagaagcaccagatgttaaaccttggctattcttgattaaaccctatgaaggggaaagcctgagccactttcttggcaggttcagacgtgccaaccatttatccgcaagtggattgggtactttggcaggaattggtgctatagtggcacgttgggaaagatttcattttaatcctcgccctagtcagcaagaattggaagcgatcgcatctgtagtagaagtggatgctcaaaggttagcccagatgttaccgcctgctggagtgggaatgcagcatgagccaattcgcttgtgtggggcttgttatgccgagtcgccttgtcaccgaattgaatggcagtacaagtcggtgtggaagtgcgatcgccatcaactcaagattttagcaaagtgtccaaactgtcaagcaccttttaaaatgcctgcgctgtgggaggatgggtgctgtcacagatgtaggatgccgtttgcagaaatggcaaagctacagaaggtttgatgataaaaccagaaaaaggtcttaaataaggagatataccatgagtcaaataactattcaagctcgacttatttcctttgaatcaaaccgccaacaactctggaagttgatggcagatttaaacacgccgttaattaacgaactgctttgccagttaggtcaacaccccgacttcgagaagtggcaacaaaagggtaaactcccgtctaccgttgtgagccagttatgtcaacctctcaaaactgaccctcgctttgcaggtcagcccagccgtttatatatgtcggcaattcatattgtggactacatctacaagtcctggctggctatacagaaacggcttcaacagcagctagatggaaagacgcgctggctagaaatgctcaatagcgatgctgaattagtagaacttagtggtgacactttagaggctattcgtgtcaaagctgctgaaattttggcaatagctatgccagcatctgagtcagatagcgcttcacctaaagggaaaaaaggtaaaaaggagaaaaaaccctcatcttctagccctaagcgtagtttatccaagacattatttgacgcttaccaagaaacggaagatattaagagccgtagcgccatcagctacctgttaaaaaatggctgcaaacttactgacaaagaagaagattcagaaaaatttgctaaacgtcgtcgtcaagttgaaatccaaattcaaaggcttaccgaaaagttaataagtcgcatgcctaaaggtcgagatttgaccaatgctaaatggttagagacactcttgactgctacaaccactgttgctgaagataacgcccaagccaaacgctggcaggatattctgttgactcgatcaagttctctcccattcccccttgtttttgaaaccaacgaggatatggtttggtcaaagaatcaaaagggtaggctgtgtgttcacttcaatggcttgagcgatttaatttttgaggtgtactgcggcaatcgtcaacttcactggtttcaacgcttcctagaagatcaacagactaaacgcaaaagcaaaaatcagcattctagcggcttgttcacactcagaaatggtcatctagtttggcttgaaggtgagggtaaaggggaaccttggaatcttcaccacttgaccctttactgctgtgttgacaatcgcttgtggacagaggagggaacagaaatcgttcgccaagagaaagcagatgaaattactaaattcatcacaaacatgaagaagaaaagcgatctaagcgatacacagcaggctttgattcaacgtaaacaatcaacacttactcgaataaacaattcctttgagcgtcctagccaacccctttatcaaggtcaatcacacattttggttggcgtaagcctgggactagaaaaacctgccacagtagcagtagtagatgcgatcgccaacaaagtcttggcttaccggagtattaaacaattacttggcgacaattacgaactgctaaatcgccagagacgacaacagcagtacctatctcacgaacgccacaaagcacaaaaaaacttctctcccaatcaatttggagcatctgagttagggcaacatatagacagattattagctaaagccattgtagcgttagcgagaacctacaaagctggcagtattgtcttgcccaagttaggggatatgcgggaggttgtccaaagtgaaattcaagctatagcagaacaaaaatttcccggttatattgaaggtcagcaaaaatatgccaaacagtaccgggttaatgttcatcggtggagctacggcagattaattcaaagcattcaaagtaaagcagctcaaacaggaattgtgattgaggagggaaaacaacctattcgaggtagtccccacgacaaagcaaaggaattagcactttctgcttacaatctccgcctaactaggcgaagctaacaaatatctgaaccttgataatagaacattaatagcgccgcaattcatgcttgacagctagctcagtcctaggtataatactagtatattaatagcgccgcaattcatgctgcttgcagcctctgaattttgttaaatgagggttagtttgactgtataaatacagtcttgctttctgaccctggtagctgctcaccctgatgctgctgtcaatagacaggataggtgcgctcccagcaataagggcgcggatgtactgctgtagtggctactgaatcacccccgatcaagggggaaccctccaaaaggtgggttgaaagatgccgatcgcgtcacactacgtgtgaatttttttgttcagaacgctcggtcttgcacaccgggcgttttttctttgtgagtccagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 9 - DNA, 8115 bp, synthetic construct
  source 1..8115: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttataagaaggagatatacatatgaacagtcagcaaaatcctgatttagctgttcatcccttggcaattcctatggaaggcttactaggagaaagtgctacaactcttgagaagaatgtaattgccacacaactctcagaggaagcccaagtaaagctagaggtaatccaaagtttactggaaccctgcgatcgcacaacttatgggcaaaagttgcgggaagcagcagagaaactaaatgtatcgttgcgaacggtacaaaggttggtgaaaaactgggaacaagatggcttagtcggcctcactcaaacaagtagggctgataaaggaaaacaccgcattggtgagttttgggaaaacttcattaccaaaacctacaaggagggtaacaagggaagtaaacgtatgacccctaaacaagttgctctcagagtcgaggctaaagcccgtgaattaaaagactctaagccgcccaattacaaaaccgtgttacgggtattagcacccattttggaaaagcaacaaaaagccaagagtatccgcagtcctggctggagaggaactacgctttcggttaaaacccgtgaaggaaaagatttatcggttgattacagtaaccatgtttggcaatgtgaccatacccgcgtggatgtgttgctggtagatcaacatggtgaaattttaagtcgtccctggctaacaacagtaattgatacttactctcgttgcattatgggtatcaacttgggctttgatgcacccagttctggggtagtagcattagcgttacgccatgcaattctaccaaagcgttacggttccgagtacaaactgcattgtgagtggggaacctatggaaaaccagaacatttttatactgatggcggtaaagactttcgctctaaccacttgagtcagattggggcgcagttgggatttgtctgtcatttacgcgatcgcccttctgaaggtggagtagtagaacgtcccttcaaaacattaaatgaccaactattttcaacgcttcctgggtacaccggatctaatgtgcaggaacgcccagaagatgcagagaaggacgcaagacttactttgcgagaactagaacagttacttgtgcgttacatcgtagatcgttacaaccaaagtattgatgcgcggatgggcgaccaaacgcgctttgagcgttgggaagcaggattgcctacagtgccagtaccaataccagaacgagatttggatatttgtttaatgaagcagtcacggcgcactgtgcaaagaggtggttgtttgcagtttcagaatttaatgtatcggggggagtatttggcaggttatgccggagaaactgtcaacttgaggtttgaccccagagacattacaacaattttggtttatcgccaggaaaacaatcaggaagtatttctgactcgcgctcacgctcaaggtttggagacagagcaactggcattagatgaggctgaggcagcaagtcgcagactccgtaccgcagggaaaactatcagtaaccaatcattattgcaagaagttgttgaccgcgatgctcttgtcgctaccaagaaaagccgtaaggagcgtcaaaaattggaacagactgttttgcgatctgctgctgttgatgaaagtaatagagaatccttgccttctcaaatagttgaaccagatgaagtggaatctacagaaacggttcactctcaatacgaggacattgaggtgtgggactatgaacaacttcgtgaagaatatgggttttaaacaatgacagaagctcaggcgatcgccaagcagttgggtggggtaaaaccggatgatgagtggttacaagctgaaattgctcgtctcaagggtaagagcattgtgcctttacagcaggtaaaaactctccatgattggttagatggcaagcgcaaggcaagaaaatcttgccgagtagttggggaatcgagaactggcaagacagttgcttgtgatgcctacagatacaggcacaaacctcagcaggaagctggacgaccgccaactgtgcctgtcgtttatattcgacctcaccaaaaatgtggccccgcggatttgtttaaaaagattactgagtacctcaagtatcgggtaacaaaagggactgtatctgattttcgagataggacgatagaagtactcaagggttgtggcgtagagatgctaattattgatgaagctgaccgtctcaagcctgaaacttttgctgatgtgcgagatattgccgaagatttaggaattgctgtggtactggtaggaacagaccgtttggatgcggtaattaagcgggatgagcaggttctcgaacgctttcgggcgcatcttcgctttggtaaattgtcgggagaggattttaagaacaccgtagaaatgtgggaacaaatggttttgaaactgccagtatcttctaatctaaagagcaaggagatgctacggattctcacgtcagcaactgaaggctacattggtcgccttgatgagattcttagggaagctgcaattcgttccttatcaagaggattgaagaagattgacaaggctgttttacaggaagtagctaaggagtacaaatgatagaagcaccagatgttaaaccttggctattcttgattaaaccctatgaaggggaaagcctgagccactttcttggcaggttcagacgtgccaaccatttatccgcaagtggattgggtactttggcaggaattggtgctatagtggcacgttgggaaagatttcattttaatcctcgccctagtcagcaagaattggaagcgatcgcatctgtagtagaagtggatgctcaaaggttagcccagatgttaccgcctgctggagtgggaatgcagcatgagccaattcgcttgtgtggggcttgttatgccgagtcgccttgtcaccgaattgaatggcagtacaagtcggtgtggaagtgcgatcgccatcaactcaagattttagcaaagtgtccaaactgtcaagcaccttttaaaatgcctgcgctgtgggaggatgggtgctgtcacagatgtaggatgccgtttgcagaaatggcaaagctacagaaggtttgatgataaaaccagaaaaaggtcttaaataaggagatataccatgagtcaaataactattcaagctcgacttatttcctttgaatcaaaccgccaacaactctggaagttgatggcagatttaaacacgccgttaattaacgaactgctttgccagttaggtcaacaccccgacttcgagaagtggcaacaaaagggtaaactcccgtctaccgttgtgagccagttatgtcaacctctcaaaactgaccctcgctttgcaggtcagcccagccgtttatatatgtcggcaattcatattgtggactacatctacaagtcctggctggctatacagaaacggcttcaacagcagctagatggaaagacgcgctggctagaaatgctcaatagcgatgctgaattagtagaacttagtggtgacactttagaggctattcgtgtcaaagctgctgaaattttggcaatagctatgccagcatctgagtcagatagcgcttcacctaaagggaaaaaaggtaaaaaggagaaaaaaccctcatcttctagccctaagcgtagtttatccaagacattatttgacgcttaccaagaaacggaagatattaagagccgtagcgccatcagctacctgttaaaaaatggctgcaaacttactgacaaagaagaagattcagaaaaatttgctaaacgtcgtcgtcaagttgaaatccaaattcaaaggcttaccgaaaagttaataagtcgcatgcctaaaggtcgagatttgaccaatgctaaatggttagagacactcttgactgctacaaccactgttgctgaagataacgcccaagccaaacgctggcaggatattctgttgactcgatcaagttctctcccattcccccttgtttttgaaaccaacgaggatatggtttggtcaaagaatcaaaagggtaggctgtgtgttcacttcaatggcttgagcgatttaatttttgaggtgtactgcggcaatcgtcaacttcactggtttcaacgcttcctagaagatcaacagactaaacgcaaaagcaaaaatcagcattctagcggcttgttcacactcagaaatggtcatctagtttggcttgaaggtgagggtaaaggggaaccttggaatcttcaccacttgaccctttactgctgtgttgacaatcgcttgtggacagaggagggaacagaaatcgttcgccaagagaaagcagatgaaattactaaattcatcacaaacatgaagaagaaaagcgatctaagcgatacacagcaggctttgattcaacgtaaacaatcaacacttactcgaataaacaattcctttgagcgtcctagccaacccctttatcaaggtcaatcacacattttggttggcgtaagcctgggactagaaaaacctgccacagtagcagtagtagatgcgatcgccaacaaagtcttggcttaccggagtattaaacaattacttggcgacaattacgaactgctaaatcgccagagacgacaacagcagtacctatctcacgaacgccacaaagcacaaaaaaacttctctcccaatcaatttggagcatctgagttagggcaacatatagacagattattagctaaagccattgtagcgttagcgagaacctacaaagctggcagtattgtcttgcccaagttaggggatatgcgggaggttgtccaaagtgaaattcaagctatagcagaacaaaaatttcccggttatattgaaggtcagcaaaaatatgccaaacagtaccgggttaatgttcatcggtggagctacggcagattaattcaaagcattcaaagtaaagcagctcaaacaggaattgtgattgaggagggaaaacaacctattcgaggtagtccccacgacaaagcaaaggaattagcactttctgcttacaatctccgcctaactaggcgaagctaacaaatatctgaaccttgataatagaacattaatagcgccgcaattcatgcttgacagctagctcagtcctaggtataatactagtatattaatagcgccgcaattcatgctgcttgcagcctctgaattttgttaaatgagggttagtttgactgtataaatacagtcttgctttctgaccctggtagctgctcaccctgatgctgctgtcaatagacaggataggtgcgctcccagcaataagggcgcggatgtactgctgtagtggctactgaatcacccccgatcaagggggaaccctccaaaaggtgggttgaaagatgccgatcgcgtcacactacgtgtgaatttttttgttcagaacgctcggtcttgcacaccgggcgttttttctttgtgagtccagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 10 - DNA, 4182 bp, synthetic construct
  source 1..4182: mol_type = other DNA
ggtatctgcgctccactaaagccagttaccaggttaagcagttccccaactgacttaaccttcgatcaaaccgcctccccaggcggttttttcgtttacagagcaggagattacgacgatcgtaaaaggatctcaagaagatcctttacggattcccgacaccatcactctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactataggggaattgtgagcggataacaattcccctgtagaaataattttgtttaactttaataaggagatatacatgacagaagctcaggcgatcgccaagcagttgggtggggtaaaaccggatgatgagtggttacaagctgaaattgctcgtctcaagggtaagagcattgtgcctttacagcaggtaaaaactctccatgattggttagatggcaagcgcaaggcaagaaaatcttgccgagtagttggggaatcgagaactggcaagacagttgcttgtgatgcctacagatacaggcacaaacctcagcaggaagctggacgaccgccaactgtgcctgtcgtttatattcgacctcaccaaaaatgtggccccaaggatttgtttaaaaagattactgagtacctcaagtatcgggtaacaaaagggactgtatctgattttcgagataggacgatagaagtactcaagggttgtggcgtagagatgctaattattgatgaagctgaccgtctcaagcctgaaacttttgctgatgtgcgagatattgccgaagatttaggaattgctgtggtactggtaggaacagaccgtttggatgcggtaattaagcgggatgagcaggttctcgaacgctttcgggcgcatcttcgctttggtaaattgtcgggagaggattttaagaacaccgtagaaatgtgggaacaaatggttttgaaactgccagtatcttctaatctaaagagcaaggagatgctacggattctcacgtcagcaactgaaggctacattggtcgccttgatgagattcttagggaagctgcaattcgttccttatcaagaggattgaagaagattgacaaggctgttttacaggaagtagctaaggagtacaaatgagctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgacaagctgacgaccgggtctccgcaagtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcggtcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggcatgctagcgcagaaacgtcctagaagatgccaggaggatacttagcagagagacaataaggccggagcgaagccgtttttccataggctccgcccccctgacgaacatcacgaaatctgacgctcaaatcagtggtggcgaaacccgacaggactataaagataccaggcgtttccccctgatggctccctcttgcgctctcctgttcccgtcctgcggcgtccgtgttgtggtggaggctttacccaaatcaccacgtcccgttccgtgtagacagttcgctccaagctgggctgtgtgcaagaaccccccgttcagcccgactgctgcgccttatccggtaactatcatcttgagtccaacccggaaagacacgacaaaacgccactggcagcagccattggtaactgagaattagtggatttagatatcgagagtcttgaagtggtggcctaacagaggctacactgaaaggacagtattt

SEQ ID NO: 11 - DNA, 8206 bp, synthetic construct
  source 1..8206: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttataagaaggagatatacatatgaacagtcagcaaaatcctgatttagctgttcatcccttggcaattcctatggaaggcttactaggagaaagtgctacaactcttgagaagaatgtaattgccacacaactctcagaggaagcccaagtaaagctagaggtaatccaaagtttactggaaccctgcgatcgcacaacttatgggcaaaagttgcgggaagcagcagagaaactaaatgtatcgttgcgaacggtacaaaggttggtgaaaaactgggaacaagatggcttagtcggcctcactcaaacaagtagggctgataaaggaaaacaccgcattggtgagttttgggaaaacttcattaccaaaacctacaaggagggtaacaagggaagtaaacgtatgacccctaaacaagttgctctcagagtcgaggctaaagcccgtgaattaaaagactctaagccgcccaattacaaaaccgtgttacgggtattagcacccattttggaaaagcaacaaaaagccaagagtatccgcagtcctggctggagaggaactacgctttcggttaaaacccgtgaaggaaaagatttatcggttgattacagtaaccatgtttggcaatgtgaccatacccgcgtggatgtgttgctggtagatcaacatggtgaaattttaagtcgtccctggctaacaacagtaattgatacttactctcgttgcattatgggtatcaacttgggctttgatgcacccagttctggggtagtagcattagcgttacgccatgcaattctaccaaagcgttacggttccgagtacaaactgcattgtgagtggggaacctatggaaaaccagaacatttttatactgatggcggtaaagactttcgctctaaccacttgagtcagattggggcgcagttgggatttgtctgtcatttacgcgatcgcccttctgaaggtggagtagtagaacgtcccttcaaaacattaaatgaccaactattttcaacgcttcctgggtacaccggatctaatgtgcaggaacgcccagaagatgcagagaaggacgcaagacttactttgcgagaactagaacagttacttgtgcgttacatcgtagatcgttacaaccaaagtattgatgcgcggatgggcgaccaaacgcgctttgagcgttgggaagcaggattgcctacagtgccagtaccaataccagaacgagatttggatatttgtttaatgaagcagtcacggcgcactgtgcaaagaggtggttgtttgcagtttcagaatttaatgtatcggggggagtatttggcaggttatgccggagaaactgtcaacttgaggtttgaccccagagacattacaacaattttggtttatcgccaggaaaacaatcaggaagtatttctgactcgcgctcacgctcaaggtttggagacagagcaactggcattagatgaggctgaggcagcaagtcgcagactccgtaccgcagggaaaactatcagtaaccaatcattattgcaagaagttgttgaccgcgatgctcttgtcgctaccaagaaaagccgtaaggagcgtcaaaaattggaacagactgttttgcgatctgctgctgttgatgaaagtaatagagaatccttgccttctcaaatagttgaaccagatgaagtggaatctacagaaacggttcactctcaatacgaggacattgaggtgtgggactatgaacaacttcgtgaagaatatgggttttaaacaatgatagaagcaccagatgttaaaccttggctattcttgattaaaccctatgaaggggaaagcctgagccactttcttggcaggttcagacgtgccaaccatttatccgcaagtggattgggtactttggcaggaattggtgctatagtggcacgttgggaaagatttcattttaatcctcgccctagtcagcaagaattggaagcgatcgcatctgtagtagaagtggatgctcaaaggttagcccagatgttaccgcctgctggagtgggaatgcagcatgagccaattcgcttgtgtggggcttgttatgccgagtcgccttgtcaccgaattgaatggcagtacaagtcggtgtggaagtgcgatcgccatcaactcaagattttagcaaagtgtccaaactgtcaagcaccttttaaaatgcctgcgctgtgggaggatgggtgctgtcacagatgtaggatgccgtttgcagaaatggcaaagctacagaaggtttgatgataaaaccagaaaaaggtcttaaataaggagatataccatgagtcaaataactattcaagctcgacttatttcctttgaatcaaaccgccaacaactctggaagttgatggcagatttaaacacgccgttaattaacgaactgctttgccagttaggtcaacaccccgacttcgagaagtggcaacaaaagggtaaactcccgtctaccgttgtgagccagttatgtcaacctctcaaaactgaccctcgctttgcaggtcagcccagccgtttatatatgtcggcaattcatattgtggactacatctacaagtcctggctggctatacagaaacggcttcaacagcagctagatggaaagacgcgctggctagaaatgctcaatagcgatgctgaattagtagaacttagtggtgacactttagaggctattcgtgtcaaagctgctgaaattttggcaatagctatgccagcatctgagtcagatagcgcttcacctaaagggaaaaaaggtaaaaaggagaaaaaaccctcatcttctagccctaagcgtagtttatccaagacattatttgacgcttaccaagaaacggaagatattaagagccgtagcgccatcagctacctgttaaaaaatggctgcaaacttactgacaaagaagaagattcagaaaaatttgctaaacgtcgtcgtcaagttgaaatccaaattcaaaggcttaccgaaaagttaataagtcgcatgcctaaaggtcgagatttgaccaatgctaaatggttagagacactcttgactgctacaaccactgttgctgaagataacgcccaagccaaacgctggcaggatattctgttgactcgatcaagttctctcccattcccccttgtttttgaaaccaacgaggatatggtttggtcaaagaatcaaaagggtaggctgtgtgttcacttcaatggcttgagcgatttaatttttgaggtgtactgcggcaatcgtcaacttcactggtttcaacgcttcctagaagatcaacagactaaacgcaaaagcaaaaatcagcattctagcggcttgttcacactcagaaatggtcatctagtttggcttgaaggtgagggtaaaggggaaccttggaatcttcaccacttgaccctttactgctgtgttgacaatcgcttgtggacagaggagggaacagaaatcgttcgccaagagaaagcagatgaaattactaaattcatcacaaacatgaagaagaaaagcgatctaagcgatacacagcaggctttgattcaacgtaaacaatcaacacttactcgaataaacaattcctttgagcgtcctagccaacccctttatcaaggtcaatcacacattttggttggcgtaagcctgggactagaaaaacctgccacagtagcagtagtagatgcgatcgccaacaaagtcttggcttaccggagtattaaacaattacttggcgacaattacgaactgctaaatcgccagagacgacaacagcagtacctatctcacgaacgccacaaagcacaaaaaaacttctctcccaatcaatttggagcatctgagttagggcaacatatagacagattattagctaaagccattgtagcgttagcgagaacctacaaagctggcagtattgtcttgcccaagttaggggatatgcgggaggttgtccaaagtgaaattcaagctatagcagaacaaaaatttcccggttatattgaaggtcagcaaaaatatgccaaacagtaccgggttaatgttcatcggtggagctacggcagattaattcaaagcattcaaagtaaagcagctcaaacaggaattgtgattgaggagggaaaacaacctattcgaggtagtccccacgacaaagcaaaggaattagcactttctgcttacaatctccgcctaactaggcgaagctccggtagcgaaacgccggggacttctgaatctgctactccggaaagcggaggctctggctcgtcaggaggttcaggaagttcaggcggtatgacagaagctcaggcgatcgccaagcagttgggtggggtaaaaccggatgatgagtggttacaagctgaaattgctcgtctcaagggtaagagcattgtgcctttacagcaggtaaaaactctccatgattggttagatggcaagcgcaaggcaagaaaatcttgccgagtagttggggaatcgagaactggcaagacagttgcttgtgatgcctacagatacaggcacaaacctcagcaggaagctggacgaccgccaactgtgcctgtcgtttatattcgacctcaccaaaaatgtggccccaaggatttgtttaaaaagattactgagtacctcaagtatcgggtaacaaaagggactgtatctgattttcgagataggacgatagaagtactcaagggttgtggcgtagagatgctaattattgatgaagctgaccgtctcaagcctgaaacttttgctgatgtgcgagatattgccgaagatttaggaattgctgtggtactggtaggaacagaccgtttggatgcggtaattaagcgggatgagcaggttctcgaacgctttcgggcgcatcttcgctttggtaaattgtcgggagaggattttaagaacaccgtagaaatgtgggaacaaatggttttgaaactgccagtatcttctaatctaaagagcaaggagatgctacggattctcacgtcagcaactgaaggctacattggtcgccttgatgagattcttagggaagctgcaattcgttccttatcaagaggattgaagaagattgacaaggctgttttacaggaagtagctaaggagtacaaatgacaaatatctgaaccttgataatagaacattaatagcgccgcaattcatgcttgacagctagctcagtcctaggtataatactagtatattaatagcgccgcaattcatgctgcttgcagcctctgaattttgttaaatgagggttagtttgactgtataaatacagtcttgctttctgaccctggtagctgctcaccctgatgctgctgtcaatagacaggataggtgcgctcccagcaataagggcgcggatgtactgctgtagtggctactgaatcacccccgatcaagggggaaccctccaaaaggtgggttgaaagatgccgatcgcgtcacactacgtgtgaatttttttgttcagaacgctcggtcttgcacaccgggcgttttttctttgtgagtccagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 12 - DNA, 5839 bp, synthetic construct
  source 1..5839: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttataagaaggagatatacatatgaacagtcagcaaaatcctgatttagctgttcatcccttggcaattcctatggaaggcttactaggagaaagtgctacaactcttgagaagaatgtaattgccacacaactctcagaggaagcccaagtaaagctagaggtaatccaaagtttactggaaccctgcgatcgcacaacttatgggcaaaagttgcgggaagcagcagagaaactaaatgtatcgttgcgaacggtacaaaggttggtgaaaaactgggaacaagatggcttagtcggcctcactcaaacaagtagggctgataaaggaaaacaccgcattggtgagttttgggaaaacttcattaccaaaacctacaaggagggtaacaagggaagtaaacgtatgacccctaaacaagttgctctcagagtcgaggctaaagcccgtgaattaaaagactctaagccgcccaattacaaaaccgtgttacgggtattagcacccattttggaaaagcaacaaaaagccaagagtatccgcagtcctggctggagaggaactacgctttcggttaaaacccgtgaaggaaaagatttatcggttgattacagtaaccatgtttggcaatgtgaccatacccgcgtggatgtgttgctggtagatcaacatggtgaaattttaagtcgtccctggctaacaacagtaattgatacttactctcgttgcattatgggtatcaacttgggctttgatgcacccagttctggggtagtagcattagcgttacgccatgcaattctaccaaagcgttacggttccgagtacaaactgcattgtgagtggggaacctatggaaaaccagaacatttttatactgatggcggtaaagactttcgctctaaccacttgagtcagattggggcgcagttgggatttgtctgtcatttacgcgatcgcccttctgaaggtggagtagtagaacgtcccttcaaaacattaaatgaccaactattttcaacgcttcctgggtacaccggatctaatgtgcaggaacgcccagaagatgcagagaaggacgcaagacttactttgcgagaactagaacagttacttgtgcgttacatcgtagatcgttacaaccaaagtattgatgcgcggatgggcgaccaaacgcgctttgagcgttgggaagcaggattgcctacagtgccagtaccaataccagaacgagatttggatatttgtttaatgaagcagtcacggcgcactgtgcaaagaggtggttgtttgcagtttcagaatttaatgtatcggggggagtatttggcaggttatgccggagaaactgtcaacttgaggtttgaccccagagacattacaacaattttggtttatcgccaggaaaacaatcaggaagtatttctgactcgcgctcacgctcaaggtttggagacagagcaactggcattagatgaggctgaggcagcaagtcgcagactccgtaccgcagggaaaactatcagtaaccaatcattattgcaagaagttgttgaccgcgatgctcttgtcgctaccaagaaaagccgtaaggagcgtcaaaaattggaacagactgttttgcgatctgctgctgttgatgaaagtaatagagaatccttgccttctcaaatagttgaaccagatgaagtggaatctacagaaacggttcactctcaatacgaggacattgaggtgtgggactatgaacaacttcgtgaagaatatgggttttaaacaatgacagaagctcaggcgatcgccaagcagttgggtggggtaaaaccggatgatgagtggttacaagctgaaattgctcgtctcaagggtaagagcattgtgcctttacagcaggtaaaaactctccatgattggttagatggcaagcgcaaggcaagaaaatcttgccgagtagttggggaatcgagaactggcaagacagttgcttgtgatgcctacagatacaggcacaaacctcagcaggaagctggacgaccgccaactgtgcctgtcgtttatattcgacctcaccaaaaatgtggccccaaggatttgtttaaaaagattactgagtacctcaagtatcgggtaacaaaagggactgtatctgattttcgagataggacgatagaagtactcaagggttgtggcgtagagatgctaattattgatgaagctgaccgtctcaagcctgaaacttttgctgatgtgcgagatattgccgaagatttaggaattgctgtggtactggtaggaacagaccgtttggatgcggtaattaagcgggatgagcaggttctcgaacgctttcgggcgcatcttcgctttggtaaattgtcgggagaggattttaagaacaccgtagaaatgtgggaacaaatggttttgaaactgccagtatcttctaatctaaagagcaaggagatgctacggattctcacgtcagcaactgaaggctacattggtcgccttgatgagattcttagggaagctgcaattcgttccttatcaagaggattgaagaagattgacaaggctgttttacaggaagtagctaaggagtacaaatgatagaagcaccagatgttaaaccttggctattcttgattaaaccctatgaaggggaaagcctgagccactttcttggcaggttcagacgtgccaaccatttatccgcaagtggattgggtactttggcaggaattggtgctatagtggcacgttgggaaagatttcattttaatcctcgccctagtcagcaagaattggaagcgatcgcatctgtagtagaagtggatgctcaaaggttagcccagatgttaccgcctgctggagtgggaatgcagcatgagccaattcgcttgtgtggggcttgttatgccgagtcgccttgtcaccgaattgaatggcagtacaagtcggtgtggaagtgcgatcgccatcaactcaagattttagcaaagtgtccaaactgtcaagcaccttttaaaatgcctgcgctgtgggaggatgggtgctgtcacagatgtaggatgccgtttgcagaaatggcaaagctacagaaggtttgatgataaaaccagaaaaaggtcttaaatgtgaatttttttgttcagaacgctcggtcttgcacaccgggcgttttttctttgtgagtccagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 13 - DNA, 8472 bp, synthetic construct
  source 1..8472: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttataagaaggagatatacatatgaacagtcagcaaaatcctgatttagctgttcatcccttggcaattcctatggaaggcttactaggagaaagtgctacaactcttgagaagaatgtaattgccacacaactctcagaggaagcccaagtaaagctagaggtaatccaaagtttactggaaccctgcgatcgcacaacttatgggcaaaagttgcgggaagcagcagagaaactaaatgtatcgttgcgaacggtacaaaggttggtgaaaaactgggaacaagatggcttagtcggcctcactcaaacaagtagggctgataaaggaaaacaccgcattggtgagttttgggaaaacttcattaccaaaacctacaaggagggtaacaagggaagtaaacgtatgacccctaaacaagttgctctcagagtcgaggctaaagcccgtgaattaaaagactctaagccgcccaattacaaaaccgtgttacgggtattagcacccattttggaaaagcaacaaaaagccaagagtatccgcagtcctggctggagaggaactacgctttcggttaaaacccgtgaaggaaaagatttatcggttgattacagtaaccatgtttggcaatgtgaccatacccgcgtggatgtgttgctggtagatcaacatggtgaaattttaagtcgtccctggctaacaacagtaattgatacttactctcgttgcattatgggtatcaacttgggctttgatgcacccagttctggggtagtagcattagcgttacgccatgcaattctaccaaagcgttacggttccgagtacaaactgcattgtgagtggggaacctatggaaaaccagaacatttttatactgatggcggtaaagactttcgctctaaccacttgagtcagattggggcgcagttgggatttgtctgtcatttacgcgatcgcccttctgaaggtggagtagtagaacgtcccttcaaaacattaaatgaccaactattttcaacgcttcctgggtacaccggatctaatgtgcaggaacgcccagaagatgcagagaaggacgcaagacttactttgcgagaactagaacagttacttgtgcgttacatcgtagatcgttacaaccaaagtattgatgcgcggatgggcgaccaaacgcgctttgagcgttgggaagcaggattgcctacagtgccagtaccaataccagaacgagatttggatatttgtttaatgaagcagtcacggcgcactgtgcaaagaggtggttgtttgcagtttcagaatttaatgtatcggggggagtatttggcaggttatgccggagaaactgtcaacttgaggtttgaccccagagacattacaacaattttggtttatcgccaggaaaacaatcaggaagtatttctgactcgcgctcacgctcaaggtttggagacagagcaactggcattagatgaggctgaggcagcaagtcgcagactccgtaccgcagggaaaactatcagtaaccaatcattattgcaagaagttgttgaccgcgatgctcttgtcgctaccaagaaaagccgtaaggagcgtcaaaaattggaacagactgttttgcgatctgctgctgttgatgaaagtaatagagaatccttgccttctcaaatagttgaaccagatgaagtggaatctacagaaacggttcactctcaatacgaggacattgaggtgtgggactatgaacaacttcgtgaagaatatgggttttaaacaatgacagaagctcaggcgatcgccaagcagttgggtggggtaaaaccggatgatgagtggttacaagctgaaattgctcgtctcaagggtaagagcattgtgcctttacagcaggtaaaaactctccatgattggttagatggcaagcgcaaggcaagaaaatcttgccgagtagttggggaatcgagaactggcaagacagttgcttgtgatgcctacagatacaggcacaaacctcagcaggaagctggacgaccgccaactgtgcctgtcgtttatattcgacctcaccaaaaatgtggccccaaggatttgtttaaaaagattactgagtacctcaagtatcgggtaacaaaagggactgtatctgattttcgagataggacgatagaagtactcaagggttgtggcgtagagatgctaattattgatgaagctgaccgtctcaagcctgaaacttttgctgatgtgcgagatattgccgaagatttaggaattgctgtggtactggtaggaacagaccgtttggatgcggtaattaagcgggatgagcaggttctcgaacgctttcgggcgcatcttcgctttggtaaattgtcgggagaggattttaagaacaccgtagaaatgtgggaacaaatggttttgaaactgccagtatcttctaatctaaagagcaaggagatgctacggattctcacgtcagcaactgaaggctacattggtcgccttgatgagattcttagggaagctgcaattcgttccttatcaagaggattgaagaagattgacaaggctgttttacaggaagtagctaaggagtacaaatgatagaagcaccagatgttaaaccttggctattcttgattaaaccctatgaaggggaaagcctgagccactttcttggcaggttcagacgtgccaaccatttatccgcaagtggattgggtactttggcaggaattggtgctatagtggcacgttgggaaagatttcattttaatcctcgccctagtcagcaagaattggaagcgatcgcatctgtagtagaagtggatgctcaaaggttagcccagatgttaccgcctgctggagtgggaatgcagcatgagccaattcgcttgtgtggggcttgttatgccgagtcgccttgtcaccgaattgaatggcagtacaagtcggtgtggaagtgcgatcgccatcaactcaagattttagcaaagtgtccaaactgtcaagcaccttttaaaatgcctgcgctgtgggaggatgggtgctgtcacagatgtaggatgccgtttgcagaaatggcaaagctacagaaggtttgatgataaaaccagaaaaaggtcttaaataaggagatataccatgagtcaaataactattcaagctcgacttatttcctttgaatcaaaccgccaacaactctggaagttgatggcagatttaaacacgccgttaattaacgaactgctttgccagttaggtcaacaccccgacttcgagaagtggcaacaaaagggtaaactcccgtctaccgttgtgagccagttatgtcaacctctcaaaactgaccctcgctttgcaggtcagcccagccgtttatatatgtcggcaattcatattgtggactacatctacaagtcctggctggctatacagaaacggcttcaacagcagctagatggaaagacgcgctggctagaaatgctcaatagcgatgctgaattagtagaacttagtggtgacactttagaggctattcgtgtcaaagctgctgaaattttggcaatagctatgccagcatctgagtcagatagcgcttcacctaaagggaaaaaaggtaaaaaggagaaaaaaccctcatcttctagccctaagcgtagtttatccaagacattatttgacgcttaccaagaaacggaagatattaagagccgtagcgccatcagctacctgttaaaaaatggctgcaaacttactgacaaagaagaagattcagaaaaatttgctaaacgtcgtcgtcaagttgaaatccaaattcaaaggcttaccgaaaagttaataagtcgcatgcctaaaggtcgagatttgaccaatgctaaatggttagagacactcttgactgctacaaccactgttgctgaagataacgcccaagccaaacgctggcaggatattctgttgactcgatcaagttctctcccattcccccttgtttttgaaaccaacgaggatatggtttggtcaaagaatcaaaagggtaggctgtgtgttcacttcaatggcttgagcgatttaatttttgaggtgtactgcggcaatcgtcaacttcactggtttcaacgcttcctagaagatcaacagactaaacgcaaaagcaaaaatcagcattctagcggcttgttcacactcagaaatggtcatctagtttggcttgaaggtgagggtaaaggggaaccttggaatcttcaccacttgaccctttactgctgtgttgacaatcgcttgtggacagaggagggaacagaaatcgttcgccaagagaaagcagatgaaattactaaattcatcacaaacatgaagaagaaaagcgatctaagcgatacacagcaggctttgattcaacgtaaacaatcaacacttactcgaataaacaattcctttgagcgtcctagccaacccctttatcaaggtcaatcacacattttggttggcgtaagcctgggactagaaaaacctgccacagtagcagtagtagatgcgatcgccaacaaagtcttggcttaccggagtattaaacaattacttggcgacaattacgaactgctaaatcgccagagacgacaacagcagtacctatctcacgaacgccacaaagcacaaaaaaacttctctcccaatcaatttggagcatctgagttagggcaacatatagacagattattagctaaagccattgtagcgttagcgagaacctacaaagctggcagtattgtcttgcccaagttaggggatatgcgggaggttgtccaaagtgaaattcaagctatagcagaacaaaaatttcccggttatattgaaggtcagcaaaaatatgccaaacagtaccgggttaatgttcatcggtggagctacggcagattaattcaaagcattcaaagtaaagcagctcaaacaggaattgtgattgaggagggaaaacaacctattcgaggtagtccccacgacaaagcaaaggaattagcactttctgcttacaatctccgcctaactaggcgaagctccggtagcgaaacgccggggacttctgaatctgctactccggaaagcggaggctctggctcgtcaggaggttcaggaagttcaggctcaatggcacttactcaggagaggaaacaagaaatcatcgtgaactaccaagtgcacgagacggataccggtagcgcagatgttcaggttgccatgctgaccgaacgtattaaccgcttgtctctgcatctgcaggctaataaaaaggaccactccagccgcagaggcctgctcaagctgattggccagcgtaagcgcttattggcgtatatccaaaaagacagccgtgagaaataccaagcgctgatcggccgtcttggtattcgtggttagcaaatatctgaaccttgataatagaacattaatagcgccgcaattcatgcttgacagctagctcagtcctaggtataatactagtatattaatagcgccgcaattcatgctgcttgcagcctctgaattttgttaaatgagggttagtttgactgtataaatacagtcttgctttctgaccctggtagctgctcaccctgatgctgctgtcaatagacaggataggtgcgctcccagcaataagggcgcggatgtactgctgtagtggctactgaatcacccccgatcaagggggaaccctccaaaaggtgggttgaaagatgccgatcgcgtcacactacgtgtgaatttttttgttcagaacgctcggtcttgcacaccgggcgttttttctttgtgagtccagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 14 - DNA, 8472 bp, synthetic construct
  source 1..8472: mol_type = other DNA
aatgtgaccatacccgcgtggatgtgttgctggtagatcaacatggtgaaattttaagtcgtccctggctaacaacagtaattgatacttactctcgttgcattatgggtatcaacttgggctttgatgcacccagttctggggtagtagcattagcgttacgccatgcaattctaccaaagcgttacggttccgagtacaaactgcattgtgagtggggaacctatggaaaaccagaacatttttatactgatggcggtaaagactttcgctctaaccacttgagtcagattggggcgcagttgggatttgtctgtcatttacgcgatcgcccttctgaaggtggagtagtagaacgtcccttcaaaacattaaatgaccaactattttcaacgcttcctgggtacaccggatctaatgtgcaggaacgcccagaagatgcagagaaggacgcaagacttactttgcgagaactagaacagttacttgtgcgttacatcgtagatcgttacaaccaaagtattgatgcgcggatgggcgaccaaacgcgctttgagcgttgggaagcaggattgcctacagtgccagtaccaataccagaacgagatttggatatttgtttaatgaagcagtcacggcgcactgtgcaaagaggtggttgtttgcagtttcagaatttaatgtatcggggggagtatttggcaggttatgccggagaaactgtcaacttgaggtttgaccccagagacattacaacaattttggtttatcgccaggaaaacaatcaggaagtatttctgactcgcgctcacgctcaaggtttggagacagagcaactggcattagatgaggctgaggcagcaagtcgcagactccgtaccgcagggaaaactatcagtaaccaatcattattgcaagaagttgttgaccgcgatgctcttgtcgctaccaagaaaagccgtaaggagcgtcaaaaattggaacagactgttttgcgatctgctgctgttgatgaaagtaatagagaatccttgccttctcaaatagttgaaccagatgaagtggaatctacagaaacggttcactctcaatacgaggacattgaggtgtgggactatgaacaacttcgtgaagaatatgggttttaaacaatgacagaagctcaggcgatcgccaagcagttgggtggggtaaaaccggatgatgagtggttacaagctgaaattgctcgtctcaagggtaagagcattgtgcctttacagcaggtaaaaactctccatgattggttagatggcaagcgcaaggcaagaaaatcttgccgagtagttggggaatcgagaactggcaagacagttgcttgtgatgcctacagatacaggcacaaacctcagcaggaagctggacgaccgccaactgtgcctgtcgtttatattcgacctcaccaaaaatgtggccccaaggatttgtttaaaaagattactgagtacctcaagtatcgggtaacaaaagggactgtatctgattttcgagataggacgatagaagtactcaagggttgtggcgtagagatgctaattattgatgaagctgaccgtctcaagcctgaaacttttgctgatgtgcgagatattgccgaagatttaggaattgctgtggtactggtaggaacagaccgtttggatgcggtaattaagcgggatgagcaggttctcgaacgctttcgggcgcatcttcgctttggtaaattgtcgggagaggattttaagaacaccgtagaaatgtgggaacaaatggttttgaaactgccagtatcttctaatctaaagagcaaggagatgctacggattctcacgtcagcaactgaaggctacattggtcgccttgatgagattcttagggaagctgcaattcgttccttatcaagaggattgaagaagattgacaaggctgttttacaggaagtagctaaggagtacaaatgatagaagcaccagatgttaaaccttggctattcttgattaaaccctatgaaggggaaagcctgagccactttcttggcaggttcagacgtgccaaccatttatccgcaagtggattgggtactttggcaggaattggtgctatagtggcacgttgggaaagatttcattttaatcctcgccctagtcagcaagaattggaagcgatcgcatctgtagtagaagtggatgctcaaaggttagcccagatgttaccgcctgctggagtgggaatgcagcatgagccaattcgcttgtgtggggcttgttatgccgagtcgccttgtcaccgaattgaatggcagtacaagtcggtgtggaagtgcgatcgccatcaactcaagattttagcaaagtgtccaaactgtcaagcaccttttaaaatgcctgcgctgtgggaggatgggtgctgtcacagatgtaggatgccgtttgcagaaatggcaaagctacagaaggtttccggtagcgaaacgccggggacttctgaatctgctactccggaaagcggaggctctggctcgtcaggaggttcaggaagttcaggctcaatggcacttactcaggagaggaaacaagaaatcatcgtgaactaccaagtgcacgagacggataccggtagcgcagatgttcaggttgccatgctgaccgaacgtattaaccgcttgtctctgcatctgcaggctaataaaaaggaccactccagccgcagaggcctgctcaagctgattggccagcgtaagcgcttattggcgtatatccaaaaagacagccgtgagaaataccaagcgctgatcggccgtcttggtattcgtggttagtgataaaaccagaaaaaggtcttaaataaggagatataccatgagtcaaataactattcaagctcgacttatttcctttgaatcaaaccgccaacaactctggaagttgatggcagatttaaacacgccgttaattaacgaactgctttgccagttaggtcaacaccccgacttcgagaagtggcaacaaaagggtaaactcccgtctaccgttgtgagccagttatgtcaacctctcaaaactgaccctcgctttgcaggtcagcccagccgtttatatatgtcggcaattcatattgtggactacatctacaagtcctggctggctatacagaaacggcttcaacagcagctagatggaaagacgcgctggctagaaatgctcaatagcgatgctgaattagtagaacttagtggtgacactttagaggctattcgtgtcaaagctgctgaaattttggcaatagctatgccagcatctgagtcagatagcgcttcacctaaagggaaaaaaggtaaaaaggagaaaaaaccctcatcttctagccctaagcgtagtttatccaagacattatttgacgcttaccaagaaacggaagatattaagagccgtagcgccatcagctacctgttaaaaaatggctgcaaacttactgacaaagaagaagattcagaaaaatttgctaaacgtcgtcgtcaagttgaaatccaaattcaaaggcttaccgaaaagttaataagtcgcatgcctaaaggtcgagatttgaccaatgctaaatggttagagacactcttgactgctacaaccactgttgctgaagataacgcccaagccaaacgctggcaggatattctgttgactcgatcaagttctctcccattcccccttgtttttgaaaccaacgaggatatggtttggtcaaagaatcaaaagggtaggctgtgtgttcacttcaatggcttgagcgatttaatttttgaggtgtactgcggcaatcgtcaacttcactggtttcaacgcttcctagaagatcaacagactaaacgcaaaagcaaaaatcagcattctagcggcttgttcacactcagaaatggtcatctagtttggcttgaaggtgagggtaaaggggaaccttggaatcttcaccacttgaccctttactgctgtgttgacaatcgcttgtggacagaggagggaacagaaatcgttcgccaagagaaagcagatgaaattactaaattcatcacaaacatgaagaagaaaagcgatctaagcgatacacagcaggctttgattcaacgtaaacaatcaacacttactcgaataaacaattcctttgagcgtcctagccaacccctttatcaaggtcaatcacacattttggttggcgtaagcctgggactagaaaaacctgccacagtagcagtagtagatgcgatcgccaacaaagtcttggcttaccggagtattaaacaattacttggcgacaattacgaactgctaaatcgccagagacgacaacagcagtacctatctcacgaacgccacaaagcacaaaaaaacttctctcccaatcaatttggagcatctgagttagggcaacatatagacagattattagctaaagccattgtagcgttagcgagaacctacaaagctggcagtattgtcttgcccaagttaggggatatgcgggaggttgtccaaagtgaaattcaagctatagcagaacaaaaatttcccggttatattgaaggtcagcaaaaatatgccaaacagtaccgggttaatgttcatcggtggagctacggcagattaattcaaagcattcaaagtaaagcagctcaaacaggaattgtgattgaggagggaaaacaacctattcgaggtagtccccacgacaaagcaaaggaattagcactttctgcttacaatctccgcctaactaggcgaagctaacaaatatctgaaccttgataatagaacattaatagcgccgcaattcatgcttgacagctagctcagtcctaggtataatactagtatattaatagcgccgcaattcatgctgcttgcagcctctgaattttgttaaatgagggttagtttgactgtataaatacagtcttgctttctgaccctggtagctgctcaccctgatgctgctgtcaatagacaggataggtgcgctcccagcaataagggcgcggatgtactgctgtagtggctactgaatcacccccgatcaagggggaaccctccaaaaggtgggttgaaagatgccgatcgcgtcacactacgtgtgaatttttttgttcagaacgctcggtcttgcacaccgggcgttttttctttgtgagtccagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttataagaaggagatatacatatgaacagtcagcaaaatcctgatttagctgttcatcccttggcaattcctatggaaggcttactaggagaaagtgctacaactcttgagaagaatgtaattgccacacaactctcagaggaagcccaagtaaagctagaggtaatccaaagtttactggaaccctgcgatcgcacaacttatgggcaaaagttgcgggaagcagcagagaaactaaatgtatcgttgcgaacggtacaaaggttggtgaaaaactgggaacaagatggcttagtcggcctcactcaaacaagtagggctgataaaggaaaacaccgcattggtgagttttgggaaaacttcattaccaaaacctacaaggagggtaacaagggaagtaaacgtatgacccctaaacaagttgctctcagagtcgaggctaaagcccgtgaattaaaagactctaagccgcccaattacaaaaccgtgttacgggtattagcacccattttggaaaagcaacaaaaagccaagagtatccgcagtcctggctggagaggaactacgctttcggttaaaacccgtgaaggaaaagatttatcggttgattacagtaaccatgtttggc

SEQ ID NO: 15 - DNA, 8476 bp, synthetic construct
  source 1..8476: mol_type = other DNA
gagtcaaataactattcaagctcgacttatttcctttgaatcaaaccgccaacaactctggaagttgatggcagatttaaacacgccgttaattaacgaactgctttgccagttaggtcaacaccccgacttcgagaagtggcaacaaaagggtaaactcccgtctaccgttgtgagccagttatgtcaacctctcaaaactgaccctcgctttgcaggtcagcccagccgtttatatatgtcggcaattcatattgtggactacatctacaagtcctggctggctatacagaaacggcttcaacagcagctagatggaaagacgcgctggctagaaatgctcaatagcgatgctgaattagtagaacttagtggtgacactttagaggctattcgtgtcaaagctgctgaaattttggcaatagctatgccagcatctgagtcagatagcgcttcacctaaagggaaaaaaggtaaaaaggagaaaaaaccctcatcttctagccctaagcgtagtttatccaagacattatttgacgcttaccaagaaacggaagatattaagagccgtagcgccatcagctacctgttaaaaaatggctgcaaacttactgacaaagaagaagattcagaaaaatttgctaaacgtcgtcgtcaagttgaaatccaaattcaaaggcttaccgaaaagttaataagtcgcatgcctaaaggtcgagatttgaccaatgctaaatggttagagacactcttgactgctacaaccactgttgctgaagataacgcccaagccaaacgctggcaggatattctgttgactcgatcaagttctctcccattcccccttgtttttgaaaccaacgaggatatggtttggtcaaagaatcaaaagggtaggctgtgtgttcacttcaatggcttgagcgatttaatttttgaggtgtactgcggcaatcgtcaacttcactggtttcaacgcttcctagaagatcaacagactaaacgcaaaagcaaaaatcagcattctagcggcttgttcacactcagaaatggtcatctagtttggcttgaaggtgagggtaaaggggaaccttggaatcttcaccacttgaccctttactgctgtgttgacaatcgcttgtggacagaggagggaacagaaatcgttcgccaagagaaagcagatgaaattactaaattcatcacaaacatgaagaagaaaagcgatctaagcgatacacagcaggctttgattcaacgtaaacaatcaacacttactcgaataaacaattcctttgagcgtcctagccaacccctttatcaaggtcaatcacacattttggttggcgtaagcctgggactagaaaaacctgccacagtagcagtagtagatgcgatcgccaacaaagtcttggcttaccggagtattaaacaattacttggcgacaattacgaactgctaaatcgccagagacgacaacagcagtacctatctcacgaacgccacaaagcacaaaaaaacttctctcccaatcaatttggagcatctgagttagggcaacatatagacagattattagctaaagccattgtagcgttagcgagaacctacaaagctggcagtattgtcttgcccaagttaggggatatgcgggaggttgtccaaagtgaaattcaagctatagcagaacaaaaatttcccggttatattgaaggtcagcaaaaatatgccaaacagtaccgggttaatgttcatcggtggagctacggcagattaattcaaagcattcaaagtaaagcagctcaaacaggaattgtgattgaggagggaaaacaacctattcgaggtagtccccacgacaaagcaaaggaattagcactttctgcttacaatctccgcctaactaggcgaagctaacaaatatctgaaccttgataatagaacattaatagcgccgcaattcatgcttgacagctagctcagtcctaggtataatactagtatattaatagcgccgcaattcatgctgcttgcagcctctgaattttgttaaatgagggttagtttgactgtataaatacagtcttgctttctgaccctggtagctgctcaccctgatgctgctgtcaatagacaggataggtgcgctcccagcaataagggcgcggatgtactgctgtagtggctactgaatcacccccgatcaagggggaaccctccaaaaggtgggttgaaagatgccgatcgcgtcacactacgtgtgaatttttttgttcagaacgctcggtcttgcacaccgggcgttttttctttgtgagtccagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttataagaaggagatatacatatgaacagtcagcaaaatcctgatttagctgttcatcccttggcaattcctatggaaggcttactaggagaaagtgctacaactcttgagaagaatgtaattgccacacaactctcagaggaagcccaagtaaagctagaggtaatccaaagtttactggaaccctgcgatcgcacaacttatgggcaaaagttgcgggaagcagcagagaaactaaatgtatcgttgcgaacggtacaaaggttggtgaaaaactgggaacaagatggcttagtcggcctcactcaaacaagtagggctgataaaggaaaacaccgcattggtgagttttgggaaaacttcattaccaaaacctacaaggagggtaacaagggaagtaaacgtatgacccctaaacaagttgctctcagagtcgaggctaaagcccgtgaattaaaagactctaagccgcccaattacaaaaccgtgttacgggtattagcacccattttggaaaagcaacaaaaagccaagagtatccgcagtcctggctggagaggaactacgctttcggttaaaacccgtgaaggaaaagatttatcggttgattacagtaaccatgtttggcaatgtgaccatacccgcgtggatgtgttgctggtagatcaacatggtgaaattttaagtcgtccctggctaacaacagtaattgatacttactctcgttgcattatgggtatcaacttgggctttgatgcacccagttctggggtagtagcattagcgttacgccatgcaattctaccaaagcgttacggttccgagtacaaactgcattgtgagtggggaacctatggaaaaccagaacatttttatactgatggcggtaaagactttcgctctaaccacttgagtcagattggggcgcagttgggatttgtctgtcatttacgcgatcgcccttctgaaggtggagtagtagaacgtcccttcaaaacattaaatgaccaactattttcaacgcttcctgggtacaccggatctaatgtgcaggaacgcccagaagatgcagagaaggacgcaagacttactttgcgagaactagaacagttacttgtgcgttacatcgtagatcgttacaaccaaagtattgatgcgcggatgggcgaccaaacgcgctttgagcgttgggaagcaggattgcctacagtgccagtaccaataccagaacgagatttggatatttgtttaatgaagcagtcacggcgcactgtgcaaagaggtggttgtttgcagtttcagaatttaatgtatcggggggagtatttggcaggttatgccggagaaactgtcaacttgaggtttgaccccagagacattacaacaattttggtttatcgccaggaaaacaatcaggaagtatttctgactcgcgctcacgctcaaggtttggagacagagcaactggcattagatgaggctgaggcagcaagtcgcagactccgtaccgcagggaaaactatcagtaaccaatcattattgcaagaagttgttgaccgcgatgctcttgtcgctaccaagaaaagccgtaaggagcgtcaaaaattggaacagactgttttgcgatctgctgctgttgatgaaagtaatagagaatccttgccttctcaaatagttgaaccagatgaagtggaatctacagaaacggttcactctcaatacgaggacattgaggtgtgggactatgaacaacttcgtgaagaatatgggttttaaacaatgacagaagctcaggcgatcgccaagcagttgggtggggtaaaaccggatgatgagtggttacaagctgaaattgctcgtctcaagggtaagagcattgtgcctttacagcaggtaaaaactctccatgattggttagatggcaagcgcaaggcaagaaaatcttgccgagtagttggggaatcgagaactggcaagacagttgcttgtgatgcctacagatacaggcacaaacctcagcaggaagctggacgaccgccaactgtgcctgtcgtttatattcgacctcaccaaaaatgtggccccaaggatttgtttaaaaagattactgagtacctcaagtatcgggtaacaaaagggactgtatctgattttcgagataggacgatagaagtactcaagggttgtggcgtagagatgctaattattgatgaagctgaccgtctcaagcctgaaacttttgctgatgtgcgagatattgccgaagatttaggaattgctgtggtactggtaggaacagaccgtttggatgcggtaattaagcgggatgagcaggttctcgaacgctttcgggcgcatcttcgctttggtaaattgtcgggagaggattttaagaacaccgtagaaatgtgggaacaaatggttttgaaactgccagtatcttctaatctaaagagcaaggagatgctacggattctcacgtcagcaactgaaggctacattggtcgccttgatgagattcttagggaagctgcaattcgttccttatcaagaggattgaagaagattgacaaggctgttttacaggaagtagctaaggagtacaaatgaatggcacttactcaggagaggaaacaagaaatcatcgtgaactaccaagtgcacgagacggataccggtagcgcagatgttcaggttgccatgctgaccgaacgtattaaccgcttgtctctgcatctgcaggctaataaaaaggaccactccagccgcagaggcctgctcaagctgattggccagcgtaagcgcttattggcgtatatccaaaaagacagccgtgagaaataccaagcgctgatcggccgtcttggtattcgtggttccggtagcgaaacgccggggacttctgaatctgctactccggaaagcggaggctctggctcgtcaggaggttcaggaagttcaggctcaatgatagaagcaccagatgttaaaccttggctattcttgattaaaccctatgaaggggaaagcctgagccactttcttggcaggttcagacgtgccaaccatttatccgcaagtggattgggtactttggcaggaattggtgctatagtggcacgttgggaaagatttcattttaatcctcgccctagtcagcaagaattggaagcgatcgcatctgtagtagaagtggatgctcaaaggttagcccagatgttaccgcctgctggagtgggaatgcagcatgagccaattcgcttgtgtggggcttgttatgccgagtcgccttgtcaccgaattgaatggcagtacaagtcggtgtggaagtgcgatcgccatcaactcaagattttagcaaagtgtccaaactgtcaagcaccttttaaaatgcctgcgctgtgggaggatgggtgctgtcacagatgtaggatgccgtttgcagaaatggcaaagctacagaaggtttgatgataaaaccagaaaaaggtcttaaataaggagatataccat

SEQ ID NO: 16 - DNA, 8717 bp, synthetic construct
  source 1..8717: mol_type = other DNA
ggtatctgcgctccactaaagccagttaccaggttaagcagttccccaactgacttaaccttcgatcaaaccgcctccccaggcggttttttcgtttacagagcaggagattacgacgatcgtaaaaggatctcaagaagatcctttacggattcccgacaccatcactctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactataggggaattgtgagcggataacaattcccctgtagaaataattttgtttaactttaataaggagatatacatggataagaaatactcaataggcttagctatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatgccattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactccggtagcgaaacgccggggacttctgaatctgctactccggaaagcggaggctctggctcgtcaggaggttcaggaagttcaggcggtatgacagaagctcaggcgatcgccaagcagttgggtggggtaaaaccggatgatgagtggttacaagctgaaattgctcgtctcaagggtaagagcattgtgcctttacagcaggtaaaaactctccatgattggttagatggcaagcgcaaggcaagaaaatcttgccgagtagttggggaatcgagaactggcaagacagttgcttgtgatgcctacagatacaggcacaaacctcagcaggaagctggacgaccgccaactgtgcctgtcgtttatattcgacctcaccaaaaatgtggccccaaggatttgtttaaaaagattactgagtacctcaagtatcgggtaacaaaagggactgtatctgattttcgagataggacgatagaagtactcaagggttgtggcgtagagatgctaattattgatgaagctgaccgtctcaagcctgaaacttttgctgatgtgcgagatattgccgaagatttaggaattgctgtggtactggtaggaacagaccgtttggatgcggtaattaagcgggatgagcaggttctcgaacgctttcgggcgcatcttcgctttggtaaattgtcgggagaggattttaagaacaccgtagaaatgtgggaacaaatggttttgaaactgccagtatcttctaatctaaagagcaaggagatgctacggattctcacgtcagcaactgaaggctacattggtcgccttgatgagattcttagggaagctgcaattcgttccttatcaagaggattgaagaagattgacaaggctgttttacaggaagtagctaaggagtacaaatgatctcgacgctctcccttatgcgactcctgcattaggaaattaatacgactcactatagtgatttcagccgcgctgtacgttttagagctagaaatagcaagttaaaataaggctagtccgttatcaacttgaaaaagtggcaccgagtcggtgctttttttggtagtgcagcgcgatcgtaatcaggatcccatggtacgcgtgctagaggcatcaaataaaacgaaaggctcagtcgaaagactgggcctttcgttttatctgttgtttgtcggtgaacgctctcctgagtaggacaaatggatccgaattcgagctcggcgcgcctgcaggtcgacaaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgacaagctgacgaccgggtctccgcaagtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcggtcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggcatgctagcgcagaaacgtcctagaagatgccaggaggatacttagcagagagacaataaggccggagcgaagccgtttttccataggctccgcccccctgacgaacatcacgaaatctgacgctcaaatcagtggtggcgaaacccgacaggactataaagataccaggcgtttccccctgatggctccctcttgcgctctcctgttcccgtcctgcggcgtccgtgttgtggtggaggctttacccaaatcaccacgtcccgttccgtgtagacagttcgctccaagctgggctgtgtgcaagaaccccccgttcagcccgactgctgcgccttatccggtaactatcatcttgagtccaacccggaaagacacgacaaaacgccactggcagcagccattggtaactgagaattagtggatttagatatcgagagtcttgaagtggtggcctaacagaggctacactgaaaggacagtattt

SEQ ID NO: 17 - DNA, 8717 bp, synthetic construct
  source 1..8717: mol_type = other DNA
ggtatctgcgctccactaaagccagttaccaggttaagcagttccccaactgacttaaccttcgatcaaaccgcctccccaggcggttttttcgtttacagagcaggagattacgacgatcgtaaaaggatctcaagaagatcctttacggattcccgacaccatcactctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactataggggaattgtgagcggataacaattcccctgtagaaataattttgtttaactttaataaggagatatacatgacagaagctcaggcgatcgccaagcagttgggtggggtaaaaccggatgatgagtggttacaagctgaaattgctcgtctcaagggtaagagcattgtgcctttacagcaggtaaaaactctccatgattggttagatggcaagcgcaaggcaagaaaatcttgccgagtagttggggaatcgagaactggcaagacagttgcttgtgatgcctacagatacaggcacaaacctcagcaggaagctggacgaccgccaactgtgcctgtcgtttatattcgacctcaccaaaaatgtggccccaaggatttgtttaaaaagattactgagtacctcaagtatcgggtaacaaaagggactgtatctgattttcgagataggacgatagaagtactcaagggttgtggcgtagagatgctaattattgatgaagctgaccgtctcaagcctgaaacttttgctgatgtgcgagatattgccgaagatttaggaattgctgtggtactggtaggaacagaccgtttggatgcggtaattaagcgggatgagcaggttctcgaacgctttcgggcgcatcttcgctttggtaaattgtcgggagaggattttaagaacaccgtagaaatgtgggaacaaatggttttgaaactgccagtatcttctaatctaaagagcaaggagatgctacggattctcacgtcagcaactgaaggctacattggtcgccttgatgagattcttagggaagctgcaattcgttccttatcaagaggattgaagaagattgacaaggctgttttacaggaagtagctaaggagtacaaatccggtagcgaaacgccggggacttctgaatctgctactccggaaagcggaggctctggctcgtcaggaggttcaggaagttcaggcggtatggataagaaatactcaataggcttagctatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatgccattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactaatctcgacgctctcccttatgcgactcctgcattaggaaattaatacgactcactatagtgatttcagccgcgctgtacgttttagagctagaaatagcaagttaaaataaggctagtccgttatcaacttgaaaaagtggcaccgagtcggtgctttttttggtagtgcagcgcgatcgtaatcaggatcccatggtacgcgtgctagaggcatcaaataaaacgaaaggctcagtcgaaagactgggcctttcgttttatctgttgtttgtcggtgaacgctctcctgagtaggacaaatggatccgaattcgagctcggcgcgcctgcaggtcgacaaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgacaagctgacgaccgggtctccgcaagtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcggtcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggcatgctagcgcagaaacgtcctagaagatgccaggaggatacttagcagagagacaataaggccggagcgaagccgtttttccataggctccgcccccctgacgaacatcacgaaatctgacgctcaaatcagtggtggcgaaacccgacaggactataaagataccaggcgtttccccctgatggctccctcttgcgctctcctgttcccgtcctgcggcgtccgtgttgtggtggaggctttacccaaatcaccacgtcccgttccgtgtagacagttcgctccaagctgggctgtgtgcaagaaccccccgttcagcccgactgctgcgccttatccggtaactatcatcttgagtccaacccggaaagacacgacaaaacgccactggcagcagccattggtaactgagaattagtggatttagatatcgagagtcttgaagtggtggcctaacagaggctacactgaaaggacagtattt

SEQ ID NO: 18 - DNA, 5339 bp, synthetic construct
  source 1..5339: mol_type = other DNA
cccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttataagaaggagatatacatatgaacagtcagcaaaatcctgatttagctgttcatcccttggcaattcctatggaaggcttactaggagaaagtgctacaactcttgagaagaatgtaattgccacacaactctcagaggaagcccaagtaaagctagaggtaatccaaagtttactggaaccctgcgatcgcacaacttatgggcaaaagttgcgggaagcagcagagaaactaaatgtatcgttgcgaacggtacaaaggttggtgaaaaactgggaacaagatggcttagtcggcctcactcaaacaagtagggctgataaaggaaaacaccgcattggtgagttttgggaaaacttcattaccaaaacctacaaggagggtaacaagggaagtaaacgtatgacccctaaacaagttgctctcagagtcgaggctaaagcccgtgaattaaaagactctaagccgcccaattacaaaaccgtgttacgggtattagcacccattttggaaaagcaacaaaaagccaagagtatccgcagtcctggctggagaggaactacgctttcggttaaaacccgtgaaggaaaagatttatcggttgattacagtaaccatgtttggcaatgtgaccatacccgcgtggatgtgttgctggtagatcaacatggtgaaattttaagtcgtccctggctaacaacagtaattgatacttactctcgttgcattatgggtatcaacttgggctttgatgcacccagttctggggtagtagcattagcgttacgccatgcaattctaccaaagcgttacggttccgagtacaaactgcattgtgagtggggaacctatggaaaaccagaacatttttatactgatggcggtaaagactttcgctctaaccacttgagtcagattggggcgcagttgggatttgtctgtcatttacgcgatcgcccttctgaaggtggagtagtagaacgtcccttcaaaacattaaatgaccaactattttcaacgcttcctgggtacaccggatctaatgtgcaggaacgcccagaagatgcagagaaggacgcaagacttactttgcgagaactagaacagttacttgtgcgttacatcgtagatcgttacaaccaaagtattgatgcgcggatgggcgaccaaacgcgctttgagcgttgggaagcaggattgcctacagtgccagtaccaataccagaacgagatttggatatttgtttaatgaagcagtcacggcgcactgtgcaaagaggtggttgtttgcagtttcagaatttaatgtatcggggggagtatttggcaggttatgccggagaaactgtcaacttgaggtttgaccccagagacattacaacaattttggtttatcgccaggaaaacaatcaggaagtatttctgactcgcgctcacgctcaaggtttggagacagagcaactggcattagatgaggctgaggcagcaagtcgcagactccgtaccgcagggaaaactatcagtaaccaatcattattgcaagaagttgttgaccgcgatgctcttgtcgctaccaagaaaagccgtaaggagcgtcaaaaattggaacagactgttttgcgatctgctgctgttgatgaaagtaatagagaatccttgccttctcaaatagttgaaccagatgaagtggaatctacagaaacggttcactctcaatacgaggacattgaggtgtgggactatgaacaacttcgtgaagaatatgggttttaaacaatgacagaagctcaggcgatcgccaagcagttgggtggggtaaaaccggatgatgagtggttacaagctgaaattgctcgtctcaagggtaagagcattgtgcctttacagcaggtaaaaactctccatgattggttagatggcaagcgcaaggcaagaaaatcttgccgagtagttggggaatcgagaactggcaagacagttgcttgtgatgcctacagatacaggcacaaacctcagcaggaagctggacgaccgccaactgtgcctgtcgtttatattcgacctcaccaaaaatgtggccccaaggatttgtttaaaaagattactgagtacctcaagtatcgggtaacaaaagggactgtatctgattttcgagataggacgatagaagtactcaagggttgtggcgtagagatgctaattattgatgaagctgaccgtctcaagcctgaaacttttgctgatgtgcgagatattgccgaagatttaggaattgctgtggtactggtaggaacagaccgtttggatgcggtaattaagcgggatgagcaggttctcgaacgctttcgggcgcatcttcgctttggtaaattgtcgggagaggattttaagaacaccgtagaaatgtgggaacaaatggttttgaaactgccagtatcttctaatctaaagagcaaggagatgctacggattctcacgtcagcaactgaaggctacattggtcgccttgatgagattcttagggaagctgcaattcgttccttatcaagaggattgaagaagattgacaaggctgttttacaggaagtagctaaggagtacaaatgatgataaaaccagaaaaaggtcttaaatgtgaatttttttgttcagaacgctcggtcttgcacaccgggcgttttttctttgtgagtccagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggttt

SEQ ID NO: 19 - DNA, 5687 bp, synthetic construct
  source 1..5687: mol_type = other DNA
ggtatctgcgctccactaaagccagttaccaggttaagcagttccccaactgacttaaccttcgatcaaaccgcctccccaggcggttttttcgtttacagagcaggagattacgacgatcgtaaaaggatctcaagaagatcctttacggattcccgacaccatcactctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattttacactttatgcttccggctcgtatgttgtgtggaattgtgagcggataacaacctgtagaaataattttgtttaactttaataaggagatatacatgagtcaaataactattcaagctcgacttatttcctttgaatcaaaccgccaacaactctggaagttgatggcagatttaaacacgccgttaattaacgaactgctttgccagttaggtcaacaccccgacttcgagaagtggcaacaaaagggtaaactcccgtctaccgttgtgagccagttatgtcaacctctcaaaactgaccctcgctttgcaggtcagcccagccgtttatatatgtcggcaattcatattgtggactacatctacaagtcctggctggctatacagaaacggcttcaacagcagctagatggaaagacgcgctggctagaaatgctcaatagcgatgctgaattagtagaacttagtggtgacactttagaggctattcgtgtcaaagctgctgaaattttggcaatagctatgccagcatctgagtcagatagcgcttcacctaaagggaaaaaaggtaaaaaggagaaaaaaccctcatcttctagccctaagcgtagtttatccaagacattatttgacgcttaccaagaaacggaagatattaagagccgtagcgccatcagctacctgttaaaaaatggctgcaaacttactgacaaagaagaagattcagaaaaatttgctaaacgtcgtcgtcaagttgaaatccaaattcaaaggcttaccgaaaagttaataagtcgcatgcctaaaggtcgagatttgaccaatgctaaatggttagagacactcttgactgctacaaccactgttgctgaagataacgcccaagccaaacgctggcaggatattctgttgactcgatcaagttctctcccattcccccttgtttttgaaaccaacgaggatatggtttggtcaaagaatcaaaagggtaggctgtgtgttcacttcaatggcttgagcgatttaatttttgaggtgtactgcggcaatcgtcaacttcactggtttcaacgcttcctagaagatcaacagactaaacgcaaaagcaaaaatcagcattctagcggcttgttcacactcagaaatggtcatctagtttggcttgaaggtgagggtaaaggggaaccttggaatcttcaccacttgaccctttactgctgtgttgacaatcgcttgtggacagaggagggaacagaaatcgttcgccaagagaaagcagatgaaattactaaattcatcacaaacatgaagaagaaaagcgatctaagcgatacacagcaggctttgattcaacgtaaacaatcaacacttactcgaataaacaattcctttgagcgtcctagccaacccctttatcaaggtcaatcacacattttggttggcgtaagcctgggactagaaaaacctgccacagtagcagtagtagatgcgatcgccaacaaagtcttggcttaccggagtattaaacaattacttggcgacaattacgaactgctaaatcgccagagacgacaacagcagtacctatctcacgaacgccacaaagcacaaaaaaacttctctcccaatcaatttggagcatctgagttagggcaacatatagacagattattagctaaagccattgtagcgttagcgagaacctacaaagctggcagtattgtcttgcccaagttaggggatatgcgggaggttgtccaaagtgaaattcaagctatagcagaacaaaaatttcccggttatattgaaggtcagcaaaaatatgccaaacagtaccgggttaatgttcatcggtggagctacggcagattaattcaaagcattcaaagtaaagcagctcaaacaggaattgtgattgaggagggaaaacaacctattcgaggtagtccccacgacaaagcaaaggaattagcactttctgcttacaatctccgcctaactaggcgaagctaacaaatatctgaaccttgataatagaacattaatagcgccgcaattcatgcttgacagctagctcagtcctaggtataatactagtatattaatagcgccgcaattcatgctgcttgcagcctctgaattttgttaaatgagggttagtttgactgtataaatacagtcttgctttctgaccctggtagctgctcaccctgatgctgctgtcaatagacaggataggtgcgctcccagcaataagggcgcggatgtactgctgtagtggctactgaatcacccccgatcaagggggaaccctccaaaaggtgggttgaaagatgccgatcgcgtcacactacgtgtgaatttttttgttcagaacgctcggtcttgcacaccgggcgttttttctttgtgagtccagctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgacaagctgacgaccgggtctccgcaagtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcggtcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggcatgctagcgcagaaacgtcctagaagatgccaggaggatacttagcagagagacaataaggccggagcgaagccgtttttccataggctccgcccccctgacgaacatcacgaaatctgacgctcaaatcagtggtggcgaaacccgacaggactataaagataccaggcgtttccccctgatggctccctcttgcgctctcctgttcccgtcctgcggcgtccgtgttgtggtggaggctttacccaaatcaccacgtcccgttccgtgtagacagttcgctccaagctgggctgtgtgcaagaaccccccgttcagcccgactgctgcgccttatccggtaactatcatcttgagtccaacccggaaagacacgacaaaacgccactggcagcagccattggtaactgagaattagtggatttagatatcgagagtcttgaagtggtggcctaacagaggctacactgaaaggacagtattt

SEQ ID NO: 20 - DNA, 5687 bp, synthetic construct; identical to SEQ ID NO: 19
  source 1..5687: mol_type = other DNA

SEQ ID NO: 21 - DNA, 5667 bp, synthetic construct
  source 1..5667: mol_type = other DNA
ggtatctgcgctccactaaagccagttaccaggttaagcagttccccaactgacttaaccttcgatcaaaccgcctccccaggcggttttttcgtttacagagcaggagattacgacgatcgtaaaaggatctcaagaagatcctttacggattcccgacaccatcactctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattttatggctagctcagtcctaggtacaatgctagccctgtagaaataattttgtttaactttaataaggagatatacatgagtcaaataactattcaagctcgacttatttcctttgaatcaaaccgccaacaactctggaagttgatggcagatttaaacacgccgttaattaacgaactgctttgccagttaggtcaacaccccgacttcgagaagtggcaacaaaagggtaaactcccgtctaccgttgtgagccagttatgtcaacctctcaaaactgaccctcgctttgcaggtcagcccagccgtttatatatgtcggcaattcatattgtggactacatctacaagtcctggctggctatacagaaacggcttcaacagcagctagatggaaagacgcgctggctagaaatgctcaatagcgatgctgaattagtagaacttagtggtgacactttagaggctattcgtgtcaaagctgctgaaattttggcaatagctatgccagcatctgagtcagatagcgcttcacctaaagggaaaaaaggtaaaaaggagaaaaaaccctcatcttctagccctaagcgtagtttatccaagacattatttgacgcttaccaagaaacggaagatattaagagccgtagcgccatcagctacctgttaaaaaatggctgcaaacttactgacaaagaagaagattcagaaaaatttgctaaacgtcgtcgtcaagttgaaatccaaattcaaaggcttaccgaaaagttaataagtcgcatgcctaaaggtcgagatttgaccaatgctaaatggttagagacactcttgactgctacaaccactgttgctgaagataacgcccaagccaaacgctggcaggatattctgttgactcgatcaagttctctcccattcccccttgtttttgaaaccaacgaggatatggtttggtcaaagaatcaaaagggtaggctgtgtgttcacttcaatggcttgagcgatttaatttttgaggtgtactgcggcaatcgtcaacttcactggtttcaacgcttcctagaagatcaacagactaaacgcaaaagcaaaaatcagcattctagcggcttgttcacactcagaaatggtcatctagtttggcttgaaggtgagggtaaaggggaaccttggaatcttcaccacttgaccctttactgctgtgttgacaatcgcttgtggacagaggagggaacagaaatcgttcgccaagagaaagcagatgaaattactaaattcatcacaaacatgaagaagaaaagcgatctaagcgatacacagcaggctttgattcaacgtaaacaatcaacacttactcgaataaacaattcctttgagcgtcctagccaacccctttatcaaggtcaatcacacattttggttggcgtaagcctgggactagaaaaacctgccacagtagcagtagtagatgcgatcgccaacaaagtcttggcttaccggagtattaaacaattacttggcgacaattacgaactgctaaatcgccagagacgacaacagcagtacctatctcacgaacgccacaaagcacaaaaaaacttctctcccaatcaatttggagcatctgagttagggcaacatatagacagattattagctaaagccattgtagcgttagcgagaacctacaaagctggcagtattgtcttgcccaagttaggggatatgcgggaggttgtccaaagtgaaattcaagctatagcagaacaaaaatttcccggttatattgaaggtcagcaaaaatatgccaaacagtaccgggttaatgttcatcggtggagctacggcagattaattcaaagcattcaaagtaaagcagctcaaacaggaattgtgattgaggagggaaaacaacctattcgaggtagtccccacgacaaagcaaaggaattagcactttctgcttacaatctccgcctaactaggcgaagctaacaaatatctgaaccttgataatagaacattaatagcgccgcaattcatgcttgacagctagctcagtcctaggtataatactagtatattaatagcgccgcaattcatgctgcttgcagcctctgaattttgttaaatgagggttagtttgactgtataaatacagtcttgctttctgaccctggtagctgctcaccctgatgctgctgtcaatagacaggataggtgcgctcccagcaataagggcgcggatgtactgctgtagtggctactgaatcacccccgatcaagggggaaccctccaaaaggtgggttgaaagatgccgatcgcgtcacactacgtgtgaatttttttgttcagaacgctcggtcttgcacaccgggcgttttttctttgtgagtccagctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgacaagctgacgaccgggtctccgcaagtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcggtcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggcatgctagcgcagaaacgtcctagaagatgccaggaggatacttagcagagagacaataaggccggagcgaagccgtttttccataggctccgcccccctgacgaacatcacgaaatctgacgctcaaatcagtggtggcgaaacccgacaggactataaagataccaggcgtttccccctgatggctccctcttgcgctctcctgttcccgtcctgcggcgtccgtgttgtggtggaggctttacccaaatcaccacgtcccgttccgtgtagacagttcgctccaagctgggctgtgtgcaagaaccccccgttcagcccgactgctgcgccttatccggtaactatcatcttgagtccaacccggaaagacacgacaaaacgccactggcagcagccattggtaactgagaattagtggatttagatatcgagagtcttgaagtggtggcctaacagaggctacactgaaaggacagtattt

SEQ ID NO: 22 - DNA, 5667 bp, synthetic construct
  source 1..5667: mol_type = other DNA
ggtatctgcgctccactaaagccagttaccaggttaagcagttccccaactgacttaaccttcgatcaaaccgcctccccaggcggttttttcgtttacagagcaggagattacgacgatcgtaaaaggatctcaagaagatcctttacggattcccgacaccatcactctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattttacggctagctcagtcctaggtactatgctagccctgtagaaataattttgtttaactttaataaggagatatacatgagtcaaataactattcaagctcgacttatttcctttgaatcaaaccgccaacaactctggaagttgatggcagatttaaacacgccgttaattaacgaactgctttgccagttaggtcaacaccccgacttcgagaagtggcaacaaaagggtaaactcccgtctaccgttgtgagccagttatgtcaacctctcaaaactgaccctcgctttgcaggtcagcccagccgtttatatatgtcggcaattcatattgtggactacatctacaagtcctggctggctatacagaaacggcttcaacagcagctagatggaaagacgcgctggctagaaatgctcaatagcgatgctgaattagtagaacttagtggtgacactttagaggctattcgtgtcaaagctgctgaaattttggcaatagctatgccagcatctgagtcagatagcgcttcacctaaagggaaaaaaggtaaaaaggagaaaaaaccctcatcttctagccctaagcgtagtttatccaagacattatttgacgcttaccaagaaacggaagatattaagagccgtagcgccatcagctacctgttaaaaaatggctgcaaacttactgacaaagaagaagattcagaaaaatttgctaaacgtcgtcgtcaagttgaaatccaaattcaaaggcttaccgaaaagttaataagtcgcatgcctaaaggtcgagatttgaccaatgctaaatggttagagacactcttgactgctacaaccactgttgctgaagataacgcccaagccaaacgctggcaggatattctgttgactcgatcaagttctctcccattcccccttgtttttgaaaccaacgaggatatggtttggtcaaagaatcaaaagggtaggctgtgtgttcacttcaatggcttgagcgatttaatttttgaggtgtactgcggcaatcgtcaacttcactggtttcaacgcttcctagaagatcaacagactaaacgcaaaagcaaaaatcagcattctagcggcttgttcacactcagaaatggtcatctagtttggcttgaaggtgagggtaaaggggaaccttggaatcttcaccacttgaccctttactgctgtgttgacaatcgcttgtggacagaggagggaacagaaatcgttcgccaagagaaagcagatgaaattactaaattcatcacaaacatgaagaagaaaagcgatctaagcgatacacagcaggctttgattcaacgtaaacaatcaacacttactcgaataaacaattcctttgagcgtcctagccaacccctttatcaaggtcaatcacacattttggttggcgtaagcctgggactagaaaaacctgccacagtagcagtagtagatgcgatcgccaacaaagtcttggcttaccggagtattaaacaattacttggcgacaattacgaactgctaaatcgccagagacgacaacagcagtacctatctcacgaacgccacaaagcacaaaaaaacttctctcccaatcaatttggagcatctgagttagggcaacatatagacagattattagctaaagccattgtagcgttagcgagaacctacaaagctggcagtattgtcttgcccaagttaggggatatgcgggaggttgtccaaagtgaaattcaagctatagcagaacaaaaatttcccggttatattgaaggtcagcaaaaatatgccaaacagtaccgggttaatgttcatcggtggagctacggcagattaattcaaagcattcaaagtaaagcagctcaaacaggaattgtgattgaggagggaaaacaacctattcgaggtagtccccacgacaaagcaaaggaattagcactttctgcttacaatctccgcctaactaggcgaagctaacaaatatctgaaccttgataatagaacattaatagcgccgcaattcatgcttgacagctagctcagtcctaggtataatactagtatattaatagcgccgcaattcatgctgcttgcagcctctgaattttgttaaatgagggttagtttgactgtataaatacagtcttgctttctgaccctggtagctgctcaccctgatgctgctgtcaatagacaggataggtgcgctcccagcaataagggcgcggatgtactgctgtagtggctactgaatcacccccgatcaagggggaaccctccaaaaggtgggttgaaagatgccgatcgcgtcacactacgtgtgaatttttttgttcagaacgctcggtcttgcacaccgggcgttttttctttgtgagtccagctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgacaagctgacgaccgggtctccgcaagtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcggtcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggcatgctagcgcagaaacgtcctagaagatgccaggaggatacttagcagagagacaataaggccggagcgaagccgtttttccataggctccgcccccctgacgaacatcacgaaatctgacgctcaaatcagtggtggcgaaacccgacaggactataaagataccaggcgtttccccctgatggctccctcttgcgctctcctgttcccgtcctgcggcgtccgtgttgtggtggaggctttacccaaatcaccacgtcccgttccgtgtagacagttcgctccaagctgggctgtgtgcaagaaccccccgttcagcccgactgctgcgccttatccggtaactatcatcttgagtccaacccggaaagacacgacaaaacgccactggcagcagccattggtaactgagaattagtggatttagatatcgagagtcttgaagtggtggcctaacagaggctacactgaaaggacagtattt

SEQ ID NO: 23 - DNA, 5667 bp, synthetic construct
  source 1..5667: mol_type = other DNA
ggtatctgcgctccactaaagccagttaccaggttaagcagttccccaactgacttaaccttcgatcaaaccgcctccccaggcggttttttcgtttacagagcaggagattacgacgatcgtaaaaggatctcaagaagatcctttacggattcccgacaccatcactctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaatttgacagctagctcagtcctaggtataatactagccctgtagaaataattttgtttaactttaataaggagatatacatgagtcaaataactattcaagctcgacttatttcctttgaatcaaaccgccaacaactctggaagttgatggcagatttaaacacgccgttaattaacgaactgctttgccagttaggtcaacaccccgacttcgagaagtggcaacaaaagggtaaactcccgtctaccgttgtgagccagttatgtcaacctctcaaaactgaccctcgctttgcaggtcagcccagccgtttatatatgtcggcaattcatattgtggactacatctacaagtcctggctggctatacagaaacggcttcaacagcagctagatggaaagacgcgctggctagaaatgctcaatagcgatgctgaattagtagaacttagtggtgacactttagaggctattcgtgtcaaagctgctgaaattttggcaatagctatgccagcatctgagtcagatagcgcttcacctaaagggaaaaaaggtaaaaaggagaaaaaaccctcatcttctagccctaagcgtagtttatccaagacattatttgacgcttaccaagaaacggaagatattaagagccgtagcgccatcagctacctgttaaaaaatggctgcaaacttactgacaaagaagaagattcagaaaaatttgctaaacgtcgtcgtcaagttgaaatccaaattcaaaggcttaccgaaaagttaataagtcgcatgcctaaaggtcgagatttgaccaatgctaaatggttagagacactcttgactgctacaaccactgttgctgaagataacgcccaagccaaacgctggcaggatattctgttgactcgatcaagttctctcccattcccccttgtttttgaaaccaacgaggatatggtttggtcaaagaatcaaaagggtaggctgtgtgttcacttcaatggcttgagcgatttaatttttgaggtgtactgcggcaatcgtcaacttcactggtttcaacgcttcctagaagatcaacagactaaacgcaaaagcaaaaatcagcattctagcggcttgttcacactcagaaatggtcatctagtttggcttgaaggtgagggtaaaggggaaccttggaatcttcaccacttgaccctttactgctgtgttgacaatcgcttgtggacagaggagggaacagaaatcgttcgccaagagaaagcagatgaaattactaaattcatcacaaacatgaagaagaaaagcgatctaagcgatacacagcaggctttgattcaacgtaaacaatcaacacttactcgaataaacaattcctttgagcgtcctagccaacccctttatcaaggtcaatcacacattttggttggcgtaagcctgggactagaaaaacctgccacagtagcagtagtagatgcgatcgccaacaaagtcttggcttaccggagtattaaacaattacttggcgacaattacgaactgctaaatcgccagagacgacaacagcagtacctatctcacgaacgccacaaagcacaaaaaaacttctctcccaatcaatttggagcatctgagttagggcaacatatagacagattattagctaaagccattgtagcgttagcgagaacctacaaagctggcagtattgtcttgcccaagttaggggatatgcgggaggttgtccaaagtgaaattcaagctatagcagaacaaaaatttcccggttatattgaaggtcagcaaaaatatgccaaacagtaccgggttaatgttcatcggtggagctacggcagattaattcaaagcattcaaagtaaagcagctcaaacaggaattgtgattgaggagggaaaacaacctattcgaggtagtccccacgacaaagcaaaggaattagcactttctgcttacaatctccgcctaactaggcgaagctaacaaatatctgaaccttgataatagaacattaatagcgccgcaattcatgcttgacagctagctcagtcctaggtataatactagtatattaatagcgccgcaattcatgctgcttgcagcctctgaattttgttaaatgagggttagtttgactgtataaatacagtcttgctttctgaccctggtagctgctcaccctgatgctgctgtcaatagacaggataggtgcgctcccagcaataagggcgcggatgtactgctgtagtggctactgaatcacccccgatcaagggggaaccctccaaaaggtgggttgaaagatgccgatcgcgtcacactacgtgtgaatttttttgttcagaacgctcggtcttgcacaccgggcgttttttctttgtgagtccagctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgacaagctgacgaccgggtctccgcaagtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcggtcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggcatgctagcgcagaaacgtcctagaagatgccaggaggatacttagcagagagacaataaggccggagcgaagccgtttttccataggctccgcccccctgacgaacatcacgaaatctgacgctcaaatcagtggtggcgaaacccgacaggactataaagataccaggcgtttccccctgatggctccctcttgcgctctcctgttcccgtcctgcggcgtccgtgttgtggtggaggctttacccaaatcaccacgtcccgttccgtgtagacagttcgctccaagctgggctgtgtgcaagaaccccccgttcagcccgactgctgcgccttatccggtaactatcatcttgagtccaacccggaaagacacgacaaaacgccactggcagcagccattggtaactgagaattagtggatttagatatcgagagtcttgaagtggtggcctaacagaggctacactgaaaggacagtattt

SEQ ID NO: 24 - DNA, 7288 bp, synthetic construct
  source 1..7288: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttataagaaggagatatacatatgaacagtcagcaaaatcctgatttagctgttcatcccttggcaattcctatggaaggcttactaggagaaagtgctacaactcttgagaagaatgtaattgccacacaactctcagaggaagcccaagtaaagctagaggtaatccaaagtttactggaaccctgcgatcgcacaacttatgggcaaaagttgcgggaagcagcagagaaactaaatgtatcgttgcgaacggtacaaaggttggtgaaaaactgggaacaagatggcttagtcggcctcactcaaacaagtagggctgataaaggaaaacaccgcattggtgagttttgggaaaacttcattaccaaaacctacaaggagggtaacaagggaagtaaacgtatgacccctaaacaagttgctctcagagtcgaggctaaagcccgtgaattaaaagactctaagccgcccaattacaaaaccgtgttacgggtattagcacccattttggaaaagcaacaaaaagccaagagtatccgcagtcctggctggagaggaactacgctttcggttaaaacccgtgaaggaaaagatttatcggttgattacagtaaccatgtttggcaatgtgaccatacccgcgtggatgtgttgctggtagatcaacatggtgaaattttaagtcgtccctggctaacaacagtaattgatacttactctcgttgcattatgggtatcaacttgggctttgatgcacccagttctggggtagtagcattagcgttacgccatgcaattctaccaaagcgttacggttccgagtacaaactgcattgtgagtggggaacctatggaaaaccagaacatttttatactgatggcggtaaagactttcgctctaaccacttgagtcagattggggcgcagttgggatttgtctgtcatttacgcgatcgcccttctgaaggtggagtagtagaacgtcccttcaaaacattaaatgaccaactattttcaacgcttcctgggtacaccggatctaatgtgcaggaacgcccagaagatgcagagaaggacgcaagacttactttgcgagaactagaacagttacttgtgcgttacatcgtagatcgttacaaccaaagtattgatgcgcggatgggcgaccaaacgcgctttgagcgttgggaagcaggattgcctacagtgccagtaccaataccagaacgagatttggatatttgtttaatgaagcagtcacggcgcactgtgcaaagaggtggttgtttgcagtttcagaatttaatgtatcggggggagtatttggcaggttatgccggagaaactgtcaacttgaggtttgaccccagagacattacaacaattttggtttatcgccaggaaaacaatcaggaagtatttctgactcgcgctcacgctcaaggtttggagacagagcaactggcattagatgaggctgaggcagcaagtcgcagactccgtaccgcagggaaaactatcagtaaccaatcattattgcaagaagttgttgaccgcgatgctcttgtcgctaccaagaaaagccgtaaggagcgtcaaaaattggaacagactgttttgcgatctgctgctgttgatgaaagtaatagagaatccttgccttctcaaatagttgaaccagatgaagtggaatctacagaaacggttcactctcaatacgaggacattgaggtgtgggactatgaacaacttcgtgaagaatatgggttttaaacaatgatagaagcaccagatgttaaaccttggctattcttgattaaaccctatgaaggggaaagcctgagccactttcttggcaggttcagacgtgccaaccatttatccgcaagtggattgggtactttggcaggaattggtgctatagtggcacgttgggaaagatttcattttaatcctcgccctagtcagcaagaattggaagcgatcgcatctgtagtagaagtggatgctcaaaggttagcccagatgttaccgcctgctggagtgggaatgcagcatgagccaattcgcttgtgtggggcttgttatgccgagtcgccttgtcaccgaattgaatggcagtacaagtcggtgtggaagtgcgatcgccatcaactcaagattttagcaaagtgtccaaactgtcaagcaccttttaaaatgcctgcgctgtgggaggatgggtgctgtcacagatgtaggatgccgtttgcagaaatggcaaagctacagaaggtttgatgataaaaccagaaaaaggtcttaaataaggagatataccatgagtcaaataactattcaagctcgacttatttcctttgaatcaaaccgccaacaactctggaagttgatggcagatttaaacacgccgttaattaacgaactgctttgccagttaggtcaacaccccgacttcgagaagtggcaacaaaagggtaaactcccgtctaccgttgtgagccagttatgtcaacctctcaaaactgaccctcgctttgcaggtcagcccagccgtttatatatgtcggcaattcatattgtggactacatctacaagtcctggctggctatacagaaacggcttcaacagcagctagatggaaagacgcgctggctagaaatgctcaatagcgatgctgaattagtagaacttagtggtgacactttagaggctattcgtgtcaaagctgctgaaattttggcaatagctatgccagcatctgagtcagatagcgcttcacctaaagggaaaaaaggtaaaaaggagaaaaaaccctcatcttctagccctaagcgtagtttatccaagacattatttgacgcttaccaagaaacggaagatattaagagccgtagcgccatcagctacctgttaaaaaatggctgcaaacttactgacaaagaagaagattcagaaaaatttgctaaacgtcgtcgtcaagttgaaatccaaattcaaaggcttaccgaaaagttaataagtcgcatgcctaaaggtcgagatttgaccaatgctaaatggttagagacactcttgactgctacaaccactgttgctgaagataacgcccaagccaaacgctggcaggatattctgttgactcgatcaagttctctcccattcccccttgtttttgaaaccaacgaggatatggtttggtcaaagaatcaaaagggtaggctgtgtgttcacttcaatggcttgagcgatttaatttttgaggtgtactgcggcaatcgtcaacttcactggtttcaacgcttcctagaagatcaacagactaaacgcaaaagcaaaaatcagcattctagcggcttgttcacactcagaaatggtcatctagtttggcttgaaggtgagggtaaaggggaaccttggaatcttcaccacttgaccctttactgctgtgttgacaatcgcttgtggacagaggagggaacagaaatcgttcgccaagagaaagcagatgaaattactaaattcatcacaaacatgaagaagaaaagcgatctaagcgatacacagcaggctttgattcaacgtaaacaatcaacacttactcgaataaacaattcctttgagcgtcctagccaacccctttatcaaggtcaatcacacattttggttggcgtaagcctgggactagaaaaacctgccacagtagcagtagtagatgcgatcgccaacaaagtcttggcttaccggagtattaaacaattacttggcgacaattacgaactgctaaatcgccagagacgacaacagcagtacctatctcacgaacgccacaaagcacaaaaaaacttctctcccaatcaatttggagcatctgagttagggcaacatatagacagattattagctaaagccattgtagcgttagcgagaacctacaaagctggcagtattgtcttgcccaagttaggggatatgcgggaggttgtccaaagtgaaattcaagctatagcagaacaaaaatttcccggttatattgaaggtcagcaaaaatatgccaaacagtaccgggttaatgttcatcggtggagctacggcagattaattcaaagcattcaaagtaaagcagctcaaacaggaattgtgattgaggagggaaaacaacctattcgaggtagtccccacgacaaagcaaaggaattagcactttctgcttacaatctccgcctaactaggcgaagctaacaaatatctgaaccttgataatagaacattaatagcgccgcaattcatgcttgacagctagctcagtcctaggtataatactagtatattaatagcgccgcaattcatgctgcttgcagcctctgaattttgttaaatgagggttagtttgactgtataaatacagtcttgctttctgaccctggtagctgctcaccctgatgctgctgtcaatagacaggataggtgcgctcccagcaataagggcgcggatgtactgctgtagtggctactgaatcacccccgatcaagggggaaccctccaaaaggtgggttgaaagatgccgatcgcgtcacactacgtgtgaatttttttgttcagaacgctcggtcttgcacaccgggcgttttttctttgtgagtccagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 25 - DNA, 6360 bp, synthetic construct
  source 1..6360: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttataagaaggagatatacatacaatgacagaagctcaggcgatcgccaagcagttgggtggggtaaaaccggatgatgagtggttacaagctgaaattgctcgtctcaagggtaagagcattgtgcctttacagcaggtaaaaactctccatgattggttagatggcaagcgcaaggcaagaaaatcttgccgagtagttggggaatcgagaactggcaagacagttgcttgtgatgcctacagatacaggcacaaacctcagcaggaagctggacgaccgccaactgtgcctgtcgtttatattcgacctcaccaaaaatgtggccccaaggatttgtttaaaaagattactgagtacctcaagtatcgggtaacaaaagggactgtatctgattttcgagataggacgatagaagtactcaagggttgtggcgtagagatgctaattattgatgaagctgaccgtctcaagcctgaaacttttgctgatgtgcgagatattgccgaagatttaggaattgctgtggtactggtaggaacagaccgtttggatgcggtaattaagcgggatgagcaggttctcgaacgctttcgggcgcatcttcgctttggtaaattgtcgggagaggattttaagaacaccgtagaaatgtgggaacaaatggttttgaaactgccagtatcttctaatctaaagagcaaggagatgctacggattctcacgtcagcaactgaaggctacattggtcgccttgatgagattcttagggaagctgcaattcgttccttatcaagaggattgaagaagattgacaaggctgttttacaggaagtagctaaggagtacaaatgatagaagcaccagatgttaaaccttggctattcttgattaaaccctatgaaggggaaagcctgagccactttcttggcaggttcagacgtgccaaccatttatccgcaagtggattgggtactttggcaggaattggtgctatagtggcacgttgggaaagatttcattttaatcctcgccctagtcagcaagaattggaagcgatcgcatctgtagtagaagtggatgctcaaaggttagcccagatgttaccgcctgctggagtgggaatgcagcatgagccaattcgcttgtgtggggcttgttatgccgagtcgccttgtcaccgaattgaatggcagtacaagtcggtgtggaagtgcgatcgccatcaactcaagattttagcaaagtgtccaaactgtcaagcaccttttaaaatgcctgcgctgtgggaggatgggtgctgtcacagatgtaggatgccgtttgcagaaatggcaaagctacagaaggtttgatgataaaaccagaaaaaggtcttaaataaggagatataccatgagtcaaataactattcaagctcgacttatttcctttgaatcaaaccgccaacaactctggaagttgatggcagatttaaacacgccgttaattaacgaactgctttgccagttaggtcaacaccccgacttcgagaagtggcaacaaaagggtaaactcccgtctaccgttgtgagccagttatgtcaacctctcaaaactgaccctcgctttgcaggtcagcccagccgtttatatatgtcggcaattcatattgtggactacatctacaagtcctggctggctatacagaaacggcttcaacagcagctagatggaaagacgcgctggctagaaatgctcaatagcgatgctgaattagtagaacttagtggtgacactttagaggctattcgtgtcaaagctgctgaaattttggcaatagctatgccagcatctgagtcagatagcgcttcacctaaagggaaaaaaggtaaaaaggagaaaaaaccctcatcttctagccctaagcgtagtttatccaagacattatttgacgcttaccaagaaacggaagatattaagagccgtagcgccatcagctacctgttaaaaaatggctgcaaacttactgacaaagaagaagattcagaaaaatttgctaaacgtcgtcgtcaagttgaaatccaaattcaaaggcttaccgaaaagttaataagtcgcatgcctaaaggtcgagatttgaccaatgctaaatggttagagacactcttgactgctacaaccactgttgctgaagataacgcccaagccaaacgctggcaggatattctgttgactcgatcaagttctctcccattcccccttgtttttgaaaccaacgaggatatggtttggtcaaagaatcaaaagggtaggctgtgtgttcacttcaatggcttgagcgatttaatttttgaggtgtactgcggcaatcgtcaacttcactggtttcaacgcttcctagaagatcaacagactaaacgcaaaagcaaaaatcagcattctagcggcttgttcacactcagaaatggtcatctagtttggcttgaaggtgagggtaaaggggaaccttggaatcttcaccacttgaccctttactgctgtgttgacaatcgcttgtggacagaggagggaacagaaatcgttcgccaagagaaagcagatgaaattactaaattcatcacaaacatgaagaagaaaagcgatctaagcgatacacagcaggctttgattcaacgtaaacaatcaacacttactcgaataaacaattcctttgagcgtcctagccaacccctttatcaaggtcaatcacacattttggttggcgtaagcctgggactagaaaaacctgccacagtagcagtagtagatgcgatcgccaacaaagtcttggcttaccggagtattaaacaattacttggcgacaattacgaactgctaaatcgccagagacgacaacagcagtacctatctcacgaacgccacaaagcacaaaaaaacttctctcccaatcaatttggagcatctgagttagggcaacatatagacagattattagctaaagccattgtagcgttagcgagaacctacaaagctggcagtattgtcttgcccaagttaggggatatgcgggaggttgtccaaagtgaaattcaagctatagcagaacaaaaatttcccggttatattgaaggtcagcaaaaatatgccaaacagtaccgggttaatgttcatcggtggagctacggcagattaattcaaagcattcaaagtaaagcagctcaaacaggaattgtgattgaggagggaaaacaacctattcgaggtagtccccacgacaaagcaaaggaattagcactttctgcttacaatctccgcctaactaggcgaagctaacaaatatctgaaccttgataatagaacattaatagcgccgcaattcatgcttgacagctagctcagtcctaggtataatactagtatattaatagcgccgcaattcatgctgcttgcagcctctgaattttgttaaatgagggttagtttgactgtataaatacagtcttgctttctgaccctggtagctgctcaccctgatgctgctgtcaatagacaggataggtgcgctcccagcaataagggcgcggatgtactgctgtagtggctactgaatcacccccgatcaagggggaaccctccaaaaggtgggttgaaagatgccgatcgcgtcacactacgtgtgaatttttttgttcagaacgctcggtcttgcacaccgggcgttttttctttgtgagtccagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 26 - DNA, 2713 bp, synthetic construct
  source 1..2713: mol_type = other DNA
gatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttaaaaaaaaaaaaaaaaaaaaaaaaaaaaaaacagcgctaaaggctgcatttaatattgcgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttggttagcaagttactaccggattttgtcacacaggaaacagctatgaccatgattacgccaagcttgcatgcctgcaggtcgactctagaggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaa

SEQ ID NO: 27 - DNA, 2713 bp, synthetic construct
  source 1..2713: mol_type = other DNA
gatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttaatatatataatataattaaattaatttaaacagcgctaaaggctgcatttaatattgcgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttggttagcaagttactaccggattttgtcacacaggaaacagctatgaccatgattacgccaagcttgcatgcctgcaggtcgactctagaggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaa

SEQ ID NO: 28 - DNA, 2713 bp, synthetic construct
  source 1..2713: mol_type = other DNA
gatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttagccgcgcgcgcgcccgcccggcccgcgccgcagcgctaaaggctgcatttaatattgcgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttggttagcaagttactaccggattttgtcacacaggaaacagctatgaccatgattacgccaagcttgcatgcctgcaggtcgactctagaggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaa

SEQ ID NO: 29 - DNA, 2713 bp, synthetic construct
  source 1..2713: mol_type = other DNA
gatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttaaaaaaaaaaaaaaaaaaaaaaaaaaaaaaatggcgctagagatcatacttaatactgcgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttggttagcaagttactaccggattttgtcacacaggaaacagctatgaccatgattacgccaagcttgcatgcctgcaggtcgactctagaggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaa

SEQ ID NO: 30 - DNA, 2713 bp, synthetic construct
  source 1..2713: mol_type = other DNA
gatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttagccgcgcgcgcgcccgcccggcccgcgccgtggcgctagagatcatacttaatactgcgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttggttagcaagttactaccggattttgtcacacaggaaacagctatgaccatgattacgccaagcttgcatgcctgcaggtcgactctagaggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaa

SEQ ID NO: 31 - DNA, 4447 bp, synthetic construct
  source 1..4447: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctaatacgactcactataggggaattgtgagcggataacaattcccctgtagaaataattttgtttaactttaataaggagatatacatgaacagtcagcaaaatcctgatttagctgttcatcccttggcaattcctatggaaggcttactaggagaaagtgctacaactcttgagaagaatgtaattgccacacaactctcagaggaagcccaagtaaagctagaggtaatccaaagtttactggaaccctgcgatcgcacaacttatgggcaaaagttgcgggaagcagcagagaaactaaatgtatcgttgcgaacggtacaaaggttggtgaaaaactgggaacaagatggcttagtcggcctcactcaaacaagtagggctgataaaggaaaacaccgcattggtgagttttgggaaaacttcattaccaaaacctacaaggagggtaacaagggaagtaaacgtatgacccctaaacaagttgctctcagagtcgaggctaaagcccgtgaattaaaagactctaagccgcccaattacaaaaccgtgttacgggtattagcacccattttggaaaagcaacaaaaagccaagagtatccgcagtcctggctggagaggaactacgctttcggttaaaacccgtgaaggaaaagatttatcggttgattacagtaaccatgtttggcaatgtgaccatacccgcgtggatgtgttgctggtagatcaacatggtgaaattttaagtcgtccctggctaacaacagtaattgatacttactctcgttgcattatgggtatcaacttgggctttgatgcacccagttctggggtagtagcattagcgttacgccatgcaattctaccaaagcgttacggttccgagtacaaactgcattgtgagtggggaacctatggaaaaccagaacatttttatactgatggcggtaaagactttcgctctaaccacttgagtcagattggggcgcagttgggatttgtctgtcatttacgcgatcgcccttctgaaggtggagtagtagaacgtcccttcaaaacattaaatgaccaactattttcaacgcttcctgggtacaccggatctaatgtgcaggaacgcccagaagatgcagagaaggacgcaagacttactttgcgagaactagaacagttacttgtgcgttacatcgtagatcgttacaaccaaagtattgatgcgcggatgggcgaccaaacgcgctttgagcgttgggaagcaggattgcctacagtgccagtaccaataccagaacgagatttggatatttgtttaatgaagcagtcacggcgcactgtgcaaagaggtggttgtttgcagtttcagaatttaatgtatcggggggagtatttggcaggttatgccggagaaactgtcaacttgaggtttgaccccagagacattacaacaattttggtttatcgccaggaaaacaatcaggaagtatttctgactcgcgctcacgctcaaggtttggagacagagcaactggcattagatgaggctgaggcagcaagtcgcagactccgtaccgcagggaaaactatcagtaaccaatcattattgcaagaagttgttgaccgcgatgctcttgtcgctaccaagaaaagccgtaaggagcgtcaaaaattggaacagactgttttgcgatctgctgctgttgatgaaagtaatagagaatccttgccttctcaaatagttgaaccagatgaagtggaatctacagaaacggttcactctcaatacgaggacattgaggtgtgggactatgaacaacttcgtgaagaatatgggttttaagtgaatttttttgttcagaacgctcggtcttgcacaccgggcgttttttctttgtgagtccagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 32 - DNA, 7288 bp, synthetic construct
  source 1..7288: mol_type = other DNA
gtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttataagaaggagatatacatatgaacagtcagcaaaatcctgatttagctgttcatcccttggcaattcctatggaaggcttactaggagaaagtgctacaactcttgagaagaatgtaattgccacacaactctcagaggaagcccaagtaaagctagaggtaatccaaagtttactggaaccctgcgatcgcacaacttatgggcaaaagttgcgggaagcagcagagaaactaaatgtatcgttgcgaacggtacaaaggttggtgaaaaactgggaacaagatggcttagtcggcctcactcaaacaagtagggctgataaaggaaaacaccgcattggtgagttttgggaaaacttcattaccaaaacctacaaggagggtaacaagggaagtaaacgtatgacccctaaacaagttgctctcagagtcgaggctaaagcccgtgaattaaaagactctaagccgcccaattacaaaaccgtgttacgggtattagcacccattttggaaaagcaacaaaaagccaagagtatccgcagtcctggctggagaggaactacgctttcggttaaaacccgtgaaggaaaagatttatcggttgattacagtaaccatgtttggcaatgtgaccatacccgcgtggatgtgttgctggtagatcaacatggtgaaattttaagtcgtccctggctaacaacagtaattgatacttactctcgttgcattatgggtatcaacttgggctttgatgcacccagttctggggtagtagcattagcgttacgccatgcaattctaccaaagcgttacggttccgagtacaaactgcattgtgagtggggaacctatggaaaaccagaacatttttatactgatggcggtaaagactttcgctctaaccacttgagtcagattggggcgcagttgggatttgtctgtcatttacgcgatcgcccttctgaaggtggagtagtagaacgtcccttcaaaacattaaatgaccaactattttcaacgcttcctgggtacaccggatctaatgtgcaggaacgcccagaagatgcagagaaggacgcaagacttactttgcgagaactagaacagttacttgtgcgttacatcgtagatcgttacaaccaaagtattgatgcgcggatgggcgaccaaacgcgctttgagcgttgggaagcaggattgcctacagtgccagtaccaataccagaacgagatttggatatttgtttaatgaagcagtcacggcgcactgtgcaaagaggtggttgtttgcagtttcagaatttaatgtatcggggggagtatttggcaggttatgccggagaaactgtcaacttgaggtttgaccccagagacattacaacaattttggtttatcgccaggaaaacaatcaggaagtatttctgactcgcgctcacgctcaaggtttggagacagagcaactggcattagatgaggctgaggcagcaagtcgcagactccgtaccgcagggaaaactatcagtaaccaatcattattgcaagaagttgttgaccgcgatgctcttgtcgctaccaagaaaagccgtaaggagcgtcaaaaattggaacagactgttttgcgatctgctgctgttgatgaaagtaatagagaatccttgccttctcaaatagttgaaccagatgaagtggaatctacagaaacggttcactctcaatacgaggacattgaggtgtgggactatgaacaacttcgtgaagaatatgggttttaaacaatgatagaagcaccagatgttaaaccttggctattcttgattaaaccctatgaaggggaaagcctgagccactttcttggcaggttcagacgtgccaaccatttatccgcaagtggattgggtactttggcaggaattggtgctatagtggcacgttgggaaagatttcattttaatcctcgccctagtcagcaagaattggaagcgatcgcatctgtagtagaagtggatgctcaaaggttagcccagatgttaccgcctgctggagtgggaatgcagcatgagccaattcgcttgtgtggggcttgttatgccgagtcgccttgtcaccgaattgaatggcagtacaagtcggtgtggaagtgcgatcgccatcaactcaagattttagcaaagtgtccaaactgtcaagcaccttttaaaatgcctgcgctgtgggaggatgggtgctgtcacagatgtaggatgccgtttgcagaaatggcaaagctacagaaggtttgatgataaaaccagaaaaaggtcttaaataaggagatataccatgagtcaaataactattcaagctcgacttatttcctttgaatcaaaccgccaacaactctggaagttgatggcagatttaaacacgccgttaattaacgaactgctttgccagttaggtcaacaccccgacttcgagaagtggcaacaaaagggtaaactcccgtctaccgttgtgagccagttatgtcaacctctcaaaactgaccctcgctttgcaggtcagcccagccgtttatatatgtcggcaattcatattgtggactacatctacaagtcctggctggctatacagaaacggcttcaacagcagctagatggaaagacgcgctggctagaaatgctcaatagcgatgctgaattagtagaacttagtggtgacactttagaggctattcgtgtcaaagctgctgaaattttggcaatagctatgccagcatctgagtcagatagcgcttcacctaaagggaaaaaaggtaaaaaggagaaaaaaccctcatcttctagccctaagcgtagtttatccaagacattatttgacgcttaccaagaaacggaagatattaagagccgtagcgccatcagctacctgttaaaaaatggctgcaaacttactgacaaagaagaagattcagaaaaatttgctaaacgtcgtcgtcaagttgaaatccaaattcaaaggcttaccgaaaagttaataagtcgcatgcctaaaggtcgagatttgaccaatgctaaatggttagagacactcttgactgctacaaccactgttgctgaagataacgcccaagccaaacgctggcaggatattctgttgactcgatcaagttctctcccattcccccttgtttttgaaaccaacgaggatatggtttggtcaaagaatcaaaagggtaggctgtgtgttcacttcaatggcttgagcgatttaatttttgaggtgtactgcggcaatcgtcaacttcactggtttcaacgcttcctagaagatcaacagactaaacgcaaaagcaaaaatcagcattctagcggcttgttcacactcagaaatggtcatctagtttggcttgaaggtgagggtaaaggggaaccttggaatcttcaccacttgaccctttactgctgtgttgacaatcgcttgtggacagaggagggaacagaaatcgttcgccaagagaaagcagatgaaattactaaattcatcacaaacatgaagaagaaaagcgatctaagcgatacacagcaggctttgattcaacgtaaacaatcaacacttactcgaataaacaattcctttgagcgtcctagccaacccctttatcaaggtcaatcacacattttggttggcgtaagcctgggactagaaaaacctgccacagtagcagtagtagatgcgatcgccaacaaagtcttggcttaccggagtattaaacaattacttggcgacaattacgaactgctaaatcgccagagacgacaacagcagtacctatctcacgaacgccacaaagcacaaaaaaacttctctcccaatcaatttggagcatctgagttagggcaacatatagacagattattagctaaagccattgtagcgttagcgagaacctacaaagctggcagtattgtcttgcccaagttaggggatatgcgggaggttgtccaaagtgaaattcaagctatagcagaacaaaaatttcccggttatattgaaggtcagcaaaaatatgccaaacagtaccgggttaatgttcatcggtggagctacggcagattaattcaaagcattcaaagtaaagcagctcaaacaggaattgtgattgaggagggaaaacaacctattcgaggtagtccccacgacaaagcaaaggaattagcactttctgcttacaatctccgcctaactaggcgaagctaacaaatatctgaaccttgataatagaacattaatagcgccgcaattcatgcttgacagctagctcagtcctaggtataatactagtatattaatagcgccgcaattcatgctgcttgcagcctctgaattttgttaaatgagggttagtttgactgtataaatacagtcttgctttctgaccctggtagctgctcaccctgatgctgctgtcaatagacaggataggtgcgctcccagcaataagggcgcggatgtactgctgtagtggctactgaatcacccccgatcaagggggaaccctccaaaaggtgggttgaaagttgtcagatattacgcctgtgtggtgaatttttttgttcagaacgctcggtcttgcacaccgggcgttttttctttgtgagtccagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctca

SEQ ID NO: 33 - DNA, 7288 bp, synthetic construct
  source 1..7288: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttataagaaggagatatacatatgaacagtcagcaaaatcctgatttagctgttcatcccttggcaattcctatggaaggcttactaggagaaagtgctacaactcttgagaagaatgtaattgccacacaactctcagaggaagcccaagtaaagctagaggtaatccaaagtttactggaaccctgcgatcgcacaacttatgggcaaaagttgcgggaagcagcagagaaactaaatgtatcgttgcgaacggtacaaaggttggtgaaaaactgggaacaagatggcttagtcggcctcactcaaacaagtagggctgataaaggaaaacaccgcattggtgagttttgggaaaacttcattaccaaaacctacaaggagggtaacaagggaagtaaacgtatgacccctaaacaagttgctctcagagtcgaggctaaagcccgtgaattaaaagactctaagccgcccaattacaaaaccgtgttacgggtattagcacccattttggaaaagcaacaaaaagccaagagtatccgcagtcctggctggagaggaactacgctttcggttaaaacccgtgaaggaaaagatttatcggttgattacagtaaccatgtttggcaatgtgaccatacccgcgtggatgtgttgctggtagatcaacatggtgaaattttaagtcgtccctggctaacaacagtaattgatacttactctcgttgcattatgggtatcaacttgggctttgatgcacccagttctggggtagtagcattagcgttacgccatgcaattctaccaaagcgttacggttccgagtacaaactgcattgtgagtggggaacctatggaaaaccagaacatttttatactgatggcggtaaagactttcgctctaaccacttgagtcagattggggcgcagttgggatttgtctgtcatttacgcgatcgcccttctgaaggtggagtagtagaacgtcccttcaaaacattaaatgaccaactattttcaacgcttcctgggtacaccggatctaatgtgcaggaacgcccagaagatgcagagaaggacgcaagacttactttgcgagaactagaacagttacttgtgcgttacatcgtagatcgttacaaccaaagtattgatgcgcggatgggcgaccaaacgcgctttgagcgttgggaagcaggattgcctacagtgccagtaccaataccagaacgagatttggatatttgtttaatgaagcagtcacggcgcactgtgcaaagaggtggttgtttgcagtttcagaatttaatgtatcggggggagtatttggcaggttatgccggagaaactgtcaacttgaggtttgaccccagagacattacaacaattttggtttatcgccaggaaaacaatcaggaagtatttctgactcgcgctcacgctcaaggtttggagacagagcaactggcattagatgaggctgaggcagcaagtcgcagactccgtaccgcagggaaaactatcagtaaccaatcattattgcaagaagttgttgaccgcgatgctcttgtcgctaccaagaaaagccgtaaggagcgtcaaaaattggaacagactgttttgcgatctgctgctgttgatgaaagtaatagagaatccttgccttctcaaatagttgaaccagatgaagtggaatctacagaaacggttcactctcaatacgaggacattgaggtgtgggactatgaacaacttcgtgaagaatatgggttttaaacaatgatagaagcaccagatgttaaaccttggctattcttgattaaaccctatgaaggggaaagcctgagccactttcttggcaggttcagacgtgccaaccatttatccgcaagtggattgggtactttggcaggaattggtgctatagtggcacgttgggaaagatttcattttaatcctcgccctagtcagcaagaattggaagcgatcgcatctgtagtagaagtggatgctcaaaggttagcccagatgttaccgcctgctggagtgggaatgcagcatgagccaattcgcttgtgtggggcttgttatgccgagtcgccttgtcaccgaattgaatggcagtacaagtcggtgtggaagtgcgatcgccatcaactcaagattttagcaaagtgtccaaactgtcaagcaccttttaaaatgcctgcgctgtgggaggatgggtgctgtcacagatgtaggatgccgtttgcagaaatggcaaagctacagaaggtttgatgataaaaccagaaaaaggtcttaaataaggagatataccatgagtcaaataactattcaagctcgacttatttcctttgaatcaaaccgccaacaactctggaagttgatggcagatttaaacacgccgttaattaacgaactgctttgccagttaggtcaacaccccgacttcgagaagtggcaacaaaagggtaaactcccgtctaccgttgtgagccagttatgtcaacctctcaaaactgaccctcgctttgcaggtcagcccagccgtttatatatgtcggcaattcatattgtggactacatctacaagtcctggctggctatacagaaacggcttcaacagcagctagatggaaagacgcgctggctagaaatgctcaatagcgatgctgaattagtagaacttagtggtgacactttagaggctattcgtgtcaaagctgctgaaattttggcaatagctatgccagcatctgagtcagatagcgcttcacctaaagggaaaaaaggtaaaaaggagaaaaaaccctcatcttctagccctaagcgtagtttatccaagacattatttgacgcttaccaagaaacggaagatattaagagccgtagcgccatcagctacctgttaaaaaatggctgcaaacttactgacaaagaagaagattcagaaaaatttgctaaacgtcgtcgtcaagttgaaatccaaattcaaaggcttaccgaaaagttaataagtcgcatgcctaaaggtcgagatttgaccaatgctaaatggttagagacactcttgactgctacaaccactgttgctgaagataacgcccaagccaaacgctggcaggatattctgttgactcgatcaagttctctcccattcccccttgtttttgaaaccaacgaggatatggtttggtcaaagaatcaaaagggtaggctgtgtgttcacttcaatggcttgagcgatttaatttttgaggtgtactgcggcaatcgtcaacttcactggtttcaacgcttcctagaagatcaacagactaaacgcaaaagcaaaaatcagcattctagcggcttgttcacactcagaaatggtcatctagtttggcttgaaggtgagggtaaaggggaaccttggaatcttcaccacttgaccctttactgctgtgttgacaatcgcttgtggacagaggagggaacagaaatcgttcgccaagagaaagcagatgaaattactaaattcatcacaaacatgaagaagaaaagcgatctaagcgatacacagcaggctttgattcaacgtaaacaatcaacacttactcgaataaacaattcctttgagcgtcctagccaacccctttatcaaggtcaatcacacattttggttggcgtaagcctgggactagaaaaacctgccacagtagcagtagtagatgcgatcgccaacaaagtcttggcttaccggagtattaaacaattacttggcgacaattacgaactgctaaatcgccagagacgacaacagcagtacctatctcacgaacgccacaaagcacaaaaaaacttctctcccaatcaatttggagcatctgagttagggcaacatatagacagattattagctaaagccattgtagcgttagcgagaacctacaaagctggcagtattgtcttgcccaagttaggggatatgcgggaggttgtccaaagtgaaattcaagctatagcagaacaaaaatttcccggttatattgaaggtcagcaaaaatatgccaaacagtaccgggttaatgttcatcggtggagctacggcagattaattcaaagcattcaaagtaaagcagctcaaacaggaattgtgattgaggagggaaaacaacctattcgaggtagtccccacgacaaagcaaaggaattagcactttctgcttacaatctccgcctaactaggcgaagctaacaaatatctgaaccttgataatagaacattaatagcgccgcaattcatgcttgacagctagctcagtcctaggtataatactagtatattaatagcgccgcaattcatgctgcttgcagcctctgaattttgttaaatgagggttagtttgactgtataaatacagtcttgctttctgaccctggtagctgctcaccctgatgctgctgtcaatagacaggataggtgcgctcccagcaataagggcgcggatgtactgctgtagtggctactgaatcacccccgatcaagggggaaccctccaaaaggtgggttgaaagactgcccgtttcgagagtttctcgtgaatttttttgttcagaacgctcggtcttgcacaccgggcgttttttctttgtgagtccagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 34 - DNA, 7288 bp, synthetic construct
  source 1..7288: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttataagaaggagatatacatatgaacagtcagcaaaatcctgatttagctgttcatcccttggcaattcctatggaaggcttactaggagaaagtgctacaactcttgagaagaatgtaattgccacacaactctcagaggaagcccaagtaaagctagaggtaatccaaagtttactggaaccctgcgatcgcacaacttatgggcaaaagttgcgggaagcagcagagaaactaaatgtatcgttgcgaacggtacaaaggttggtgaaaaactgggaacaagatggcttagtcggcctcactcaaacaagtagggctgataaaggaaaacaccgcattggtgagttttgggaaaacttcattaccaaaacctacaaggagggtaacaagggaagtaaacgtatgacccctaaacaagttgctctcagagtcgaggctaaagcccgtgaattaaaagactctaagccgcccaattacaaaaccgtgttacgggtattagcacccattttggaaaagcaacaaaaagccaagagtatccgcagtcctggctggagaggaactacgctttcggttaaaacccgtgaaggaaaagatttatcggttgattacagtaaccatgtttggcaatgtgaccatacccgcgtggatgtgttgctggtagatcaacatggtgaaattttaagtcgtccctggctaacaacagtaattgatacttactctcgttgcattatgggtatcaacttgggctttgatgcacccagttctggggtagtagcattagcgttacgccatgcaattctaccaaagcgttacggttccgagtacaaactgcattgtgagtggggaacctatggaaaaccagaacatttttatactgatggcggtaaagactttcgctctaaccacttgagtcagattggggcgcagttgggatttgtctgtcatttacgcgatcgcccttctgaaggtggagtagtagaacgtcccttcaaaacattaaatgaccaactattttcaacgcttcctgggtacaccggatctaatgtgcaggaacgcccagaagatgcagagaaggacgcaagacttactttgcgagaactagaacagttacttgtgcgttacatcgtagatcgttacaaccaaagtattgatgcgcggatgggcgaccaaacgcgctttgagcgttgggaagcaggattgcctacagtgccagtaccaataccagaacgagatttggatatttgtttaatgaagcagtcacggcgcactgtgcaaagaggtggttgtttgcagtttcagaatttaatgtatcggggggagtatttggcaggttatgccggagaaactgtcaacttgaggtttgaccccagagacattacaacaattttggtttatcgccaggaaaacaatcaggaagtatttctgactcgcgctcacgctcaaggtttggagacagagcaactggcattagatgaggctgaggcagcaagtcgcagactccgtaccgcagggaaaactatcagtaaccaatcattattgcaagaagttgttgaccgcgatgctcttgtcgctaccaagaaaagccgtaaggagcgtcaaaaattggaacagactgttttgcgatctgctgctgttgatgaaagtaatagagaatccttgccttctcaaatagttgaaccagatgaagtggaatctacagaaacggttcactctcaatacgaggacattgaggtgtgggactatgaacaacttcgtgaagaatatgggttttaaacaatgatagaagcaccagatgttaaaccttggctattcttgattaaaccctatgaaggggaaagcctgagccactttcttggcaggttcagacgtgccaaccatttatccgcaagtggattgggtactttggcaggaattggtgctatagtggcacgttgggaaagatttcattttaatcctcgccctagtcagcaagaattggaagcgatcgcatctgtagtagaagtggatgctcaaaggttagcccagatgttaccgcctgctggagtgggaatgcagcatgagccaattcgcttgtgtggggcttgttatgccgagtcgccttgtcaccgaattgaatggcagtacaagtcggtgtggaagtgcgatcgccatcaactcaagattttagcaaagtgtccaaactgtcaagcaccttttaaaatgcctgcgctgtgggaggatgggtgctgtcacagatgtaggatgccgtttgcagaaatggcaaagctacagaaggtttgatgataaaaccagaaaaaggtcttaaataaggagatataccatgagtcaaataactattcaagctcgacttatttcctttgaatcaaaccgccaacaactctggaagttgatggcagatttaaacacgccgttaattaacgaactgctttgccagttaggtcaacaccccgacttcgagaagtggcaacaaaagggtaaactcccgtctaccgttgtgagccagttatgtcaacctctcaaaactgaccctcgctttgcaggtcagcccagccgtttatatatgtcggcaattcatattgtggactacatctacaagtcctggctggctatacagaaacggcttcaacagcagctagatggaaagacgcgctggctagaaatgctcaatagcgatgctgaattagtagaacttagtggtgacactttagaggctattcgtgtcaaagctgctgaaattttggcaatagctatgccagcatctgagtcagatagcgcttcacctaaagggaaaaaaggtaaaaaggagaaaaaaccctcatcttctagccctaagcgtagtttatccaagacattatttgacgcttaccaagaaacggaagatattaagagccgtagcgccatcagctacctgttaaaaaatggctgcaaacttactgacaaagaagaagattcagaaaaatttgctaaacgtcgtcgtcaagttgaaatccaaattcaaaggcttaccgaaaagttaataagtcgcatgcctaaaggtcgagatttgaccaatgctaaatggttagagacactcttgactgctacaaccactgttgctgaagataacgcccaagccaaacgctggcaggatattctgttgactcgatcaagttctctcccattcccccttgtttttgaaaccaacgaggatatggtttggtcaaagaatcaaaagggtaggctgtgtgttcacttcaatggcttgagcgatttaatttttgaggtgtactgcggcaatcgtcaacttcactggtttcaacgcttcctagaagatcaacagactaaacgcaaaagcaaaaatcagcattctagcggcttgttcacactcagaaatggtcatctagtttggcttgaaggtgagggtaaaggggaaccttggaatcttcaccacttgaccctttactgctgtgttgacaatcgcttgtggacagaggagggaacagaaatcgttcgccaagagaaagcagatgaaattactaaattcatcacaaacatgaagaagaaaagcgatctaagcgatacacagcaggctttgattcaacgtaaacaatcaacacttactcgaataaacaattcctttgagcgtcctagccaacccctttatcaaggtcaatcacacattttggttggcgtaagcctgggactagaaaaacctgccacagtagcagtagtagatgcgatcgccaacaaagtcttggcttaccggagtattaaacaattacttggcgacaattacgaactgctaaatcgccagagacgacaacagcagtacctatctcacgaacgccacaaagcacaaaaaaacttctctcccaatcaatttggagcatctgagttagggcaacatatagacagattattagctaaagccattgtagcgttagcgagaacctacaaagctggcagtattgtcttgcccaagttaggggatatgcgggaggttgtccaaagtgaaattcaagctatagcagaacaaaaatttcccggttatattgaaggtcagcaaaaatatgccaaacagtaccgggttaatgttcatcggtggagctacggcagattaattcaaagcattcaaagtaaagcagctcaaacaggaattgtgattgaggagggaaaacaacctattcgaggtagtccccacgacaaagcaaaggaattagcactttctgcttacaatctccgcctaactaggcgaagctaacaaatatctgaaccttgataatagaacattaatagcgccgcaattcatgcttgacagctagctcagtcctaggtataatactagtatattaatagcgccgcaattcatgctgcttgcagcctctgaattttgttaaatgagggttagtttgactgtataaatacagtcttgctttctgaccctggtagctgctcaccctgatgctgctgtcaatagacaggataggtgcgctcccagcaataagggcgcggatgtactgctgtagtggctactgaatcacccccgatcaagggggaaccctccaaaaggtgggttgaaagatagcgatcccttgctgaaaatagtgaatttttttgttcagaacgctcggtcttgcacaccgggcgttttttctttgtgagtccagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 35 - DNA, 7288 bp, synthetic construct
  source 1..7288: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttataagaaggagatatacatatgaacagtcagcaaaatcctgatttagctgttcatcccttggcaattcctatggaaggcttactaggagaaagtgctacaactcttgagaagaatgtaattgccacacaactctcagaggaagcccaagtaaagctagaggtaatccaaagtttactggaaccctgcgatcgcacaacttatgggcaaaagttgcgggaagcagcagagaaactaaatgtatcgttgcgaacggtacaaaggttggtgaaaaactgggaacaagatggcttagtcggcctcactcaaacaagtagggctgataaaggaaaacaccgcattggtgagttttgggaaaacttcattaccaaaacctacaaggagggtaacaagggaagtaaacgtatgacccctaaacaagttgctctcagagtcgaggctaaagcccgtgaattaaaagactctaagccgcccaattacaaaaccgtgttacgggtattagcacccattttggaaaagcaacaaaaagccaagagtatccgcagtcctggctggagaggaactacgctttcggttaaaacccgtgaaggaaaagatttatcggttgattacagtaaccatgtttggcaatgtgaccatacccgcgtggatgtgttgctggtagatcaacatggtgaaattttaagtcgtccctggctaacaacagtaattgatacttactctcgttgcattatgggtatcaacttgggctttgatgcacccagttctggggtagtagcattagcgttacgccatgcaattctaccaaagcgttacggttccgagtacaaactgcattgtgagtggggaacctatggaaaaccagaacatttttatactgatggcggtaaagactttcgctctaaccacttgagtcagattggggcgcagttgggatttgtctgtcatttacgcgatcgcccttctgaaggtggagtagtagaacgtcccttcaaaacattaaatgaccaactattttcaacgcttcctgggtacaccggatctaatgtgcaggaacgcccagaagatgcagagaaggacgcaagacttactttgcgagaactagaacagttacttgtgcgttacatcgtagatcgttacaaccaaagtattgatgcgcggatgggcgaccaaacgcgctttgagcgttgggaagcaggattgcctacagtgccagtaccaataccagaacgagatttggatatttgtttaatgaagcagtcacggcgcactgtgcaaagaggtggttgtttgcagtttcagaatttaatgtatcggggggagtatttggcaggttatgccggagaaactgtcaacttgaggtttgaccccagagacattacaacaattttggtttatcgccaggaaaacaatcaggaagtatttctgactcgcgctcacgctcaaggtttggagacagagcaactggcattagatgaggctgaggcagcaagtcgcagactccgtaccgcagggaaaactatcagtaaccaatcattattgcaagaagttgttgaccgcgatgctcttgtcgctaccaagaaaagccgtaaggagcgtcaaaaattggaacagactgttttgcgatctgctgctgttgatgaaagtaatagagaatccttgccttctcaaatagttgaaccagatgaagtggaatctacagaaacggttcactctcaatacgaggacattgaggtgtgggactatgaacaacttcgtgaagaatatgggttttaaacaatgatagaagcaccagatgttaaaccttggctattcttgattaaaccctatgaaggggaaagcctgagccactttcttggcaggttcagacgtgccaaccatttatccgcaagtggattgggtactttggcaggaattggtgctatagtggcacgttgggaaagatttcattttaatcctcgccctagtcagcaagaattggaagcgatcgcatctgtagtagaagtggatgctcaaaggttagcccagatgttaccgcctgctggagtgggaatgcagcatgagccaattcgcttgtgtggggcttgttatgccgagtcgccttgtcaccgaattgaatggcagtacaagtcggtgtggaagtgcgatcgccatcaactcaagattttagcaaagtgtccaaactgtcaagcaccttttaaaatgcctgcgctgtgggaggatgggtgctgtcacagatgtaggatgccgtttgcagaaatggcaaagctacagaaggtttgatgataaaaccagaaaaaggtcttaaataaggagatataccatgagtcaaataactattcaagctcgacttatttcctttgaatcaaaccgccaacaactctggaagttgatggcagatttaaacacgccgttaattaacgaactgctttgccagttaggtcaacaccccgacttcgagaagtggcaacaaaagggtaaactcccgtctaccgttgtgagccagttatgtcaacctctcaaaactgaccctcgctttgcaggtcagcccagccgtttatatatgtcggcaattcatattgtggactacatctacaagtcctggctggctatacagaaacggcttcaacagcagctagatggaaagacgcgctggctagaaatgctcaatagcgatgctgaattagtagaacttagtggtgacactttagaggctattcgtgtcaaagctgctgaaattttggcaatagctatgccagcatctgagtcagatagcgcttcacctaaagggaaaaaaggtaaaaaggagaaaaaaccctcatcttctagccctaagcgtagtttatccaagacattatttgacgcttaccaagaaacggaagatattaagagccgtagcgccatcagctacctgttaaaaaatggctgcaaacttactgacaaagaagaagattcagaaaaatttgctaaacgtcgtcgtcaagttgaaatccaaattcaaaggcttaccgaaaagttaataagtcgcatgcctaaaggtcgagatttgaccaatgctaaatggttagagacactcttgactgctacaaccactgttgctgaagataacgcccaagccaaacgctggcaggatattctgttgactcgatcaagttctctcccattcccccttgtttttgaaaccaacgaggatatggtttggtcaaagaatcaaaagggtaggctgtgtgttcacttcaatggcttgagcgatttaatttttgaggtgtactgcggcaatcgtcaacttcactggtttcaacgcttcctagaagatcaacagactaaacgcaaaagcaaaaatcagcattctagcggcttgttcacactcagaaatggtcatctagtttggcttgaaggtgagggtaaaggggaaccttggaatcttcaccacttgaccctttactgctgtgttgacaatcgcttgtggacagaggagggaacagaaatcgttcgccaagagaaagcagatgaaattactaaattcatcacaaacatgaagaagaaaagcgatctaagcgatacacagcaggctttgattcaacgtaaacaatcaacacttactcgaataaacaattcctttgagcgtcctagccaacccctttatcaaggtcaatcacacattttggttggcgtaagcctgggactagaaaaacctgccacagtagcagtagtagatgcgatcgccaacaaagtcttggcttaccggagtattaaacaattacttggcgacaattacgaactgctaaatcgccagagacgacaacagcagtacctatctcacgaacgccacaaagcacaaaaaaacttctctcccaatcaatttggagcatctgagttagggcaacatatagacagattattagctaaagccattgtagcgttagcgagaacctacaaagctggcagtattgtcttgcccaagttaggggatatgcgggaggttgtccaaagtgaaattcaagctatagcagaacaaaaatttcccggttatattgaaggtcagcaaaaatatgccaaacagtaccgggttaatgttcatcggtggagctacggcagattaattcaaagcattcaaagtaaagcagctcaaacaggaattgtgattgaggagggaaaacaacctattcgaggtagtccccacgacaaagcaaaggaattagcactttctgcttacaatctccgcctaactaggcgaagctaacaaatatctgaaccttgataatagaacattaatagcgccgcaattcatgcttgacagctagctcagtcctaggtataatactagtatattaatagcgccgcaattcatgctgcttgcagcctctgaattttgttaaatgagggttagtttgactgtataaatacagtcttgctttctgaccctggtagctgctcaccctgatgctgctgtcaatagacaggataggtgcgctcccagcaataagggcgcggatgtactgctgtagtggctactgaatcacccccgatcaagggggaaccctccaaaaggtgggttgaaaggccactcgctttaatgatgatttgtgaatttttttgttcagaacgctcggtcttgcacaccgggcgttttttctttgtgagtccagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 36 - DNA, 8115 bp, synthetic construct
  source 1..8115: mol_type = other DNA
gagcggataacaatttcacacaggaaacagctatgaccatgattacgccaagctttataagaaggagatatacatatgaacagtcagcaaaatcctgatttagctgttcatcccttggcaattcctatggaaggcttactaggagaaagtgctacaactcttgagaagaatgtaattgccacacaactctcagaggaagcccaagtaaagctagaggtaatccaaagtttactggaaccctgcgatcgcacaacttatgggcaaaagttgcgggaagcagcagagaaactaaatgtatcgttgcgaacggtacaaaggttggtgaaaaactgggaacaagatggcttagtcggcctcactcaaacaagtagggctgataaaggaaaacaccgcattggtgagttttgggaaaacttcattaccaaaacctacaaggagggtaacaagggaagtaaacgtatgacccctaaacaagttgctctcagagtcgaggctaaagcccgtgaattaaaagactctaagccgcccaattacaaaaccgtgttacgggtattagcacccattttggaaaagcaacaaaaagccaagagtatccgcagtcctggctggagaggaactacgctttcggttaaaacccgtgaaggaaaagatttatcggttgattacagtaaccatgtttggcaatgtgaccatacccgcgtggatgtgttgctggtagatcaacatggtgaaattttaagtcgtccctggctaacaacagtaattgatacttactctcgttgcattatgggtatcaacttgggctttgatgcacccagttctggggtagtagcattagcgttacgccatgcaattctaccaaagcgttacggttccgagtacaaactgcattgtgagtggggaacctatggaaaaccagaacatttttatactgatggcggtaaagactttcgctctaaccacttgagtcagattggggcgcagttgggatttgtctgtcatttacgcgatcgcccttctgaaggtggagtagtagaacgtcccttcaaaacattaaatgaccaactattttcaacgcttcctgggtacaccggatctaatgtgcaggaacgcccagaagatgcagagaaggacgcaagacttactttgcgagaactagaacagttacttgtgcgttacatcgtagatcgttacaaccaaagtattgatgcgcggatgggcgaccaaacgcgctttgagcgttgggaagcaggattgcctacagtgccagtaccaataccagaacgagatttggatatttgtttaatgaagcagtcacggcgcactgtgcaaagaggtggttgtttgcagtttcagaatttaatgtatcggggggagtatttggcaggttatgccggagaaactgtcaacttgaggtttgaccccagagacattacaacaattttggtttatcgccaggaaaacaatcaggaagtatttctgactcgcgctcacgctcaaggtttggagacagagcaactggcattagatgaggctgaggcagcaagtcgcagactccgtaccgcagggaaaactatcagtaaccaatcattattgcaagaagttgttgaccgcgatgctcttgtcgctaccaagaaaagccgtaaggagcgtcaaaaattggaacagactgttttgcgatctgctgctgttgatgaaagtaatagagaatccttgccttctcaaatagttgaaccagatgaagtggaatctacagaaacggttcactctcaatacgaggacattgaggtgtgggactatgaacaacttcgtgaagaatatgggttttaaacaatgacagaagctcaggcgatcgccaagcagttgggtggggtaaaaccggatgatgagtggttacaagctgaaattgctcgtctcaagggtaagagcattgtgcctttacagcaggtaaaaactctccatgattggttagatggcaagcgcaaggcaagaaaatcttgccgagtagttggggaatcgagaactggcaagacagttgcttgtgatgcctacagatacaggcacaaacctcagcaggaagctggacgaccgccaactgtgcctgtcgtttatattcgacctcaccaaaaatgtggccccaaggatttgtttaaaaagattactgagtacctcaagtatcgggtaacaaaagggactgtatctgattttcgagataggacgatagaagtactcaagggttgtggcgtagagatgctaattattgatgaagctgaccgtctcaagcctgaaacttttgctgatgtgcgagatattgccgaagatttaggaattgctgtggtactggtaggaacagaccgtttggatgcggtaattaagcgggatgagcaggttctcgaacgctttcgggcgcatcttcgctttggtaaattgtcgggagaggattttaagaacaccgtagaaatgtgggaacaaatggttttgaaactgccagtatcttctaatctaaagagcaaggagatgctacggattctcacgtcagcaactgaaggctacattggtcgccttgatgagattcttagggaagctgcaattcgttccttatcaagaggattgaagaagattgacaaggctgttttacaggaagtagctaaggagtacaaatgatagaagcaccagatgttaaaccttggctattcttgattaaaccctatgaaggggaaagcctgagccactttcttggcaggttcagacgtgccaaccatttatccgcaagtggattgggtactttggcaggaattggtgctatagtggcacgttgggaaagatttcattttaatcctcgccctagtcagcaagaattggaagcgatcgcatctgtagtagaagtggatgctcaaaggttagcccagatgttaccgcctgctggagtgggaatgcagcatgagccaattcgcttgtgtggggcttgttatgccgagtcgccttgtcaccgaattgaatggcagtacaagtcggtgtggaagtgcgatcgccatcaactcaagattttagcaaagtgtccaaactgtcaagcaccttttaaaatgcctgcgctgtgggaggatgggtgctgtcacagatgtaggatgccgtttgcagaaatggcaaagctacagaaggtttgatgataaaaccagaaaaaggtcttaaataaggagatataccatgagtcaaataactattcaagctcgacttatttcctttgaatcaaaccgccaacaactctggaagttgatggcagatttaaacacgccgttaattaacgaactgctttgccagttaggtcaacaccccgacttcgagaagtggcaacaaaagggtaaactcccgtctaccgttgtgagccagttatgtcaacctctcaaaactgaccctcgctttgcaggtcagcccagccgtttatatatgtcggcaattcatattgtggactacatctacaagtcctggctggctatacagaaacggcttcaacagcagctagatggaaagacgcgctggctagaaatgctcaatagcgatgctgaattagtagaacttagtggtgacactttagaggctattcgtgtcaaagctgctgaaattttggcaatagctatgccagcatctgagtcagatagcgcttcacctaaagggaaaaaaggtaaaaaggagaaaaaaccctcatcttctagccctaagcgtagtttatccaagacattatttgacgcttaccaagaaacggaagatattaagagccgtagcgccatcagctacctgttaaaaaatggctgcaaacttactgacaaagaagaagattcagaaaaatttgctaaacgtcgtcgtcaagttgaaatccaaattcaaaggcttaccgaaaagttaataagtcgcatgcctaaaggtcgagatttgaccaatgctaaatggttagagacactcttgactgctacaaccactgttgctgaagataacgcccaagccaaacgctggcaggatattctgttgactcgatcaagttctctcccattcccccttgtttttgaaaccaacgaggatatggtttggtcaaagaatcaaaagggtaggctgtgtgttcacttcaatggcttgagcgatttaatttttgaggtgtactgcggcaatcgtcaacttcactggtttcaacgcttcctagaagatcaacagactaaacgcaaaagcaaaaatcagcattctagcggcttgttcacactcagaaatggtcatctagtttggcttgaaggtgagggtaaaggggaaccttggaatcttcaccacttgaccctttactgctgtgttgacaatcgcttgtggacagaggagggaacagaaatcgttcgccaagagaaagcagatgaaattactaaattcatcacaaacatgaagaagaaaagcgatctaagcgatacacagcaggctttgattcaacgtaaacaatcaacacttactcgaataaacaattcctttgagcgtcctagccaacccctttatcaaggtcaatcacacattttggttggcgtaagcctgggactagaaaaacctgccacagtagcagtagtagatgcgatcgccaacaaagtcttggcttaccggagtattaaacaattacttggcgacaattacgaactgctaaatcgccagagacgacaacagcagtacctatctcacgaacgccacaaagcacaaaaaaacttctctcccaatcaatttggagcatctgagttagggcaacatatagacagattattagctaaagccattgtagcgttagcgagaacctacaaagctggcagtattgtcttgcccaagttaggggatatgcgggaggttgtccaaagtgaaattcaagctatagcagaacaaaaatttcccggttatattgaaggtcagcaaaaatatgccaaacagtaccgggttaatgttcatcggtggagctacggcagattaattcaaagcattcaaagtaaagcagctcaaacaggaattgtgattgaggagggaaaacaacctattcgaggtagtccccacgacaaagcaaaggaattagcactttctgcttacaatctccgcctaactaggcgaagctaacaaatatctgaaccttgataatagaacattaatagcgccgcaattcatgcttgacagctagctcagtcctaggtataatactagtatattaatagcgccgcaattcatgctgcttgcagcctctgaattttgttaaatgagggttagtttgactgtataaatacagtcttgctttctgaccctggtagctgctcaccctgatgctgctgtcaatagacaggataggtgcgctcccagcaataagggcgcggatgtactgctgtagtggctactgaatcacccccgatcaagggggaaccctccaaaaggtgggttgaaagttgtcagatattacgcctgtgtggtgaatttttttgttcagaacgctcggtcttgcacaccgggcgttttttctttgtgagtccagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgt

SEQ ID NO: 37 - DNA, 8115 bp, synthetic construct
  source 1..8115: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttataagaaggagatatacatatgaacagtcagcaaaatcctgatttagctgttcatcccttggcaattcctatggaaggcttactaggagaaagtgctacaactcttgagaagaatgtaattgccacacaactctcagaggaagcccaagtaaagctagaggtaatccaaagtttactggaaccctgcgatcgcacaacttatgggcaaaagttgcgggaagcagcagagaaactaaatgtatcgttgcgaacggtacaaaggttggtgaaaaactgggaacaagatggcttagtcggcctcactcaaacaagtagggctgataaaggaaaacaccgcattggtgagttttgggaaaacttcattaccaaaacctacaaggagggtaacaagggaagtaaacgtatgacccctaaacaagttgctctcagagtcgaggctaaagcccgtgaattaaaagactctaagccgcccaattacaaaaccgtgttacgggtattagcacccattttggaaaagcaacaaaaagccaagagtatccgcagtcctggctggagaggaactacgctttcggttaaaacccgtgaaggaaaagatttatcggttgattacagtaaccatgtttggcaatgtgaccatacccgcgtggatgtgttgctggtagatcaacatggtgaaattttaagtcgtccctggctaacaacagtaattgatacttactctcgttgcattatgggtatcaacttgggctttgatgcacccagttctggggtagtagcattagcgttacgccatgcaattctaccaaagcgttacggttccgagtacaaactgcattgtgagtggggaacctatggaaaaccagaacatttttatactgatggcggtaaagactttcgctctaaccacttgagtcagattggggcgcagttgggatttgtctgtcatttacgcgatcgcccttctgaaggtggagtagtagaacgtcccttcaaaacattaaatgaccaactattttcaacgcttcctgggtacaccggatctaatgtgcaggaacgcccagaagatgcagagaaggacgcaagacttactttgcgagaactagaacagttacttgtgcgttacatcgtagatcgttacaaccaaagtattgatgcgcggatgggcgaccaaacgcgctttgagcgttgggaagcaggattgcctacagtgccagtaccaataccagaacgagatttggatatttgtttaatgaagcagtcacggcgcactgtgcaaagaggtggttgtttgcagtttcagaatttaatgtatcggggggagtatttggcaggttatgccggagaaactgtcaacttgaggtttgaccccagagacattacaacaattttggtttatcgccaggaaaacaatcaggaagtatttctgactcgcgctcacgctcaaggtttggagacagagcaactggcattagatgaggctgaggcagcaagtcgcagactccgtaccgcagggaaaactatcagtaaccaatcattattgcaagaagttgttgaccgcgatgctcttgtcgctaccaagaaaagccgtaaggagcgtcaaaaattggaacagactgttttgcgatctgctgctgttgatgaaagtaatagagaatccttgccttctcaaatagttgaaccagatgaagtggaatctacagaaacggttcactctcaatacgaggacattgaggtgtgggactatgaacaacttcgtgaagaatatgggttttaaacaatgacagaagctcaggcgatcgccaagcagttgggtggggtaaaaccggatgatgagtggttacaagctgaaattgctcgtctcaagggtaagagcattgtgcctttacagcaggtaaaaactctccatgattggttagatggcaagcgcaaggcaagaaaatcttgccgagtagttggggaatcgagaactggcaagacagttgcttgtgatgcctacagatacaggcacaaacctcagcaggaagctggacgaccgccaactgtgcctgtcgtttatattcgacctcaccaaaaatgtggccccaaggatttgtttaaaaagattactgagtacctcaagtatcgggtaacaaaagggactgtatctgattttcgagataggacgatagaagtactcaagggttgtggcgtagagatgctaattattgatgaagctgaccgtctcaagcctgaaacttttgctgatgtgcgagatattgccgaagatttaggaattgctgtggtactggtaggaacagaccgtttggatgcggtaattaagcgggatgagcaggttctcgaacgctttcgggcgcatcttcgctttggtaaattgtcgggagaggattttaagaacaccgtagaaatgtgggaacaaatggttttgaaactgccagtatcttctaatctaaagagcaaggagatgctacggattctcacgtcagcaactgaaggctacattggtcgccttgatgagattcttagggaagctgcaattcgttccttatcaagaggattgaagaagattgacaaggctgttttacaggaagtagctaaggagtacaaatgatagaagcaccagatgttaaaccttggctattcttgattaaaccctatgaaggggaaagcctgagccactttcttggcaggttcagacgtgccaaccatttatccgcaagtggattgggtactttggcaggaattggtgctatagtggcacgttgggaaagatttcattttaatcctcgccctagtcagcaagaattggaagcgatcgcatctgtagtagaagtggatgctcaaaggttagcccagatgttaccgcctgctggagtgggaatgcagcatgagccaattcgcttgtgtggggcttgttatgccgagtcgccttgtcaccgaattgaatggcagtacaagtcggtgtggaagtgcgatcgccatcaactcaagattttagcaaagtgtccaaactgtcaagcaccttttaaaatgcctgcgctgtgggaggatgggtgctgtcacagatgtaggatgccgtttgcagaaatggcaaagctacagaaggtttgatgataaaaccagaaaaaggtcttaaataaggagatataccatgagtcaaataactattcaagctcgacttatttcctttgaatcaaaccgccaacaactctggaagttgatggcagatttaaacacgccgttaattaacgaactgctttgccagttaggtcaacaccccgacttcgagaagtggcaacaaaagggtaaactcccgtctaccgttgtgagccagttatgtcaacctctcaaaactgaccctcgctttgcaggtcagcccagccgtttatatatgtcggcaattcatattgtggactacatctacaagtcctggctggctatacagaaacggcttcaacagcagctagatggaaagacgcgctggctagaaatgctcaatagcgatgctgaattagtagaacttagtggtgacactttagaggctattcgtgtcaaagctgctgaaattttggcaatagctatgccagcatctgagtcagatagcgcttcacctaaagggaaaaaaggtaaaaaggagaaaaaaccctcatcttctagccctaagcgtagtttatccaagacattatttgacgcttaccaagaaacggaagatattaagagccgtagcgccatcagctacctgttaaaaaatggctgcaaacttactgacaaagaagaagattcagaaaaatttgctaaacgtcgtcgtcaagttgaaatccaaattcaaaggcttaccgaaaagttaataagtcgcatgcctaaaggtcgagatttgaccaatgctaaatggttagagacactcttgactgctacaaccactgttgctgaagataacgcccaagccaaacgctggcaggatattctgttgactcgatcaagttctctcccattcccccttgtttttgaaaccaacgaggatatggtttggtcaaagaatcaaaagggtaggctgtgtgttcacttcaatggcttgagcgatttaatttttgaggtgtactgcggcaatcgtcaacttcactggtttcaacgcttcctagaagatcaacagactaaacgcaaaagcaaaaatcagcattctagcggcttgttcacactcagaaatggtcatctagtttggcttgaaggtgagggtaaaggggaaccttggaatcttcaccacttgaccctttactgctgtgttgacaatcgcttgtggacagaggagggaacagaaatcgttcgccaagagaaagcagatgaaattactaaattcatcacaaacatgaagaagaaaagcgatctaagcgatacacagcaggctttgattcaacgtaaacaatcaacacttactcgaataaacaattcctttgagcgtcctagccaacccctttatcaaggtcaatcacacattttggttggcgtaagcctgggactagaaaaacctgccacagtagcagtagtagatgcgatcgccaacaaagtcttggcttaccggagtattaaacaattacttggcgacaattacgaactgctaaatcgccagagacgacaacagcagtacctatctcacgaacgccacaaagcacaaaaaaacttctctcccaatcaatttggagcatctgagttagggcaacatatagacagattattagctaaagccattgtagcgttagcgagaacctacaaagctggcagtattgtcttgcccaagttaggggatatgcgggaggttgtccaaagtgaaattcaagctatagcagaacaaaaatttcccggttatattgaaggtcagcaaaaatatgccaaacagtaccgggttaatgttcatcggtggagctacggcagattaattcaaagcattcaaagtaaagcagctcaaacaggaattgtgattgaggagggaaaacaacctattcgaggtagtccccacgacaaagcaaaggaattagcactttctgcttacaatctccgcctaactaggcgaagctaacaaatatctgaaccttgataatagaacattaatagcgccgcaattcatgcttgacagctagctcagtcctaggtataatactagtatattaatagcgccgcaattcatgctgcttgcagcctctgaattttgttaaatgagggttagtttgactgtataaatacagtcttgctttctgaccctggtagctgctcaccctgatgctgctgtcaatagacaggataggtgcgctcccagcaataagggcgcggatgtactgctgtagtggctactgaatcacccccgatcaagggggaaccctccaaaaggtgggttgaaagactgcccgtttcgagagtttctcgtgaatttttttgttcagaacgctcggtcttgcacaccgggcgttttttctttgtgagtccagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 38 - DNA, 8115 bp, synthetic construct
  source 1..8115: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttataagaaggagatatacatatgaacagtcagcaaaatcctgatttagctgttcatcccttggcaattcctatggaaggcttactaggagaaagtgctacaactcttgagaagaatgtaattgccacacaactctcagaggaagcccaagtaaagctagaggtaatccaaagtttactggaaccctgcgatcgcacaacttatgggcaaaagttgcgggaagcagcagagaaactaaatgtatcgttgcgaacggtacaaaggttggtgaaaaactgggaacaagatggcttagtcggcctcactcaaacaagtagggctgataaaggaaaacaccgcattggtgagttttgggaaaacttcattaccaaaacctacaaggagggtaacaagggaagtaaacgtatgacccctaaacaagttgctctcagagtcgaggctaaagcccgtgaattaaaagactctaagccgcccaattacaaaaccgtgttacgggtattagcacccattttggaaaagcaacaaaaagccaagagtatccgcagtcctggctggagaggaactacgctttcggttaaaacccgtgaaggaaaagatttatcggttgattacagtaaccatgtttggcaatgtgaccatacccgcgtggatgtgttgctggtagatcaacatggtgaaattttaagtcgtccctggctaacaacagtaattgatacttactctcgttgcattatgggtatcaacttgggctttgatgcacccagttctggggtagtagcattagcgttacgccatgcaattctaccaaagcgttacggttccgagtacaaactgcattgtgagtggggaacctatggaaaaccagaacatttttatactgatggcggtaaagactttcgctctaaccacttgagtcagattggggcgcagttgggatttgtctgtcatttacgcgatcgcccttctgaaggtggagtagtagaacgtcccttcaaaacattaaatgaccaactattttcaacgcttcctgggtacaccggatctaatgtgcaggaacgcccagaagatgcagagaaggacgcaagacttactttgcgagaactagaacagttacttgtgcgttacatcgtagatcgttacaaccaaagtattgatgcgcggatgggcgaccaaacgcgctttgagcgttgggaagcaggattgcctacagtgccagtaccaataccagaacgagatttggatatttgtttaatgaagcagtcacggcgcactgtgcaaagaggtggttgtttgcagtttcagaatttaatgtatcggggggagtatttggcaggttatgccggagaaactgtcaacttgaggtttgaccccagagacattacaacaattttggtttatcgccaggaaaacaatcaggaagtatttctgactcgcgctcacgctcaaggtttggagacagagcaactggcattagatgaggctgaggcagcaagtcgcagactccgtaccgcagggaaaactatcagtaaccaatcattattgcaagaagttgttgaccgcgatgctcttgtcgctaccaagaaaagccgtaaggagcgtcaaaaattggaacagactgttttgcgatctgctgctgttgatgaaagtaatagagaatccttgccttctcaaatagttgaaccagatgaagtggaatctacagaaacggttcactctcaatacgaggacattgaggtgtgggactatgaacaacttcgtgaagaatatgggttttaaacaatgacagaagctcaggcgatcgccaagcagttgggtggggtaaaaccggatgatgagtggttacaagctgaaattgctcgtctcaagggtaagagcattgtgcctttacagcaggtaaaaactctccatgattggttagatggcaagcgcaaggcaagaaaatcttgccgagtagttggggaatcgagaactggcaagacagttgcttgtgatgcctacagatacaggcacaaacctcagcaggaagctggacgaccgccaactgtgcctgtcgtttatattcgacctcaccaaaaatgtggccccaaggatttgtttaaaaagattactgagtacctcaagtatcgggtaacaaaagggactgtatctgattttcgagataggacgatagaagtactcaagggttgtggcgtagagatgctaattattgatgaagctgaccgtctcaagcctgaaacttttgctgatgtgcgagatattgccgaagatttaggaattgctgtggtactggtaggaacagaccgtttggatgcggtaattaagcgggatgagcaggttctcgaacgctttcgggcgcatcttcgctttggtaaattgtcgggagaggattttaagaacaccgtagaaatgtgggaacaaatggttttgaaactgccagtatcttctaatctaaagagcaaggagatgctacggattctcacgtcagcaactgaaggctacattggtcgccttgatgagattcttagggaagctgcaattcgttccttatcaagaggattgaagaagattgacaaggctgttttacaggaagtagctaaggagtacaaatgatagaagcaccagatgttaaaccttggctattcttgattaaaccctatgaaggggaaagcctgagccactttcttggcaggttcagacgtgccaaccatttatccgcaagtggattgggtactttggcaggaattggtgctatagtggcacgttgggaaagatttcattttaatcctcgccctagtcagcaagaattggaagcgatcgcatctgtagtagaagtggatgctcaaaggttagcccagatgttaccgcctgctggagtgggaatgcagcatgagccaattcgcttgtgtggggcttgttatgccgagtcgccttgtcaccgaattgaatggcagtacaagtcggtgtggaagtgcgatcgccatcaactcaagattttagcaaagtgtccaaactgtcaagcaccttttaaaatgcctgcgctgtgggaggatgggtgctgtcacagatgtaggatgccgtttgcagaaatggcaaagctacagaaggtttgatgataaaaccagaaaaaggtcttaaataaggagatataccatgagtcaaataactattcaagctcgacttatttcctttgaatcaaaccgccaacaactctggaagttgatggcagatttaaacacgccgttaattaacgaactgctttgccagttaggtcaacaccccgacttcgagaagtggcaacaaaagggtaaactcccgtctaccgttgtgagccagttatgtcaacctctcaaaactgaccctcgctttgcaggtcagcccagccgtttatatatgtcggcaattcatattgtggactacatctacaagtcctggctggctatacagaaacggcttcaacagcagctagatggaaagacgcgctggctagaaatgctcaatagcgatgctgaattagtagaacttagtggtgacactttagaggctattcgtgtcaaagctgctgaaattttggcaatagctatgccagcatctgagtcagatagcgcttcacctaaagggaaaaaaggtaaaaaggagaaaaaaccctcatcttctagccctaagcgtagtttatccaagacattatttgacgcttaccaagaaacggaagatattaagagccgtagcgccatcagctacctgttaaaaaatggctgcaaacttactgacaaagaagaagattcagaaaaatttgctaaacgtcgtcgtcaagttgaaatccaaattcaaaggcttaccgaaaagttaataagtcgcatgcctaaaggtcgagatttgaccaatgctaaatggttagagacactcttgactgctacaaccactgttgctgaagataacgcccaagccaaacgctggcaggatattctgttgactcgatcaagttctctcccattcccccttgtttttgaaaccaacgaggatatggtttggtcaaagaatcaaaagggtaggctgtgtgttcacttcaatggcttgagcgatttaatttttgaggtgtactgcggcaatcgtcaacttcactggtttcaacgcttcctagaagatcaacagactaaacgcaaaagcaaaaatcagcattctagcggcttgttcacactcagaaatggtcatctagtttggcttgaaggtgagggtaaaggggaaccttggaatcttcaccacttgaccctttactgctgtgttgacaatcgcttgtggacagaggagggaacagaaatcgttcgccaagagaaagcagatgaaattactaaattcatcacaaacatgaagaagaaaagcgatctaagcgatacacagcaggctttgattcaacgtaaacaatcaacacttactcgaataaacaattcctttgagcgtcctagccaacccctttatcaaggtcaatcacacattttggttggcgtaagcctgggactagaaaaacctgccacagtagcagtagtagatgcgatcgccaacaaagtcttggcttaccggagtattaaacaattacttggcgacaattacgaactgctaaatcgccagagacgacaacagcagtacctatctcacgaacgccacaaagcacaaaaaaacttctctcccaatcaatttggagcatctgagttagggcaacatatagacagattattagctaaagccattgtagcgttagcgagaacctacaaagctggcagtattgtcttgcccaagttaggggatatgcgggaggttgtccaaagtgaaattcaagctatagcagaacaaaaatttcccggttatattgaaggtcagcaaaaatatgccaaacagtaccgggttaatgttcatcggtggagctacggcagattaattcaaagcattcaaagtaaagcagctcaaacaggaattgtgattgaggagggaaaacaacctattcgaggtagtccccacgacaaagcaaaggaattagcactttctgcttacaatctccgcctaactaggcgaagctaacaaatatctgaaccttgataatagaacattaatagcgccgcaattcatgcttgacagctagctcagtcctaggtataatactagtatattaatagcgccgcaattcatgctgcttgcagcctctgaattttgttaaatgagggttagtttgactgtataaatacagtcttgctttctgaccctggtagctgctcaccctgatgctgctgtcaatagacaggataggtgcgctcccagcaataagggcgcggatgtactgctgtagtggctactgaatcacccccgatcaagggggaaccctccaaaaggtgggttgaaagatagcgatcccttgctgaaaatagtgaatttttttgttcagaacgctcggtcttgcacaccgggcgttttttctttgtgagtccagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 39 - DNA, 4182 bp, synthetic construct
  source 1..4182: mol_type = other DNA
accgcctccccaggcggttttttcgtttacagagcaggagattacgacgatcgtaaaaggatctcaagaagatcctttacggattcccgacaccatcactctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactataggggaattgtgagcggataacaattcccctgtagaaataattttgtttaactttaataaggagatatacatgacagaagctcaggcgatcgccaagcagttgggtggggtaaaaccggatgatgagtggttacaagctgaaattgctcgtctcaagggtaagagcattgtgcctttacagcaggtaaaaactctccatgattggttagatggcaagcgcaaggcaagaaaatcttgccgagtagttggggaatcgagaactggcaagacagttgcttgtgatgcctacagatacaggcacaaacctcagcaggaagctggacgaccgccaactgtgcctgtcgtttatattcgacctcaccaaaaatgtggccccaaggatttgtttaaaaagattactgagtacctcaagtatcgggtaacaaaagggactgtatctgattttcgagataggacgatagaagtactcaagggttgtggcgtagagatgctaattattgatgaagctgaccgtctcaagcctgaaacttttgctgatgtgcgagatattgccgaagatttaggaattgctgtggtactggtaggaacagaccgtttggatgcggtaattaagcgggatgagcaggttctcgaagaatttcgggcgcatcttcgctttggtaaattgtcgggagaggattttaagaacaccgtagaaatgtgggaacaaatggttttgaaactgccagtatcttctaatctaaagagcaaggagatgctacggattctcacgtcagcaactgaaggctacattggtcgccttgatgagattcttagggaagctgcaattcgttccttatcaagaggattgaagaagattgacaaggctgttttacaggaagtagctaaggagtacaaatgagctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgacaagctgacgaccgggtctccgcaagtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcggtcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggcatgctagcgcagaaacgtcctagaagatgccaggaggatacttagcagagagacaataaggccggagcgaagccgtttttccataggctccgcccccctgacgaacatcacgaaatctgacgctcaaatcagtggtggcgaaacccgacaggactataaagataccaggcgtttccccctgatggctccctcttgcgctctcctgttcccgtcctgcggcgtccgtgttgtggtggaggctttacccaaatcaccacgtcccgttccgtgtagacagttcgctccaagctgggctgtgtgcaagaaccccccgttcagcccgactgctgcgccttatccggtaactatcatcttgagtccaacccggaaagacacgacaaaacgccactggcagcagccattggtaactgagaattagtggatttagatatcgagagtcttgaagtggtggcctaacagaggctacactgaaaggacagtatttggtatctgcgctccactaaagccagttaccaggttaagcagttccccaactgacttaaccttcgatcaa

SEQ ID NO: 40 - DNA, 4266 bp, synthetic construct
  source 1..4266: mol_type = other DNA
ggtatctgcgctccactaaagccagttaccaggttaagcagttccccaactgacttaaccttcgatcaaaccgcctccccaggcggttttttcgtttacagagcaggagattacgacgatcgtaaaaggatctcaagaagatcctttacggattcccgacaccatcactctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactataggggaattgtgagcggataacaattcccctgtagaaataattttgtttaactttaataaggagatatacatggactacaaagaccatgacggtgattataaagatcatgacatcgattacaaggatgacgatgacaagggaggaagcggatcgatgacagaagctcaggcgatcgccaagcagttgggtggggtaaaaccggatgatgagtggttacaagctgaaattgctcgtctcaagggtaagagcattgtgcctttacagcaggtaaaaactctccatgattggttagatggcaagcgcaaggcaagaaaatcttgccgagtagttggggaatcgagaactggcaagacagttgcttgtgatgcctacagatacaggcacaaacctcagcaggaagctggacgaccgccaactgtgcctgtcgtttatattcgacctcaccaaaaatgtggccccaaggatttgtttaaaaagattactgagtacctcaagtatcgggtaacaaaagggactgtatctgattttcgagataggacgatagaagtactcaagggttgtggcgtagagatgctaattattgatgaagctgaccgtctcaagcctgaaacttttgctgatgtgcgagatattgccgaagatttaggaattgctgtggtactggtaggaacagaccgtttggatgcggtaattaagcgggatgagcaggttctcgaacgctttcgggcgcatcttcgctttggtaaattgtcgggagaggattttaagaacaccgtagaaatgtgggaacaaatggttttgaaactgccagtatcttctaatctaaagagcaaggagatgctacggattctcacgtcagcaactgaaggctacattggtcgccttgatgagattcttagggaagctgcaattcgttccttatcaagaggattgaagaagattgacaaggctgttttacaggaagtagctaaggagtacaaatgagctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgacaagctgacgaccgggtctccgcaagtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcggtcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggcatgctagcgcagaaacgtcctagaagatgccaggaggatacttagcagagagacaataaggccggagcgaagccgtttttccataggctccgcccccctgacgaacatcacgaaatctgacgctcaaatcagtggtggcgaaacccgacaggactataaagataccaggcgtttccccctgatggctccctcttgcgctctcctgttcccgtcctgcggcgtccgtgttgtggtggaggctttacccaaatcaccacgtcccgttccgtgtagacagttcgctccaagctgggctgtgtgcaagaaccccccgttcagcccgactgctgcgccttatccggtaactatcatcttgagtccaacccggaaagacacgacaaaacgccactggcagcagccattggtaactgagaattagtggatttagatatcgagagtcttgaagtggtggcctaacagaggctacactgaaaggacagtattt

SEQ ID NO: 41 - DNA, 4277 bp, synthetic construct
  source 1..4277: mol_type = other DNA
ggtatctgcgctccactaaagccagttaccaggttaagcagttccccaactgacttaaccttcgatcaaaccgcctccccaggcggttttttcgtttacagagcaggagattacgacgatcgtaaaaggatctcaagaagatcctttacggattcccgacaccatcactctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattttacactttatgcttccggctcgtatgttgtgtggaattgtgagcggataacaacctgtagaaataattttgtttaactttaataaggagatatacatggactacaaagaccatgacggtgattataaagatcatgacatcgattacaaggatgacgatgacaagggaggaagcggatcgatgacagaagctcaggcgatcgccaagcagttgggtggggtaaaaccggatgatgagtggttacaagctgaaattgctcgtctcaagggtaagagcattgtgcctttacagcaggtaaaaactctccatgattggttagatggcaagcgcaaggcaagaaaatcttgccgagtagttggggaatcgagaactggcaagacagttgcttgtgatgcctacagatacaggcacaaacctcagcaggaagctggacgaccgccaactgtgcctgtcgtttatattcgacctcaccaaaaatgtggccccaaggatttgtttaaaaagattactgagtacctcaagtatcgggtaacaaaagggactgtatctgattttcgagataggacgatagaagtactcaagggttgtggcgtagagatgctaattattgatgaagctgaccgtctcaagcctgaaacttttgctgatgtgcgagatattgccgaagatttaggaattgctgtggtactggtaggaacagaccgtttggatgcggtaattaagcgggatgagcaggttctcgaacgctttcgggcgcatcttcgctttggtaaattgtcgggagaggattttaagaacaccgtagaaatgtgggaacaaatggttttgaaactgccagtatcttctaatctaaagagcaaggagatgctacggattctcacgtcagcaactgaaggctacattggtcgccttgatgagattcttagggaagctgcaattcgttccttatcaagaggattgaagaagattgacaaggctgttttacaggaagtagctaaggagtacaaatgagctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgacaagctgacgaccgggtctccgcaagtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcggtcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggcatgctagcgcagaaacgtcctagaagatgccaggaggatacttagcagagagacaataaggccggagcgaagccgtttttccataggctccgcccccctgacgaacatcacgaaatctgacgctcaaatcagtggtggcgaaacccgacaggactataaagataccaggcgtttccccctgatggctccctcttgcgctctcctgttcccgtcctgcggcgtccgtgttgtggtggaggctttacccaaatcaccacgtcccgttccgtgtagacagttcgctccaagctgggctgtgtgcaagaaccccccgttcagcccgactgctgcgccttatccggtaactatcatcttgagtccaacccggaaagacacgacaaaacgccactggcagcagccattggtaactgagaattagtggatttagatatcgagagtcttgaagtggtggcctaacagaggctacactgaaaggacagtattt

SEQ ID NO: 42 - DNA, 5676 bp, synthetic construct
  source 1..5676: mol_type = other DNA
ggtatctgcgctccactaaagccagttaccaggttaagcagttccccaactgacttaaccttcgatcaaaccgcctccccaggcggttttttcgtttacagagcaggagattacgacgatcgtaaaaggatctcaagaagatcctttacggattcccgacaccatcactctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactataggggaattgtgagcggataacaattcccctgtagaaataattttgtttaactttaataaggagatatacatgagtcaaataactattcaagctcgacttatttcctttgaatcaaaccgccaacaactctggaagttgatggcagatttaaacacgccgttaattaacgaactgctttgccagttaggtcaacaccccgacttcgagaagtggcaacaaaagggtaaactcccgtctaccgttgtgagccagttatgtcaacctctcaaaactgaccctcgctttgcaggtcagcccagccgtttatatatgtcggcaattcatattgtggactacatctacaagtcctggctggctatacagaaacggcttcaacagcagctagatggaaagacgcgctggctagaaatgctcaatagcgatgctgaattagtagaacttagtggtgacactttagaggctattcgtgtcaaagctgctgaaattttggcaatagctatgccagcatctgagtcagatagcgcttcacctaaagggaaaaaaggtaaaaaggagaaaaaaccctcatcttctagccctaagcgtagtttatccaagacattatttgacgcttaccaagaaacggaagatattaagagccgtagcgccatcagctacctgttaaaaaatggctgcaaacttactgacaaagaagaagattcagaaaaatttgctaaacgtcgtcgtcaagttgaaatccaaattcaaaggcttaccgaaaagttaataagtcgcatgcctaaaggtcgagatttgaccaatgctaaatggttagagacactcttgactgctacaaccactgttgctgaagataacgcccaagccaaacgctggcaggatattctgttgactcgatcaagttctctcccattcccccttgtttttgaaaccaacgaggatatggtttggtcaaagaatcaaaagggtaggctgtgtgttcacttcaatggcttgagcgatttaatttttgaggtgtactgcggcaatcgtcaacttcactggtttcaacgcttcctagaagatcaacagactaaacgcaaaagcaaaaatcagcattctagcggcttgttcacactcagaaatggtcatctagtttggcttgaaggtgagggtaaaggggaaccttggaatcttcaccacttgaccctttactgctgtgttgacaatcgcttgtggacagaggagggaacagaaatcgttcgccaagagaaagcagatgaaattactaaattcatcacaaacatgaagaagaaaagcgatctaagcgatacacagcaggctttgattcaacgtaaacaatcaacacttactcgaataaacaattcctttgagcgtcctagccaacccctttatcaaggtcaatcacacattttggttggcgtaagcctgggactagaaaaacctgccacagtagcagtagtagatgcgatcgccaacaaagtcttggcttaccggagtattaaacaattacttggcgacaattacgaactgctaaatcgccagagacgacaacagcagtacctatctcacgaacgccacaaagcacaaaaaaacttctctcccaatcaatttggagcatctgagttagggcaacatatagacagattattagctaaagccattgtagcgttagcgagaacctacaaagctggcagtattgtcttgcccaagttaggggatatgcgggaggttgtccaaagtgaaattcaagctatagcagaacaaaaatttcccggttatattgaaggtcagcaaaaatatgccaaacagtaccgggttaatgttcatcggtggagctacggcagattaattcaaagcattcaaagtaaagcagctcaaacaggaattgtgattgaggagggaaaacaacctattcgaggtagtccccacgacaaagcaaaggaattagcactttctgcttacaatctccgcctaactaggcgaagctaacaaatatctgaaccttgataatagaacattaatagcgccgcaattcatgcttgacagctagctcagtcctaggtataatactagtatattaatagcgccgcaattcatgctgcttgcagcctctgaattttgttaaatgagggttagtttgactgtataaatacagtcttgctttctgaccctggtagctgctcaccctgatgctgctgtcaatagacaggataggtgcgctcccagcaataagggcgcggatgtactgctgtagtggctactgaatcacccccgatcaagggggaaccctccaaaaggtgggttgaaagatgccgatcgcgtcacactacgtgtgaatttttttgttcagaacgctcggtcttgcacaccgggcgttttttctttgtgagtccagctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgacaagctgacgaccgggtctccgcaagtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcggtcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggcatgctagcgcagaaacgtcctagaagatgccaggaggatacttagcagagagacaataaggccggagcgaagccgtttttccataggctccgcccccctgacgaacatcacgaaatctgacgctcaaatcagtggtggcgaaacccgacaggactataaagataccaggcgtttccccctgatggctccctcttgcgctctcctgttcccgtcctgcggcgtccgtgttgtggtggaggctttacccaaatcaccacgtcccgttccgtgtagacagttcgctccaagctgggctgtgtgcaagaaccccccgttcagcccgactgctgcgccttatccggtaactatcatcttgagtccaacccggaaagacacgacaaaacgccactggcagcagccattggtaactgagaattagtggatttagatatcgagagtcttgaagtggtggcctaacagaggctacactgaaaggacagtattt

SEQ ID NO: 43 - DNA, 5106 bp, synthetic construct
  source 1..5106: mol_type = other DNA
ggtatctgcgctccactaaagccagttaccaggttaagcagttccccaactgacttaaccttcgatcaaaccgcctccccaggcggttttttcgtttacagagcaggagattacgacgatcgtaaaaggatctcaagaagatcctttacggattcccgacaccatcactctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactataggggaattgtgagcggataacaattcccctgtagaaataattttgtttaactttaataaggagatatacatgaacagtcagcaaaatcctgatttagctgttcatcccttggcaattcctatggaaggcttactaggagaaagtgctacaactcttgagaagaatgtaattgccacacaactctcagaggaagcccaagtaaagctagaggtaatccaaagtttactggaaccctgcgatcgcacaacttatgggcaaaagttgcgggaagcagcagagaaactaaatgtatcgttgcgaacggtacaaaggttggtgaaaaactgggaacaagatggcttagtcggcctcactcaaacaagtagggctgataaaggaaaacaccgcattggtgagttttgggaaaacttcattaccaaaacctacaaggagggtaacaagggaagtaaacgtatgacccctaaacaagttgctctcagagtcgaggctaaagcccgtgaattaaaagactctaagccgcccaattacaaaaccgtgttacgggtattagcacccattttggaaaagcaacaaaaagccaagagtatccgcagtcctggctggagaggaactacgctttcggttaaaacccgtgaaggaaaagatttatcggttgattacagtaaccatgtttggcaatgtgaccatacccgcgtggatgtgttgctggtagatcaacatggtgaaattttaagtcgtccctggctaacaacagtaattgatacttactctcgttgcattatgggtatcaacttgggctttgatgcacccagttctggggtagtagcattagcgttacgccatgcaattctaccaaagcgttacggttccgagtacaaactgcattgtgagtggggaacctatggaaaaccagaacatttttatactgatggcggtaaagactttcgctctaaccacttgagtcagattggggcgcagttgggatttgtctgtcatttacgcgatcgcccttctgaaggtggagtagtagaacgtcccttcaaaacattaaatgaccaactattttcaacgcttcctgggtacaccggatctaatgtgcaggaacgcccagaagatgcagagaaggacgcaagacttactttgcgagaactagaacagttacttgtgcgttacatcgtagatcgttacaaccaaagtattgatgcgcggatgggcgaccaaacgcgctttgagcgttgggaagcaggattgcctacagtgccagtaccaataccagaacgagatttggatatttgtttaatgaagcagtcacggcgcactgtgcaaagaggtggttgtttgcagtttcagaatttaatgtatcggggggagtatttggcaggttatgccggagaaactgtcaacttgaggtttgaccccagagacattacaacaattttggtttatcgccaggaaaacaatcaggaagtatttctgactcgcgctcacgctcaaggtttggagacagagcaactggcattagatgaggctgaggcagcaagtcgcagactccgtaccgcagggaaaactatcagtaaccaatcattattgcaagaagttgttgaccgcgatgctcttgtcgctaccaagaaaagccgtaaggagcgtcaaaaattggaacagactgttttgcgatctgctgctgttgatgaaagtaatagagaatccttgccttctcaaatagttgaaccagatgaagtggaatctacagaaacggttcactctcaatacgaggacattgaggtgtgggactatgaacaacttcgtgaagaatatgggttttaagctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgacaagctgacgaccgggtctccgcaagtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcggtcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggcatgctagcgcagaaacgtcctagaagatgccaggaggatacttagcagagagacaataaggccggagcgaagccgtttttccataggctccgcccccctgacgaacatcacgaaatctgacgctcaaatcagtggtggcgaaacccgacaggactataaagataccaggcgtttccccctgatggctccctcttgcgctctcctgttcccgtcctgcggcgtccgtgttgtggtggaggctttacccaaatcaccacgtcccgttccgtgtagacagttcgctccaagctgggctgtgtgcaagaaccccccgttcagcccgactgctgcgccttatccggtaactatcatcttgagtccaacccggaaagacacgacaaaacgccactggcagcagccattggtaactgagaattagtggatttagatatcgagagtcttgaagtggtggcctaacagaggctacactgaaaggacagtattt

SEQ ID NO: 44 - DNA, 6493 bp, synthetic construct
  source 1..6493: mol_type = other DNA
tcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgataccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggttcttctcaccatcaccatcaccatggttcttctatggctagcatgtcggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaagattcttgtacgacggtattagaattcaagctgatcagacccctgaagatttggacatggaggataacgatattattgaggctcacagagaacagattggtgggatcgaggaaaacctgtacttccaatccaatgcaacagaggcccaggccattgccaaacagctcggcggagtgaagcccgacgatgaatggctgcaggccgagatcgctagactgaagggcaagagcatcgtgcccctgcagcaagtgaaaaccctgcacgattggctggacggcaagcggaaggccagaaagtcctgtagagtcgtgggcgagagcagaaccggaaagaccgtggcctgtgacgcctaccggtacagacacaagcctcagcaagaagccggcagacctcctacagtgcccgtggtgtatatcagaccccaccagaagtgcggccccaaggacctgttcaagaagatcaccgagtacctgaagtaccgcgtgaccaagggcaccgtgtccgacttcagagacagaaccatcgaggtgctgaaaggctgcggcgtggaaatgctgatcatcgacgaggccgaccggctgaagcctgagacatttgctgacgtgcgggatatcgccgaggacctgggaattgctgtggtgctcgtgggcaccgatagactggatgccgtgatcaagcgggacgaacaggtgctggaacggttcagagcccacctgagattcggcaagctgagcggcgaggacttcaagaacacagtggaaatgtgggagcagatggtgctgaagctgcccgtgtccagcaacctgaagtccaaagagatgctgcggatcctgaccagcgccaccgagggatatatcggcagactggacgagatcctgagagaggccgccatcagaagcctgtccagaggcctgaagaaaatcgacaaggccgtgctgcaagaggtggccaaagagtacaagtaataacattggaagtggataacggatccgaattcgagcgccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaactagtaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatgttttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgc

SEQ ID NO: 45 - DNA, 7410 bp, synthetic construct
  source 1..7410: mol_type = other DNA
ttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggttcttctcaccatcaccatcaccatggttcttctatggctagcatgtcggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaagattcttgtacgacggtattagaattcaagctgatcagacccctgaagatttggacatggaggataacgatattattgaggctcacagagaacagattggtgggatcgaggaaaacctgtacttccaatccaataacagccagcagaaccccgatctggccgtgcatcctctggctatccctatggaaggactgctgggcgagagcgccaccacactggaaaagaacgtgatcgccacacagctgagcgaagaggcccaagtgaagctggaagtgatccagagcctgctggaaccctgcgacagaaccacatacggccagaagctgagagaggccgccgagaagctgaacgtgtccctgagaacagtgcagcggctggtcaagaactgggagcaagatggactcgtgggcctgacacagaccagcagagccgataagggcaagcacagaatcggcgagttctgggagaacttcatcaccaagacctacaaagagggcaacaagggcagcaagcggatgacccctaaacaggtggccctgagagtggaagccaaggccagagaactgaaggacagcaagcctcctaactacaagaccgtgctgagagtgctggcccctatcctggaaaagcagcagaaggccaagagcatcagaagccctggctggcggggaacaaccctgagcgtgaaaaccagagagggcaaagacctgtccgtggactacagcaaccacgtgtggcagtgcgaccacaccagagtggatgtgctgctggtggatcagcacggcgagatcctgtctagaccttggctgaccaccgtgatcgacacctacagcagatgcatcatgggcatcaacctgggcttcgacgccccttctagcggagttgttgctctggctctgcggcacgccatcctgcctaagagatacggcagcgagtacaagctgcactgcgagtggggcacctatggcaagcctgagcacttctacaccgacggcggcaaggacttcagaagcaaccacctgtctcagatcggcgcacagctgggctttgtgtgccacctgagagacagacctagcgaaggcggcgtggtggaaagacccttcaagaccctgaacgaccagctgttcagcaccctgcctggctacacaggcagcaacgtgcaagaaagacccgaggatgccgagaaggacgccagactgacactgagagagctggaacagctgcttgtgcggtacatcgtggaccggtacaaccagagcatcgacgccagaatgggcgatcagaccagattcgagagatgggaggccggactgcctacagtgcctgtgcctattcctgagcgcgacctggacatctgcctgatgaagcagagcagacggacagtgcagagaggcggctgtctgcagttccagaacctgatgtaccggggcgagtacctggctggatatgccggcgagacagtgaacctgagattcgaccccagagacatcaccaccatcctggtgtaccggcaagagaacaatcaagaggtgttcctgaccagggctcacgcccagggactcgaaacagaacaactggccctggatgaagccgaagccgcttctcggagactgagaaccgccggcaagaccatcagcaatcagtccctgctgcaagaggtggtggacagagatgccctggtggccactaagaagtcccggaaagagcggcagaaactggaacagacagtgctgcgctctgccgccgtggacgagagcaatagagagagcctgcctagccagatcgtggaacccgatgaggtggaaagcaccgaaaccgtgcacagccagtacgaggacatcgaagtgtgggactacgagcagctgcgggaagagtacggcttctgaattggaagtggataacggatccgaattcgagcgccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaactagtaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatgttttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgataccctcattcagcatttgcatggtttgttgaaaaccggacatggcactccagtcgcc

SEQ ID NO: 46 - DNA, 6159 bp, synthetic construct
  source 1..6159: mol_type = other DNA
ttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggttcttctcaccatcaccatcaccatggttcttctatggctagcatgtcggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaagattcttgtacgacggtattagaattcaagctgatcagacccctgaagatttggacatggaggataacgatattattgaggctcacagagaacagattggtgggatcgaggaaaacctgtacttccaatccaatatcgaggcccctgacgtgaagccttggctgttcctgatcaagccctacgagggcgagagcctgtctcacttcctgggcagattcagacgggccaatcacctgtctgcctctggcctgggaacactggctggaatcggagccattgtggccagatgggagagattccacttcaaccccagacctagccagcaagagctggaagccattgccagcgtggtggaagtggatgcccagagactggctcagatgcttcctcctgctggcgtgggaatgcagcacgagcctattagactgtgcggcgcctgttacgccgagtctccttgtcacagaatcgagtggcagtacaagtccgtgtggaagtgcgaccggcaccagctgaagatcctggccaagtgtcccaactgtcaggcccctttcaagatgcccgctctgtgggaggatggctgctgccacagatgcagaatgccctttgccgagatggccaagctgcagaaagtgtgaattggaagtggataacggatccgaattcgagcgccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaactagtaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatgttttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgataccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgtt

SEQ ID NO: 47 - DNA, 7578 bp, synthetic construct
  source 1..7578: mol_type = other DNA
ttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggttcttctcaccatcaccatcaccatggttcttctatggctagcatgtcggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaagattcttgtacgacggtattagaattcaagctgatcagacccctgaagatttggacatggaggataacgatattattgaggctcacagagaacagattggtgggatcgaggaaaacctgtacttccaatccaatatgagccagatcaccatccaggccagactgatcagcttcgagagcaacagacagcagctgtggaagctgatggccgacctgaacacccctctgatcaacgagctgctgtgtcagctgggacagcaccccgatttcgagaagtggcagcagaagggcaagctgcctagcaccgtggtgtctcagctgtgccagcctctgaaaacagaccccagatttgccggccagcctagcagactgtacatgagcgccatccacatcgtggactacatctacaagagctggctggccatccagaagcggctgcagcaacagctggatggcaagaccagatggctggaaatgctgaacagcgacgccgagctggtggaactgtctggcgatacactggaagccatcagagtgaaggccgccgagatcctggccattgctatgcctgccagcgagagcgatagcgctagccccaaaggcaaaaagggcaagaaagagaagaagccctccagcagcagccccaagagaagcctgagcaagaccctgttcgacgcctaccaagagacagaggacatcaagagcagaagcgccatctcctacctgctgaagaacggctgcaagctgaccgacaaagaagaggacagcgagaagttcgccaagcggcggagacaggtggaaatccagatccagagactgaccgagaagctgattagcagaatgcccaagggccgcgacctgaccaatgccaagtggctcgaaacactgctgaccgccacaaccacagtggccgaggataatgcccaggccaagagatggcaggacatcctgctgaccagaagcagctccctgccttttccactggtgttcgagacaaacgaggacatggtctggtccaagaaccagaaaggccggctgtgcgtgcacttcaacggcctgagcgatctgatcttcgaggtgtactgcggcaaccggcagctgcactggttccagcggtttctggaagatcagcagaccaagcgcaagagcaagaatcagcacagcagcggcctgttcaccctgagaaatggacacctcgtgtggctggaaggcgaaggcaaaggcgagccttggaacctgcaccacctgacactgtactgctgcgtggacaaccggctgtggacagaagagggcacagagatcgtgcggcaagagaaggccgatgagatcaccaagttcatcaccaacatgaagaagaagtccgacctgagcgacacccagcaggccctgattcagagaaagcagagcaccctgaccaggatcaacaactccttcgagaggcccagccagccactgtatcagggccagtctcatatcctcgtgggcgtgtcactgggcctcgagaaacctgctacagtcgccgtggtggacgccattgccaacaaggttctggcctaccggtccatcaaacagctgctcggcgacaactatgagctgctgaaccgtcagcgacggcagcagcagtatctgagccacgagagacacaaggcccagaagaacttcagccccaaccagtttggcgcctctgaactgggccagcacatcgatagactgctggccaaggccattgtggccctggccagaacatacaaggccggcagtatcgtgctgcccaagctgggcgatatgagagaagtggtgcagagcgagatccaggctatcgccgaacagaagttccccggctatatcgagggccagcagaagtatgccaagcagtatagagtgaacgtgcaccggtggtcctacggcaggctgatccagagcatccagtctaaggctgcccagaccggcatcgtgatcgaggaaggcaagcagcccattagaggcagccctcacgacaaggctaaagaactggctctgagcgcctacaacctgaggctgacaagaagaagctgaattggaagtggataacggatccgaattcgagcgccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaactagtaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatgttttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgataccctcattcagcatttgcatggtttgttgaaaaccggacatggcactccagtcgcc

SEQ ID NO: 48 - DNA, 7216 bp, synthetic construct
  source 1..7216: mol_type = other DNA
tcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgataccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggttcttctcaccatcaccatcaccatggttcttctatggctagcatgtcggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaagattcttgtacgacggtattagaattcaagctgatcagacccctgaagatttggacatggaggataacgatattattgaggctcacagagaacagattggtgggatcgaggaaaacctgtacttccaatccaatgcaatggtgagcaagggcgaggaggataacatggcctctctcccagcgacacatgagttacacatctttggctccatcaacggtgtggactttgacatggtgggtcagggcaccggcaatccaaatgatggttatgaggagttaaacctgaagtccaccaagggtgacctccagttctccccctggattctggtccctcatatcgggtatggcttccatcagtacctgccctaccctgacgggatgtcgcctttccaggccgccatggtagatggctccggctaccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagcgctaccggagcactgcgcggaccacctacacctttgccaagccaatggcggctaactatctgaagaaccagccgatgtacgtgttccgtaagacggagctcaagcactccaagaccgagctcaacttcaaggagtggcaaaaggcctttaccgatgtgatgggcatggacgagctgtacaagggaggaagcggatcgacagaggcccaggccattgccaaacagctcggcggagtgaagcccgacgatgaatggctgcaggccgagatcgctagactgaagggcaagagcatcgtgcccctgcagcaagtgaaaaccctgcacgattggctggacggcaagcggaaggccagaaagtcctgtagagtcgtgggcgagagcagaaccggaaagaccgtggcctgtgacgcctaccggtacagacacaagcctcagcaagaagccggcagacctcctacagtgcccgtggtgtatatcagaccccaccagaagtgcggccccaaggacctgttcaagaagatcaccgagtacctgaagtaccgcgtgaccaagggcaccgtgtccgacttcagagacagaaccatcgaggtgctgaaaggctgcggcgtggaaatgctgatcatcgacgaggccgaccggctgaagcctgagacatttgctgacgtgcgggatatcgccgaggacctgggaattgctgtggtgctcgtgggcaccgatagactggatgccgtgatcaagcgggacgaacaggtgctggaacggttcagagcccacctgagattcggcaagctgagcggcgaggacttcaagaacacagtggaaatgtgggagcagatggtgctgaagctgcccgtgtccagcaacctgaagtccaaagagatgctgcggatcctgaccagcgccaccgagggatatatcggcagactggacgagatcctgagagaggccgccatcagaagcctgtccagaggcctgaagaaaatcgacaaggccgtgctgcaagaggtggccaaagagtacaagtaataacattggaagtggataacggatccgaattcgagcgccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaactagtaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatgttttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgc

SEQ ID NO: 49 - DNA, 5928 bp, synthetic construct
  source 1..5928: mol_type = other DNA
ttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggttcttctcaccatcaccatcaccatggttcttctatggctagcatgtcggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaagattcttgtacgacggtattagaattcaagctgatcagacccctgaagatttggacatggaggataacgatattattgaggctcacagagaacagattggtgggatcgaggaaaacctgtacttccaatccaatatggcacttactcaggagaggaaacaagaaatcatcgtgaactaccaagtgcacgagacggataccggtagcgcagatgttcaggttgccatgctgaccgaacgtattaaccgcttgtctctgcatctgcaggctaataaaaaggaccactccagccgcagaggcctgctcaagctgattggccagcgtaagcgcttattggcgtatatccaaaaagacagccgtgagaaataccaagcgctgatcggccgtcttggtattcgtggttagattggaagtggataacggatccgaattcgagcgccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaactagtaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatgttttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgataccctcattcagcatttgcatggtttgttgaaaaccggacatggcactccagtcgcc

SEQ ID NO: 50 - protein, 641 aa, Scytonema hofmannii
SNMSQITIQARLISFESNRQQLWKLMADLNTPLINELLCQLGQHPDFEKWQQKGKLPSTVVSQLCQPLKTDPRFAGQPSRLYMSAIHIVDYIYKSWLAIQKRLQQQLDGKTRWLEMLNSDAELVELSGDTLEAIRVKAAEILAIAMPASESDSASPKGKKGKKEKKPSSSSPKRSLSKTLFDAYQETEDIKSRSAISYLLKNGCKLTDKEEDSEKFAKRRRQVEIQIQRLTEKLISRMPKGRDLTNAKWLETLLTATTTVAEDNAQAKRWQDILLTRSSSLPFPLVFETNEDMVWSKNQKGRLCVHFNGLSDLIFEVYCGNRQLHWFQRFLEDQQTKRKSKNQHSSGLFTLRNGHLVWLEGEGKGEPWNLHHLTLYCCVDNRLWTEEGTEIVRQEKADEITKFITNMKKKSDLSDTQQALIQRKQSTLTRINNSFERPSQPLYQGQSHILVGVSLGLEKPATVAVVDAIANKVLAYRSIKQLLGDNYELLNRQRRQQQYLSHERHKAQKNFSPNQFGASELGQHIDRLLAKAIVALARTYKAGSIVLPKLGDMREVVQSEIQAIAEQKFPGYIEGQQKYAKQYRVNVHRWSYGRLIQSIQSKAAQTGIVIEEGKQPIRGSPHDKAKELALSAYNLRLTRRS

SEQ ID NO: 51 - protein, 585 aa, Scytonema hofmannii
SNNSQQNPDLAVHPLAIPMEGLLGESATTLEKNVIATQLSEEAQVKLEVIQSLLEPCDRTTYGQKLREAAEKLNVSLRTVQRLVKNWEQDGLVGLTQTSRADKGKHRIGEFWENFITKTYKEGNKGSKRMTPKQVALRVEAKARELKDSKPPNYKTVLRVLAPILEKQQKAKSIRSPGWRGTTLSVKTREGKDLSVDYSNHVWQCDHTRVDVLLVDQHGEILSRPWLTTVIDTYSRCIMGINLGFDAPSSGVVALALRHAILPKRYGSEYKLHCEWGTYGKPEHFYTDGGKDFRSNHLSQIGAQLGFVCHLRDRPSEGGVVERPFKTLNDQLFSTLPGYTGSNVQERPEDAEKDARLTLRELEQLLVRYIVDRYNQSIDARMGDQTRFERWEAGLPTVPVPIPERDLDICLMKQSRRTVQRGGCLQFQNLMYRGEYLAGYAGETVNLRFDPRDITTILVYRQENNQEVFLTRAHAQGLETEQLALDEAEAASRRLRTAGKTISNQSLLQEVVDRDALVATKKSRKERQKLEQTVLRSAAVDESNRESLPSQIVEPDEVESTETVHSQYEDIEVWDYEQLREEYGF

SEQ ID NO: 52 - protein, 278 aa, Scytonema hofmannii
SNATEAQAIAKQLGGVKPDDEWLQAEIARLKGKSIVPLQQVKTLHDWLDGKRKARKSCRVVGESRTGKTVACDAYRYRHKPQQEAGRPPTVPVVYIRPHQKCGPKDLFKKITEYLKYRVTKGTVSDFRDRTIEVLKGCGVEMLIIDEADRLKPETFADVRDIAEDLGIAVVLVGTDRLDAVIKRDEQVLERFRAHLRFGKLSGEDFKNTVEMWEQMVLKLPVSSNLKSKEMLRILTSATEGYIGRLDEILREAAIRSLSRGLKKIDKAVLQEVAKEYK

SEQ ID NO: 53 - protein, 168 aa, Scytonema hofmannii
SNIEAPDVKPWLFLIKPYEGESLSHFLGRFRRANHLSASGLGTLAGIGAIVARWERFHFNPRPSQQELEAIASVVEVDAQRLAQMLPPAGVGMQHEPIRLCGACYAESPCHRIEWQYKSVWKCDRHQLKILAKCPNCQAPFKMPALWEDGCCHRCRMPFAEMAKLQKV

SEQ ID NO: 54 - protein, 91 aa, Scytonema hofmannii
SNMALTQERKQEIIVNYQVHETDTGSADVQVAMLTERINRLSLHLQANKKDHSSRRGLLKLIGQRKRLLAYIQKDSREKYQALIGRLGIRG

SEQ ID NO: 55 - protein, 519 aa, Scytonema hofmannii
SNAMVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKGGSGSTEAQAIAKQLGGVKPDDEWLQAEIARLKGKSIVPLQQVKTLHDWLDGKRKARKSCRVVGESRTGKTVACDAYRYRHKPQQEAGRPPTVPVVYIRPHQKCGPKDLFKKITEYLKYRVTKGTVSDFRDRTIEVLKGCGVEMLIIDEADRLKPETFADVRDIAEDLGIAVVLVGTDRLDAVIKRDEQVLERFRAHLRFGKLSGEDFKNTVEMWEQMVLKLPVSSNLKSKEMLRILTSATEGYIGRLDEILREAAIRSLSRGLKKIDKAVLQEVAKEYK

SEQ ID NO: 56 - DNA, 141 bp, synthetic construct
  source 1..141: mol_type = other DNA
gtcacaatgacattaatctgtcaccgacgacagataatttgtcactgtacagtagaatatagatgcgcatctatatagatgcaaattgagtggccttattaaatgacttctcaaccagtcagcacgcccagaccagggcac

SEQ ID NO: 57 - DNA, 71 bp, synthetic construct
  source 1..71: mol_type = other DNA
gcgtgctgactggttctcttcagtaaattattggccactcaatttgcatctatatagatgcgcatctatat

SEQ ID NO: 58 - DNA, 51 bp, synthetic construct
  source 1..51: mol_type = other DNA
tgtacagtgacaaattatctgtcgtcggtgacagattaatgtcattgtgac

SEQ ID NO: 59 - DNA, 75 bp, synthetic construct
  source 1..75: mol_type = other DNA
ttactgatgacaataatttgtcacaacgacatataattagtcactgtacatctacgatacgtagcggccgacgcg

SEQ ID NO: 60 - DNA, 50 bp, synthetic construct
  source 1..50: mol_type = other DNA
tgtacagtgactaattatatgtcgttgtgacaaattattgtcatcagtaa

SEQ ID NO: 61 - DNA, 20 bp, synthetic construct
  source 1..20: mol_type = other DNA
cgcgtcggccgctacgtatc

SEQ ID NO: 62 - DNA, 55 bp, synthetic construct
  source 1..55: mol_type = other DNA
accaacgtgacctatcccattacggtcaatccgccgtttgttcccacggagaatc

SEQ ID NO: 63 - DNA, 55 bp, synthetic construct
  source 1..55: mol_type = other DNA
gattctccgtgggaacaaacggcggattgaccgtaatgggataggtcacgttggt

SEQ ID NO: 64 - DNA, 55 bp, synthetic construct
  source 1..55: mol_type = other DNA
gaggagggctgtttttacaaaatccggtagtaacttgctaaccaattcctaggca

SEQ ID NO: 65 - DNA, 55 bp, synthetic construct
  source 1..55: mol_type = other DNA
tgcctaggaattggttagcaagttactaccggattttgtaaaaacagccctcctc

SEQ ID NO: 66 - DNA, 280 bp, synthetic construct
  source 1..280: mol_type = other DNA
gcgtaatacgactcactataggatattaatagcgccgcaattcatgctgcttgcagcctctgaattttgttaaatgagggttagtttgactgtataaatacagtcttgctttctgaccctggtagctgctcaccctgatgctgctgtcaatagacaggataggtgcgctcccagcaataagggcgcggatgtactgctgtagtggctactgaatcacccccgatcaagggggaaccctccaaaaggtgggttgaaagagcaagttactaccggattttgt

SEQ ID NO: 67 - DNA, 280 bp, synthetic construct
  source 1..280: mol_type = other DNA
acaaaatccggtagtaacttgctctttcaacccaccttttggagggttcccccttgatcgggggtgattcagtagccactacagcagtacatccgcgcccttattgctgggagcgcacctatcctgtctattgacagcagcatcagggtgagcagctaccagggtcagaaagcaagactgtatttatacagtcaaactaaccctcatttaacaaaattcagaggctgcaagcagcatgaattgcggcgctattaatatcctatagtgagtcgtattacgc

SEQ ID NO: 68 - DNA, 32 bp, synthetic construct
  source 1..32: mol_type = other DNA
gtataataattgcagagcatattatattgatg

SEQ ID NO: 69 - DNA, 28 bp, synthetic construct
  source 1..28: mol_type = other DNA
gataacaatttggttagcaagttactac

SEQ ID NO: 70 - DNA, 23 bp, synthetic construct
  source 1..23: mol_type = other DNA
caattaattaagcaacgctgatg

SEQ ID NO: 71 - DNA, 20 bp, synthetic construct
  source 1..20: mol_type = other DNA
cgatgagcgtggtggttatg

SEQ ID NO: 72 - DNA, 21 bp, synthetic construct
  source 1..21: mol_type = other DNA
gtaatgacgatttccgcactg

SEQ ID NO: 73 - DNA, 23 bp, synthetic construct
  source 1..23: mol_type = other DNA
cacctcttttaacccttgaagtc

SEQ ID NO: 74 - DNA, 23 bp, synthetic construct
  source 1..23: mol_type = other DNA
ggtgttggcctgatgagttatag

SEQ ID NO: 75 - DNA, 18 bp, synthetic construct
  source 1..18: mol_type = other DNA
cagcgatttccatgttgc

SEQ ID NO: 76 - DNA, 30 bp, synthetic construct
  source 1..30: mol_type = other DNA
cattgtgactatttaattgtcgtcgtgacc

SEQ ID NO: 77 - DNA, 21 bp, synthetic construct
  source 1..21: mol_type = other DNA
catgcagtattccaggactca

SEQ ID NO: 78 - DNA, 21 bp, synthetic construct
  source 1..21: mol_type = other DNA
aaggagagcaaatcttgttgc

SEQ ID NO: 79 - DNA, 30 bp, synthetic construct
  source 1..30: mol_type = other DNA
cagtcgttaacccaattcctatttccctca

SEQ ID NO: 80 - DNA, 23 bp, synthetic construct
  source 1..23: mol_type = other DNA
agcaagttactaccggattttgt

SEQ ID NO: 81 - DNA, 21 bp, synthetic construct
  source 1..21: mol_type = other DNA
tacggatggcatgacagtaag

SEQ ID NO: 82 - DNA, 21 bp, synthetic construct
  source 1..21: mol_type = other DNA
tcaaggcgagttacatgatcc

SEQ ID NO: 83 - DNA, 54 bp, synthetic construct
  source 1..54: mol_type = other DNA
ctggagttcagacgtgtgctcttccgatctcaatttggttagcaagttactacc

SEQ ID NO: 84 - DNA, 50 bp, synthetic construct
  source 1..50: mol_type = other DNA
ctctttccctacacgacgctcttccgatctgacattaatctgtcaccgac

SEQ ID NO: 85 - DNA, 59 bp, synthetic construct
  source 1..59: mol_type = other DNA
aatgatacggcgaccaccgagatctacacnnnnnnnnacactctttccctacacgacgc

SEQ ID NO: 86 - DNA, 55 bp, synthetic construct
  source 1..55: mol_type = other DNA
caagcagaagacggcatacgagatnnnnnnnngtgactggagttcagacgtgtgc

SEQ ID NO: 87 - DNA, 53 bp, synthetic construct
  source 1..53: mol_type = other DNA
ctctttccctacacgacgctcttccgatctgacattaatctgtcaccgacgac

SEQ ID NO: 88 - DNA, 54 bp, synthetic construct
  source 1..54: mol_type = other DNA
ctctttccctacacgacgctcttccgatctagacattaatctgtcaccgacgac

SEQ ID NO: 89 - DNA, 55 bp, synthetic construct
  source 1..55: mol_type = other DNA
ctctttccctacacgacgctcttccgatctatgacattaatctgtcaccgacgac

SEQ ID NO: 90 - DNA, 17 bp, synthetic construct
  source 1..17: mol_type = other DNA
tagtctcgtgggctcgg

SEQ ID NO: 91 - DNA, 47 bp, synthetic construct
  source 1..47: mol_type = other DNA
caagcagaagacggcatacgagatnnnnnnnngtctcgtgggctcgg

SEQ ID NO: 92 - RNA, 258 nt, synthetic construct
  source 1..258: mol_type = other RNA
atattaatagcgccgcaattcatgctgcttgcagcctctgaattttgttaaatgagggttagtttgactgtataaatacagtcttgctttctgaccctggtagctgctcaccctgatgctgctgtcaatagacaggataggtgcgctcccagcaataagggcgcggatgtactgctgtagtggctactgaatcacccccgatcaagggggaaccctccaaaaggtgggttgaaagcggtcttctcgacgagaagacac

SEQ ID NO: 93 - RNA, 258 nt, synthetic construct
  source 1..258: mol_type = other RNA
atattaatagcgccgcaattcatgctgcttgcagcctctgaattttgttaaatgagggttagtttgactgtataaatacagtcttgctttctgaccctggtagctgctcaccctgatgctgctgtcaatagacaggataggtgcgctcccagcaataagggcgcggatgtactgctgtagtggctactgaatcacccccgatcaagggggaaccctccaaaaggtgggttgaaagatgccgatcgcgtcacactacgt

SEQ ID NO: 94 - RNA, 258 nt, synthetic construct
  source 1..258: mol_type = other RNA
atattaatagcgccgcaattcatgctgcttgcagcctctgaattttgttaaatgagggttagtttgactgtataaatacagtcttgctttctgaccctggtagctgctcaccctgatgctgctgtcaatagacaggataggtgcgctcccagcaataagggcgcggatgtactgctgtagtggctactgaatcacccccgatcaagggggaaccctccaaaaggtgggttgaaagttgtcagatattacgcctgtgtg

SEQ ID NO: 95 - RNA, 258 nt, synthetic construct
  source 1..258: mol_type = other RNA
atattaatagcgccgcaattcatgctgcttgcagcctctgaattttgttaaatgagggttagtttgactgtataaatacagtcttgctttctgaccctggtagctgctcaccctgatgctgctgtcaatagacaggataggtgcgctcccagcaataagggcgcggatgtactgctgtagtggctactgaatcacccccgatcaagggggaaccctccaaaaggtgggttgaaagactgcccgtttcgagagtttctc

SEQ ID NO: 96 - RNA, 258 nt, synthetic construct
  source 1..258: mol_type = other RNA
atattaatagcgccgcaattcatgctgcttgcagcctctgaattttgttaaatgagggttagtttgactgtataaatacagtcttgctttctgaccctggtagctgctcaccctgatgctgctgtcaatagacaggataggtgcgctcccagcaataagggcgcggatgtactgctgtagtggctactgaatcacccccgatcaagggggaaccctccaaaaggtgggttgaaagatagcgatcccttgctgaaaata

SEQ ID NO: 97 - RNA, 258 nt, synthetic construct
  source 1..258: mol_type = other RNA
atattaatagcgccgcaattcatgctgcttgcagcctctgaattttgttaaatgagggttagtttgactgtataaatacagtcttgctttctgaccctggtagctgctcaccctgatgctgctgtcaatagacaggataggtgcgctcccagcaataagggcgcggatgtactgctgtagtggctactgaatcacccccgatcaagggggaaccctccaaaaggtgggttgaaaggccactcgctttaatgatgattt

SEQ ID NO: 98 - RNA, 260 nt, synthetic construct
  source 1..260: mol_type = other RNA
ggatattaatagcgccgcaattcatgctgcttgcagcctctgaattttgttaaatgagggttagtttgactgtataaatacagtcttgctttctgaccctggtagctgctcaccctgatgctgctgtcaatagacaggataggtgcgctcccagcaataagggcgcggatgtactgctgtagtggctactgaatcacccccgatcaagggggaaccctccaaaaggtgggttgaaagagcaagttactaccggattttgt

SEQ ID NO: 99 - RNA, 372 nt, synthetic construct
  source 1..372: mol_type = other RNA
gtactaatagcgccgcagttcatgctctttaagagtctctgtactgtggaaaatctgggttagtttgacggttggaaaaccgttttgctttctgaccctggtagctgcccgcttctcatgctctgacttttcacgttatgtggaaaaagtaacgtaatttcgttagttaagacttaccgtaaaaagtcagttctgatgctgctgtcgcaagacaggataggtgcgctcccagcaaaaggagtatgtcttgaaaaagactagccgttctagtaacggtgcggattaccgcagtggtggctactgaatcacccccttcgtcgggggaaccctccaaaaggtgggttgaaagcagcgcggctgaaatcatcatta

SEQ ID NO: 100 - RNA, 96 nt, synthetic construct
  source 1..96: mol_type = other RNA
tgatttcagccgcgctgtacgttttagagctagaaatagcaagttaaaataaggctagtccgttatcaacttgaaaaagtggcaccgagtcggtgc

SEQ ID NO: 101 - RNA, 23 nt, synthetic construct
  source 1..23: mol_type = other RNA
cggtcttctcgacgagaagacac

SEQ ID NO: 102 - RNA, 23 nt, synthetic construct
  source 1..23: mol_type = other RNA
atgccgatcgcgtcacactacgt

SEQ ID NO: 103 - RNA, 23 nt, synthetic construct
  source 1..23: mol_type = other RNA
ttgtcagatattacgcctgtgtg

SEQ ID NO: 104 - RNA, 23 nt, synthetic construct
  source 1..23: mol_type = other RNA
actgcccgtttcgagagtttctc

SEQ ID NO: 105 - RNA, 23 nt, synthetic construct
  source 1..23: mol_type = other RNA
atagcgatcccttgctgaaaata

SEQ ID NO: 106 - RNA, 23 nt, synthetic construct
  source 1..23: mol_type = other RNA
gccactcgctttaatgatgattt

SEQ ID NO: 107 - RNA, 23 nt, synthetic construct
  source 1..23: mol_type = other RNA
agcaagttactaccggattttgt

SEQ ID NO: 108 - RNA, 23 nt, synthetic construct
  source 1..23: mol_type = other RNA
cagcgcggctgaaatcatcatta

SEQ ID NO: 109 - RNA, 20 nt, synthetic construct
  source 1..20: mol_type = other RNA
tgatttcagccgcgctgtac

SEQ ID NO: 110 - DNA, 23 bp, synthetic construct
  source 1..23: mol_type = other DNA
gacagataatttgtcactgtaca

SEQ ID NO: 111 - protein, 10 aa, synthetic construct
PKKKRKVEDP

SEQ ID NO: 112 - protein, 9 aa, synthetic construct
PAAKRVKLD

SEQ ID NO: 113 - protein, 16 aa, synthetic construct
KRPAATKKAGQAKKKK

SEQ ID NO: 114 - protein, 25 aa, synthetic construct
MSRRRKANPTKLSENAKKLAKEVEN

SEQ ID NO: 115 - protein, 18 aa, synthetic construct
KRTADGSEFESPKKKRKV

(19) United States
(12) Patent Application Publication
(10) Pub. No.: US 2026/0139278 A1
(43) Pub. Date: May 21, 2026

(54) SPECIFICITY OF CRISPR-TRANSPOSON SYSTEMS IN DNA MODIFICATION

(71) Applicant: The Trustees of Columbia University in the City of New York, New York, NY (US)

(72) Inventors: Samuel Henry Sternberg, New York, NY (US); Jerrin Thomas George, New York, NY (US)

(21) Appl. No.: 19/447,798

(22) Filed: Jan. 13, 2026

Related U.S. Application Data
(63) Continuation of application No. PCT/US2024/037837, filed on Jul. 12, 2024.
(60) Provisional application No. 63/513,496, filed on Jul. 13, 2023.

(51) Int. Cl. C12N 15/90; C12N 9/22; C12N 15/11

(52) U.S. Cl. CPC C12N 15/907; C12N 9/226; C12N 15/11; C12N 2310/20; C12N 2800/90

(57) ABSTRACT

The present disclosure relates to methods and systems for improved specificity and/or efficiency of Clustered Regularly Interspaced Short Palindromic Repeats (CRISPR)-associated transposon (CAST) system. In particular, the present disclosure provides systems and methods for increasing specificity and efficiency of CAST system by: modulating TnsC function and abundance; modulating TnsB function and abundance; and influencing CAST target preference.

### STATEMENT REGARDING FEDERALLY SPONSORED RESEARCH OR DEVELOPMENT

This invention was made with government support under grant numbers HG011650, AI168976, and EB031935 awarded by the National Institutes of Health. The government has certain rights in the invention.

### FIELD

The present disclosure relates to methods and systems for improved specificity and/or efficiency of Clustered Regularly Interspaced Short Palindromic Repeats (CRISPR)-associated transposon (CAST) system.

### SEQUENCE LISTING STATEMENT

The content of the electronic sequence listing titled COLUM_42218_601_SequenceListing.xml (Size: 411,690 bytes; and Date of Creation: Jul. 9, 2024) is herein incorporated by reference in its entirety.

### CROSS-REFERENCE TO RELATED APPLICATIONS

This application is a continuation of PCT International Application No. PCT/US2024/037837, filed Jul. 12, 2024, which claims the benefit of U.S. Provisional Application No. 63/513,496, filed Jul. 13, 2023, the contents of which are herein incorporated by reference in its entirety.

### BACKGROUND

CRISPR-Cas systems can be used for programmable DNA integration, in which the nuclease-deficient CRISPR-Cas machinery (either Cascade from Type I systems, or Cas12 from Type V systems) coordinates with Tn7 transposon-associated proteins to mediate RNA-guided DNA targeting and DNA integration, respectively. This activity may be leveraged in bacterial or eukaryotic cells for the targeted integration of user-defined genetic payloads at user-defined genomic loci, via a mechanism that obviates requirements for DNA double-strand breaks (DSBs) necessary for homology-directed repair.

### SUMMARY

Disclosed herein are systems, methods, and kits for enhancing the specificity of Clustered Regularly Interspaced Short Palindromic Repeats (CRISPR)-associated transposon (CAST) systems. In some embodiments, the system, methods, and kits comprise TnsC with modulated function and abundance, TnsB with modulated function and abundance, and/or modified CAST target preference.

In some embodiments, the systems comprise a CAST system or one or more nucleic acids encoding the engineered CAST system, wherein the CAST system comprises: one or more Cas proteins; one or more transposon associated proteins comprising at least TnsC; and optionally, at least one gRNA complementary to at least a portion of the target nucleic acid sequence.

In some embodiments, the systems comprise less TnsC, or a nucleic acid encoding TnsC, compared to any or all of the remaining components of the CAST system or nucleic acids encoding thereof. In some embodiments, TnsC is encoded by an individual nucleic acid under a promoter with lower copy number compared to the promoter utilized for expressing any or all of remaining components of the CAST system. In some embodiments, TnsC comprises: one or more mutations to decrease affinity for DNA binding at non-specific sites and/or increase affinity for target site DNA binding; one or more mutations to disrupt the formation of TnsC filaments; and/or an N-terminal moiety.

In some embodiments, the CAST system is derived from a Type I CRISPR-Cas system or a Type V CRISPR-Cas system. In some embodiments, the CAST system is derived from a type V-K, type I-F, type I-B, or type I-D CRISPR-Cas system.

In some embodiments, the one or more Cas proteins comprise one or more of Cas12, Cas5, Cas6, Cas7, and Cas8.

In some embodiments, the one or more transposon associated proteins further comprises one or both of TnsB and TniQ.

Also disclosed herein are methods for modifying a target nucleic acid comprising contacting a target nucleic acid sequence with the systems disclosed herein. In some embodiments, the methods results in less off-target modification than a method using a non-engineered CAST system.

Further disclosed herein are methods method for enhancing the specificity of CRISPR-associated transposon (CAST) system. In some embodiments, the methods comprise decreasing levels of TnsC in the CAST system, perturbing TnsC N-terminus, and/or modulating the affinity of TnsC for DNA binding. In some embodiments, the CAST system comprises: one or more Cas proteins; one or more transposon associated proteins comprising at least TnsC; and optionally, at least one gRNA complementary to at least a portion of the target nucleic acid sequence, or one or more nucleic acid encoding the one or more Cas proteins, one or more transposon associated proteins, and the at least one gRNA.

In some embodiments, the CAST system is derived from a Type I CRISPR-Cas system or a Type V CRISPR-Cas system. In some embodiments, the CAST system is derived from a type V-K, type I-F, type I-B, or type I-D CRISPR-Cas system.

In some embodiments, the one or more Cas proteins comprise one or more of: Cas12, Cas5, Cas6, Cas7, and Cas8.

In some embodiments, the one or more transposon associated proteins further comprises one or both of TnsB and TniQ.

In some embodiments, modulating the affinity of TnsC for DNA binding comprises introducing mutations in TnsC to decrease TnsC affinity for DNA binding at non-specific sites and/or increase affinity for target site DNA binding. In some embodiments, modulating the affinity of TnsC for DNA binding comprises introducing mutations in TnsC to disrupt the formation of TnsC filaments. In some embodiments, modulating the affinity of TnsC for DNA binding comprises adding an agent to disrupt or control formation of TnsC filaments.

In some embodiments, modulating the affinity of TnsC for DNA binding comprises perturbing TnsC N-terminus comprises fusing a moiety to the TnsC N-terminus or introducing to the CAST system an N-terminal TnsC fusion protein. In some embodiments, modulating the affinity of TnsC for DNA binding comprises the moiety comprises an effector domain, one of the one or more Cas proteins or other components of a CAST system, or an exogenous protein or protein domain.

In some embodiments, modulating the affinity of TnsC for DNA binding comprises the CAST system is in a cell and decreasing the levels of TnsC in the CAST system comprises decreasing TnsC expression levels in the cell. In some embodiments, modulating the affinity of TnsC for DNA binding comprises decreasing TnsC expression levels comprises expressing TnsC from a lower copy number promoter.

Additionally disclosed are methods for RNA-guided tagmentation of DNA. In some embodiments, the methods comprise at least one or all of: conducting RNA-guided site specific transposition of a sample to integrate a donor nucleic acid into nucleic acids in the sample; incubating purified nucleic acids from the sample with a tagmentation reaction mixture to generate tagmented nucleic acid fragments; amplifying the tagmented nucleic acid fragments with transposon end specific primers and adaptor-containing primers to generate a library of amplicons; sequencing the library of amplicons; and analyzing sequences of the amplicons for on-target integration, off-target integration, and orientation of the integration.

In some embodiments, the methods further comprise removing non-integrated donor nucleic acids. In some embodiments, the removing is completed prior to generation of tagmented nucleic acids. In some embodiments, the polynucleotide comprising the donor nucleic acid comprises a digestion site outside of the donor nucleic acid and transposon end sequences. In some embodiments, removing comprises digesting the nucleic acids from the sample with an enzyme directed to the digestion site.

In some embodiments, the RNA-guided site specific transposition is in vitro. In some embodiments, the RNA-guided site specific transposition is carried out using high amounts of TnsB and low amounts of TnsC.

In some embodiments, the analyzing further comprises normalizing quantitation of on-target integration and off-target integration with the total transposon-end containing reads. In some embodiments, the methods further comprise analyzing the genetic neighborhood for off-target integrations.

Other aspects and embodiments of the disclosure will be apparent in light of the following detailed description and accompanying figures.

### BRIEF DESCRIPTION OF THE DRAWINGS

FIGS. 1A-1G show type V-K CASTs direct frequent Cas12k- and RNA-independent transposition events. FIG. 1A is a schematic of type V-K CAST transposition occurring at on-target sites (RNA-dependent) and untargeted sites (RNA-independent). FIG. 1B is an experimental pipeline used for tagmentation-based transposon insertion sequencing (TagTn-seq) for in vitro and genomic samples. FIG. 1C shows the fraction of total genome-mapping integration reads detected at on-target and untargeted sites for the wildtype pHelper expression plasmid across multiple guides (sgRNA-1 to sgRNA-5) plotted above on-target transposition efficiencies for the same sgRNAs as measured by Taqman qPCR. FIG. 1D shows total genome-mapping reads detected for WT pHelper or pHelper with the indicated deletions, normalized and scaled. FIG. 1E is a zoomed-in view of integration reads comprising 1% or less of E. coli genome-mapping reads, in an experiment performed without the Cas12k and guide RNA. FIG. 1F is cryo-EM reconstruction of the untargeted transpososome reveals the assembly of TniQ (orange), TnsC (green), and TnsB (purple) in a strand-transfer complex (STC). The target DNA and transposon DNA are represented in light blue and dark blue, respectively. For visualization, a composite map was generated using two local-resolution filtered reconstructions from the focused refinements. Zoomed-in and cutaway view showing TnsC forming a helical assembly on the target DNA, positioning residues K103 and T121 (pink) adjacent to one strand of the target DNA (dark blue). 5′ and 3′ ends of the TnsC-interacting DNA strand are indicated. Two turns of TnsC and TnsB footprint on DNA until TSD cover approximately 25 and 13 base pairs (bp), respectively. Only selected TnsC monomers are represented in the cutaway for clarity. Cas12k and the sgRNA were cloned onto a separate vector, and the promoter driving Cas12k expression was varied. FIG. 1G shows reads detected at on-target and untargeted sites during transposition assays were normalized and scaled. For FIGS. 1C-1E and 1G, the mean is shown from n=2 independent biological replicates.

FIGS. 2A-2G show biochemical reconstitution of transposition reveals distinct efficiencies at on-target and untargeted sites. FIG. 2A is growth curves upon induction of WT or mutant TnsC, with or without TnsB. The data shown are mean±s.d. for n=2 independent biological replicates, inoculated from individual colonies. FIG. 2B is an assay schematic for probing in vitro plasmid-to-plasmid transposition events using recombinantly expressed CAST components. FIG. 2C shows in vitro integration reads mapping to pTarget, from experiments in which TnsC was titrated from 0.1-2 μM. Data were normalized and scaled to highlight untargeted integration events, relative to on-target insertions (Methods). FIG. 2D shows on-target specificity from biochemical transposition assays at varying TnsC concentrations, calculated as the fraction of on-target reads divided by total plasmid-mapping reads (bottom). Total integration activity also decreased as a function of TnsC concentration, as seen by the normalized plasmid-mapping reads (top). FIG. 2E is a scatter plot showing reproducibility between untargeted integration reads observed in vitro at two high TnsC concentrations; each data point represents transposition events mapping to a single-bp position within pTarget. The Pearson linear correlation coefficient is shown (two tailed P<0.0001); on-target events were masked. FIG. 2F is normalized integration reads detected at a representative untargeted site (left) and at the on-target site (right), with 1 μM TnsC and the indicated TnsB concentration. Note the differing y-axis ranges. FIG. 2G shows on-target specificity from biochemical transposition assays at 1 μM TnsC and the indicated TnsB concentration, shown as in FIG. 2D.

FIGS. 3A-3J show RNA-independent integration events occur at preferred sequence motifs. FIG. 3A is a schematic for single-molecule DNA curtains assay to visualize TnsC binding. λ phage DNA substrates are double-tethered between chrome pedestals and visualized used TIRF microscopy. FIG. 3B shows mNG-labeled TnsC preferentially binds AT-rich sequences on the λ-DNA substrate near the 3′ (pedestal) end. FIG. 3C is the correlation between AT content and mNG-TnsC fluorescence intensity visualized along the length of λ DNA. The Pearson linear correlation coefficient is shown (two-tailed P<0.0001); data shown represent the mean±s.d. for n=66 molecules. FIG. 3D shows binding kinetics for mNG-TnsC at AT-rich and AT-poor regions of the λ-DNA substrate. Apparent k_{obs} at AT-rich sites≈0.37 min⁻¹, 95% C.I. [0.35, 0.39] and at AT-poor sites≈0.28 min⁻¹, 95% C.I. [0.27, 0.30]. The data shown represent mean±s.d. for n=87 molecules. FIG. 3E is the cumulative frequency distributions for the AT content within a 100-bp window flanking integration events, using ShCAST with WT TnsC and sgRNA-1 (n=5,505 unique integration events), compared to random sampling of the E. coli genome (n=50,000 counts). The distributions were significantly different, based on results of a Mann-Whitny U test (P=1.48×10⁻¹³⁵). FIG. 3F is the cumulative frequency distribution comparison as in FIG. 3E, but with a K103A TnsC mutant (n=1,932 unique integration events), which revealed a loss of AT bias (P=0.1349). FIG. 3G shows meta-analysis of untargeted transposition specificity was performed by extracting sequences from a 140-bp window flanking the integration site and generating a consensus logo. FIG. 3H is a WebLogo from a meta-analysis of untargeted genomic transposition (n=5,855 unique integration events) with a modified pHelper lacking Cas12k and sgRNA. The site of integration is noted with a maroon triangle. An AT-rich sequence spanning ˜25 bp likely reflects the footprint of two turns of a TnsC filament (black), whereas motifs within/near the target-site duplication (TSD) represent TnsB-specific sequence motifs (green). Specific TnsB residues/domains contacting the indicated nucleotides are shown. The zoomed-in inset highlights periodicity in the sequence bound by TnsC. FIG. 3I is a schematic showing the relative spacing of sequence features bound by Cas12k, TnsC, and TnsB in both on-target (RNA-dependent) and untargeted (RNA-independent) DNA transposition. In both cases, the TnsC footprint covers ˜25-bp of DNA and directs polarized, unidirectional integration downstream in a L-R orientation. FIG. 3J is a zoom-in view of the ShCAST transpososome structure, highlighting sequence-specific contacts between TnsB and the target DNA that were observed in the WebLogo in FIG. 3H. PDB ID: 8EA3 (Park, J.-U. et al. Nature 613, 775-782 (2023).

FIGS. 4A-4B show artificial induction of semi-targeted RNA-independent transposition at preferred motifs. A region on pTarget exhibiting low integration activity (original, blue) was substituted with rationally engineered sequences (colored) based on TnsC and TnsB binding preferences, generating the indicated pTarget variants (pT-1-6) (FIG. 4A). A performing biochemical transposition assays with the indicated pTarget substrates, integration reads were normalized and mapped to either the forward strand (fwd, red) or reverse strand (rev, black) (FIG. 4B). The intended ‘untargeted’ integration site based on optimized poly-A and TnsB consensus motifs is marked with a maroon triangle and dotted line; the representative region at right (850-900 bp) is shown to highlight consistency in integration events observed elsewhere on pTarget.

FIGS. 5A-5E show the fidelity of RNA-guided DNA integration is controlled by TnsC concentration. FIG. 5A is a schematic of alternative ShCAST expression strategy, in which TnsC was encoded on a separate plasmid (pTnsC) driven by a Lac or T7 promoter. Distinct cellular expression levels were confirmed by Western blot against a 3×FLAG epitope tag fused to TnsC (bottom). FIG. 5B shows the fraction of total genome-mapping integration reads detected at on-target and untargeted sites upon TnsC expression with a Lac or T7 promoter. FIG. 5C is a genome-wide view of E. coli genome-mapping reads for the original/WT ShCAST system as compared to a modified ShCAST system with low TnsC expression; the zoomed-in view visualizes reads comprising 1% or less of genome-mapping reads. The target site is marked with a green triangle. FIG. 5D is a graph of the fraction of total genome-mapping integration reads detected at on-target and untargeted sites, with the original ShCAST system or modified ShCAST system with low TnsC expression. Data for five sgRNAs are shown. For FIGS. 5B and 5D, the mean is shown from n=2 independent biological replicates. FIG. 5E is an exemplary model for target-site selection and transpososome assembly during on-target, RNA-dependent transposition (right) or untargeted, RNA-independent transposition (left) by type V-K CAST systems. Within the untargeted pathway, TnsC preferentially forms filaments at A/T-rich regions and is capped by TniQ, leading to the downstream site being selected by TnsB for integration. Cas12k-bound targets may better nucleate TnsC filament formation, and it is hypothesized that TnsC filaments loaded at Cas12k-bound targets serve as better substrates for DNA integration, compared to untargeted sites. All structures of TnsC filaments representing untargeted sites including the ‘BCQ’ transpososome, reveal K103 residues of the TnsC monomers forming the filament proximal to TnsB, contacting DNA with opposite strand polarity compared to on-target structures. This could be decisive for the distinct efficiencies observed at these sites. Increased residence time of TnsC filaments and/or increased catalytic selection by TnsB results in higher integration efficiency.

FIGS. 6A-6G show type V-K CASTs maintain a distinct, RNA-independent pathway. FIG. 6A is a genome-wide view of E. coli genome-mapping reads for the original/WT ShCAST system encoding the indicated sgRNAs; the zoomed-in views visualize reads comprising 1% or less of genome-mapping reads. Target sites are marked with a green triangle. FIG. 6B is integration site distributions determined from the NGS data, plotted as the distance from the PAM sequence to the first transposon bp. FIG. 6C is a schematic depicting the neighborhood analysis used to explore PAM (5′-GTN-3′) and sgRNA complementarity enrichment flanking off-target ShCAST integration events. Insertions were presumed to occur in the (target-left-right) T-LR, and a 60-66 bp window upstream of the insertion site was analyzed. For sites with a cognate PAM, the adjacent 23 bp were analyzed for complementarity to the sgRNA spacer sequence. Controls analyses used random samplings of the E. coli genome. FIG. 6D shows off-target ShCAST integration events from five distinct sgRNAs are not enriched in cognate PAMs, relative to a randomly sampled control dataset. Percentage of integration events (for n=2 biological replicates) detected with or without a PAM when compared to random regions of the E. coli genome. FIG. 6E shows off-target ShCAST integration events from five distinct sgRNAs are not enriched in the number of complementary matches to the sgRNA, relative to a randomly sampled control datasets (Methods). FIG. 6F shows TniQ selectively binds TnsC-DNA filaments, but not naked DNA, as observed using fluorescence polarization experiments with a 55-bp FAM-labeled DNA substrate. Data shown represent mean±s.d. for n=2 technical replicates. FIG. 6G shows normalized integration reads detected at the on-target site (green arrow, left) and a representative untargeted site (right), with varying Cas12k promoter strength. Note the differing y-axis ranges.

FIGS. 7A-7K show cryo-EM image analysis of the untargeted, RNA-independent ‘BCQ’ transpososome. FIG. 7A is a representative cryo-EM micrograph of the ‘BCQ’ transpososome sample, generated by incubating TnsB, TnsC, TniQ with target-LE and target-RE DNA substrates. The black bar represents 100 nm. IFG. 7B shows a computational workflow used for analyzing the ‘BCQ’ transpososome cryo-EM dataset. Topaz-extracted particles were first subjected to 2D classification in cryoSPARC. 2D classes with cryo-EM density for the TnsB strand-transfer complex (STC) and TnsC oligomer were selected for downstream heterogeneous refinement in cryoSPARC. Particles classified as the TnsB-TnsC complex were then subjected to non-uniform refinement (NUrefine) in cryoSPARC using a mask that covers the TniQ-TnsC region. The aligned particle stacks were then further classified using 3D classification in RELION, focusing on TniQ and the adjacent two subunits of TnsC. One class showing the best resolved TniQ density was selected for another round of 3D classification in cryoSPARC focusing on TniQ. The final particle stack with stronger TniQ density was then subjected to homogeneous refinement. Focused refinement was done on the TniQ-TnsC complex or the TnsB-STC region of the reconstruction. FIGS. 7D-7E show gold-standard Fourier shell correlation (GSFSC) curves from the consensus refinement (FIG. 7C) and the focused refinements of the TniQ-TnsC region (FIG. 7D) or the TnsB-STC region (FIG. 7E) of the reconstruction. The 0.143 GSFSC cutoff is indicated as a blue horizontal line. FIGS. 7F-7H show estimated local resolution from the consensus refinement (FIG. 7F) and the focused refinements of the TniQ-TnsC region (FIG. 7G) or the TnsB-STC region (FIG. 7H). Local-resolution filtered reconstructions were colored based on the estimated local resolution, as indicated in the legend. FIGS. 7I-7K show angular distribution heatmap of the particle stack in the final consensus refinement (FIG. 7I) and focused refinements of the TniQ-TnsC region (FIG. 7J) and the TnsB-STC region (FIG. 7K). Colors indicate particle count, as indicated in the legend.

FIGS. 8A-8G show ShCAST biochemical reconstitution. FIG. 8A is an E. coli growth curves of seven biological replicate cultures of a TnsC over-expression strain under inducible conditions, with or without TnsB. The majority of cultures without TnsB fail to grow. FIG. 8B shows pictures of transformation plates after attempts to clone TnsC or Cas12k expression plasmids with a Lac (weak) or J23119 (strong) promoter, providing extra evidence that strong TnsC over-expression leads to cellular toxicity. The same competent cells and input DNA amounts were used in all cases, and surviving colonies with a strong promoter driving TnsC frequently exhibited mutations in the promoter or open reading frame (ORF). FIG. 7C shows total genome-mapping reads detected for the indicated expression construct, normalized, and scaled; the mean is shown from n=2 independent biological replicates. FIG. 7D shows SDS-PAGE analysis of purified ShCAST components used in biochemical transposition assays. FIG. 7E shows DNA binding by TnsC is ATP dependent under 200 mM NaCl conditions, as observed using fluorescence polarization experiments with a 55-bp FAM-labeled DNA substrate. Data shown represent mean±s.d. for n=3 technical replicates. FIG. 7F shows that under low-salt conditions (50 mM NaCl), DNA binding by TnsC is nucleotide-independent. Data are shown as in FIG. 7E. FIG. 7G shows TnsB stimulates the ATP hydrolysis activity of TnsC in a DNA-dependent reaction, as determined using a Malachite Green assay. The mean is shown from n=2 technical replicates; ND, not detected.

FIGS. 9A-9K show relative stoichiometry of CAST components controls pathway choice. FIG. 9A is a schematic of biochemical transposition assays that were performed in vitro with purified ShCAST components, followed by PCR-based amplification of the junction between pTarget and the transposon left end (LE) using the indicated primers (red). Note that ShCAST generates Shapiro intermediate products in these experiments; NNNNN denotes the single-stranded target-site duplication (TSD) region. FIG. 9B shows transposition products were analyzed by PCR amplification and 1.5% agarose gel electrophoresis for the indicated conditions. The on-target product (black arrow) is only efficiently generated in the presence of all ShCAST components, though TniQ is partially dispensable for in vitro transposition under these conditions, as previously reported. FIG. 9C shows integration site distribution for the on-target products in FIG. 9B, determined by high-throughput amplicon sequencing. FIG. 9D is a zoomed-in view of integration reads comprising 1% or less of pDonor-(top) and pTarget-(bottom) mapping reads, in a biochemical transposition assay performed without Cas12k or sgRNA. FIG. 9E is normalized integration reads detected at on-target and untargeted sites from biochemical transposition assays, at varying TnsC concentrations. FIG. 9F is normalized integration reads detected at on-target and untargeted sites from biochemical transposition assays, with 0.1 μM TnsC and varying TniQ concentrations. FIG. 9G shows on-target transposition efficiency from biochemical transposition assays as detected by qPCR, with 0.1 μM TnsC and varying S15 concentrations. FIG. 9H shows normalized integration reads detected at on-target and untargeted sites from biochemical transposition assays with 1 μM TnsC, with or without S15. FIG. 9I shows on-target transposition efficiency from biochemical transposition assays as detected by qPCR, with 0.1 μM TnsC and varying TnsB concentrations. FIG. 9J shows normalized integration reads detected at on-target and untargeted sites from biochemical transposition assays, with 0.1 μM TnsC and varying TnsB concentrations. FIG. 9K shows normalized integration reads detected at on-target and untargeted sites from biochemical transposition assays, with 1 μM TnsC and varying TnsB concentrations.

FIGS. 10A-10L show TnsC binding and TnsC-mediated integration exhibit preferential AT bias. FIG. 10A shows normalized integration reads detected at on-target and untargeted sites from cellular transposition assays, with either WT TnsC or an mNeonGreen (mNG)-TnsC fusion. FIG. 10B shows a representative kymograph from a DNA curtains assay with 100 nM mNG-TnsC, showing the time-dependent evolution λ-DNA binding. B, barrier; P, pedestal. FIG. 10C shows λ-DNA was completely coated with TnsC at high (500 nM) concentrations, as exemplified by a representative DNA curtains image. FIG. 10D outlines an exemplary analytical workflow to investigate the AT content surrounding untargeted integration events in transposition assays. Two windows (50-bp for biochemical or 100-bp for genomic integration) flanking each unique event were analyzed, and the highest AT content was retained for subsequent analyses. A similar workflow was performed for random sampling of the same DNA substrate. FIG. 10E shows the cumulative frequency distributions for the AT content within a 50-bp window flanking unique integration events on pTarget (in biochemical integration assay), using ShCAST with WT TnsC and sgRNA-6 (n=504 unique integration events), compared to random sampling of pTarget (n=50,000 counts; Methods). The distributions were significantly different, based on results of a Mann-Whitney U test (P=3.64×10⁻¹¹). FIG. 10F is a graph comparing the distribution of AT content (46-bp bins) and integration reads on pTarget, with indicated Spearman correlation coefficient and results from a two-tailed significance test. FIG. 10G shows the cumulative frequency distributions for the AT content within a 50-bp window flanking unique integration events on λ-DNA, using ShCAST with WT TnsC and sgRNA-6 (n=190 unique integration events), compared to random sampling of λ-DNA (n=50,000 counts; Methods). The distributions were significantly different, based on results of a Mann-Whitney U test (P=4.77×10⁻¹¹). FIG. 10H is a graph comparing the distribution of mNG-TnsC fluorescence intensity (1,078-bp bins) and integration reads on λ-DNA, with indicated Spearman correlation coefficient and results from a two-tailed significance test. FIG. 10I is a graph comparing the distribution of AT content (1,078-bp bins) and integration reads on λ-DNA, with indicated Spearman correlation coefficient and results from a two-tailed significance test. FIG. 10J shows the cumulative frequency distributions for the AT content within a 100-bp window flanking unique integration events in the E. coli genome, using ShCAST in the absence of Cas12k and sgRNA (n=3,103 unique integration events), compared to random sampling of the E. coli genome (n=50,000 counts; Methods). The distributions were significantly different, based on results of a Mann-Whitney Utest (P=4.40×10⁻⁸³). FIG. 10K is genome-wide view of E. coli genome-mapping reads for ShCAST with a K103A TnsC mutation; the zoomed-in view visualizes reads comprising 1% or less of genome-mapping reads. The target site is marked with a green triangle, and the E. coli oriC is marked with a maroon triangle. FIG. 10L shows untargeted genomic integration events are depleted in essential genes. The observed percentage of integration reads occurring within essential genes was quantified and plotted for each of the indicated datasets, from n=2 independent biological replicates. The percentage of sequences in the E. coli genome denoted as essential is marked with a red line.

FIGS. 11A-11C show TnsC selects AT-rich features upstream of the integration site. FIG. 11A is WebLogo from a meta-analysis of untargeted genomic transposition (n=6,800 unique integration events) from previously published data with pHelper and sgRNA-1. The site of integration is noted with a maroon triangle. As with the dataset from this study (FIG. 3H), these results reveal an AT-rich sequence spanning ˜25 bp that likely reflects the footprint of two turns of a TnsC filament (black), next to motifs within/near the target-site duplication (TSD) region that represent TnsB-specific sequence motifs (green). Specific TnsB residues/domains contacting the indicated nucleotides are shown. The zoomed-in inset highlights periodicity in the sequence bound by TnsC. FIG. 11B is WebLogo from a meta-analysis of untargeted genomic transposition (n=6,800 unique integration events) with a modified pHelper encoding a TnsC K103A mutation and sgRNA-1, visualized as in FIG. 11A. The AT-rich motif is conspicuously absent, as compared to experiments with WT TnsC. FIG. 11C is WebLogo from a meta-analysis of untargeted genomic transposition (n=6,800 unique integration events) with the divergent ShoCAST system encoding sgRNA-7, visualized as in FIG. 11A. The AT-rich motif is conspicuously shorter in size with ShoCAST as compared to ShCAST, in agreement with the distinct integration distance distribution for both systems.

FIGS. 12A-12F show engineering strategies to rationally alter ShCAST specificity. FIG. 12A shows the fraction of total genome-mapping integration reads detected at on-target and untargeted sites, with WT ShCAST and sgRNA-1, ShCAST and a non-targeting sgRNA control (sgRNA-NT), or the listed ShCAST fusion constructs with sgRNA-1. In some cases, a fusion was supplemented with an additional plasmid encoding unfused protein components, as indicated. FIG. 12B shows normalized genome-mapping integration reads detected at on-target and untargeted sites, for WT ShCAST and N- and C-terminal dCas9-TnsC fusions. No on-target reads were obtained for these fusion constructs. FIG. 12C shows normalized integration reads detected at the on-target site (left), the T7 RNAP locus (middle), and a representative untargeted region (right), using either a TnsC expression plasmid driven by a Lac or T7 promoter. A high density of untargeted insertions was observed within the T7 RNAP gene, likely due to positive selection for loss-of-function transposition events. Note the differing y-axis ranges. FIG. 12D shows normalized genome-mapping integration reads detected at on-target and untargeted sites, when TnsC was expressed with a Lac or T7 promoter. FIG. 12E shows the fraction of total genome-mapping integration reads detected at on-target and untargeted sites from experiments with T7 promoter-driven TnsC, with or without computational masking of insertions within the T7 RNAP gene. FIG. 12F shows the relative on-target transposition efficiency measured for the modified design in which TnsC was expressed from a low-strength promoter compared to the original pHelper. The guide RNA are indicated sgRNA 1-5. Measurements were made by Taqman qPCR and normalized against the rssA locus. For FIGS. 12A, 12B, and 12D-12F, the mean is shown from n=2 independent biological replicates.

FIG. 13 shows the fraction of total genome-mapping integration reads detected at RNA-dependent and RNA-independent sites for wildtype ShCAST (WT ShCAST), non-targeting sgRNA control (sgRNA-NT) and N-terminal fusion of TnsC with mNeonGreen (mNG-TnsC).

FIGS. 14A and 14B show TnsC adopts a similar overall architecture across various partial and complete transpososome complexes. FIG. 14A shows structures of TnsC-associated complexes from ShCAST in various configurations. The structures being compared are, from left to right: Cas12k-S15-TniQ-TnsC recruitment complex (PDB: 8BD5), Cas12k-transpososome (PDB: 8EA3), TnsC filament (PDB: 7M99), TnsC-TniQ-TnsBCTD complex (PDB: 7SVU), and BCQ transpososome (this study). Components associated with each structure include Cas12k (pink), S15 (tan), TnsC (green), TniQ (orange), and TnsB (purple). FIG. 14B shows interactions between TnsC and DNA from each of the structures shown in FIG. 14A. TnsC residues K103 and T121 (pink) are highlighted alongside both DNA strands (blue).

### DETAILED DESCRIPTION

The disclosed systems, kits, and methods provide systems and methods for nucleic acid integration utilizing engineered CRISPR-associated transposon (CAST) systems. The disclosed systems, kits, and methods provide systems and methods for RNA-guided DNA integration utilizing engineered CAST systems.

Bacteria encode diverse mobile genetic elements that exhibit a wide spectrum of mobilization behaviors, ranging from selective targeting of fixed attachment sites to promiscuous insertion into degenerate sequence motifs. Although insertion specificity is often dictated by a single recombinase enzyme, some transposons encode heteromeric transposase complexes that distribute DNA target and DNA integration activities across multiple distinct molecular components. Tn7-like transposons are unique in this regard, in that they have evolved to exploit diverse molecular pathways for target site selection, including site-specific DNA binding proteins, replication fork-specific DNA binding proteins, CRISPR RNA-guided DNA binding complexes, and additional DNA targeting pathways that have yet to be characterized. CASTs represent both a fascinating example of CRISPR-Cas exaptation as well as an opportune starting point for the development of next-generation tools for programmable, large-scale DNA insertion.

CAST systems fall within either type I or type V classes, which differ in their reliance on either Cascade or Cas12k effector complexes, respectively. Although the core transposition machinery is conserved across CAST families, including a DDE-family transposase for integration (TnsB), a AAA+ ATPase for target site selection (TnsC), and an adaptor protein for CRISPR-transposition coupling (TniQ), key molecular features distinguish the integration behavior of archetypal type I-F and type V-K systems. Whereas second-strand cleavage is catalyzed by the TnsA endonuclease in type I-F CASTs, leading to cut-and-paste transposition products, type V-K CASTs lack TnsA and instead mobilize through a copy-and-paste process, yielding cointegrate products. Additionally, heterologous expression of the CAST machinery from both systems yields vastly different integration specificities, with VchCAST (I-F) exhibiting mostly on-target activity in bacterial cells, as compared to an abundance of off-target insertions catalyzed by ShCAST (V-K). Despite these differences, it is to be noted that type V-K CASTs have a compact coding sequence composed of four components compared to type I-F CAST (1666 vs. 2748 amino acids) and integrates predominantly in a unidirectional orientation. The molecular basis underlying these distinguishing properties remains largely unexplored, particularly for type V-K CAST systems, limiting their practical application.

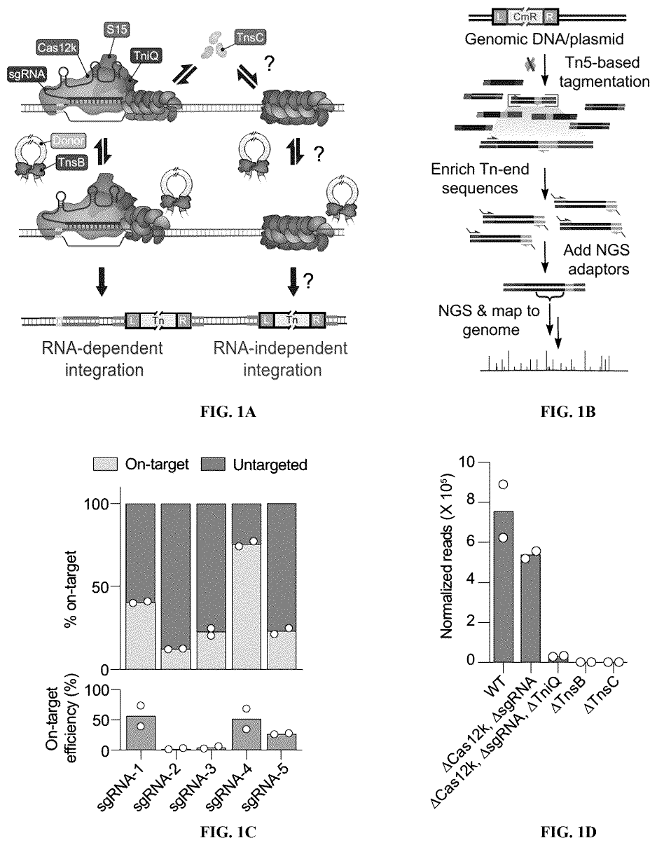

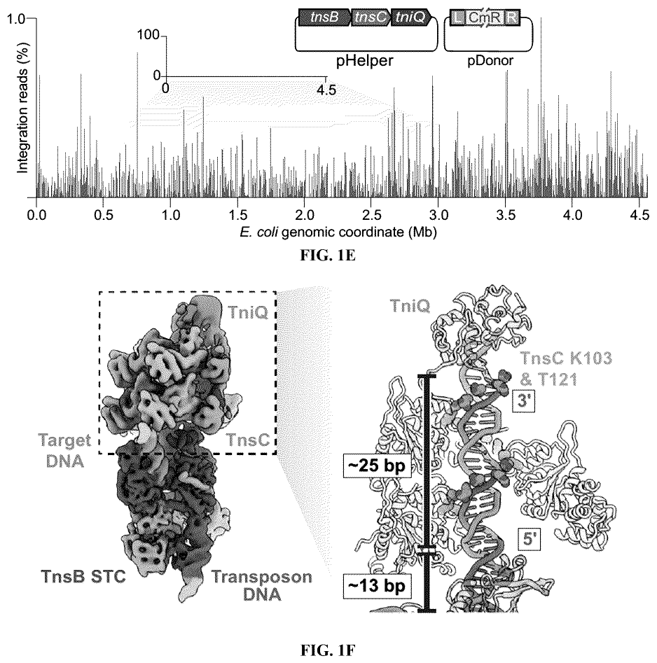

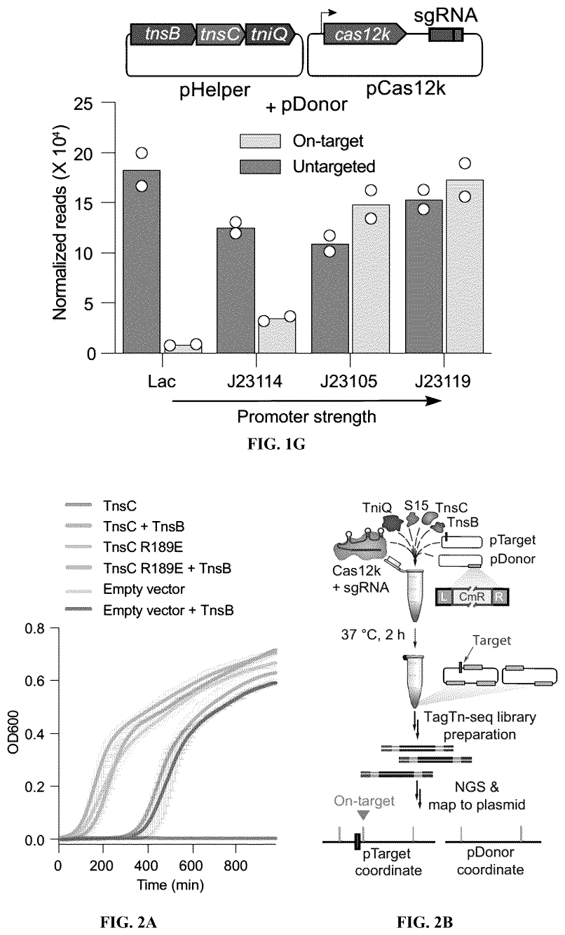

Recent structural studies have provided some insights into the overall architecture of RNA-guided, ShCAST transpososome complexes. Target sites are marked by Cas12k binding, in conjunction with TniQ and ribosomal protein S15, which engages the tracrRNA component, leading to stable R-loop formation reminiscent of other CRISPR effectors. The next step that is still poorly understood, TnsC assembles into filaments around double-stranded DNA, which can form adjacent to bound Cas12k-TniQ complexes or on naked DNA, acting as a platform for the subsequent recruitment of the TnsB transposase that is scaffolded along conserved binding sites in the transposon left and right ends. DNA integration then occurs via a concerted transesterification at sites exposed by the TnsC filament, leading to transposons inserted at a fixed spacing downstream of the Cas12k-bound target site. Whether a similar assembly pathway is operational at the many off-target integration events observed with ShCAST expression in cells, or whether these represent an alternative transposition pathway, has not been systematically explored (FIG. 1A). All the recently discovered Tn7-like CAST systems, including type I-F, I-B, I-D, and V-K, use TnsC for site-specific insertion of large genetic cargos. A similar ATPase, namely MuB, is also thought to be employed by recently predicted Mu-like CAST systems.

Remarkably, as shown herein, it was found that an archetypal type V-K CAST system from Scytonema hofmannii (ShCAST) was prone to extensive, RNA-independent transposition through a pathway that requires only TnsB, TnsC, and TniQ. Although these untargeted integration events initially appear random, analysis of high-throughput sequencing data revealed a significant bias for AT-rich sites, which was corroborated by single-molecule biophysical studies of TnsC DNA-binding behavior. By modulating DNA substrates in biochemical transposition assays, the preference for AT-rich sequences could lead to predictable reaction outcomes. Furthermore, transposition specificity could be substantially improved by limiting the cytoplasmic TnsC levels, further highlighting the role of TnsC filament formation in the pathway choice between RNA-dependent and RNA-independent transposition.

Section headings as used in this section and the entire disclosure herein are merely for organizational purposes and are not intended to be limiting.

### Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. As used herein, comprising a certain sequence or a certain SEQ ID NO usually implies that at least one copy of said sequence is present in recited peptide or polynucleotide. However, two or more copies are also contemplated. The singular forms “a,” “and” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. For example, any nomenclature used in connection with, and techniques of cell and tissue culture, molecular biology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

As used herein, “nucleic acid” or “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41(14): 4503-4510 (2002)) and U.S. Pat. No. 5,034,506), locked nucleic acid (LNA; see Wahlestedt et al., Proc. Natl. Acad. Sci. U.S.A., 97:5633-5638 (2000)), cyclohexenyl nucleic acids (see Wang, J. Am. Chem. Soc., 122:8595-8602 (2000)), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

Nucleic acid or amino acid sequence “identity,” as described herein, can be determined by comparing a nucleic acid or amino acid sequence of interest to a reference nucleic acid or amino acid sequence. The percent identity is the number of nucleotides or amino acid residues that are the same (e.g., that are identical) as between the sequence of interest and the reference sequence divided by the length of the longest sequence (e.g., the length of either the sequence of interest or the reference sequence, whichever is longer). A number of mathematical algorithms for obtaining the optimal alignment and calculating identity between two or more sequences are known and incorporated into a number of available software programs. Examples of such programs include CLUSTAL-W, T-Coffee, and ALIGN (for alignment of nucleic acid and amino acid sequences), BLAST programs (e.g., BLAST 2.1, BL2SEQ, and later versions thereof) and FASTA programs (e.g., FASTA3×, FAS™, and SSEARCH) (for sequence alignment and sequence similarity searches). Sequence alignment algorithms also are disclosed in, for example, Altschul et al., J. Molecular Biol., 215(3): 403-410(1990), Beigert et al., Proc. Natl. Acad. Sci. USA, 106(10): 3770-3775(2009), Durbin et al., eds., Biological Sequence Analysis: Probabilistic Models of Proteins and Nucleic Acids, Cambridge University Press, Cambridge, UK (2009), Soding, Bioinformatics, 21(7): 951-960(2005), Altschul et al., Nucleic Acids Res., 25(17): 3389-3402(1997), and Gusfield, Algorithms on Strings, Trees and Sequences, Cambridge University Press, Cambridge UK (1997)).

The term “homology” and “homologous” refers to a degree of identity. There may be partial homology or complete homology. A partially homologous sequence is one that is less than 100% identical to another sequence.

As used herein, the term “hybridization” is used in reference to the pairing of complementary nucleic acids. Hybridization and the strength of hybridization (e.g., the strength of the association between the nucleic acids) is influenced by such factors as the degree of complementary between the nucleic acids, stringency of the conditions involved, and the Tₘ of the formed hybrid. Hybridization methods involve the annealing of one nucleic acid to another, complementary nucleic acid, e.g., a nucleic acid having a complementary nucleotide sequence. The ability of two polymers of nucleic acid containing complementary sequences to find each other and “anneal” or “hybridize” through base pairing interaction is a well-recognized phenomenon. The initial observations of the “hybridization” process by Marmur and Lane, Proc. Natl. Acad. Sci. USA, 46:453 (1960) and Doty et al., Proc. Natl. Acad. Sci. USA, 46:461 (1960), have been followed by the refinement of this process into an essential tool of modern biology. For example, hybridization and washing conditions are now well known and exemplified in Sambrook et al., supra. The conditions of temperature and ionic strength determine the “stringency” of the hybridization.

As used herein, a “double-stranded nucleic acid” may be a portion of a nucleic acid, a region of a longer nucleic acid, or an entire nucleic acid. A “double-stranded nucleic acid” may be, e.g., without limitation, a double-stranded DNA, a double-stranded RNA, a double-stranded DNA/RNA hybrid, etc. A single-stranded nucleic acid having secondary structure (e.g., base-paired secondary structure) and/or higher order structure (e.g., a stem-loop structure) may also be considered a “double-stranded nucleic acid.” For example, triplex structures are considered to be “double-stranded.” In some embodiments, any base-paired nucleic acid is a “double-stranded nucleic acid.”

The term “gene” refers to a DNA sequence that comprises control and coding sequences necessary for the production of an RNA having a non-coding function (e.g., a ribosomal or transfer RNA), a polypeptide, or a precursor of any of the foregoing. The RNA or polypeptide can be encoded by a full length coding sequence or by any portion of the coding sequence so long as the desired activity or function is retained. Thus, a “gene” refers to a DNA or RNA, or portion thereof, that encodes a polypeptide or an RNA chain that has functional role to play in an organism. For the purpose of this disclosure, it may be considered that genes include regions that regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The terms “non-naturally occurring,” “engineered,” and “synthetic” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

A “vector” or “expression vector” is a replicon, such as plasmid, phage, virus, or cosmid, to which another DNA segment, e.g., an “insert,” may be attached or incorporated so as to bring about the replication of the attached segment in a cell.

A cell has been “genetically modified,” “transformed,” or “transfected” by exogenous DNA, e.g., a recombinant expression vector, when such DNA has been introduced inside the cell. The presence of the exogenous DNA results in permanent or transient genetic change. The transforming DNA may or may not be integrated (covalently linked) into the genome of the cell. For example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment of the methods and compositions provided herein, the mammal is a human.

The term “contacting” as used herein refers to bring or put in contact, to be in or come into contact. The term “contact” as used herein refers to a state or condition of touching or of immediate or local proximity. Contacting a composition to a target destination, such as, but not limited to, an organ, tissue, cell, or tumor, may occur by any means of administration known to the skilled artisan.

As used herein, the terms “providing,” “administering,” and “introducing,” are used interchangeably herein and refer to the placement of the systems of the disclosure into a cell, organism, or subject by a method or route which results in at least partial localization of the system to a desired site. The systems can be administered by any appropriate route which results in delivery to a desired location in the cell, organism, or subject.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### Systems & Methods for Increasing CAST System Specificity and Efficiency

In bacteria and archaea, CRISPR/Cas systems provide immunity by incorporating fragments of invading phage, virus, and plasmid DNA into CRISPR loci and using corresponding CRISPR RNAs (“crRNAs”) to guide the degradation of homologous sequences. Transcription of a CRISPR locus produces a “pre-crRNA,” which is processed to yield crRNAs containing spacer-repeat fragments that guide effector nuclease complexes to cleave dsDNA sequences complementary to the spacer. Several different types of CRISPR systems are known, (e.g., type I, type II, or type III), and classified based on the Cas protein type and the use of a proto-spacer-adjacent motif (PAM) for selection of proto-spacers in invading DNA.

Although RNA-guided targeting typically leads to endonucleolytic cleavage of the bound substrate, recent studies have uncovered a range of noncanonical pathways in which CRISPR protein-RNA effector complexes have been naturally repurposed for alternative functions. For example, some Type I (Cascade) and Type II (Cas9) systems leverage truncated guide RNAs to achieve potent transcriptional repression without cleavage and other Type I (Cascade) and Type V (Cas12) systems lie inside unusual bacterial Tn7-like transposons and lack nuclease components altogether.

Disclosed herein are systems and methods for increasing the specificity and efficiency Clustered Regularly Interspaced Short Palindromic Repeats (CRISPR)-CRISPR associated (Cas) transposon (CAST) systems. The described systems and methods represent advancements in the field of genome editing by enhancing the precision, specificity, and efficiency of CAST systems. To increase specificity and efficiency of CAST systems, the methods and systems take advantage of any one or all of: modulating TnsC function and abundance; modulating TnsB function and abundance; and influencing CAST target preference.

Specificity refers to the relationship between on-target or RNA-guided functionality versus off-target or RNA-independent functionality. Increasing the specificity of a CAST system results in greater percentage of on-target or RNA-guided functionality as compared to off-target or RNA-independent functionality. Increasing the specificity encompasses any increase in specificity, e.g., at least 1%, at least 5%, at least 10%, at least 20%, at least 30%, at least 40%, at least 50%, or more increase in specificity.

Efficiency refers to the degree of achieving the desired DNA modification of the CAST system. For example, out of a certain number of possible sites of modification, efficiency is a measure of how many of those are modified, e.g., receive the desired insert if the CAST system is being utilized for RNA-guided integration. Efficiency can be calculated as described in the present Examples.

The methods and systems are not limited to any particular CAST system. CRISPR-Cas systems are currently grouped into two classes (1-2), six types (I-VI) and dozens of subtypes, depending on the signature and accessory genes that accompany the CRISPR array. The engineered CAST system herein may be derived from a Class 1 CRISPR-Cas system or a Class 2 CRISPR-Cas system. The CAST system may be derived from a Type I CRISPR-Cas system or a Type V CRISPR-Cas system. In select embodiments, the CAST system is derived from a type V-K CRISPR-Cas system. In select embodiments, the CAST system is derived from a type V-K CRISPR-Cas system from Scytonema hofmannii. In select embodiments, the CAST system is derived from a type I-F, I-B, or I-D CRISPR-Cas system. In select embodiments, the CAST system is derived from a Mu-like system.

In some embodiments, the one or more Cas proteins comprise Cas5, Cas6, Cas7, Cas8, or any combination thereof. In some embodiments, the one or more Cas proteins comprise a Cas8-Cas5 fusion protein. In some embodiments, the one or more Cas proteins comprise Cas5, Cas6, Cas7, and Cas8. In some embodiments, the one or more Cas proteins comprise a Cas12 protein. In some embodiments, the one or more Cas proteins comprise Cas12k, previously known as C2c5.

A CAST system of the present invention may comprise one or more transposon associated proteins (e.g., transposases or other components of a transposon). In some embodiments, the transposon associated proteins are derived from a Tn7 or Tn7-like transposon. Tn7 and Tn7-like transposons may be categorized based on the presence of the hallmark DDE-like transposase gene, tnsB (also referred to as tniA), the presence of a gene encoding a protein within the AAA+ ATPase family, tnsC (also referred to as tniB), one or more targeting factors that define integration sites (which may include a protein within the tniQ) family, also referred to as tnsD, but sometimes includes other distinct targeting factors), and inverted repeat transposon ends that typically comprise multiple binding sites thought to be specifically recognized by the TnsB transposase protein. Whereas Tn7 comprises tnsD and tnsE target selectors, related transposons comprise other genes for targeting. For example, Tn5090/Tn5053 encode a member of the tniQ family (a homolog of E. coli tnsD) as well as a resolvase gene tniR; Tn6230 encodes the protein TnsF; and Tn6022 encodes two uncharacterized open reading frames orf2 and orf3; Tn6677 and related transposons encode variant Type I-F and Type I-B CRISPR-Cas systems that work together with TniQ for RNA-guided mobilization; and other transposons encode Type V-U5 CRISPR-Cas systems that work together with TniQ for random and RNA-guided mobilization. Any of the above transposon systems are compatible with the systems and methods described herein.

In some embodiments, the one or more transposon associated proteins comprise TnsC. In some embodiments, the one or more transposon associated proteins comprise TnsB and TnsC. In some embodiments, the one or more transposon associated proteins comprise TniQ. In certain embodiments, the one or more transposon associated proteins comprise TnsB, TnsC, and TniQ. In some embodiments, the one or more transposon associated proteins comprise TnsA, TnsB, TnsC, TnsD and/or TniQ.

Sequences of exemplary Cas proteins and transposon-associated proteins can be found herein and also in International Patent Application Publications WO2020181264 and WO2022261122, incorporated herein by reference. However, the described systems and methods are not limited to the disclosed or referenced exemplary sequences. Genetic sequences can vary between different strains, and this natural scope of allelic variation is included within the scope of the invention.

In some embodiments at least one of the one or more Cas proteins and the one or more transposon associated proteins are provided as a fusion protein. Alternatively, at least two of the disclosed modified Cas proteins or transposon associated proteins may be linked in a fusion protein. In some embodiments, each of the one or more Cas proteins and the one or more transposon-associated proteins are provided as a single fusion protein.

The term “fusion protein” as used herein refers to a polypeptide which comprises at least two different proteins or at least two protein domains from two different proteins. The fusion protein is not limited by orientation of the at least two different proteins. For example, the arrangement of the first protein in the fusion protein may be N-terminal or C-terminal to the second protein.

The fusion protein may comprise a linker polypeptide between the first amino acid sequence and the second amino acid sequence. The linker polypeptide may have any of a variety of amino acid sequences. Proteins can be joined by a linker polypeptide, generally of a flexible nature, although other chemical linkages are not excluded. Suitable linkers include polypeptides of between 4 amino acids and 40 amino acids in length, or between 4 amino acids and 25 amino acids in length. The linking peptides may have virtually any amino acid sequence, bearing in mind that the preferred linkers will have a sequence that results in a generally flexible peptide. Small amino acids, such as glycine and alanine, are useful in creating a flexible peptide linker. A variety of different linkers are considered suitable for use, including but not limited to, glycine-serine polymers, glycine-alanine polymers, and alanine-serine polymers.

In the systems disclosed herein, at least one of the one or more Cas proteins and the one or more transposon-associated proteins may comprise at least one effector domain. The at least one effector domain may be appended to one or more of the at least one Cas protein and the at least one transposon-associated protein at a N-terminus, a C-terminus, internally, or a combination thereof. The effector domains may be fused in any orientation in relationship to the at least one Cas protein and the at least one transposon-associated protein.

Effector domains contain any protein or fragments thereof that can modify, regulate, or tag a target nucleic acid. The effector domain may comprise a number of functionalities, including but not limited to, nuclease function, recombinase function, epigenetic modifying function, transposase function, integrase function, resolvase function, invertase function, protease function, DNA methyltransferase function, DNA demethylase function, histone acetylase function, histone deacetylase function, transcriptional repressor function, transcriptional activator function, DNA binding protein function, transcription factor recruiting protein function, nuclear-localization signal function, DNA editing function (e.g., deaminase) or any combination thereof. For example, some effector domains function in transcriptional regulation via their ability to interact with the basal transcriptional machinery and general co-activators, interact with other transcription factors to allow cooperative binding, and/or directly or indirectly recruit histone and chromatin modifying enzymes. In some embodiments, any additional domains or proteins necessary for the functionality of the effector domain may be provided as a fusion to the one or more of the at least one Cas protein and the at least one transposon-associated protein or separately. Additional descriptions of Cas proteins and transposon-associated proteins fused to effector domains can be found in International Patent Application Publication WO2022266492, incorporated herein by reference. However, the described systems and methods are not limited to the disclosed or referenced exemplary domains.

The protein components of the disclosed system (e.g., the Cas proteins or the transposon associated proteins) may comprise at least one nuclear localization sequence (NLS). The at least one nuclear localization sequence may be appended to at least one of the one or more Cas protein and the one or more transposon-associated protein at a N-terminus, a C-terminus, embedded in the protein (e.g., inserted internally within the open reading frame (ORF)), or a combination thereof.

The protein components of the disclosed system (e.g., the Cas proteins or the transposon associated proteins) may further comprise an epitope tag (e.g., 3×FLAG tag, an HA tag, a Myc tag, and the like). In some embodiments, the epitope tag may be adjacent, either upstream or downstream, to a nuclear localization sequence. The epitope tags may be at the N-terminus, a C-terminus, or a combination thereof of the corresponding protein.

A CAST system of the present invention may further comprise a gRNA complementary to at least a portion of the target nucleic acid sequence, or a nucleic acid encoding the at least one gRNA. In some embodiments, one or more of the at least one Cas protein are part of a ribonucleoprotein (RNP) complex with the gRNA. In some embodiments, one or more of the at least one Cas protein and one or more of the transposon associated proteins are part of a ribonucleoprotein (RNP) complex with the gRNA.

The gRNA may be a crRNA, crRNA/tracrRNA (or single guide RNA, sgRNA). The terms “gRNA,” “guide RNA,” “crRNA,” and “CRISPR guide sequence” may be used interchangeably throughout and refer to a nucleic acid comprising a sequence that determines the binding specificity of the CAST system. A gRNA hybridizes to (complementary to, partially or completely) a target nucleic acid sequence (e.g., the genome in a host cell). In some embodiments, the at least one gRNA is encoded in a CRISPR RNA (crRNA) array.

The gRNA or portion thereof that hybridizes to the target nucleic acid (a target site) may be any length. In some embodiments, the gRNA sequence that hybridizes to the target nucleic acid is 15, 16, 17, 18, 19, 20, 21, 22, 23, 24, 25, 26, 27, 28, 29, 30, 31, 32, 33, 34, 35, 36, 37, 38, 39, or 40 nucleotides in length. gRNAs or sgRNA(s) used in the present disclosure can be between about 5 and 100 nucleotides long, or longer (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 91 92, 93, 94, 95, 96, 97, 98, 99, or 100 nucleotides in length, or longer).

To facilitate gRNA design, many computational tools have been developed (See Prykhozhij et al. (PLOS ONE, 10(3): (2015)); Zhu et al. (PLOS ONE, 9(9) (2014)); Xiao et al. (Bioinformatics. Jan. 21 (2014)); Heigwer et al. (Nat Methods, 11(2): 122-123 (2014)). Methods and tools for guide RNA design are discussed by Zhu (Frontiers in Biology, 10 (4) pp 289-296 (2015)), which is incorporated by reference herein. Additionally, there are many publicly available software tools that can be used to facilitate the design of sgRNA(s); including but not limited to, Genscript Interactive CRISPR gRNA Design Tool, WU-CRISPR, and Broad Institute GPP sgRNA Designer. There are also publicly available pre-designed gRNA sequences to target many genes and locations within the genomes of many species (human, mouse, rat, zebrafish, (′. elegans), including but not limited to, IDT DNA Predesigned Alt-R CRISPR-Cas9 guide RNAs, Addgene Validated gRNA Target Sequences, and GenScript Genome-wide gRNA databases.

In addition to a sequence that binds to a target nucleic acid, in some embodiments, the gRNA may also comprise a scaffold sequence (e.g., tracrRNA). In some embodiments, such a chimeric gRNA may be referred to as a single guide RNA (sgRNA). Exemplary scaffold sequences will be evident to one of skill in the art and can be found, for example, in Jinek, et al. Science (2012) 337(6096): 816-821, and Ran, et al. Nature Protocols (2013) 8:2281-2308, incorporated herein by reference in their entireties.

In some embodiments, the gRNA sequence does not comprise a scaffold sequence and a scaffold sequence is expressed as a separate transcript. In such embodiments, the gRNA sequence further comprises an additional sequence that is complementary to a portion of the scaffold sequence and functions to bind (hybridize) the scaffold sequence.

The gRNA can comprise spacer sequence. The space sequence can be any length. In some embodiments, the space sequence is 30-40 nucleotides long (e.g., 30, 31, 32, 33, 34, 35, 36, 37, 38, 39, or 40).

In some embodiments, the gRNA sequence is at least 50%, 55%, 60%, 65%, 70%, 75%, 80%, 85%, 90%, 95%, 96%, 97%, 98%, 99%, or at least 100% complementary to a target nucleic acid. In some embodiments, the gRNA sequence is at least 50%, 55%, 60%, 65%, 70%, 75%, 80%, 85%, 90%, 95%, 96%, 97%, 98%, 99%, or at least 100% complementary to the 3′ end of the target nucleic acid (e.g., the last 5, 6, 7, 8, 9, or 10 nucleotides of the 3′ end of the target nucleic acid).

The gRNA may be a non-naturally occurring gRNA.

The system may further comprise a target nucleic acid. The terms “target sequence,” “target nucleic acid,” and “target site” (e.g., a “target genomic DNA sequence”) are used interchangeably herein to refer to a polynucleotide (nucleic acid, gene, chromosome, genome, etc.) to which a guide sequence (e.g., a synthetic guide RNA) is designed to have complementarity, wherein hybridization between the target sequence and a guide sequence promotes the formation of a CRISPR complex, provided sufficient conditions for binding exist. The target sequence and guide sequence need not exhibit complete complementarity, provided that there is sufficient complementarity to cause hybridization and promote formation of a CRISPR complex. A target sequence may comprise any polynucleotide, such as DNA or RNA. Suitable DNA/RNA binding conditions include physiological conditions normally present in a cell. Other suitable DNA/RNA binding conditions (e.g., conditions in a cell-free system) are known in the art.

The target sequence may or may not be flanked by a protospacer adjacent motif (PAM) sequence. In certain embodiments, a nucleic acid-guided nuclease can only cleave a target sequence if an appropriate PAM is present, see, for example Doudna et al., Science, 2014, 346(6213): 1258096, incorporated herein by reference. A PAM can be 5′ or 3′ of a target sequence. A PAM can be upstream or downstream of a target sequence. In one embodiment, the target sequence is immediately flanked on the 3′ end by a PAM sequence. A PAM can be 1, 2, 3, 4, 5, 6, 7, 8, 9, 10 or more nucleotides in length. In certain embodiments, a PAM is between 2-6 nucleotides in length. The target sequence may or may not be located adjacent to a PAM sequence (e.g., PAM sequence located immediately 3′ of the target sequence) (e.g., for Type I CRISPR/Cas systems). In some embodiments, e.g., Type I systems, the PAM is on the alternate side of the protospacer (the 5′ end). Makarova et al. describes the nomenclature for all the classes, types, and subtypes of CRISPR systems (Nature Reviews Microbiology 13:722-736 (2015)). Guide structures and PAMs are described in by R. Barrangou (Genome Biol. 16:247 (2015)).

Non-limiting examples of the PAM sequences include: CC, CA, AG, GT, TA, AC, CA, GC, CG, GG, CT, TG, GA, AGG, TGG, T-rich PAMs (such as TTT, TTG, TTC, etc.), NGG, NGA, NAG, NGGNG and NNAGAAW (W=A or T), NNNNGATT, NAAR (R=A or G), NNGRR (R=A or G), NNAGAA, and NAAAAC, where N is any nucleotide. In some embodiments, the PAM may comprise a sequence of CN, in which N is any nucleotide. In select embodiments, the PAM may comprise a sequence of CC.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule, which can form hydrogen bonds (e.g., Watson-Crick base pairing) with a second nucleic acid sequence. Full complementarity is not necessarily required, provided there is sufficient complementarity to cause hybridization. There may be mismatches distal from the PAM.

The system may further include a donor nucleic acid. The donor nucleic acid may be a part of a bacterial plasmid, bacteriophage, a virus, autonomously replicating extra chromosomal DNA element, linear plasmid, linear DNA, linear covalently closed DNA, mitochondrial or other organellar DNA, chromosomal DNA, and the like. In some embodiments, the donor nucleic acid comprises a cargo nucleic acid sequence.

The donor nucleic acid may be flanked by at least one transposon end sequence. In some embodiments, the donor nucleic acid is flanked on the 5′ and the 3′ end with a transposon end sequence. The term “transposon end sequence” refers to any nucleic acid comprising a sequence capable of forming a complex with the transposase enzymes thus designating the nucleic acid between the two ends for rearrangement. Usually, these sequences contain inverted repeats and may be about 10-150 base pairs long, however the exact sequence requirements differ for the specific transposase enzymes. Transposon end sequences are well known in the art. Transposon ends sequences may or may not include additional sequences that promote or augment transposition.

The transposon end sequences on either end may be the same or different. The transposon end sequence may be the endogenous CRISPR-transposon end sequences or may include deletions, substitutions, or insertions. The endogenous CRISPR-transposon end sequences may be truncated. In some embodiments, the transposon end sequence includes an about 40 base pair (bp) deletion relative to the endogenous CRISPR-transposon end sequence. In some embodiments, the transposon end sequence includes an about 100 base pair deletion relative to the endogenous CRISPR-transposon end sequence. The deletion may be in the form of a truncation at the distal (in relation to the cargo) end of the transposon end sequences.

The donor nucleic acid, and by extension the cargo nucleic acid, may of any suitable length, including, for example, about 50-100 bp (base pairs), about 100-1000 bp, at least or about 10 bp, at least or about 20 bp, at least or about 25 bp, at least or about 30 bp, at least or about 35 bp, at least or about 40 bp, at least or about 45 bp, at least or about 50 bp, at least or about 55 bp, at least or about 60 bp, at least or about 65 bp, at least or about 70 bp, at least or about 75 bp, at least or about 80 bp, at least or about 85 bp, at least or about 90 bp, at least or about 95 bp, at least or about 100 bp, at least or about 200 bp, at least or about 300 bp, at least or about 400 bp, at least or about 500 bp, at least or about 600 bp, at least or about 700 bp, at least or about 800 bp, at least or about 900 bp, at least or about 1 kb (kilobase pair), at least or about 2 kb, at least or about 3 kb, at least or about 4 kb, at least or about 5 kb, at least or about 6 kb, at least or about 7 kb, at least or about 8 kb, at least or about 9 kb, at least or about 10 kb, or greater.

In some embodiments, the systems further comprise one or more additional genome engineering tools. For example, the systems may further comprise nucleases, such as zinc finger nucleases (ZFNs) and/or transcription activator like effector nucleases (TALENs); transcriptional activators, transcriptional repressors, histone-modifying proteins, integrases, and recombinases.

In some embodiments, the system comprises components from or derived from different CAST systems. In some embodiments, at least one of the one or more Cas proteins and the one or more transposon associated proteins may be derived from a homologous CAST systems compared to the other protein components in the system.

The one or more nucleic acids encoding the engineered CAST system may be any nucleic acid including DNA, RNA, or combinations thereof. In some embodiments, the one or more nucleic acids comprise one or more messenger RNAs, one or more vectors, or any combination thereof.

The one or more Cas proteins, the one or more transposon-associated proteins, the at least one gRNA, and the donor nucleic acid may be on the same or different nucleic acids (e.g., vector(s)). In some embodiments, the one or more Cas proteins are encoded by a single nucleic acid. In some embodiments, the one or more transposon-associated proteins are encoded by a single nucleic acid. In some embodiments, the nucleic acid encoding the one or more Cas proteins also encodes the one or more transposon-associated proteins. In some embodiments, the one or more Cas proteins are encoded by a different nucleic acid from the one or more transposon-associated proteins.

In some embodiments, the at least one gRNA is encoded by a nucleic acid different from the nucleic acid(s) encoding the one or more Cas proteins and the one or more transposon-associated proteins. In some embodiments, the at least one gRNA is encoded by a nucleic acid also encoding at least one Cas protein, at least one transposon-associated protein, or both. In some embodiments, the one or more Cas proteins, the one or more transposon-associated proteins, and the at least one gRNA are encoded by a single nucleic acid. The gRNA may be encoded anywhere in the nucleic acid encoding the one or more Cas proteins or the one or more transposon-associated proteins. In some embodiments, the gRNA is encoded in the 3′ UTR of a protein coding nucleic acid.

In some embodiments, a nucleic acid encoding the one or more Cas proteins, the one or more transposon-associated protein, the at least one gRNA, or any combination thereof further comprises the donor nucleic acid.

The systems may be a cell-free system. Also disclosed is a cell comprising the system described herein. In some embodiments, the cell is a prokaryotic cell. In some embodiments, the cell is a eukaryotic cell. In some embodiments, the cell is a mammalian cell (e.g., a cell of a non-human primate or a human cell).

As disclosed herein, the stoichiometry of TnsC, TnsB, and TniQ influences the balance of RNA-dependent and RNA-independent integration. Higher TnsC levels or its increased affinity for DNA can lead to an upsurge in RNA-independent integration. The increased preference of TnsB for integration at the on-target site can be harnessed to enhance the efficiency of RNA-dependent integration and control the specificity of CAST systems by lowering TnsC levels. Certain fusion constructs can be employed to improve both the specificity and efficiency of CAST systems. In addition, the sequence preferences of TnsC and TnsB can be used to engineer CAST systems that deliver high precise insertion at the on-target site. Furthermore, the methods can also be applied to engineered versions of CAST systems wherein transposase components are fused to engineered DNA binding proteins, such as nuclease-dead Cas9 (dCas9), nuclease-dead Cas12 using homologs other than Cas12k, such as Cas12m, dCas12a, dCas12b, dCas12c, dCas12f, and other inactivated Cas12 homologs, nuclease-dead IscB, nuclease-dead TnpB, or other fixed or programmable DNA targeting modalities.

### a) Modulation and Engineering of TnsC

In some embodiments, the systems and methods comprise engineering or modulating the TnsC in the CAST system. The AAA+ ATPase from CAST systems, TnsC, is known to form multimeric assemblies on DNA and is preferentially recruited by the targeting factor, TniQ, at sites occupied by the CRISPR effector complex, which may be Cascade (Type I systems) or Cas12k (Type V systems). TnsC also exhibits the ability to bind to DNA independently and is not site-specific or targeted like the CRISPR effector, which is guided by crRNA/sgRNA. Upon stable binding at either RNA-dependent or independent sites, TnsC recruits the transposase, TnsB, and the cargo DNA to the binding site, leading to either TnsC disassembly or integration. As shown herein, while TnsB preferentially integrates at RNA-dependent sites, it is the non-specific TnsC binding that influences the levels of RNA-independent integration. TniQ capping also influences RNA-independent integration.

Although referred to herein as TnsC, the methods and systems described herein in reference to TnsC modulation and engineering can encompass the AAA+ ATPase from CAST and CAST-like systems, some of which may not be referred to as TnsC but carry out the same structure and function roles within the CAST system.

In some embodiments, the methods and systems comprise decreasing levels of TnsC. By reducing TnsC levels in a biochemical reaction or by modulating TnsC expression levels in cells for type V-K CASTs, the on-target specificity can be substantially increased, for example, up to 99.7% in vitro and 98.1% in cells from below 50% under un-optimized conditions. As described herein, lowering TnsC expression resulted in increased efficiency and specificity for multiple independently tested guide RNAs. Importantly, lowering TnsC levels did not compromise the efficiency of RNA-dependent integration.

In some embodiments, the system comprise less TnsC, or a nucleic acid encoding TnsC, compared to any or all of remaining components of the CAST system or nucleic acids encoding thereof. In some embodiments, the system provide TnsC on an individual nucleic acid as compared to any or all of remaining components of the CAST system. Thus, the quantity of that individual nucleic acid can be modulated to be decreased in comparison to the remaining components of the CAST system.

In some embodiments, for example when the desired use of the system is for DNA modification in a cell, the TnsC is expressed under a different promoter which results in lower expression of TnsC. Accordingly, the nucleic acid encoding TnsC may utilize a lower copy number promoter than the promoter for any or all of remaining components of the CAST system. Alternatively, the methods may utilize other means for decreasing TnsC expression levels in a cell which are known in the art, including, for example the use of downregulating oligonucleotides (e.g., small interfering RNAs (siRNAs), an antisense oligonucleotides, short-hairpin RNAs, microRNAs (miRNA), PIWI-interacting RNAs (piRNAs), dicer-substrate RNAs, DNAzymes, small circular RNAs, aptamers targeting a gene or messenger RNAs), which can be used to modulate gene expression.

In some embodiments, the methods and systems comprise modulating the affinity of TnsC for DNA binding. In some embodiments, the methods and system comprise introducing specific mutations to TnsC or disrupting the formation of long TnsC filaments to attenuating the innate affinity of TnsC for DNA binding at random sites and/or increasing its affinity for RNA-dependent sites.

In some embodiments, the methods and systems comprise modulating the electrostatics in the DNA-binding pore of many CAST TnsCs, which possess a positive DNA binding pore, further enhances CAST system specificity. In some embodiments, the methods and system comprise engineered or mutated TnsC proteins. In some embodiments, the methods and systems comprise an engineered or mutated TnsC protein comprising one or more mutations to decrease affinity for DNA binding at non-specific sites and/or increase affinity for target site DNA binding. In some embodiments, the methods and systems comprise an engineered or mutated TnsC protein comprising one or more mutations to disrupt the formation of TnsC filaments.

In some embodiments, the methods and systems may comprise utilization of an exogenous agent to the system which disrupt or control formation of TnsC filaments. For example, in cell-free systems DNA intercalating agents can disrupt TnsC filaments.

Conversely, in some embodiments, the methods of system can be engineered to decrease specificity and increase off-target functionality. In select embodiments, the methods and system comprise a TnsC protein having mutations which result in an energetically favored/active state capable for facilitating integration anywhere in the genome without sequence requirements, e.g., type V-K ShCAST K103A TnsC. For example, fusing K103A to any high-affinity DNA binding effector, (for e.g., dCas9 or the other nuclease-dead targeting proteins listed above), and, optionally, decreasing the overall cytoplasmic concentration of the fusion to levels facilitate binding directed by the effector's DNA affinity rather than that of TnsC, could permit efficient integration without compromising on-target specificity.

In some embodiments, mutation of DNA contacting residues in type V-K ShCAST TnsC, such as K103, T121, K150, R182, and K119, and/or inter-subunit residues such as K51 and R52 (relative to SEQ ID NO: 52), are employed to tune TnsC availability at RNA-dependent versus RNA-independent sites, thereby enhancing specificity.

In some embodiments, the specificity of CAST in cells can be increased by perturbing the TnsC N-terminus. For example, the specificity of CAST in cells can be increased by fusing a moiety (e.g., protein or protein domain) to the N-terminus of TnsC. However, the methods and systems are not limited by the type of moiety. The moiety may include any perturbation to the N-terminus of TnsC. Fusion constructs of other components of the CAST system, which limit the steps involved in CAST component recruitment for RNA-dependent integration, could also regulate TnsC recruitment at RNA-dependent sites.

Other perturbations include effector domains, as described elsewhere herein, or other exogenous proteins or protein domains. In select embodiments, a fusion of ribosomal protein S15 to TniQ with an XTEN-GS linker (S15-XTEN-GS-TniQ), which improved specificity and also result in higher on-target efficiency at low-TnsC conditions in cells.

### b) Precision at the Site of DNA Integration

In general, CAST integration occurs within a window of bases downstream of the target site, rather than at a precise location. For instance, type I-F VchCAST integrates approximately 36-54 bp downstream of the protospacer, or when measured from the PAM, about 77-87 bp downstream of the PAM. Type I-B AvCAST typically integrates 43-49 bp downstream of the protospacer, or 80-86 bp from the PAM. Type V-K ShCASTs are found to integrate roughly 35-45 bp downstream of the protospacer, or 58-68 bp from the PAM. Similarly, type V-K ShoCAST (also referred to as ShoINTEGRATE, or ShoINT) and AcCAST integrate around 25-35 bp downstream of the protospacer, or 48-58 bp from the PAM.

In some embodiments, the methods and systems comprise engineering the target nucleic acid. Thus, the disclosure provides methods and systems which exploit the sequence features that influence CAST target preference to facilitate precise positioning of integration at a fixed location rather than within a range. In some embodiments, the disclosure provides methods to probe and explore which sequence feature influence CAST target preference.

Previous research using library-based approaches has only partially characterized sequence features corresponding to TnsB DNA recognition. As disclosed herein, computational analysis of unique integration sites revealed features corresponding to the sequence preference of both TnsC and TnsB at the target DNA, as disclosed below. By applying these unique sequence preferences and features in the design of target locations, the disclosed systems and methods improve the accuracy and efficiency of gene editing using CAST systems. For example, engineering target sites for RNA-independent activity may involve use of AT rich sequences as disclosed herein as AT-rich sites on DNA are preferentially bound by TnsC, and thus preferentially ‘targeted’ for RNA-independent transposition. Additionally, ‘targeted’, RNA-independent insertions may include both the poly-A stretch and flanking TnsB consensus motifs as described in Example 4.

### c) TnsB Function and Abundance

As shown herein, TnsB exhibits an inherent bias to favor integration at RNA-dependent sites over RNA-independent sites. In some embodiments, the methods and systems comprise increasing TnsB levels. Such increases in TnsB levels can enhance efficiency at RNA-dependent sites.

In certain embodiments, TnsB or the nucleic acid encoding TnsB is in greater abundance compared to the remaining protein components or nucleic acids encoding thereof. For example, multiple copies of a nucleic acid encoding TnsB may be provided for each copy of any of the other components (e.g., Cas6, Cas5, Cas8, TnsA, TnsB, or TnsC). In some embodiments, TnsB is encoded on a nucleic acid separate from any of the other components such that it can be provided in the system and methods herein at a higher abundance or dosage (e.g., under control of a higher copy number plasmid or under control of a promoter region under the influence of a transcription activator) than the other components. Analogously, higher concentrations of the TnsB protein can be provided in the systems and methods compared to the other proteins.

In some embodiments, the methods and systems increasing the nuclear concentration of TnsB, e.g., in eukaryotic cells. The methods and system may comprise transfection of cells with purified TnsB tagged with at least one nuclease localization sequence (NLS). The nuclear localization sequence may comprise any amino acid sequence known in the art to functionally tag or direct a protein for import into a cell's nucleus (e.g., for nuclear transport). Usually, a nuclear localization sequence comprises one or more positively charged amino acids, such as lysine and arginine.

In some embodiments, the NLS is a monopartite sequence. A monopartite NLS comprises a single cluster of positively charged or basic amino acids. In some embodiments, the monopartite NLS comprises a sequence of K-K/R-X-K/R, wherein X can be any amino acid. Exemplary monopartite NLSs include, without limitation, those from the SV40 large T-antigen (PKKKRKVEDP; SEQ ID NO: 111), c-Myc (PAAKRVKLD; SEQ ID NO: 112), and TUS-proteins (Kaczmarczyk S J et al. 2010). In select embodiments, the NLS comprises a c-Myc NLS.

In some embodiments, the NLS is a bipartite sequence. Bipartite NLSs comprise two clusters of basic amino acids, separated by a spacer of about 9-12 amino acids. Exemplary bipartite NLSs include the NLS of nucleoplasmin, KR[PAATKKAGQA]KKKK (SEQ ID NO: 113), the NLS of EGL-13, MSRRRKANPTKLSENAKKLAKEVEN (SEQ ID NO: 114), the bipartite SV40 NLS, KRTADGSEFESPKKKRKV (SEQ ID NO: 115). In some embodiments, the NLS comprises a bipartite SV40 NLS.

Additionally, supplementing ribosomal protein S15 at high TnsB concentrations has demonstrated an increase in in vitro integration efficiency up to 33%. Thus, in some embodiments, the methods and systems further comprise adding S15. The addition of S15 may be particularly used in methods and system which have an increased abundance of TnsB. The S15 protein may be derived from any organism. In select embodiments, the S15 protein is derived from S. hofmannii. In some embodiments, the methods or systems comprise an exogenous S15 protein. In some embodiments, the exogenous S15 protein is a bacterial S15 protein. In some embodiments, the methods or system comprise expressing or increasing expression of the host S15 protein.

The observed sequence biases on target DNA as a consequence of TnsB binding, indicate potential mutations in Type V-K ShCAST TnsB residues K290, R416, T417, Q425, and N428 could boost the efficiency of RNA-guided integration and improve CAST specificity. In some embodiments, the systems and methods comprise an engineered TnsB which preferentially favors integration at RNA-dependent sites. In some embodiments, the engineered TnsB comprises mutations at one or more residues selected from K290, R416, T417, Q425, and N428.

### Nucleic Acids and Delivery

The present disclosure also provides for nucleic acids encoding the components of the disclosed systems, compositions comprising nucleic acids encoding the components of the disclosed systems, systems comprising nucleic acids encoding the components of the disclosed systems, and vectors containing or encoding these nucleic acids. The vectors may be used to propagate the nucleic acid in an appropriate cell and/or to allow expression from the nucleic acid (e.g., an expression vector). The person of ordinary skill in the art would be aware of the various vectors available for propagation and expression of a nucleic acid sequence.

The present disclosure further provides engineered, non-naturally occurring vectors and vector systems, which can encode one or more of the peptides or components of the present systems. The vector(s) can be introduced into a cell that is capable of expressing the polypeptide encoded thereby, including any suitable prokaryotic or eukaryotic cell.

The vectors of the present disclosure may be delivered to a eukaryotic cell in a subject. Modification of the eukaryotic cells via the present system can take place in a cell culture, where the method comprises isolating the eukaryotic cell from a subject prior to the modification. In some embodiments, the method further comprises returning said eukaryotic cell and/or cells derived therefrom to the subject.

Viral and non-viral based gene transfer methods can be used to introduce nucleic acids encoding the disclosed polypeptides or components of the present system into cells, tissues, or a subject. Such methods can be used to administer nucleic acids encoding the disclosed polypeptides or components of the present system to cells in culture, or in a host organism. Non-viral vector delivery systems include DNA plasmids, cosmids, RNA (e.g., a transcript of a vector described herein), a nucleic acid, and a nucleic acid complexed with a delivery vehicle. Viral vector delivery systems include DNA and RNA viruses, which have either episomal or integrated genomes after delivery to the cell. Viral vectors include, for example, retroviral, lentiviral, adenoviral, adeno-associated and herpes simplex viral vectors.

In certain embodiments, plasmids that are non-replicative, or plasmids that can be cured by high temperature may be used, such that any or all of the necessary components of the system may be removed from the cells under certain conditions. For example, this may allow for DNA integration by transforming bacteria of interest, but then being left with engineered strains that have no memory of the plasmids or vectors used for the integration. Drug selection strategies may be adopted for positively selecting for cells. A donor nucleic acid may contain one or more drug-selectable markers within the cargo. Then presuming that the original donor plasmid is removed, drug selection may be used to enrich for integrated clones. Colony screenings may be used to isolate clonal events.

A variety of viral constructs may be used to deliver the disclosed polypeptides or components of the present system (such as one or more Cas proteins and/or transposon associated proteins, gRNA(s), donor DNA, etc.) to the targeted cells and/or a subject. Nonlimiting examples of such recombinant viruses include recombinant adeno-associated virus (AAV), recombinant adenoviruses, recombinant lentiviruses, recombinant retroviruses, recombinant herpes simplex viruses, recombinant poxviruses, phages, etc. The present disclosure provides vectors capable of integration in the host genome, such as retrovirus or lentivirus. See, e.g., Ausubel et al., Current Protocols in Molecular Biology, John Wiley & Sons, New York, 1989; Kay, M. A., et al., 2001 Nat. Medic. 7(1): 33-40; and Walther W. and Stein U., 2000 Drugs, 60(2): 249-71, incorporated herein by reference.

In one embodiment, a nucleic acid encoding the disclosed polypeptides or components of the present system is contained in a plasmid vector that allows expression of the disclosed polypeptides or components of the present system and subsequent isolation and purification of from the recombinant vector. Accordingly, the disclosed polypeptides or components of the present system disclosed herein can be purified following expression, obtained by chemical synthesis, or obtained by recombinant methods.

To construct cells that express the disclosed polypeptides or components of the present system, expression vectors for stable or transient expression of the disclosed polypeptides or components of the present system may be constructed via conventional methods as described herein and introduced into host cells. For example, nucleic acids encoding the components of the disclosed polypeptides or components of the present system may be cloned into a suitable expression vector, such as a plasmid or a viral vector in operable linkage to a suitable promoter. The selection of expression vectors/plasmids/viral vectors should be suitable for integration and replication in eukaryotic cells.

In certain embodiments, vectors of the present disclosure can drive the expression of one or more sequences in prokaryotic cells. Promoters that may be used include T7 RNA polymerase promoters, constitutive E. coli promoters, and promoters that could be broadly recognized by transcriptional machinery in a wide range of bacterial organisms. The system may be used with various bacterial hosts.

In certain embodiments, vectors of the present disclosure can drive the expression of one or more sequences in mammalian cells using a mammalian expression vector. Examples of mammalian expression vectors include pCDM8 (Seed, Nature (1987) 329:840, incorporated herein by reference) and pMT2PC (Kaufman, et al., EMBO J. (1987) 6:187, incorporated herein by reference). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd eds., Cold Spring Harbor Laboratory, Cold Spring Harbor Laboratory Press, Cold Spring Harbor, N.Y., 1989, incorporated herein by reference.

Vectors of the present disclosure can comprise any of a number of promoters known to the art, wherein the promoter is constitutive, regulatable or inducible, cell type specific, tissue-specific, or species specific. In addition to the sequence sufficient to direct transcription, a promoter sequence of the invention can also include sequences of other regulatory elements that are involved in modulating transcription (e.g., enhancers, Kozak sequences and introns). Many promoter/regulatory sequences useful for driving constitutive expression of a gene are available in the art and include, but are not limited to, for example, CMV (cytomegalovirus promoter), EF1a (human elongation factor 1 alpha promoter), SV40 (simian vacuolating virus 40 promoter), PGK (mammalian phosphoglycerate kinase promoter), Ubc (human ubiquitin C promoter), human beta-actin promoter, rodent beta-actin promoter, CBh (chicken beta-actin promoter), CAG (hybrid promoter contains CMV enhancer, chicken beta actin promoter, and rabbit beta-globin splice acceptor), TRE (Tetracycline response element promoter), H1 (human polymerase III RNA promoter), U6 (human U6 small nuclear promoter), and the like. Additional promoters that can be used for expression of the components of the present system, include, without limitation, cytomegalovirus (CMV) intermediate early promoter, a viral LTR such as the Rous sarcoma virus LTR, HIV-LTR, HTLV-1 LTR, Maloney murine leukemia virus (MMLV) LTR, myeoloproliferative sarcoma virus (MPSV) LTR, spleen focus-forming virus (SFFV) LTR, the simian virus 40 (SV40) early promoter, herpes simplex tk virus promoter, elongation factor 1-alpha (EF1-α) promoter with or without the EF1-α intron. Additional promoters include any constitutively active promoter. Alternatively, any regulatable promoter may be used, such that its expression can be modulated within a cell.

Moreover, inducible and tissue specific expression of a RNA, transmembrane proteins, or other proteins can be accomplished by placing the nucleic acid encoding such a molecule under the control of an inducible or tissue specific promoter/regulatory sequence. Examples of tissue specific or inducible promoter/regulatory sequences which are useful for this purpose include, but are not limited to, the rhodopsin promoter, the MMTV LTR inducible promoter, the SV40 late enhancer/promoter, synapsin 1 promoter, ET hepatocyte promoter, GS glutamine synthase promoter and many others. Various commercially available ubiquitous as well as tissue-specific promoters and tumor-specific are available, for example from InvivoGen. In addition, promoters which are well known in the art can be induced in response to inducing agents such as metals, glucocorticoids, tetracycline, hormones, and the like, are also contemplated for use with the invention. Thus, it will be appreciated that the present disclosure includes the use of any promoter/regulatory sequence known in the art that is capable of driving expression of the desired protein operably linked thereto.

The vectors of the present disclosure may direct expression of the nucleic acid in a particular cell type (e.g., tissue-specific regulatory elements are used to express the nucleic acid). Such regulatory elements include promoters that may be tissue specific or cell specific. The term “tissue specific” as it applies to a promoter refers to a promoter that is capable of directing selective expression of a nucleotide sequence of interest to a specific type of tissue (e.g., seeds) in the relative absence of expression of the same nucleotide sequence of interest in a different type of tissue. The term “cell type specific” as applied to a promoter refers to a promoter that is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., immunohistochemical staining.

Additionally, the vector may contain, for example, some or all of the following: a selectable marker gene, such as the neomycin gene for selection of stable or transient transfectants in host cells; enhancer/promoter sequences from the immediate early gene of human CMV for high levels of transcription; transcription termination and RNA processing signals from SV40 for mRNA stability; 5′- and 3′-untranslated regions for mRNA stability and translation efficiency from highly-expressed genes like a-globin or β-globin; SV40 polyoma origins of replication and ColE1 for proper episomal replication; internal ribosome binding sites (IRESes), versatile multiple cloning sites; T7 and SP6 RNA promoters for in vitro transcription of sense and antisense RNA; a “suicide switch” or “suicide gene” which when triggered causes cells carrying the vector to die (e.g., HSV thymidine kinase, an inducible caspase such as iCasp9), and reporter gene for assessing expression of the chimeric receptor. Suitable vectors and methods for producing vectors containing transgenes are well known and available in the art. Selectable markers also include chloramphenicol resistance, tetracycline resistance, spectinomycin resistance, streptomycin resistance, erythromycin resistance, rifampicin resistance, bleomycin resistance, thermally adapted kanamycin resistance, gentamycin resistance, hygromycin resistance, trimethoprim resistance, dihydrofolate reductase (DHFR), GPT; the URA3, HIS4, LEU2, and TRP1 genes of S. cerevisiae.

When introduced into the cell, the vectors may be maintained as an autonomously replicating sequence or extrachromosomal element or may be integrated into host DNA.

In one embodiment, the donor DNA may be delivered using the same gene transfer system as used to deliver the Cas protein, and/or transposon associated proteins (included on the same vector) or may be delivered using a different delivery system. In another embodiment, the donor DNA may be delivered using the same transfer system as used to deliver gRNA(s).

In one embodiment, the present disclosure comprises integration of exogenous DNA into the endogenous gene. Alternatively, an exogenous DNA is not integrated into the endogenous gene. The DNA may be packaged into an extrachromosomal or episomal vector (such as AAV vector), which persists in the nucleus in an extrachromosomal state, and offers donor-template delivery and expression without integration into the host genome. Use of extrachromosomal gene vector technologies has been discussed in detail by Wade-Martins R (Methods Mol Biol. 2011; 738:1-17, incorporated herein by reference).

The disclosed polypeptides or components of the present system (e.g., proteins, polynucleotides encoding these proteins, donor polynucleotides and compositions comprising the proteins and/or polynucleotides described herein) may be delivered by any suitable means. In certain embodiments, the polypeptides or system is delivered in vivo. In other embodiments, the polypeptides or system is delivered to isolated/cultured cells (e.g., autologous iPS cells) in vitro to provide modified cells useful for in vivo delivery to patients afflicted with a disease or condition.

Vectors according to the present disclosure can be transformed, transfected, or otherwise introduced into a wide variety of cells. Transfection refers to the taking up of a vector by a cell whether or not any coding sequences are in fact expressed. Numerous methods of transfection are known to the ordinarily skilled artisan, for example, lipofectamine, calcium phosphate co-precipitation, electroporation, DEAE-dextran treatment, microinjection, viral infection, and other methods known in the art. Transduction refers to entry of a virus into the cell and expression (e.g., transcription and/or translation) of sequences delivered by the viral vector genome. In the case of a recombinant vector, “transduction” generally refers to entry of the recombinant viral vector into the cell and expression of a nucleic acid of interest delivered by the vector genome.

Any of the vectors comprising a nucleic acid sequence that encodes the disclosed polypeptides or components of the present system is also within the scope of the present disclosure. Such a vector may be delivered into host cells by a suitable method. Methods of delivering vectors to cells are well known in the art and may include DNA or RNA electroporation, transfection reagents such as liposomes or nanoparticles to delivery DNA or RNA; delivery of DNA, RNA, or protein by mechanical deformation (see, e.g., Sharei et al. Proc. Natl. Acad. Sci. USA (2013) 110(6): 2082-2087, incorporated herein by reference); or viral transduction. In some embodiments, the vectors are delivered to host cells by viral transduction. Nucleic acids can be delivered as part of a larger construct, such as a plasmid or viral vector, or directly, e.g., by electroporation, lipid vesicles, viral transporters, microinjection, and biolistics (high-speed particle bombardment). Similarly, the construct containing the one or more transgenes can be delivered by any method appropriate for introducing nucleic acids into a cell. In some embodiments, the construct or the nucleic acid encoding the disclosed polypeptides or components of the present system is a DNA molecule. In some embodiments, the nucleic acid encoding the disclosed polypeptides or components of the present system is a DNA vector and may be electroporated to cells. In some embodiments, the nucleic acid encoding the disclosed polypeptides or components of the present system is an RNA molecule, which may be electroporated to cells.

Additionally, delivery vehicles such as nanoparticle- and lipid-based mRNA or protein delivery systems can be used. Further examples of delivery vehicles include lentiviral vectors, ribonucleoprotein (RNP) complexes, lipid-based delivery system, gene gun, hydrodynamic, electroporation or nucleofection microinjection, and biolistics. Various gene delivery methods are discussed in detail by Nayerossadat et al. (Adv Biomed Res. 2012; 1:27) and Ibraheem et al. (Int J Pharm. 2014 Jan. 1; 459(1-2): 70-83), incorporated herein by reference.

### Methods for Modifying a Target Nucleic Acid

Also disclosed herein are methods for nucleic acid modification or integration utilizing the disclosed systems. The methods may comprise contacting a target nucleic acid sequence with a system or component thereof disclosed herein. The descriptions and embodiments provided above for the systems are applicable to the methods described herein.

The phrase “modifying a nucleic acid sequence” or “nucleic acid modification” as used herein, refers to modifying at least one physical feature of a nucleic acid sequence of interest. Nucleic acid modifications include, for example, single or double strand breaks, deletion, or insertion of one or more nucleotides, and other modifications that affect the structural integrity or nucleotide sequence of the nucleic acid sequence.

The target nucleic acid sequence may be in a cell. In some embodiments, contacting a target nucleic acid sequence comprises introducing the system into the cell. As described above the system may be introduced into eukaryotic or prokaryotic cells by methods known in the art. In some embodiments, the cell is a mammalian cell. In some embodiments, the cell is a human cell.

In some embodiments, the target nucleic acid is a nucleic acid endogenous to a target cell. In some embodiments, the target nucleic acid is a genomic DNA sequence. The term “genomic,” as used herein, refers to a nucleic acid sequence (e.g., a gene or locus) that is located on a chromosome in a cell.

In some embodiments, the target nucleic acid encodes a gene or gene product. The term “gene product,” as used herein, refers to any biochemical product resulting from expression of a gene. Gene products may be RNA or protein. RNA gene products include non-coding RNA, such as tRNA, rRNA, micro RNA (miRNA), and small interfering RNA (siRNA), and coding RNA, such as messenger RNA (mRNA). In some embodiments, the target nucleic acid sequence encodes a protein or polypeptide.

The method may comprise administering to the subject, in vivo, or by transplantation of ex vivo treated cells, an effective amount of the described system or components thereof. In some embodiments, the vector(s) is delivered to the tissue of interest by, for example, an intramuscular, intravenous, transdermal, intranasal, oral, mucosal, or other delivery methods.

The components of the present system, or ex vivo treated cells may be administered with a pharmaceutically acceptable carrier or excipient as a pharmaceutical composition. In some embodiments, the components of the present system may be mixed, individually or in any combination, with a pharmaceutically acceptable carrier to form pharmaceutical compositions, which are also within the scope of the present disclosure.

In some embodiments, an effective amount of the components of the present system, or compositions as described herein can be administered. As used herein the term “effective amount” may be used interchangeably with the term “therapeutically effective amount” and refers to that quantity that is sufficient to result in a desired activity upon administration to a subject in need thereof. Within the context of the present disclosure, the term “effective amount” refers to that quantity of the components of the system such that successful DNA integration or modification is achieved.

When utilized as a method of treatment, the effective amount may depend on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of any disease or disorder in the subject. In some embodiments, the subject is a human.

In the context of the present disclosure insofar as it relates to any of the disease conditions recited herein, the terms “treat,” “treatment,” and the like mean to relieve or alleviate at least one symptom associated with such condition, or to slow or reverse the progression of such condition. Within the meaning of the present disclosure, the term “treat” also denotes to arrest, delay the onset (e.g., the period prior to clinical manifestation of a disease) and/or reduce the risk of developing or worsening a disease. For example, in connection with cancer the term “treat” may mean eliminate or reduce a patient's tumor burden, or prevent, delay, or inhibit metastasis, etc.

The phrase “pharmaceutically acceptable,” as used in connection with compositions and/or cells of the present disclosure, refers to molecular entities and other ingredients of such compositions that are physiologically tolerable and do not typically produce untoward reactions when administered to a subject (e.g., a mammal, a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans. “Acceptable” means that the carrier is compatible with the active ingredient of the composition (e.g., the nucleic acids, vectors, cells, or therapeutic antibodies) and does not negatively affect the subject to which the composition(s) are administered. Any of the pharmaceutical compositions and/or cells to be used in the present methods can comprise pharmaceutically acceptable carriers, excipients, or stabilizers in the form of lyophilized formations or aqueous solutions.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g., Remington: The Science and Practice of Pharmacy 20th Ed. (2000) Lippincott Williams and Wilkins, Ed. K. E. Hoover.

The methods may be used for a variety of purposes. For example, the methods may include, but are not limited to, inactivation of a microbial gene, RNA-guided DNA integration in a plant or animal cell, methods of treating a subject suffering from a disease or disorder (e.g., cancer, Duchenne muscular dystrophy (DMD), sickle cell disease (SCD), B-thalassemia, and hereditary tyrosinemia type I (HT1)), and methods of treating a diseased cell (e.g., a cell deficient in a gene which causes cancer).

The disclosed methods may modify a target DNA sequence in a cell so as to modulate expression of the target DNA sequence, e.g., expression of the target DNA sequence is increased, decreased, or completely eliminated (e.g., via deletion of a gene). The modifications of the target sequence may lead to, for example, gene correction, gene replacement, gene tagging, transgene insertion, nucleotide deletion, gene disruption, gene mutation, gene knock-down, etc.

In some embodiments, the methods described herein may be used to correct one or more defects or mutations in a gene (referred to as “gene correction”). In such cases, the target sequence encodes a defective version of a gene, and the disclosed compositions and systems further comprise a donor nucleic acid molecule which encodes a wild-type or corrected version of the gene. Accordingly, in some embodiments, the methods described herein may be used to insert a gene or fragment thereof into a cell.

In another embodiment, the method of modifying a target sequence can be used to delete nucleic acids from a target sequence in a host cell by cleaving the target sequence and allowing the host cell to repair the cleaved sequence in the absence of an exogenously provided donor nucleic acid molecule. Deletion of a nucleic acid sequence in this manner can be used in a variety of applications, such as, for example, to remove disease-causing trinucleotide repeat sequences in neurons, to create gene knock-outs or knock-downs, and to generate mutations for disease models in research.

In some embodiments, the methods described herein may be used to genetically modify a plant or plant cell. As used herein, genetically modified plants include a plant into which has been introduced an exogenous polynucleotide. Genetically modified plants also include a plant that has been genetically manipulated such that endogenous nucleotides have been altered to include a mutation, such as a deletion, an insertion, a transition, a transversion, or a combination thereof. For instance, an endogenous coding region could be deleted. Such mutations may result in a polypeptide having a different amino acid sequence than was encoded by the endogenous polynucleotide. Another example of a genetically modified plant is one having an altered regulatory sequence, such as a promoter, to result in increased or decreased expression of an operably linked endogenous coding region. The genetically modified plant may promote a desired phenotypic or genotypic plant trait.

Genetically modified plants can potentially have improved crop yields, enhanced nutritional value, and increased shelf life. They can also be resistant to unfavorable environmental conditions, insects, and pesticides. The present systems and methods have broad applications in gene discovery and validation, mutational and cisgenic breeding, and hybrid breeding. The present methods may facilitate the production of a new generation of genetically modified crops with various improved agronomic traits such as herbicide resistance, herbicide tolerance, drought tolerance, male sterility, insect resistance, abiotic stress tolerance, modified fatty acid metabolism, modified carbohydrate metabolism, modified seed yield, modified oil percent, modified protein percent, resistance to bacterial disease, disease (e.g. bacterial, fungal, and viral) resistance, high yield, and superior quality. The present methods may also facilitate the production of a new generation of genetically modified crops with optimized fragrance, nutritional value, shelf-life, pigmentations (e.g., lycopene content), starch content (e.g., low-gluten wheat), toxin levels, propagation and/or breeding and growth time. See, for example, CRISPR/Cas Genome Editing and Precision Plant Breeding in Agriculture (Chen et al., Annu Rev Plant Biol. 2019 Apr. 29; 70:667-69), incorporated herein by reference.

The present method may confer one or more of the following traits to the plant cell: herbicide tolerance, drought tolerance, male sterility, insect resistance, abiotic stress tolerance, modified fatty acid metabolism, modified carbohydrate metabolism, modified seed yield, modified oil percent, modified protein percent, resistance to bacterial disease, resistance to fungal disease, and resistance to viral disease.

The present disclosure provides for a modified plant cell produced by the present method, a plant comprising the plant cell, and a seed, fruit, plant part, or propagation material of the plant. Transformed or genetically modified plant cells of the present disclosure may be as populations of cells, or as a tissue, seed, whole plant, stem, fruit, leaf, root, flower, stem, tuber, grain, animal feed, a field of plants, and the like. The present disclosure provides a transgenic plant. The transgenic plant may be homozygous or heterozygous for the genetic modification. Also provided by the present disclosure are transformed or genetically modified plant cells, tissues, plants, and products that contain the transformed or genetically modified plant cells. The present disclosure further encompasses the progeny, clones, cell lines or cells of the transgenic plants.

The present system and method may be used to modify a plant stem cell. The present disclosure further provides progeny of a genetically modified cell, where the progeny can comprise the same genetic modification as the genetically modified cell from which it was derived. The present disclosure further provides a composition comprising a genetically modified cell.

In one embodiment, the transformed or genetically modified cells, and tissues and products comprise a nucleic acid integrated into the genome, and production by plant cells of a gene product due to the transformation or genetic modification.

Methods of introducing exogenous nucleic acids into plant cells are well known in the art. Such plant cells are considered “transformed.” DNA constructs can be introduced into plant cells by various methods, including, but not limited to PEG- or electroporation-mediated protoplast transformation, tissue culture or plant tissue transformation by biolistic bombardment, or the Agrobacterium-mediated transient and stable transformation. The transformation can be transient or stable transformation. Suitable methods also include viral infection (such as double stranded DNA viruses), transfection, conjugation, protoplast fusion, electroporation, particle gun technology, calcium phosphate precipitation, direct microinjection, silicon carbide whiskers technology, Agrobacterium-mediated transformation, and the like. The choice of method is generally dependent on the type of cell being transformed and the circumstances under which the transformation is taking place (e.g., in vitro, ex vivo, or in vivo). Transformation methods based upon the soil bacterium Agrobacterium tumefaciens are useful for introducing an exogenous nucleic acid molecule into a vascular plant. The wild-type form of Agrobacterium contains a Ti (tumor-inducing) plasmid that directs production of tumorigenic crown gall growth on host plants. Transfer of the tumor-inducing T-DNA region of the Ti plasmid to a plant genome requires the Ti plasmid-encoded virulence genes as well as T-DNA borders, which are a set of direct DNA repeats that delineate the region to be transferred. An Agrobacterium-based vector is a modified form of a Ti plasmid, in which the tumor inducing functions are replaced by the nucleic acid sequence of interest to be introduced into the plant host.

Agrobacterium-mediated transformation generally employs cointegrate vectors or binary vector systems, in which the components of the Ti plasmid are divided between a helper vector, which resides permanently in the Agrobacterium host and carries the virulence genes, and a shuttle vector, which contains the gene of interest bounded by T-DNA sequences. A variety of binary vectors are well known in the art and are commercially available, for example, from Clontech (Palo Alto, Calif.). Methods of coculturing Agrobacterium with cultured plant cells or wounded tissue such as leaf tissue, root explants, hypocotyledons, stem pieces or tubers, for example, also are well known in the art. See., e.g., Glick and Thompson, (eds.), Methods in Plant Molecular Biology and Biotechnology, Boca Raton, Fla.: CRC Press (1993), incorporated herein by reference.

Microprojectile-mediated transformation also can be used to produce a transgenic plant. This method, first described by Klein et al. (Nature 327:70-73 (1987), incorporated herein by reference), relies on microprojectiles such as gold or tungsten that are coated with the desired nucleic acid molecule by precipitation with calcium chloride, spermidine, or polyethylene glycol. The microprojectile particles are accelerated at high speed into an angiosperm tissue using a device such as the BIOLISTIC PD-1000 (Biorad; Hercules Calif.).

In one embodiment, the present methods may be adapted to use in plants. The vectors may be optimized for transient expression of the present system in plant protoplasts, or for stable integration and expression in intact plants via the Agrobacterium-mediated transformation.

In certain embodiments, the present methods use a monocot promoter to drive the expression of one or more components of the present systems (e.g., gRNA) in a monocot plant. In certain embodiments, the present methods use a dicot promoter to drive the expression of one or more components of the present systems (e.g., gRNA) in a dicot plant.

The present methods may be used with various microbial species, including human pathogens that are medically important, and bacterial pests that are key targets within the agricultural industry, as well as antibiotic resistant versions thereof. The method may be designed to target any gene or any set of genes, such as virulence or metabolic genes, for clinical and industrial applications in other embodiments. For example, the present methods may be used to target and eliminate virulence genes from the population, to perform in situ gene knockouts, or to stably introduce new genetic elements to the metagenomic pool of a microbiome. The present systems and methods may be used to treat a multi-drug resistance bacterial infection in a subject. The present systems and methods may be used for genomic engineering within complex bacterial consortia.

The present systems and methods may be used to inactivate microbial genes. In some embodiments, the gene is an antibiotic resistance gene. For example, the coding sequence of bacterial resistance genes may be disrupted in vivo by insertion of a DNA sequence, leading to non-selective re-sensitization to drug treatment.

The methods described here also provide for treating a disease or condition in a subject. The method may comprise administering to the subject, in vivo, or by transplantation of ex vivo treated cells (e.g., disclosed T cells), a therapeutically effective amount of the present system, polypeptides, or components thereof.

In some embodiments, the methods are used to treat a pathogen or parasite on or in a subject by altering the pathogen or parasite. In some embodiments, the methods target a “disease-associated” gene. The term “disease-associated gene,” refers to any gene or polynucleotide whose gene products are expressed at an abnormal level or in an abnormal form in cells obtained from a disease-affected individual as compared with tissues or cells obtained from an individual not affected by the disease. A disease-associated gene may be expressed at an abnormally high level or at an abnormally low level, where the altered expression correlates with the occurrence and/or progression of the disease. A disease-associated gene also refers to a gene, the mutation or genetic variation of which is directly responsible or is in linkage disequilibrium with a gene(s) that is responsible for the etiology of a disease. Examples of genes responsible for such “single gene” or “monogenic” diseases include, but are not limited to, adenosine deaminase, α-1 antitrypsin, cystic fibrosis transmembrane conductance regulator (CFTR), β-hemoglobin (HBB), oculocutaneous albinism II (OCA2), Huntingtin (HTT), dystrophia myotonica-protein kinase (DMPK), low-density lipoprotein receptor (LDLR), apolipoprotein B (APOB), neurofibromin 1 (NF1), polycystic kidney disease 1 (PKD1), polycystic kidney disease 2 (PKD2), coagulation factor VIII (F8), dystrophin (DMD), phosphate-regulating endopeptidase homologue, X-linked (PHEX), methyl-CpG-binding protein 2 (MECP2), and ubiquitin-specific peptidase 9Y, Y-linked (USP9Y). Other single gene or monogenic diseases are known in the art and described in, e.g., Chial, H. Rare Genetic Disorders: Learning About Genetic Disease Through Gene Mapping, SNPs, and Microarray Data, Nature Education 1(1):192 (2008); Online Mendelian Inheritance in Man (OMIM); and the Human Gene Mutation Database (HGMD). In another embodiment, the target genomic DNA sequence can comprise a gene, the mutation of which contributes to a particular disease in combination with mutations in other genes. Diseases caused by the contribution of multiple genes which lack simple (i.e., Mendelian) inheritance patterns are referred to in the art as a “multifactorial” or “polygenic” disease. Examples of multifactorial or polygenic diseases include, but are not limited to, asthma, diabetes, epilepsy, hypertension, bipolar disorder, and schizophrenia. Certain developmental abnormalities also can be inherited in a multifactorial or polygenic pattern and include, for example, cleft lip/palate, congenital heart defects, and neural tube defects. In another embodiment, the target DNA sequence can comprise a cancer oncogene. The present disclosure provides for gene editing methods that can ablate a disease-associated gene (e.g., a cancer oncogene), which in turn can be used for in vivo gene therapy for patients. In some embodiments, the gene editing methods include donor nucleic acids comprising therapeutic genes.

### Methods for RNA-Guided Tagmentation of DNA

The disclosure also provides methods for RNA-guided tagmentation of DNA in vitro for applications using purified CAST components. The methods comprise any one or all of conducting RNA-guided site specific transposition of a sample to integrate a donor nucleic acid into nucleic acids in the sample; incubating purified nucleic acids from the sample with a tagmentation reaction mixture to generate tagmented nucleic acid fragments; amplifying the tagmented nucleic acid fragments with transposon end specific primers and adaptor-containing primers to generate a library of amplicons; sequencing the library of amplicons; and analyzing sequences of the amplicons for on-target integration, off-target integration, and orientation of the integration. In some embodiments, each of the amplicons comprises a unique barcode or unique molecular identifier 9UMI). The barcode or UMI may be comprised with the primers used for amplification.

In some embodiments, the RNA guided tagmentation is site-specific. For example, amplifying the tagmented nucleic acid fragments with transposon end specific primers enriches for transposon end sequences prior to adaptor addition and sequencing of barcoded amplicons.

“Tagmentation” refers to the modification of DNA by a complex comprising transposase enzyme and adaptors comprising transposon end sequence. Essentially tagmentation is transposase mediated fragmentation and tagging. Tagmentation results in the simultaneous fragmentation of large nucleic acids (e.g., DNA) into smaller-sized fragments for use in next generation sequencing platforms which sequence, on average, about 300 base pairs in length, and ligation of the adaptors to the 5′ ends of both strands of duplex fragments. The methods may use any transposase that can accept a transposase end sequence and fragment a target nucleic acid, attaching a transferred end, but not a non-transferred end. Following a purification step to remove the transposase enzyme, additional sequences (e.g., barcodes) can be added to the ends of the adapted fragments, for example by PCR, ligation, or any other suitable methodology known to those of skill in the art. Tagmentation reactions combine random fragmentation and adapter ligation into a single step to increase the efficiency of the sequencing library preparation process. Tagmentation can be carried out in solution or on a solid support. Exemplary methods of tagmentation are disclosed in U.S. Pat. Nos. 9,115,396; 9,080,211; 9,040,256; 9,684,230; 10,920,219; 11,90,695.

In some embodiments, transposon based technology can be utilized for fragmenting DNA, for example as exemplified in the workflow for Nextera™ DNA sample preparation kits (Illumina, Inc.) wherein genomic DNA can be fragmented by an engineered transposase complex that simultaneously fragments and tags input DNA (“tagmentation”) thereby creating a population of fragmented nucleic acid molecules which comprise unique adapter sequences at the ends of the fragments.

In some embodiments, the methods further comprise removing non-integrated donor nucleic acids (e.g., prior to generation of tagmented nucleic acids to avoid contamination of non-integrated donor nucleic acids in the analysis). Removal may be completed by making use of digestion sites within the polynucleotide comprising the donor nucleic acid. For example, the polynucleotide comprising the donor nucleic acid may comprise one or more digestion sites (e.g., restriction endonuclease sites) that allow digestion of the donor nucleic acid whereas such sites are infrequent in the target plasmid of genomic DNA targeted for insertion. In some embodiments, the polynucleotide comprising the donor nucleic acid comprises a digestion site outside of the donor nucleic acid and transposon end sequences. Thus, the removing step may comprise digesting the nucleic acids from the sample with an enzyme directed to the digestion site. Methods of size separation can be used to resolve the resulting nucleic acids and isolate the nucleic acids of interest from the starting donor nucleic acid.

The RNA-guided transposition may be carried out in vitro or in vivo. In some embodiments, the RNA-guided transposition is carried out as a plasmid-to-plasmid transposition assay. Thus, the RNA-guided transposition may be completed with genomic DNA or plasmid targets.

In some embodiments, the RNA-guided transposition is carried out at low TnsC amounts or concentrations. In some embodiments, the RNA-guided transposition is carried out at high TnsB amounts or concentrations. In some embodiments, the RNA-guided transposition is carried out in vitro. Thus, the methods leverage the increased efficiency at high TnsB concentration, and specificity (99.7%) observed at low TnsC conditions in vitro biochemical reactions. The preincubation step noted on CAST literature at low Mg2+ levels for 30° C. is not required for the biochemical reconstitution of transposition.

The methods facilitate characterization of the RNA-guided transposition. For example, the analysis may allow determination of the presence or absence of on-target transposition, the presence or absence of off-target transposition, and/or donor insert orientation. In some embodiments, the methods further comprise quantifying the degree of on-target transposition versus off-target transposition and determine the quantity of total transposon-end containing reads. In some embodiments, the methods further comprise normalizing the quantitation of on-target integration and off-target integration with the total transposon-end containing reads.

In addition, the methods may further comprise analysis of the genetic neighborhood, the sequences surrounding the site of insertion. This may be read out by the sequencing itself or be done by a comparison to a reference sequence from the starting nucleic acids in the sample (e.g., determining genome/plasmid-wide coordinates for each insertion). For example, an integration site may be examined to determine if a presumed off-target site is a true off-target site or if the sequences surrounding such site may comprise a potential PAM sequence.

### Kits

Also within the scope of the present disclosure are kits that include components of the present system.

The kit may include instructions for use in any of the methods described herein. The instructions can comprise a description of administration to a subject to achieve the intended effect. The instructions generally include information as to dosage, dosing schedule, and route of administration for the intended treatment. The kit may further comprise a description of selecting a subject suitable for treatment based on identifying whether the subject is in need of the treatment.

The kits provided herein are in suitable packaging. Suitable packaging includes, but is not limited to, vials, bottles, jars, flexible packaging, and the like. A kit may have a sterile access port (for example, the container may be an intravenous solution bag or a vial having a stopper pierceable by a hypodermic injection needle). The container may also have a sterile access port.

The packaging may be unit doses, bulk packages (e.g., multi-dose packages) or subunit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

The kit may further comprise a device for holding or administering the present system, polypeptides, or composition. The device may include an infusion device, an intravenous solution bag, a hypodermic needle, a vial, and/or a syringe.

The present disclosure also provides for kits for performing nucleic acid modification and integration in vitro. Optional components of the kit include one or more of the following: buffer constituents, control plasmid, sequencing primers, cells.

### EXAMPLES

The following are examples of the present invention and are not to be construed as limiting.

### Example 1

### Type V-K CASTs Undergo RNA-Dependent and RNA-Independent Transposition Pathways

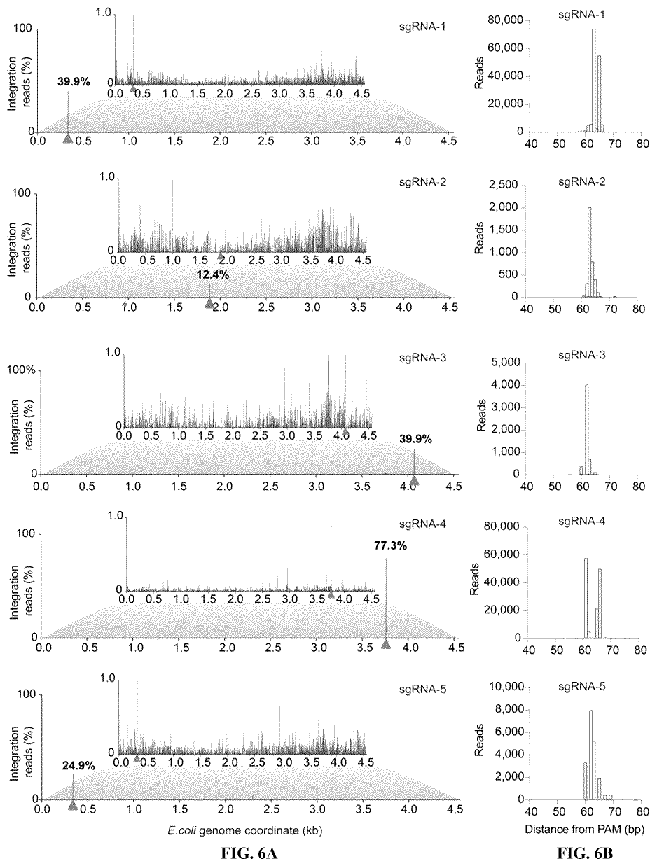

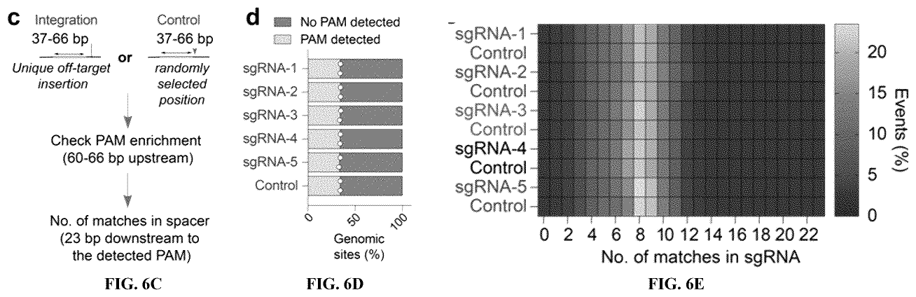

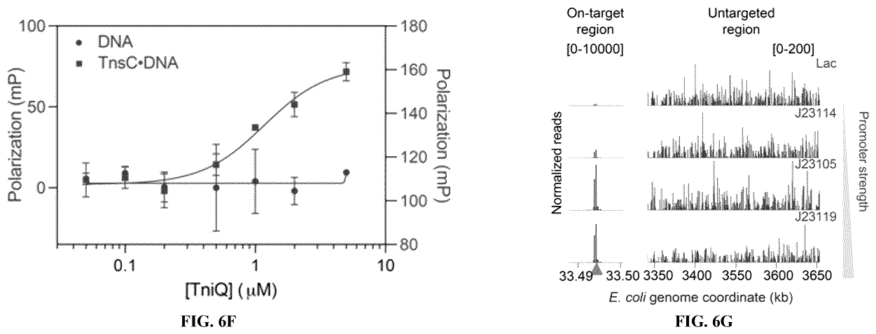

Previous studies of the type V-K ShCAST system from Scytonema hofmannii revealed that a considerable proportion of genomic integration events occur at sites distant from the target site dictated by the guide RNA. To understand the molecular basis of these events, a high-throughput sequencing approach was applied to unbiasedly capture genome-wide integration events upon ShCAST expression with various genetic perturbations (FIG. 1B). After testing five distinct single-guide RNAs (sgRNA), the fraction of on-target integration events ranged from 12-76%, and the majority of events occurred elsewhere, with DNA insertions seemingly randomly distributed across the genome at low individual frequencies (FIG. 1C, 6A, 6B). Analysis of their proximal genetic neighborhood failed to detect enriched sequence similarity to the guide RNA (FIGS. 6C-6E), suggesting that these events were not mismatched off-targets aberrantly targeted by RNA-guided Cas12k, but rather, the consequence of RNA-independent transposition; there were tentatively referred to as untargeted integration events (FIG. 1C). When cas12k and the sgRNA from the original pHelper expression plasmid was deleted, the CRISPR-lacking ShCAST system still produced efficient genome-wide transposition products (FIGS. 1D-1E). These results establish that type V-K CAST systems are capable of both RNA-dependent targeted DNA integration, and RNA-independent untargeted DNA integration.

Additional control experiments confirmed that TnsC, a AAA+ regulator, and TnsB, the DDE-family transposase, facilitate both RNA-dependent and RNA-independent transposition, as their deletion abrogated integration (FIG. 1D). It was initially hypothesized that TnsB and TnsC would comprise the minimum necessary protein components for RNA-independent transposition. However, tniQ deletion had a severe effect on untargeted transposition (FIG. 1D), suggesting a role in stabilizing and/or interacting with the TnsBC transpososome. Recent structures revealed that DNA-bound TnsC oligomers are capped on the N-terminal face by one or more TniQ protomers, and biochemical experiments similarly demonstrated that TniQ only stably associated with DNA in the presence of TnsC, as reflected by fluorescence polarization experiments (FIG. 6F). Thus, ShCAST, and perhaps type V-K CAST systems more generally, maintain a prominent ‘BCQ’ pathway that facilitates CRISPR RNA-independent, untargeted transposition.

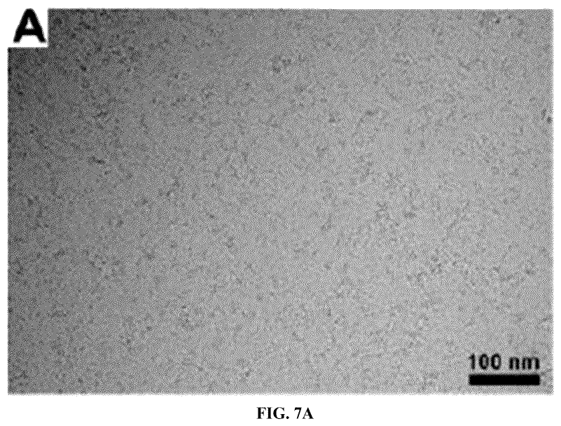

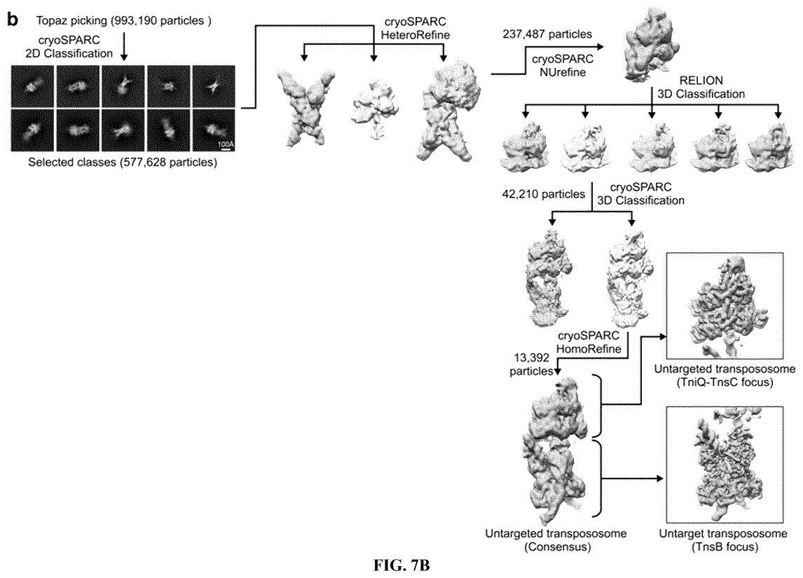

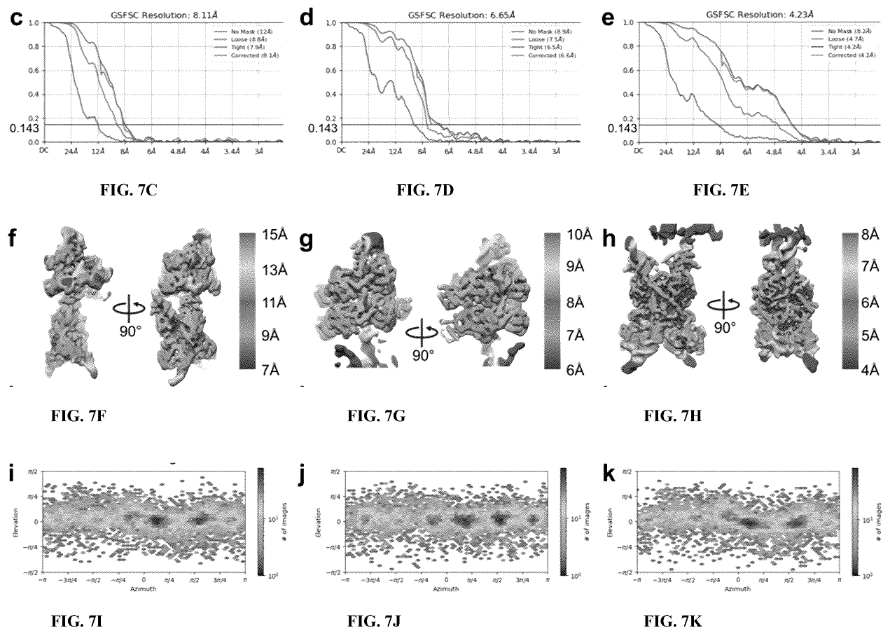

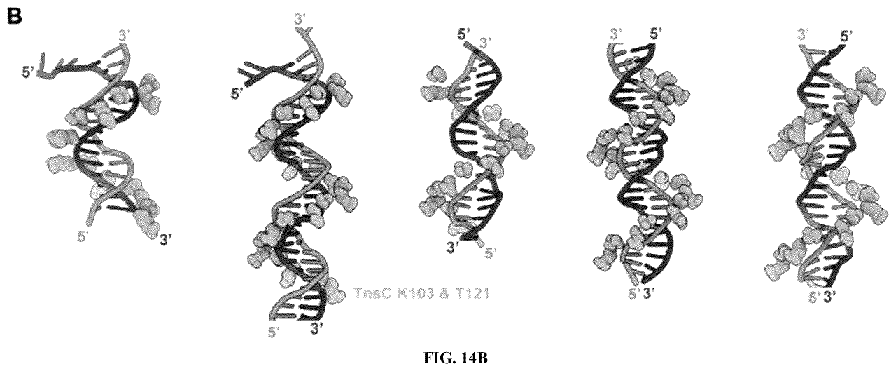

To capture the architecture of components contributing to untargeted integration, cryo-electron microscopy (cryo-EM) was used to visualize TnsB, TnsC and TniQ in a strand-transfer complex (STC). Although the cryo-EM density of TniQ was less well-resolved (˜8 Å) compared to other subunits (FIG. 7), likely due to heterogeneity of binding configurations, atomic models of all protein components were able to be unambiguously docked to DNA in the map (FIG. 1F). The overall structure of the ‘BCQ’ transpososome resembled the Cas12k-containing transpososome (FIG. 14A), with two turns of TnsC filaments preferentially selected even with free DNA available at the TniQ end. Further, DNA-interacting residues of TnsC (K103 and T121) were positioned to follow the helical symmetry of duplex DNA, as in the structure of helical TnsC filaments. However, in contrast to the Cas12k-containing transpososome, the polarity of the interacting DNA strand in the ‘BCQ’ transpososome was 3′ to 5′ following the direction of TniQ- to TnsB-binding face of TnsC, which was also noted in the structure of TniQ-TnsC (FIG. 14B). Therefore, the ‘BCQ’ transpososome structure, which represents a low energy configuration of TnsC, revealed that DNA contacts in TnsC filaments are maintained differently at on-target and untargeted sites. Yet, the overall architecture of the ‘BCQ’ transpososome, comprising the TnsB STC, two turns of a TnsC mini-filament (spanning a DNA-binding footprint of 25 bp) and TniQ, resembled the on-target Cas12k bound transpososome.

After cloning cas12k onto a separate expression plasmid and systematically varying its promoter strength, on-target integration events were proportionally increased, though without a reduction in the frequency of untargeted integration (FIG. 1G, 6G). These results indicate that, at least under these expression conditions, the availability of Cas12k-sgRNA complexes limits RNA-guided DNA integration efficiency but does not directly affect the ‘BCQ’ pathway. Type V-K CAST systems often encode a MerR-family transcriptional regulator adjacent to the Cas12k gene, and a recent study demonstrated that these Cas V-K repressor (CvkR) proteins down-regulate both Cas12k and TnsB expression, though with distinct effects. Thus, although present knowledge about CAST activity is largely limited to comparative genomics and artificial heterologous over-expression, it appears likely that CAST transposition in native contexts is regulated so as to modulate the frequency of RNA-dependent and RNA-independent target pathways.

### Example 2

### Relative TnsB and TnsC Stoichiometry Determines the Transposition Pathway Choice

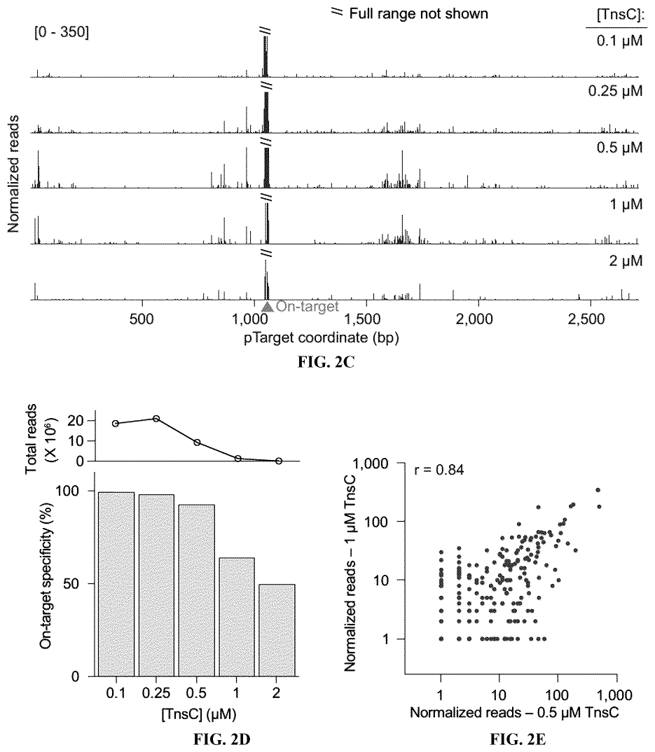

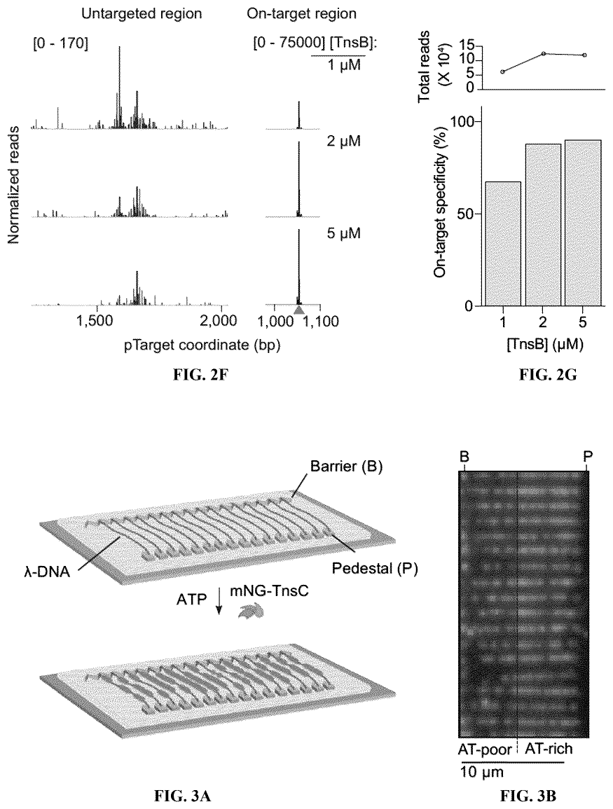

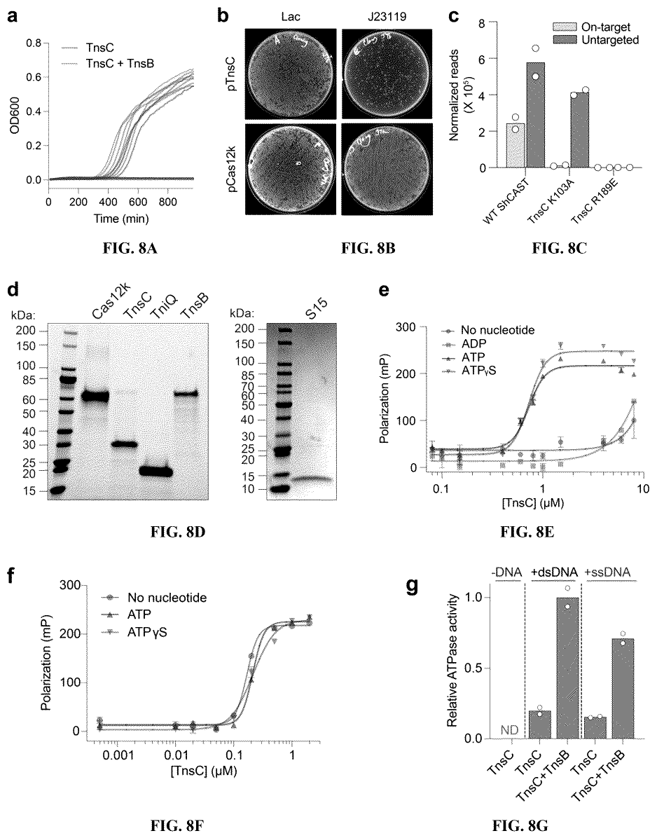

Many other bacterial transposons encode transposition proteins homologous to ShCAST, including type I CASTs, Tn7, Tn5053, IS21, and Mu. The TnsBC module is common to all. To experimentally investigate the role of TnsC in target site selection and the effect of variable stoichiometries of TnsB, TnsC and TniQ have on untargeted integration, major challenges were encountered while trying to vary the expression of transposon components in cells due to associated toxicity, particularly with the over-expression of TnsC (FIG. 2A, 8A, 8B). When liquid cultures were inoculated with a strain expressing tnsC alone from a strong promoter and induced over-expression in the lag phase, a complete growth arrest was observed for the majority of clones, with only a few strains undergoing delayed growth, likely due to suppressor mutations in the plasmid or genome (FIG. 8A). Strikingly, this cellular toxicity was completely rescued with a mutation to the arginine finger motif, which abrogates TnsC filamentation and transposition, or partially rescued by co-expression of TnsC and TnsB (FIG. 2A, 8C). These results implicate non-specific DNA filamentation as a likely source of cellular toxicity, which can be relieved in part by the ability of TnsB to disassemble TnsC filaments, as demonstrated from in vitro experiments.

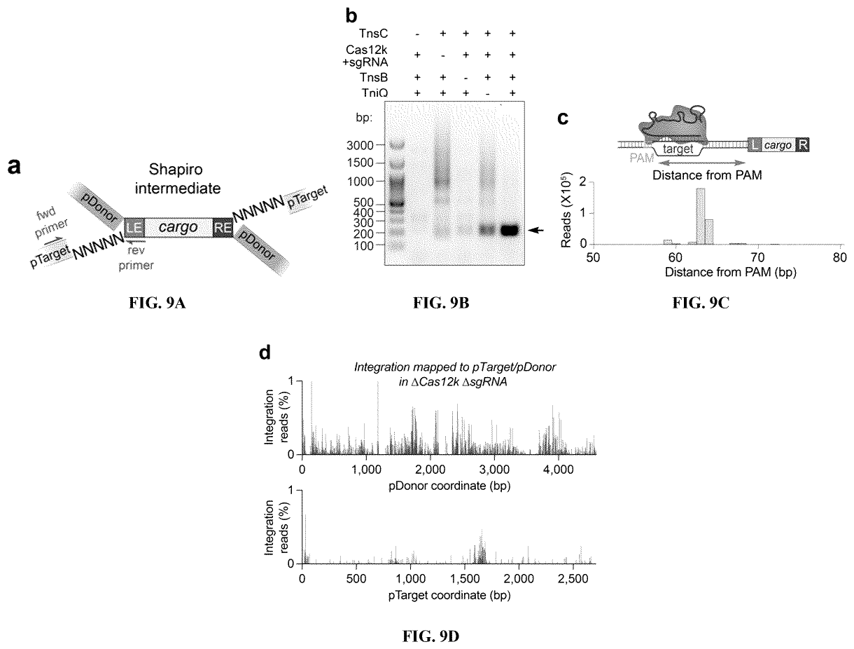

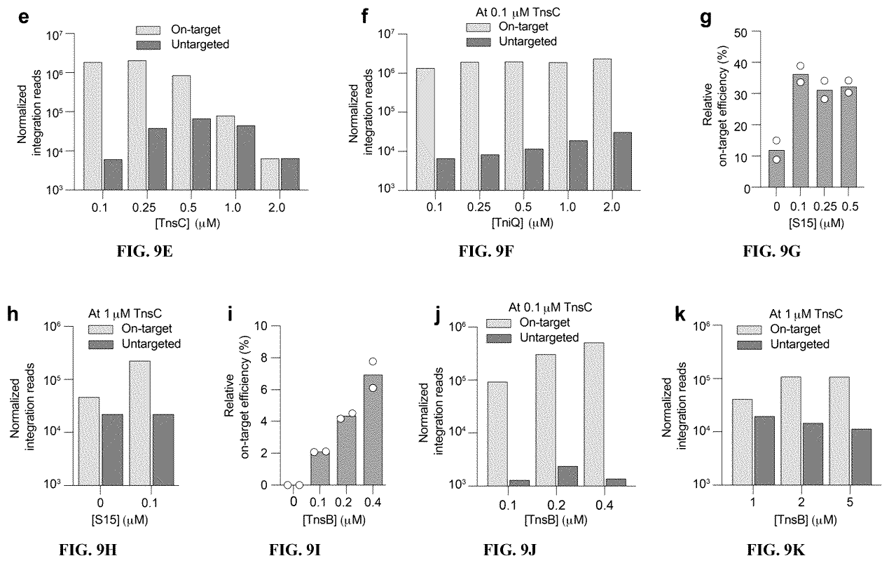

In order to modulate the stoichiometries of CAST components contributing to untargeted integration, while avoiding confounding factors such as toxicity, a biochemical approach was adopted. After recombinantly expressing and purifying ShCAST components and testing the activity of TnsC and TnsB in vitro (FIGS. 8D-8G), a plasmid-to-plasmid (pDonor-to-pTarget) transposition assay was established (FIGS. 2B, 8D-8G). In initial experiments, on-target products were amplified by targeted PCR, thereby revealing molecular requirements for each of the transpososome components and the expected distance separating the target and integration site (FIGS. 9A-9C). Next, the biochemical experiments were coupled with tagmentation-based high-throughput sequencing, in order to unbiasedly map DNA transposition events regardless of their insertion site (FIG. 2B). Remarkably, at low (0.1 μM) concentrations of TnsC, transposition was highly accurate, with >99% of reads representing on-target integration events, defined as occurring within a 100-bp window downstream of the target site (FIGS. 2C-2D). However, when the concentration of TnsC was systematically increased while keeping all other components constant, the frequency of untargeted integration events increased, approaching levels similar to those observed in cellular transposition assays (FIGS. 2D and 9E). Substantial untargeted integration events also occurred in the absence of Cas12k and sgRNA under these conditions, in agreement with in vivo experiments (FIG. 9D). TnsC concentrations of 1 μM or higher resulted in a decrease in both on-target and untargeted integration, which may be due to prohibitive coating of DNA by TnsC filaments (see below). When interpreted together with structural data, these results suggest that RNA-independent transposition is likely initiated by the formation of dsDNA-bound TnsC filaments. Importantly, untargeted integration events were not randomly distributed across pTarget but instead clustered into specific and reproducible hotspot regions (FIG. 2E), suggesting a selectivity for certain sequence features (see below).

The impact of other protein components on in vitro transposition activity was tested. Ribosomal protein S15, a recently described host factor that stimulates ShCAST transposition by binding the sgRNA, dramatically increased the frequency of on-target integration events, as measured both by deep sequencing and qPCR, but had no discernible effect on untargeted integration events (FIGS. 9G-9H). Increasing the concentration of TniQ, on the other hand, led to a monotonic increase in the frequency of untargeted integration events without a major effect on on-target integration (FIG. 9F), suggesting that the RNA-independent pathway may be more sensitive to limited TniQ availability.

The TnsB transposase has been previously shown to disassemble TnsC filaments from dsDNA, and it is therefore possible that titrating excess amounts of TnsB would lead to partial or full disassembly of TnsC filaments necessary for transposition, regardless of their molecular context. Yet, when the amount of the TnsB transposase was varied, distinct effects were observed at on-target and untargeted sites (FIGS. 2F, 9I-9K). Increasing TnsB led to a notable increase in RNA-guided integration but resulted in a slight decrease in untargeted events (FIGS. 9I-9K), leading to an overall rescue of specificity at on-target sites with high TnsC concentrations. This observation suggests that TnsC filaments at targeted versus untargeted sites are differentially susceptible to TnsB-induced disassembly, and/or react to undergo strand transfer with distinct kinetics. Transpososome structures reveal that TnsB interacts with TnsC filaments on only one face, and no major structural changes are associated with TnsC filaments at on-target and untargeted sites (FIG. 1F). Therefore, the distinct nature of DNA interactions made by TnsC at both these sites determines filament stabilization versus disassembly.

Collectively, these results suggest that the natural propensity of TnsC to form long filaments on dsDNA exerts a fitness cost on cells in the absence of accessory transposase machinery and is a driver of RNA-independent, untargeted transposition.

### Example 3

### TnsC Preferentially Targets AT-Rich DNA During RNA-Independent Transposition

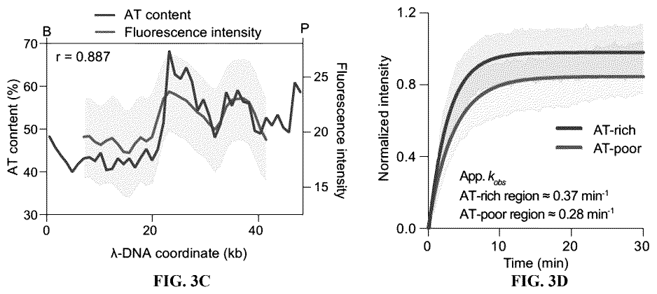

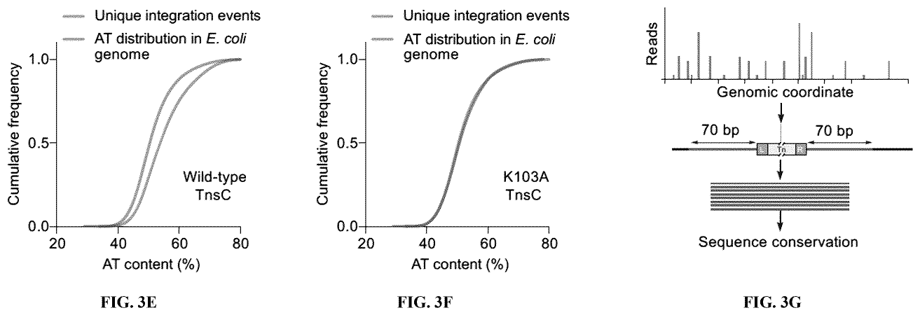

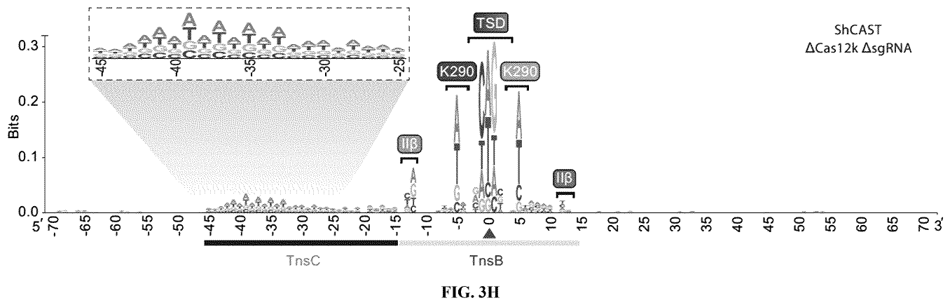

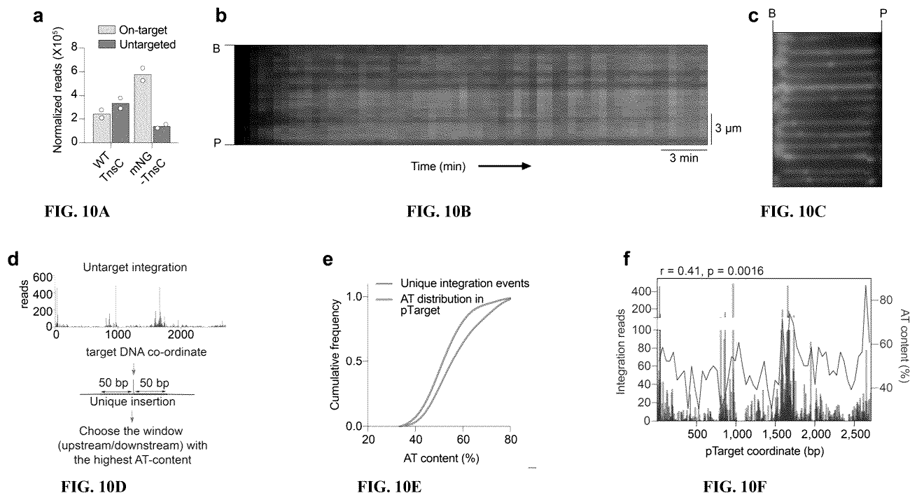

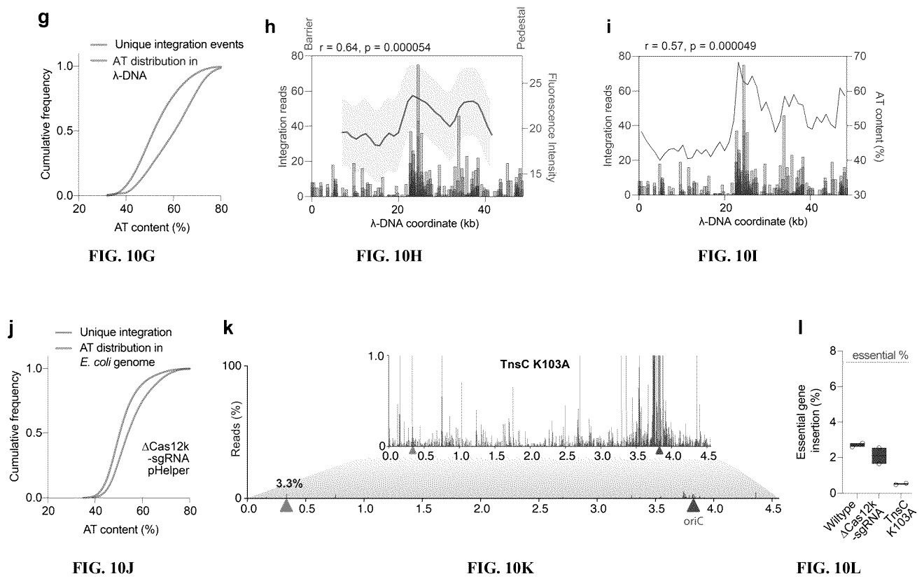

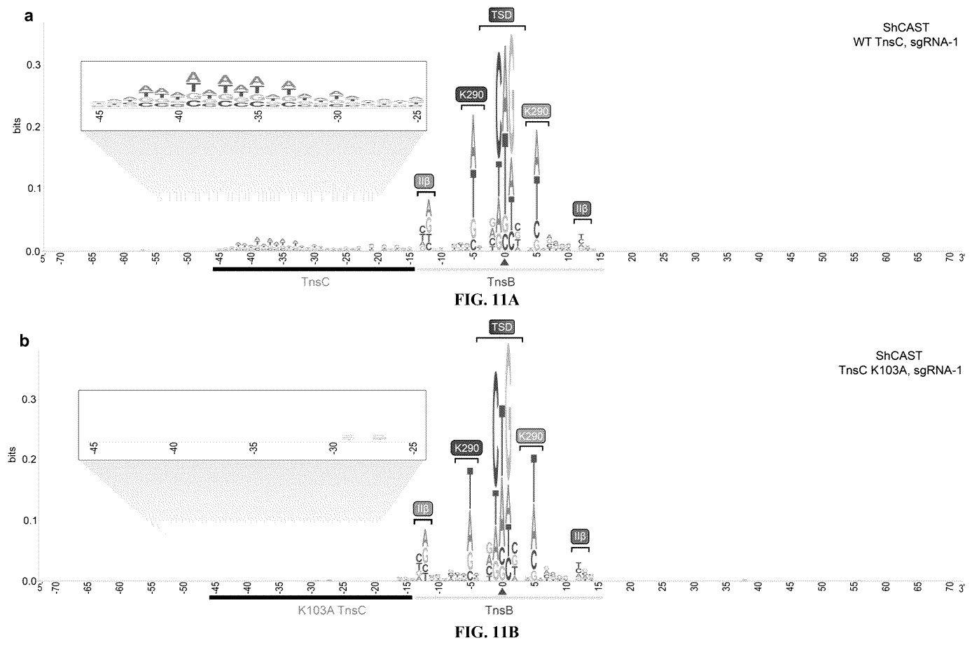

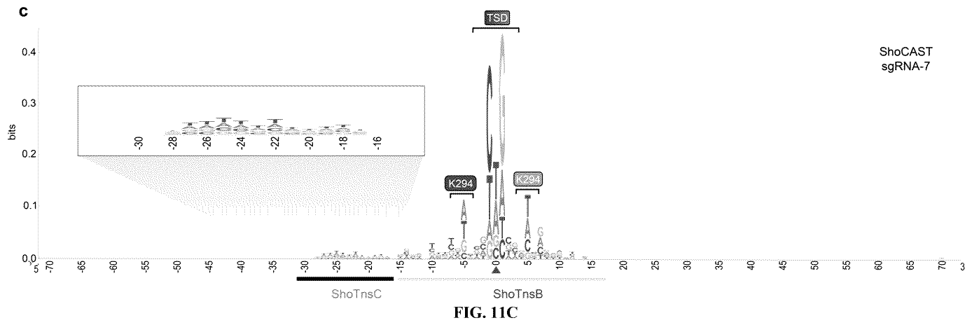

A single-molecule approach was developed to visualize DNA binding by TnsC using DNA curtains (FIG. 3A, 10A), in which λ-phage genomic DNA molecules are tethered between chrome patterns on a quartz slide and imaged by total internal reflection microscopy (TIRFM). Fluorescently labeled TnsC remained fully active for RNA-guided transposition, albeit with slightly increased specificity relative to WT (FIG. 10A), suggesting that the N-terminal appendage may subtly impact DNA binding and/or transpososome assembly. In DNA curtains experiments, TnsC exhibited stable and high-affinity binding in the presence of ATP, and the data furthermore revealed a marked preference for the 3′ half of the genome (FIG. 3B). The λ-phage genome is known to be divided into a GC-rich half and an AT-rich half, and the analyses revealed a significant correlation between AT content and TnsC localization (FIG. 3C), indicating that TnsC filaments preferentially accumulated on the AT-rich half of the λ-phage genome. Time-course experiments further revealed that TnsC binds to AT-rich regions at a faster rate, before saturating the entire λ-DNA substrate within 5-10 min of incubation (FIG. 3D, 10A, 10B). Incubation of DNA curtains with high TnsC concentrations resulted in complete coating of the λ-DNA substrate (FIG. 9C), which could explain the decrease in both on-target and untargeted integration observed in biochemical transposition assays at similarly high TnsC concentrations (FIGS. 2C and 8E).

Given the observation that untargeted transposition events in biochemical assays preferred certain hotspot regions of pTarget and were reproducible between independent experiments (FIG. 2E), AT content might be an underlying feature explaining these data. Analysis of the nucleotide composition surrounding all unique integration events on pTarget found that they were indeed skewed towards more AT-rich DNA (FIGS. 10D-10E). Direct visual superposition of AT content and DNA integration data further revealed that hotspot regions for untargeted transposition in pTarget generally correlated with regions of higher AT content (FIG. 10F). The same phenomenon was observed after performing transposition assays with a λ-DNA substrate and repeating similar analyses to assess AT-bias in the location of untargeted integration sites (FIGS. 10G-10I). Analysis of untargeted integration events in the E. coli genome, from experiments performed without Cas12k and sgRNA, found that these were also highly enriched at local regions of high AT content (FIG. 3E, 10J). These results provide evidence that AT-rich sites on DNA are preferentially bound by TnsC, and thus preferentially ‘targeted’ for RNA-independent transposition.

To uncover additional sequence features common to CRISPR-independent ShCAST transposition products, meta-analysis of all genome-wide integration sites was performed after orienting the flanking sequences based on the asymmetric transposon ends, and a consensus sequence logo of the resulting alignment was generated (FIG. 3G). This analysis revealed two notable clusters of sequence features: nucleotide preferences directly within and surrounding the target-site duplication (TSD), and an AT-rich nucleotide cluster located upstream of the integration site (FIG. 3H). Remarkably, the AT-rich region spans ˜25-bp and could thus accommodate two turns of a dsDNA-bound TnsC filament, similar to the TnsC architecture and footprint observed within the context of Cas12k-containing and Cas12k-lacking transpososomes (FIGS. 1F-1G). The observation that this region is located on only one side of all integration sites argues that RNA-independent integration events result from binding of TnsC filaments to AT-rich DNA, followed by directional recruitment of TnsB to define downstream sites for transposon insertion in the same L-R orientation as occurs at RNA-guided target sites (FIG. 3I). In general, this orientation is referred to as TnsC-LR which could be applicable to other systems employing a AAA+ ATPase for integration. The highest sequence conservation was located furthest from the site of integration, proximal to the presumed region where TnsC filaments are capped by TniQ (FIGS. 1F-1G), and the observed, di-nucleotide periodic trend is reminiscent of structures demonstrating that TnsC monomers contact every two bases of DNA.

The highest conservation in the sequence logo corresponds to sequences contacted by TnsB within the ‘BCQ’ transpososome. As with prior library-based experiments for both type I-F and V-K CAST systems, the results indicate that TnsB preferentially integrates into sites containing ‘GCWGC’ within the TSD (FIG. 3H). However, a bias for (A/T) at symmetric positions located ±5-bp from the TSD center, which is contacted by residue K290 of two TnsB monomers within the transpososome, was also uncovered (FIG. 3J). Nucleotide preferences ±12-bp from the TSD could also be explained by the proximity of these residues with the TnsB IIß DNA-binding domain (R416, T417, Q425, N428), which, also makes similar sequence contacts to the penultimate TnsB binding sites located within the transposon left and right ends. When untargeted integration events from previously published ShCAST data (Vo, P. L. H. et al. Nat Biotechnol 39, 480-489 (2021), in which HTS libraries were generated and sequenced using an alternative strategy, were analyzed the same sequence features were observed, confirming the robustness of this observation (FIG. 11A). Importantly, the absence of any conserved sequence features upstream of the A/T-rich region, where the target site would normally be located during Cas12k-mediated transposition, corroborated the earlier interpretation that the majority of cellular transposition events are RNA-independent.

Previously, it was shown that a point mutation in one of the two TnsC residues (K103A) that contact DNA increased the number of untargeted events without comprising its ability to bind DNA. In agreement with these results, when the same mutant was tested in cellular transposition assays, a severe loss of on-target events but a preservation of untargeted events, which were enriched near the E. coli origin of replication, was observed (FIGS. 8C and 10K). A meta-analysis of untargeted integration events found that the TnsC K103A mutant no longer exhibited an A/T preference, in contrast to WT TnsC (FIG. 3F, 11B). This observation, together with the loss of on-target integration (FIG. 8C), suggests that the K103A mutation results in a more promiscuous mode of DNA binding that supports integration anywhere in the genome without specific sequence requirements.

Transposition activity was previously reported for a type V-K CAST homolog also found in Scytonema hofmannii, ShoCAST (previously referred to as ShoINT), which is diverged from ShCAST and more similar to AcCAST. Meta-analyses were performed on the transposition data and highly similar motifs emerged in the resulting sequence logo, but with a major difference in the window of A/T-rich DNA located upstream of the integration site, which spanned only ˜10-bp compared to ˜25-bp observed with ShCAST (FIG. 11C). This difference is remarkably consistent with the finding that ShoCAST and AcCAST integrate ˜10-bp closer to the target site than ShCAST, suggesting that the transpososomes from this subfamily of CAST systems, for both RNA-dependent and RNA-independent transposition pathways, may comprise a shorter TnsC filament spanning only one turn of DNA.

### Example 4

### Preferred Sequence Motifs Lead to Semi-Targeted, RNA-Independent Transposition

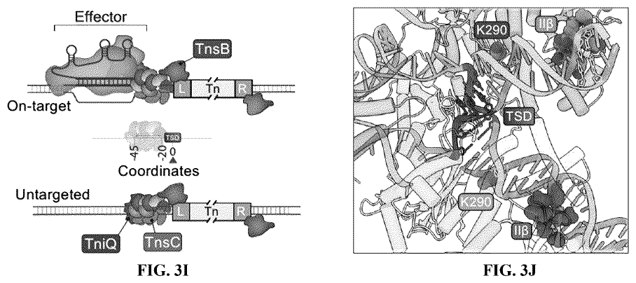

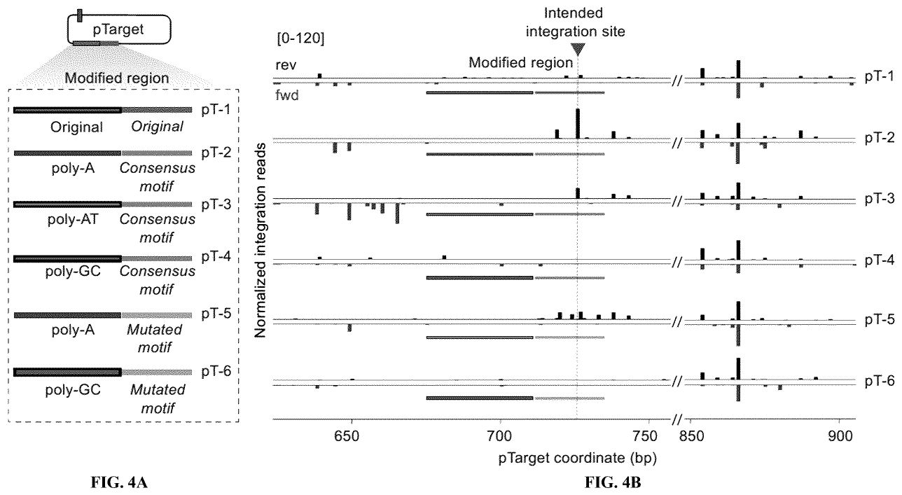

To test the importance of TnsBC-specific sequence motifs more directly for CRISPR-independent integration, biochemical transposition assays using a series of isogenic pTarget substrates that differed only in the sequence content of a select region that was poorly targeted in previous experiments were designed (FIG. 4A, substrate pT-1). It was hypothesized that one could generate ‘targeted’, RNA-independent insertions within this region if an optimal sequence were designed to include both the poly-A stretch and flanking TnsB consensus motifs observed in the sequence logo described above (substrate pT-2; FIG. 3H). As further controls, the poly-A stretch was substituted with either poly-AT or poly-GC, the TnsB consensus was mutagenized, or both motifs were replaced (FIG. 4A, substrates pT-3 through pT-6). Each substrate was tested in biochemical transposition assays and the normalized integration frequency within this window of interest was plotted.

The resulting data demonstrate that RNA-independent integration events occur in ways depending on the sequence features uncovered through the above analyses (FIG. 4B). Substrate pT-2 exhibited a predominant integration product precisely at the engineered site, in the expected T-LR orientation, whereas this integration product was entirely absent when the poly-A was replaced with poly-GC, strengthening the conclusion that favorable TnsC filamentation is important for RNA-independent integration (FIG. 4B, pT-4,6). When the poly-A stretch was retained with a mutated consensus motif favored by TnsB, integration products were more heterogeneously positioned (FIG. 4B, pT-5), suggesting that preferred TnsB-DNA interactions play an important role in dictating the insertion site. When the poly-A sequence was replaced with poly-AT (increasing the A-content in the opposite strand), the intended integration event was diminished in frequency and accompanied by an increase in upstream integration events on the opposite strand (FIG. 4B, pT-3), demonstrating that nucleotide composition can modulate the preferred directionality of TnsC filament formation and thus integration.

These experiments reveal that TnsC prefers to filament unidirectionally on A-rich DNA stretches, leading to downstream integration in the T-LR orientation. The efficiency and exact site of integration is thus a combination of TnsC filament formation propensity and local TnsB sequence preferences.

### Example 5

### TnsC Availability Controls the Specificity of Cellular ShCAST Transposition Activity

Beyond highlighting the role of TnsC in biasing untargeted integration events to occur at select hotspot regions of the genome, the results more generally implicate TnsC filament formation as a major driver of RNA-independent transposition activity. Because the in vitro results suggest that TnsB differentially selects TnsC filaments at on-target versus untargeted sites, it was hypothesized that this difference could be exploited to increase the overall on-target integration accuracy. To test this hypothesis, perturbations intended to repress TnsC filament formation at non-Cas12k-bound target sites were designed, either by fusing TnsC directly to CRISPR effector proteins or by lowering overall TnsC expression levels.

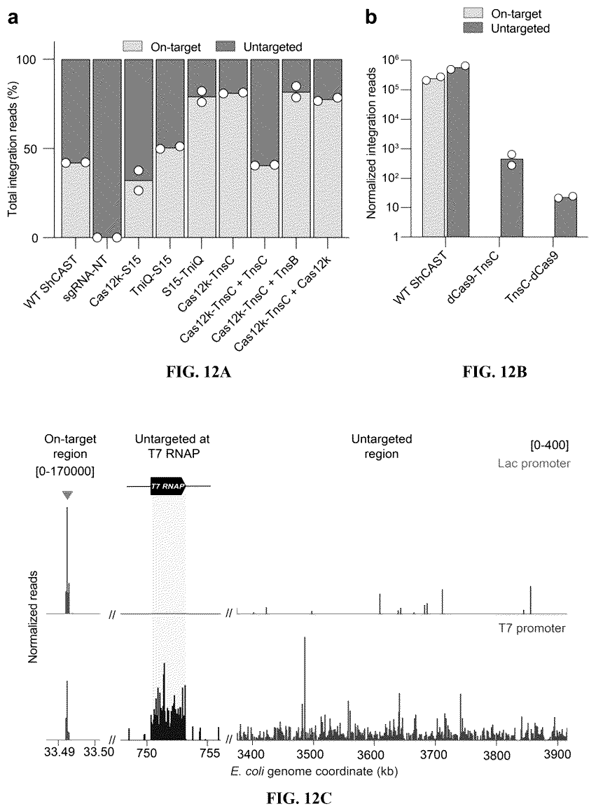

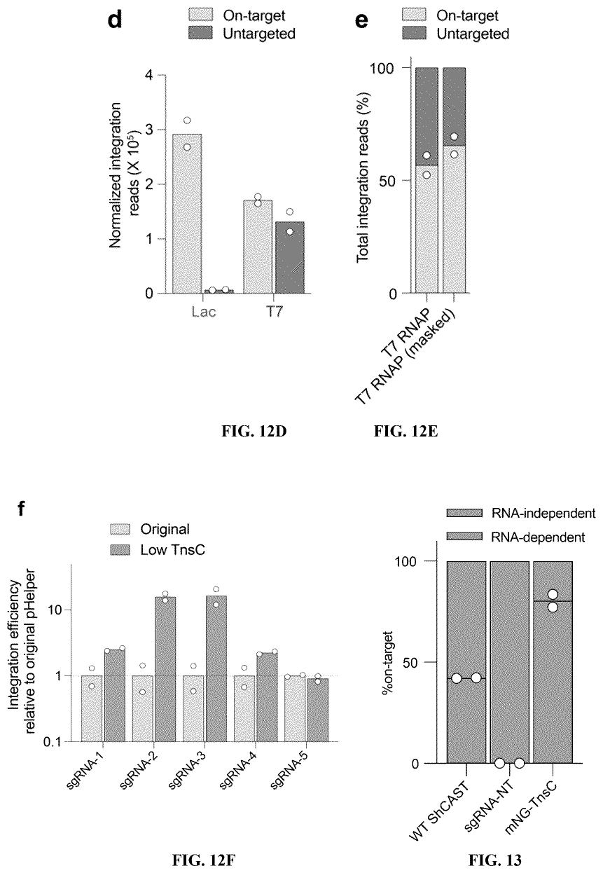

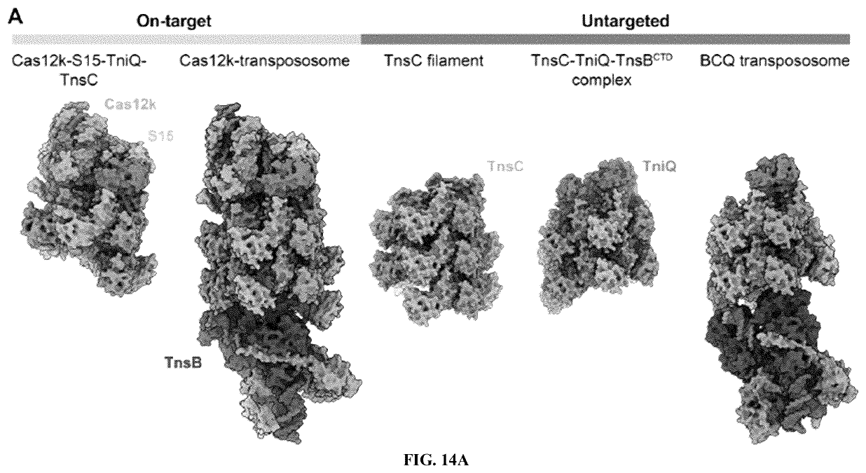

When Cas12k and TnsC were fused, an increase in on-target accuracy (FIG. 12A) was observed. Initially, it was hypothesized that this effect might result from local seeding of TnsC filaments upon Cas12k target binding, but co-expression of unfused Cas12k had no adverse effect on specificity, suggesting instead that TnsC filamentation may be partially impaired with an N-terminal adduct. Cas12k was replaced with dCas9 to generate dCas9-TnsC fusions. However, on-target integration was not observed and untargeted integration events were severely diminished (FIG. 12B), suggesting these designs were non-functional.

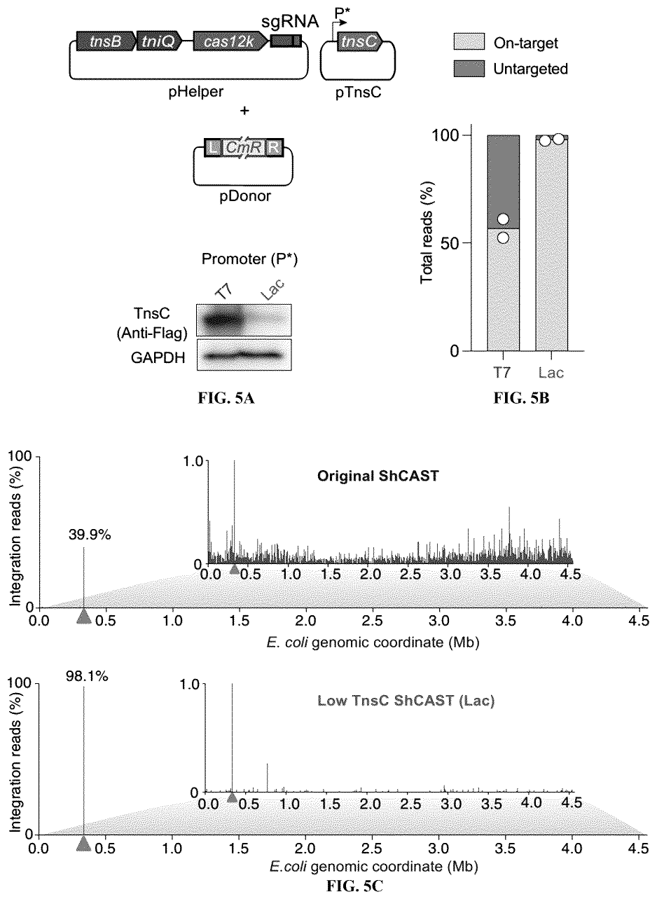

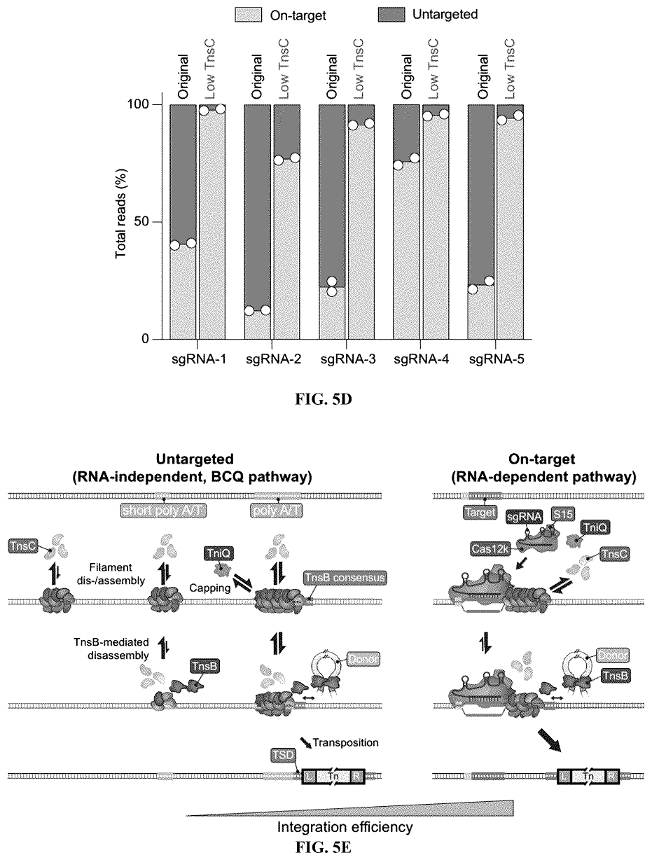

Motivated by the biochemical observation that increasing TnsC concentration tilted the balance between RNA-dependent (on-target) and RNA-independent (untargeted) transposition towards the latter pathway (FIGS. 2C-2D) an alternative strategy was pursued. To test if the same feature was applicable in cellular experiments, tnsC was relocated from the original high-copy pHelper plasmid to a separate, medium-copy plasmid where it was controlled by its own promoter (FIG. 5A). Considerable differences in on-target specificity were observed when genomic integration activity was tested under various promoter strengths (FIGS. 5A-5B). Consistent with the in vitro results, low TnsC expression from a lac promoter resulted in 98% of integration events occurring on-target, whereas high TnsC expression with a T7 promoter resulted in considerably lower accuracy (57%), akin to the original pHelper vector (FIGS. 5A-5C, 12C-12D). Cells expressing TnsC under control of a T7 promoter also showed a significant enrichment for insertion events across the T7 RNAP gene, suggesting that these clones were likely enriched within the population as a way of escaping TnsC-induced toxicity (FIGS. 12C and 12E).

To determine whether this increased specificity effect was generalizable, a range of guides previously shown to exhibit low on-target accuracy when tested with pHelper and pDonor were utilized. In all cases, a substantial increase in the relative frequency of on-target integration events was observed with the modified CAST construct (FIG. 5D). Importantly, this effect did not come at the expense of efficiency, as qPCR measurements revealed that on-target integration occurred with an equal or higher efficiency under low TnsC conditions, as compared to the original pHelper design (FIG. 12F). It is possible that at low TnsC conditions, the decreased availability of TnsC at untargeted sites presumably titrates fewer TnsB-DNA complexes away from on-target sites and might be causative to the increased on-target efficiency.

Combining structural and functional evidence, type V-K CASTs maintained a distinct RNA-independent pathway facilitated by TnsB, TnsC, and TniQ (FIG. 5E). The ability of TnsC to promiscuously form filaments on AT-rich DNA was determined to be a major driver of untargeted insertions. The results highlight the competition between TnsB recruitment at TnsC-bound RNA-guided target sites versus AT-rich untargeted sites and indicate that TnsB preferentially reacts with Cas12k-bound on-target sites compared to untargeted sites (FIG. 5E).

The structure of the ‘BCQ’ strand-transfer complex reveals two turns of TnsC filaments, an overall architecture reminiscent of the Cas12k-containing on-target transpososome, with no major structural differences associated with TnsC in either of these assemblies. However, TnsC residues K103 and T121 proximal to TnsB in the ‘BCQ’ transpososome contact the DNA in 3′ to 5′ strand polarity, following the direction of TniQ- to TnsB-binding face of TnsC (FIG. 1F). TnsC variants with mutations to the DNA-contacting residues retain the ability to filament on DNA, and maintain a significant proportion of integration at untargeted sites accompanied by a drop in on-target integration (FIG. 12C). This suggests that these residues may not be a prerequisite for TnsC-DNA binding. Rather, these residues may serve as an intrinsic regulatory feature to ensure that random TnsC filaments default to contacting in the 3′ to 5′ strand polarity. This interaction mode could represent an energetically less favored or passive TnsC configuration for TnsB-mediated integration, thereby permitting only a subset of sites scanned by TnsC in the genome to be licensed for untargeted transposition.

Poly-A tracts in the genome represent regions of altered DNA curvature, and the single-molecule experiments revealed that TnsC filamentation exhibits inherent affinity for AT-rich locations. Further meta-analyses of integration data revealed a preference for AT-rich sequences across a ˜25-bp window spanning ˜2 turns of a TnsC filament, upstream of features recognized by TnsB. AT-rich genomic regions with altered DNA curvature may resemble the bending of DNA observed between unproductive and productive Cas12k transpososomes, leading to preferential TnsC recruitment and a more favorable DNA binding mode that promotes TnsB-based DNA integration. The TnsC K103A mutation led to a complete loss of AT preference in the integration profile, suggesting that this mutant may achieve an energetically more favorable filamentation state, regardless of nucleotide composition. The loss of on-target integration for K103A may result from mutant TnsC filaments titrating TnsB-donor DNA complexes to untargeted sites in the genome more effectively than WT TnsC filaments.

Importantly, a gain-of-function TnsC mutant (A225V) was also identified for E. coli Tn7, which was capable of transposition in the absence of either of the two targeting factors, TnsD and TnsE, and which facilitated integration at AT-rich sequences. Considering the highly conserved operonic nature of TnsB, TnsC, and TniQ in Tn7-like elements, Tn5053, and type V-K CASTs, regulation in the stoichiometry of these proteins could be important for accessing an RNA-independent untargeted pathway.

Type V-K CASTs are among the most compact type of CRISPR-associated transposases, in terms of coding size, and thus offer a major potential opportunity relative to other CAST systems. However, two key properties that limit their use for genome engineering applications are low on-target specificity and the generation of cointegrate transposition products due to lack of TnsA. Recent efforts substantially decreased cointegrate formation by fusing an endonuclease to TnsB, and improved specificity using chimeric fusion proteins or supplementing additional components like pir. Yet these strategies also compromise on-target integration efficiency and do not address the root cause of promiscuity. In this regard, the results herein provide a deeper molecular understanding of how type V-K CAST components undergo both RNA-guided and RNA-independent transposition. TnsC filamentation on AT-rich DNA sequences was identified as a primary driver of untargeted integration and, under limiting TnsC concentrations, RNA-guided transposition becomes the primary pathway of choice biochemically and in cells. This rescue in specificity was generalizable for all the guides tested and importantly, resulted in equal or higher on-target integration efficiency when compared to the original ShCAST pHelper.

### Materials and Methods

Plasmid construction. All constructs used in experiments with ShCAST were cloned using the pHelper (pUC19) and pDonor (pCDFDuet1) described previously (Vo, P. L. H. et al. Nat Biotechnol 39, 480-489 (2021). These constructs were derived from a CAST system from Scytonema hofmanni UTEX B 2349 (Strecker, J. et al. Sci New York N Y 365, 48-53 (2019). Cloning was performed using a combination of Gibson assembly, inverse (around-the-horn) PCR, restriction digestion and ligation. All fragments used for cloning were amplified using Q5 DNA polymerase (NEB). TnsC and Cas12k were cloned into pCOLADuet1 vector backbone using Gibson assembly. The promoter strength was varied by cloning inducible promoters (lac, T7) or constitutive promoters (J23114, J23105, J23119) using inverse (around-the-horn) PCR. S. hofmanni S15 was cloned into expression strains and as fusions using a synthesized gene fragment (TWIST Biosciences). Different targets for pHelper or pHelper lacking TnsC was cloned in using around-the-horn PCR.

Constructs for protein expression having an N-terminal hexahistidine fused to a SUMO tag and a TEV cleavage site (His₆-SUMO-TEV) were designed by restriction digestion of p1S vector (QB3 MacroLab) with Ssp1-HF (NEB) followed by Gibson assembly of the protein of interest. Expression was performed in E. coli BL21 (DE3) cells.

pTarget was designed by cloning a target from λ-DNA into pUC19 vector. Modulating regions in pTarget for introducing artificial untargeted sites was achieved using inverse (around-the-horn) PCR.

All primers and DNA oligos for the study were ordered from IDT. Cloning was performed in NEB Turbo E. coli and plasmid was extracted using Qiagen Miniprep columns and clones were confirmed by Sanger Sequencing (GENEWIZ). Whole plasmid sequences were confirmed by nanopore long read sequencing (Plasmidsaurus). E. coli was grown in LB agar plates or in liquid LB media. Constructs with pUC19 backbone was grown in 100 μg/mL carbenicillin, pCOLADuet1 and p1S in 50 μg/mL kanamycin and pCDFDuet1 in 100 μg/mL spectinomycin. All plasmid sequences and its description are available in Table 1. Sequences of recombinantly expressed proteins used in this study are available in Table 2.

E. coli transposition assay. Transposition assays were performed in E. coli BL21 (DE3) based on a method described previously (Vo, P. L. H. et al. Nat Biotechnol 39, 480-489 (2021), using a 2-plasmid system composed of pDonor, and pHelper. Chemically competent cells having pDonor were transformed with pHelper, plated, and grown at 37° C. for 16 h in the presence of appropriate antibiotics (100 μg/mL carbenicillin and 100 μg/mL spectinomycin). Cells were scrapped off and resuspended in LB. 4×10⁹ cells were re-plated, induced and grown in the presence 0.1 mM IPTG and antibiotics for 24 h at 37° C. Cells were scrapped off and resuspended in LB. 2×10⁹ cells were used for genomic extraction using Wizard® Genomic DNA Purification Kit (Promega). For transposition with an additional plasmid pCas12k, pTnsC or pTnsB, competent cells having pDonor, pCas12k/pTnsC/pTnsB were transformed with the pHelper and grown at the same conditions in the presence of appropriate antibiotics (50 μg/mL kanamycin, 100 μg/mL carbenicillin and 100 μg/mL spectinomycin). All E. coli transposition experiments were performed in n=2 biological replicates.

Recombinant expression of CAST proteins. All CAST proteins were cloned into a pET derivative vector with an N-terminal His₆-SUMO-TEV fusion and were purified similar to previous protocols (Strecker, J. et al. Sci New York N Y 365, 48-53 (2019) and Querques, I., et al., Nature 599, 497-502 (2021). All constructs were transformed in E. coli BL21 (DE3) and grown until an OD600 of 0.5-0.6 in 2×YT media in the presence of kanamycin (50 μg/mL). Protein expression was induced in the presence of 0.5 M isopropyl ß-D-1-thiogalactopyranoside (IPTG) for 16 h at 16° C.

In order to purify TnsB and TniQ, cells were harvested by centrifuging at 6000 g for 15 min at 4° C. and lysed by sonication in lysis buffer (20 mM Tris pH 7.5, 0.5 M NaCl, 5 mM Mg(OAc)₂, 10 mM imidazole, 0.1% Triton X-100, 1 mM DTT and 5% (v/v) glycerol) supplemented with 1 mM phenylmethylsulfonyl fluoride (PMSF), 0.2 mg/mL lysozyme and 0.5× complete protease inhibitor cocktail tablet (Roche). Soluble protein was isolated by centrifuging at 35,050 g for 50 min and the supernatant was incubated with Ni-NTA agarose beads, pre-equilibrated with equilibration buffer (20 mM HEPES pH 7.5, 0.5 M NaCl, 0.5 mM PMSF, 1 mM DTT, 10 mM imidazole and 5% (v/v) glycerol) for 45 min at 4° C. The Ni-NTA beads were further washed with 20 column volume (CV) of wash buffer (20 mM HEPES pH 7.5, 0.5 M NaCl, 0.5 mM PMSF, 1 mM DTT, 30 mM imidazole and 5% glycerol) in a gravity column and protein was eluted in elution buffer (20 mM HEPES pH 7.5, 0.5 M NaCl, 0.5 mM PMSF, 1 mM DTT, 300 mM imidazole, and 5% glycerol). Next, His₆-SUMO-tag was cleaved-off using 5% (w/v) TEV protease and was simultaneously dialyzed in Slide-a-Lyzer cassette (Thermo Fischer Scientific) against dialysis buffer (20 mM HEPES pH 7.5, 0.2 M NaCl, 0.5 mM PMSF, 1 mM DTT, and 1% (v/v) glycerol) for 12 h at 4° C. Any precipitated protein was removed by centrifugation at 20,000 g for 20 min at 4° C. Further, proteins were subjected to Heparin-affinity chromatography using a 0.2-1 M NaCl gradient spanning 10 CV in a buffer containing 20 mM HEPES pH 7.5, 0.2 M NaCl, 1 mM DTT and 1% glycerol. It is to be noted that TniQ does not bind to Heparin and elute in the flowthrough, whereas contaminant proteins were removed in this step. Further, both the proteins were purified by size-exclusion chromatography in SEC Buffer (20 mM HEPES pH 7.5, 0.2 M NaCl, 1 mM DTT and 1% glycerol) using Superdex 200 pg 16/600 column. TniQ and TnsB were concentrated to 127 and 100 μM and stored in a buffer containing 20 mM HEPES pH 7.5, 0.25 M NaCl, 5% glycerol, 1 mM DTT at −80° C. There was no contamination of S15 in TniQ due to the step involving Heparin.

For Cas12k, lysis, equilibration, wash, and elution buffer contained Tris pH 8.0 instead of HEPES pH 7.5. The dialysis buffer used contained 20 mM HEPES pH 7.5, 0.25 M NaCl, 0.5 mM PMSF, 1 mM DTT, and 1% (v/v) glycerol. The SEC buffer contained 20 mM HEPES pH 7.5, 0.25 M NaCl, 1 mM DTT, and 1% (v/v) glycerol. All the rest of the protocol and buffers used were the same as in case of TnsB and TniQ. Cas12k was concentrated to 109 UM and stored at the same conditions as TnsB and TniQ.

For TnsC, lysis, equilibration, wash, and elution buffer contained 0.5 M NaCl to increase protein solubility. TEV cleavage and dialysis was performed in 20 mM HEPES pH 7.5, 1 M NaCl, 0.5 mM PMSF, 1 mM DTT, and 1% (v/v) glycerol after which the protein was subject to a second round of Ni-NTA to separate His₆-SUMO tag from the cleaved TnsC. The column was washed with 20 mM HEPES pH 7.5, 500 mM NaCl, 30 mM imidazole, 1 mM DTT, 0.5 mM PMSF, 5% (v/v) glycerol, 1 mM DTT, 0.5 mM PMSF and the flowthrough was concentrated and further purified by size exclusion chromatography in 20 mM HEPES pH 7.5, 1 M NaCl, 5% glycerol, 1 mM DTT using Superdex 200 pg 16/600 column. TnsC was concentrated to 142 μM and stored in a buffer containing 20 mM HEPES pH 7.5, 1 M NaCl, 10% (v/v) glycerol, 1 mM DTT.

For S15, lysis, equilibration, wash, and elution buffer contained Tris pH 8.0 instead of HEPES pH 7.5. The dialysis buffer contained 20 mM HEPES pH 7.5, 0.15 M NaCl, 0.5 mM PMSF, 1 mM DTT, and 1% (v/v) glycerol. S15 was eluted from Heparin using a gradient of 0.15 to 1 M NaCl in 20 mM HEPES pH 7.5, 1 mM DTT and 15% (v/v) glycerol. SEC contained 20 mM HEPES pH 7.5, 0.25 M NaCl, 1 mM DTT, and 1% (v/v) glycerol. All the rest of the protocols and buffers used were the same as in case of TnsB and TniQ. S15 was concentrated to 168 UM and stored at the same conditions as TnsB and TniQ.

Untargeted ‘BCQ’ transpososome sample preparation. The untargeted ‘BCQ’ transpososome was reconstituted as previously described for the Cas12k-bound transpososome (Park, J.-U. et al. Science 373, 768-774 (2021)), with some modifications noted below. Two separate DNA substrates were first prepared by annealing three synthetic oligonucleotides (IDT) respectively: LE1, LE2, and LE3 for target-LE DNA and RE1, RE2, and RE3 for target-RE DNA. The oligonucleotides were mixed in a 1:1:1 molar ratio and supplemented with 10× concentrated annealing buffer for the following final composition: 10 mM Tris pH 7.5, 50 mM NaCl, and 1 mM EDTA. These mixtures were then heated up to 95° C. for 5 minutes and slowly cooled to room temperature at the rate of 1° C. per minute using a thermal cycler (BioRad).

TnsB, TnsC, and TniQ proteins were purified identically as previously described (Strecker, J. et al. Sci New York N Y 365, 48-53 (2019) and Park, J.-U. et al. Structural basis for target site selection in RNA-guided DNA transposition systems. Science 373, 768-774 (2021). All the protein stocks were first buffer-exchanged into the dilution buffer (25 mM HEPES pH 7.5, 200 mM NaCl, 1 mM DTT, and 2% glycerol) using 0.5 mL centrifugal filters (Millipore). For the target pot, TnsC, TniQ, and the annealed target-LE DNA were first mixed and supplemented with ATP and MgCl₂, resulting in the final composition of the following: 45 μM TnsC, 30 μM TniQ, 3 μM target-LE DNA, 3 mM ATP, and 10 mM MgCl₂ in the dilution buffer. For the donor pot, TnsB and target-RE DNA were combined to make the following composition: 36 μM TnsB, 6 μM target-RE DNA, and 10 mM MgCl₂ in dilution buffer. Target and donor pots were incubated independently for 10 min at room temperature before being combined in a 2:1 volume ratio to reconstitute the transpososome. The composition of the final reaction condition was the following: 30 μM TnsC, 20 μM TniQ, 12 μM TnsB, 2 μM target-LE DNA, 2 μM target-RE DNA, 2 mM ATP, and 10 mM MgCl₂. The final reaction mixture was then incubated for 30 min at 37° C.

Cryo-EM sample preparation and imaging. Homemade graphene-oxide (GO) coated grids were made as previously described (Park, J.-U., et al., Proc. Natl. Acad. Sci. 119, e2202590119 (2022) and Wang, F. et al. Proc National Acad Sci 117, 24269-24273 (2020)). The untargeted transpososome sample was prepared by diluting the final reconstitution mixture three-fold using the dilution buffer (described above). Four microliters of the diluted sample were loaded on the carbon side of the GO-coated grid, which was mounted on the Mark IV Vitrobot (ThermoFisher) set to 4° C. and 100% humidity. The grid was then incubated for 20 seconds in the Vitrobot chamber to let the proteins adhere to the GO surface of the grid. Then the grid was blotted for 7 seconds with a blot force of 5 before being plunged into a slurry of liquid ethane cooled with liquid nitrogen.

The vitrified samples were imaged using 200 kV Talos Arctica (ThermoFisher) equipped with K3 direct electron detector (Gatan) and BioQuantum energy filter (Gatan). The electron beam was carefully aligned following the established protocol or parallel illumination and coma-free alignment. 3,081 micrographs were recorded using SerialEM at 63,000× magnification, corresponding to 1.33 Å per pixel scale. A three-by-three image shift was used to accelerate the image collection with a nominal defocus from −1 μm to −2.5 μm. The total electron dose was 50 electrons per 1 Å² during 3.2 seconds of recording, fractionated into 50 frames.

Cryo-EM image analysis and visualization. Warp was used for beam-induced motion correction, CTF estimation, and initial particle picking of the collected movies. The preprocessed micrographs were filtered based on the CTF-estimated resolution, resulting in 2,790 micrographs. These micrographs and corresponding particle stacks were imported to cryoSPARC or downstream image analysis. 2D classification of the particle stack resulted in a subset of particles with a cryo-EM density of both TnsC oligomer and TnsB strand-transfer complex (STC). This set of particles was used to train the Topaz neural network, which extracted 993, 190 particles. 2D classification of this initial particle stack resulted in 577,628 particles with a cryo-EM density of TnsB STC and TnsC. These particles were then subjected to heterogeneous refinement in cryoSPARC using the three initial references of the following: (1) TnsB STC only, (2) two turns of TnsC, and (3) TnsB STC bound to two turns of TnsC, which was generated using ab initio reconstruction. 237,487 particles classified into the third class were then subjected to focused refinement on the TnsC region using a mask covering the TnsC. The resulting particles were then exported to RELION or 3D classification focusing on TniQ and adjacent two TnsC subunits. Since the molecular mass of this region was relatively small (˜80 kDa), 3D classification was done without image re-alignment. One class (42,210 particles), corresponding to strong density of TniQ, was selected and re-imported into cryoSPARC for another round of 3D classification, focusing on the single subunit of TniQ. This final classification resulted in a final stack of 13,392 particles. This final particle stack was subjected to homogeneous refinement to generate a consensus map. Focused refinements were done through two separate non-uniform refinement jobs focusing on the TniQ-TnsC region or the TnsB STC region of the reconstruction. Local resolution and directional resolution were estimated using blocres53 and 3DFSC, respectively. For FIG. 1F resolution filtered maps from each focused refinement were aligned onto the consensus map and then combined using UCSF Chimera command ‘vop maximum’. The atomic model was generated through rigid-body docking of the chains from the RNA-guided transpososome structure (PDB: 8EA3). Figures describing the cryo-EM reconstruction or atomic model were generated using UCSF ChimeraX.

E. coli growth time course. E. coli B121 (DE3) cells were transformed with a plasmid containing combinations of TnsC and TnsB on an inducible promoter (T7) or an empty plasmid and grown for 16 h in the presence of appropriate antibiotics (50 μg/mL kanamycin and 100 μg/mL carbenicillin). Single colonies were picked for each biological replicate and inoculated to a primary culture and grown for 16 h with antibiotics. 1:200 dilution of primary culture was added to sterile 96-well plate black/clear bottom plates (Thermo Scientific) in a final volume of 200 μL with antibiotics. The culture was grown at 37° C. with continuous shaking and OD600 was measured for 16 h in Synergy Neo2 plate reader (BioTek). All readings were blank corrected. The error bar represents the standard deviation measured across a minimum of two biological replicates.

Fluorescence polarization. Fluorescence polarization was performed as reported earlier (Hoffmann, F. T. et al. Nature 609, 384-393 (2022). A 55 bp dsDNA labeled with a 5′ Fluorescein (6-FAM) tag on one of its strands was used for all fluorescence polarization experiments (Table 3). Recombinantly expressed TnsC (0-15 μM) was titrated with the FAM-labeled dsDNA in 1× binding buffer (20 mM HEPES pH 7.5, 2 mM MgCl₂, 200 mM NaCl, 10 μM ZnCl₂, 1 mM DTT) in the presence of 2 mM ATP or its analogs. The samples were incubated at 37° C. for 30 min in a 384-well plate. The fluorescence gain was adjusted to a sample lacking TnsC while keeping a requested polarization value of 60 mP. The error bar represents the standard deviation measured across two technical replicates. For low salt measurements, the concentration of NaCl was maintained at 50 mM.

For fluorescence polarization measurements for TniQ alone or its interaction with DNA bound to TnsC, TniQ (0-1 μM) was titrated and incubated for 30 min at 37° C. with fluorescently labeled DNA alone or DNA preincubated with 300 mM TnsC (30 min at 37° C.). Experiments were conducted in a 1× binding buffer containing 50 mM NaCl in a 25 μL final volume.

In vitro ATP hydrolysis assay. Relative ATP hydrolysis was measured using a Malachite Green Phosphate Assay Kit (Sigma-Aldrich) according to the manufacturer's protocol as previously described (Hoffmann, F. T. et al. Nature 609, 384-393 (2022). TnsC (10 μM) alone or TnsC (10 μM) together with TnsB (20 μM) was incubated with 10 μM dsDNA (Table 3) in the presence of 1×ATPase reaction buffer (20 mM HEPES pH 7.5, 2 mM MgCl₂, 180 mM NaCl, 10 μM ZnCl₂, 1 mM DTT) supplemented with 1 mM ATP in a final 5 μL volume for 120 min at 37° C. The ATP hydrolysis reaction was diluted with 40 μL water and incubated with 10 μL of working reagent for 5 min. The absorbance at 0 nm was measured in a transparent 384-well plate in plate reader. All samples were blank corrected. The ATPase activity measured was normalized to the sample containing TnsC, TnsB and dsDNA which liberated 174 μM of phosphate. Error bar represents the s.d. measured across two technical replicates (n=2).

Biochemical transposition assay. Guide RNA was transcribed in vitro using MEGAclear Transcription Clean-Up Kit (Invitrogen) using a linear dsDNA as a template (Table 3). Prior to setting the transposition reaction, the ribonucleoprotein (RNP) complex composed of Cas12k (0.75 μM) and guide RNA (sgRNA-6, 9 μM) was pre-incubation at R.T. for 5 min in a total volume of 1.4 μL. In vitro integration was performed in the presence of RNP (50 nM), TniQ (100 nM), TnsC (100 nM), TnsB (1 μM), pTarget (0.6 nM) and pDonor (1.75 nM) supplemented with ATP (2 mM), BSA (50 μg/mL), SUPERase•In Rnase Inhibitor (Invitrogen) (0.25 U/μL) in a 1×IVI Buffer (25 mM HEPES pH 7.5, 5 mM Tris pH 8.0, 0.05 mM EDTA, 20 mM MgCl₂, 30 mM NaCl, 20 mM KCl, 1 mM DTT, 5% (v/v) glycerol). The reaction having a final volume 25 μL was incubated at 37° C. for 2 h, quenched by heat denaturing at 95° C. for 3 min and flash frozen until library preparation for NGS. Transposition was performed in the presence of Mg²⁺ ion concentration of 2 mM throughout the reaction, in contrast to the conditions mentioned earlier. Pre-incubation with low Mg²⁺ at 30° C. is not necessary for the biochemical reconstitution of CAST transposition. For titration involving individual proteins, the concentration was varied as indicated (FIGS. 2 & 9), keeping all other components constant. S15 was supplemented for reactions involving the same. The products of in vitro transposition are Shapiro intermediates as opposed to resolved co-integrates in cellular experiments. The transposon-target DNA junction corresponding to the Shapiro intermediate was amplified by PCR and visualized on 1.5% agarose., qPCR, or short-read sequencing was also performed on the Shapiro intermediate. Due to linear amplification in the first cycle of PCR for the Shapiro intermediate compared to the reference used in qPCR, the efficiencies measured are relative for biochemical experiments. Without S15, a relative on-target efficiency of ˜10% was detected by qPCR whereas supplementing S15 resulted in 33% efficiency.

qPCR analysis for in vitro and genomic transpositions. qPCR for detecting on-target integration in the genome was performed with two sets of primers. A combination of: 1) genome-specific (targets for sgRNA 1-5) and left transposon-specific primers probing t-LR; and 2) genome-specific primers for detecting an E. coli reference gene rssA. Primer pairs were designed to amplify products between 100 and 250 bp and showed amplification efficiency between 100 and 110%. Probes for Taqman qPCR were designed with a 5′ FAM-label ZEN/3′ IBFQ (IDT) as a universal probe which annealed to the transposon left-end and can be displaced only upon amplification. The control probe was designed to bind to E. coli rssA gene and contained a 5′ SUN-label ZEN/3′ IBFQ (IDT). qPCR for in vitro integration was detected using SYBR Green. qPCR for genomic samples was performed using 5 ng of purified genomic DNA in the presence of 1 μL each of 18 μM forward primer and reverse primer pairs (Table 3), 5 μL of TaqMan Fast Advanced Master Mix (Thermo Fischer Scientific), 0.5 μL of each 5 μM probe (Table 3) and 1 μL of water in a final volume of 10 μL. On-target integration efficiency is measured as 100× (2\hat{ }ΔCq), where ΔCq is the Cq (control rssA)−Cq (integration).

qPCR for detecting the Shapiro intermediate resulting from on-target integration was performed with two sets of primers. A combination of: 1) pTarget and left transposon-specific primers probing t-LR; and 2) primers for detecting pTarget. qPCR measurements for in vitro integration were performed with 2 μL of 20 μL in vitro integration reaction, 1 μL each of 10 μM forward and reverse primer (Table 3), 5 μL of SsoAdvanced Universal SYBR Green 2× Supermix (BioRad), and 2 μL of water in a final volume of 10 μL. The first cycle of qPCR only results in linear amplification in comparison to the control targeting pTarget and therefore it is noted that efficiency measurements are relative. On-target integration efficiency is measured as 100×(2\hat{ }ΔCq), where ΔCq is the Cq (pTarget)−Cq (integration).

All qPCR reactions were performed on 384-well clear/white plates (BioRad) on a CFX384 Real-Time PCR Detection System (BioRad) using the following thermal cycling conditions: DNA denaturing (DNA denaturation 98° C. for 2.5 min), 40 cycles of amplification (98° C. for 10 s, 62° C. for 20 s), and terminal melt-curve analysis was performed (65-95° C. in 0.5° C. per 5 s increments). All qPCR primers used in this study are mentioned in Table 3.

TagIn Seq library preparation and sequencing. In order to get good sequencing coverage across genomic and plasmid targets, samples from biochemical and E. coli transposition assays were treated with AvrII (NEB) to reduce the contaminating pDonor. A single AvrII cut site was located 21-nt from the left transposon-end in pDonor whereas pTarget had no sites. E. coli BL21-DE3 genome contains only 16 AvrII cut sites. For in vitro integration, 1.5× μL of Mag-Bind TotalPure NGS magnetic beads (Omega) were added to each sample and the DNA was purified using the manufacturer's protocol. The sample was eluted in 10 μL volume and digested with AvrII (NEB) (5 U) in rCutSmart Buffer (NEB) for 1 h at 37° C. 1.5× magnetic beads were added to each sample and DNA was purified and eluted in a 10 μL volume. For genomic samples, 1 μg of isolated genomic DNA (having contaminating pDonor) was digested with AvrII (5 U) in a 50 μL volume for 2 h at 37° C. Total genomic DNA was purified with magnetic beads, eluted in 10 μL volume and quantified using Qubit dsDNA High Sensitivity Kit (Invitrogen) and was used for tagmentation using Nextera XT DNA Library Preparation Kit (Illumina).

4 ng of DNA from either biochemical or E. coli transposition assays were mixed with 5 μL of tagmentation DNA buffer (Illumina) and 1 μL of amplicon tagmentation mix (Illumina) in a final volume of 10 μL and incubated at 55° C. for 7 min. The tagmentation reaction was quenched and DNA was amplified by PCR-1 step, using CAST left transposon-end specific primer, Y3 mix (0.42 μM), having universal TrueSeq adaptor (15) overhang, Nextera adaptor (17) specific primer (0.42 μM) (Table 3) and 30 μL KAPA HiFi HotStart ReadyMix in a 60 μL final volume. Y3 mix is a combination of three primers with variable length between CAST transposon-end specific region and universal TrueSeq adaptor to introduce diversity during Illumina flow cell clustering (Table 3). After 20 cycles of amplification at an annealing temperature of 57° C., the amplified DNA was purified using 1.5× of magnetic beads and eluted in 10 μL volume. This DNA was next subjected to PCR-2 using TrueSeq (15) specific primers (0.42 μM) having indexed universal p5 overhang, Nextera adaptor (17) specific primer (0.42 μM) having indexed universal p7 overhang, and 30 μL KAPA HiFi HotStart ReadyMix in a 60 μL final volume. After 13 cycles of amplification at an annealing temperature of 54° C., the barcoded amplicon was purified by magnetic beads and eluted in 10 μL volume. The DNA was resolved by 1.5% agarose followed by excision and isolation within the size range of 300-600 bp using Gel Extraction Kit (Qiagen). NGS libraries were pooled and quantified by qPCR using NEBNext Library Quant Kit (NEB). Sequencing was performed with NextSeq high-output kit with 75/150-cycle single-end reads and automated adaptor trimming and demultiplexing (Illumina) was performed.

Analysis of NGS data. Analysis of TagTn Seq data was performed using a custom Python pipeline as described (Vo, P. L. H. et al. Nat Biotechnol 39, 480-489 (2021). Demultiplexed raw reads having half of the bases with a Phred quality score of less than 20, which corresponds to greater than 1% base miscalling were removed from the analysis. Reads having the last 23 bases of the transposon-end sequence (5′ GACAGATAATTTGTCACTGTACA 3′; SEQ ID NO: 110) and a 22-bp adjacent flanking genomic sequences were extracted and noted as the total-transposon-end containing reads. This 22-bp adjacent region represents the fingerprint region used to identify transposon insertions and was used to align to the reference genome or plasmid using Bowtie2. The reference genome for E. coli BL21 (DE3) was based on published data from National Center for Biotechnology Information (NCBI) genomes. Only reads which mapped perfectly and only once to the genome were chosen. Reads which did not get mapped to the genome were checked for sequences corresponding to the pDonor and noted as pDonor contamination. Alignments from Bowtie2 were used for generating genome/plasmid-wide coordinates for integration. If the read was mapped to the same strand as the input fasta file, then it was noted as on ‘fwd’ strand, and if mapped to the complementary strand as the input fasta file, then it is noted as on ‘rev’ strand. The read ‘position’ was indexed at the fifth position of target site duplication (TSD) for each event, with respect to the ‘fwd strand’. The orientation of the integration relative to the fasta file was concluded based on whether the library was sequenced from the right end or the left end of the transposon for TagTn sequencing. The orientation of the transposon insertion with respect to the protospacer at the on-target window (100 bp from the end of protospacer), was noted as target-left-right (t-LR) or target-right-left (tRL).

Untargeted reads and on-target reads were assigned using a custom Python script. For biochemical integration, events that mapped at a hundred base window after the end of the protospacer on pTarget were presumed as on-target events, whereas any other integration events on pDonor and pTarget were totaled and noted as untargeted. Reads due to contaminating pDonor and a potential PCR recombination artifact (at position 1198 and 2328 nt) were masked for all samples. For E. coli integration, reads that mapped at a hundred base window after the end of the protospacer were noted as on-targets, and reads elsewhere in the genome as untargeted. Raw reads for integration at on-target and untargeted sites for each sample was normalized with the total transposon-end containing reads (including pDonor contamination) detected for that sample and further scaled with respect to the sample having the highest transposon-end containing reads (in the experimental set). It is to be noted that there is no correction for amplification biases during NGS sequencing and for this reason, only unique insertion sites were used for reporting sequence preferences in transposition. For visualizing normalized plots comparing integration across the genome, raw reads for each coordinate for a sample were normalized and scaled in the same way as above, converted to bigwig files and visualized in IGV.

For the plot comparing untargeted integration sites across pTarget, reads at the on-target window were excluded. A continuous single-base position for the pTarget and the reads detected at that position was generated using a custom Python script. The read at each of these bases were compared between samples. Pearson correlation coefficients (r) between data sets were calculated in Prism 9.

Analysis of genetic neighborhood for integration events detected across the E. coli genome. A custom Python script was used to analyze whether the integration events detected elsewhere in the genome are RNA-dependent off-targets. The position of insertion determined by NGS analysis was used for the genetic neighborhood analysis. Events at the on-target window were removed from the analysis. For every integration site detected, the most likely RNA-dependent off-target was determined as follows. Off-target was assumed to be in the target-left-right orientation similar to an on-target event. A 7 bp window (3 bp upstream and downstream) of sequence, 63 bp upstream from the integration site in the LR orientation was checked for the enrichment of a PAM sequence (GTN). Similarly, n=10,000 random regions in the E. coli genome were chosen, and the corresponding window upstream was checked to get the proportion of random sequences having a potential PAM.

For integration events, PAM-containing sequences were further checked for the presence of matches in the protospacer for sgRNA 1-5 respectively (23 bp downstream to the potential PAM detected). The maximum matching sequence detected was selected as the potential number of spacer matches representing that insertion event. Similarly, the random region of E. coli genome sampled previously with a PAM-containing sequence was checked for matches in protospacer for sgRNA 1-5 respectively. In both cases, when more than one potential protospacer had a valid PAM, the maximum number of matches to the spacer was determined as the potential number of spacer matches for that location.

AT-enrichment analysis. AT-enrichment analysis was performed with a custom Python script. The position of insertion determined by NGS analysis was used for analyzing the AT content. Events at the on-target location were removed from the analysis if a spacer was provided. For every unique insertion site on pTarget, λ-DNA or the E. coli genome, a window of sequence either upstream or downstream was chosen (50 bp for pTarget or λ-DNA and 100 bp window for the E. coli genome). The AT content at both these regions (upstream/downstream) was calculated and the highest of the two was selected as the representative AT content of that unique insertion site. Unique events were binned to across their AT content to create a distribution of events detected at each AT bin. As a control to calculate the AT content of pTarget, λ-DNA or the E. coli genome, the regions on the DNA were randomly sampled (n=50,000) and similarly, the highest AT content of the adjacent bin to every unique sampled location was chosen as representative of that sampling event. The randomly sampled events were also binned across their AT content to create a distribution of random samplings events (counts) detected at each AT bin. A cumulative frequency distribution plot was used to compare the difference in AT content for unique integration events and randomly sampled regions on the pTarget, λ-DNA or the E. coli genome. The AT content of unique integration events and random sampling events were used to perform Mann-Whitney U test used to test the significance of the distributions. pDonor was not used to for AT-enrichment analysis due to additional factors such as target immunity.

To generate plots to visualize the AT content across the pTarget and integration, the pTarget was divided into 59 bins corresponding to 46 bp and the AT percentage was calculated using a custom Python script. The reads detected on pTarget was plotted as an overlay with AT content at that bin. For λ-DNA, the genome was into 45 bins of 1078 bp window and AT percentages were calculated. The integration events detected in λ-DNA were plotted as an overlay with the AT content. Correlation between reads detected for integration and fluorescence intensity of mNG-TnsC binding across λ-DNA (from DNA curtains) were calculated by similarly binning the reads and fluorescence intensity detected across λ-DNA into 45 bins of 1078 bp window and Spearman correlation coefficient (r) was calculated using Prism 9.

Essential gene analysis. Essential gene analysis (FIG. 10L) was performed with a custom Python script. The position of insertion determined by NGS analysis was used to determine if events landed on an essential gene or not. Events at the on-target location and at the T7RNAP locus were removed from the analysis. The remaining reads for these events were annotated with their CDS features in accordance with the NCBI-published genome. Essential genes were noted based on previous reports on E. coli K-12 and reads falling to these regions were classified as essential gene insertions. Reads landing outside these regions were noted as non-essential insertions. The percentage of the E. coli genome which was essential was calculated by summing the length of all essential genes and dividing by the genome length.

Sequence logo for untargeted events. To create sequence logos for untargeted events, every unique site of integration in the E. coli genome, and a window upstream and downstream to the position of integration on both the ‘fwd’ and ‘rev’ strand, was taken. Insertions in the on-target window were omitted for samples having a spacer. Further, TSD correction was accounted on the strands, and insertions were oriented in the left-to-right orientation based on the strand of the input fasta file and the strand to which each read was mapped. The extracted sequence for both the strands was outputted and WebLogo v2.8.2 was used for plotting the sequence logo showing per residue conservation.

For ShCAST, a 75-base window on either side of integration was chosen for building the sequence logo. The sequence logo was indexed with the third position of TSD set as position ‘0’ and 70 bases on either side was plotted. WebLogo in FIG. 3H was plotted for insertions on both the strands observed for two biological replicates of a sample having a pHelper lacking Cas12k and guide RNA. Sequence features for samples with Cas12k and guide RNA (sgRNA-1) was performed using previously reported data for ShCAST generated by random fragmentation and insertion site amplified from the transposon right-end (FIG. 11B). The plots revealed consistent sequence featured compared to samples subjected to TagTn Seq and sequencing from the transposon left end. No conservation of sequences with partial guide complementarity was noted upstream, consistent with the analysis (FIGS. 6C-6E) that the majority of events in type V-K CASTs are RNA-independent.

For ShoCAST, previously reported data (Vo, P. L. H. et al. Nat Biotechnol 39, 480-489 (2021) with a pHelper having ShoCas12k and guide RNA (sgRNA-7) was used and analyzed to inform the position and read for each integration event. WebLogo in FIG. 11C was plotted for unique insertions. A 75-base window was chosen for building the sequence logo. The sequence logo was indexed with the third position of TSD set as position ‘0’ and 70 bases on either side were plotted.

DNA curtains. Single-molecule double-tethered dsDNA curtain experiments were carried out as previously described (Meir, A., et al., J Vis Exp (2020) doi: 10.3791/61320). Custom flow cells were assembled using quartz slides, on which chromium patterns were deposited through nanofabrication. The dsDNA substrate was prepared by annealing and ligating two custom oligonucleotide handles (IDT) at the COS sites of bacteriophage λ-DNA (NEB), such that one end of the DNA contained a biotin modification while the other a digoxigenin (dig). Prior to assembly of DNA curtains, flow cells were first passivated with a lipid bilayer. The biotin ends of the DNA were tethered through a biotin-streptavidin linkage to biotin-modified lipids within the bilayer. These DNA molecules were then aligned as the chromium barriers and flow-stretched so that the dig-tagged ends were anchored to chromium pedestals coated with anti-dig antibody (Roche). These DNA molecules were then flow-stretched so that the dig-ends were anchored at chromium pedestals coated with anti-dig antibody (Roche). A pre-defined barrier-pedestal distance of 12 μm and orthogonal attachment chemistry yielded uniform and unambiguous orientation of the double-tethered dsDNA curtain. Fluorescent mNeonGreen-TnsC (mNG-TnsC) was injected into the flow cell, from a 50 μL sample loop, using a syringe pump. Sample illumination was achieved using a continuous-wave 488 nm laser (Coherent Sapphire, 200 mW), shuttered externally (Uniblitz LS6). The emission signal was visualized through a custom-built prism-type total internal reflection fluorescence (TIRF) microscopy system, based on a Nikon Eclipse TE2000-U, equipped with a 60× water immersion objective and an EMCCD camera (Andor iXon X3). All single-molecule experiments were performed in reaction buffer (20 mM HEPES pH 7.5, 2 mM MgCl₂, 200 mM NaCl, 10 μM ZnCl₂, 1 mM DTT, 0.2 mg/mL BSA, and 2 mM ATP), at room temperature (25° C.). In equilibrium binding experiments, flow was stopped after mNG-TnsC (100 nM) had reached the flow cell and data were collected at the rate of 1 frame per 30 seconds for 30 min. mNG-TnsC was injected at a concentration of 500 nM for the experiment shown in FIG. 10C.

Data analysis for DNA curtains. Files saved in ND2 format were first converted to TIFF stacks in FIJI, where all subsequent image processing steps took place. For binding and disassembly experiments, a kymograph was first generated for each DNA molecule by making a single-pixel wide slice, drawn along the length of the DNA, through the time series. Fluorescence intensity over time was then extracted by plotting the profile of the kymograph of interest in FIJI, which produced intensity values, each averaged over the entire one-pixel-wide DNA, over time. Intensity time series from >50 individual filaments were combined, normalized, and plotted as mean±s.d. over time (FIGS. 3C and 3D). Apparent rates (k_{obs}; FIG. 3D) were obtained by nonlinear regression to a one-phase association model in Prism 9. For intensity versus binding position (FIG. 3C), the last 10 frames (5 min) of a 30-minute binding experiment were averaged to remove intensity fluctuations in any given frame due to random DNA fluctuations within the evanescent field. For each DNA of interest, a signal intensity profile was plotted over a 33-pixel long region, centered over the 45-pixel total length of the DNA (12 μm), to reduce signal contamination and bleed-over from nonspecific binding of mNG-TnsC to chromium features. Intensity profiles from >60 individual DNA molecules were combined and plotted as mean±standard deviation. The A/T percentage of the λ-sequence was obtained by first segregating the entire sequence (48,502 bp) into 45 bins of 1078 bp width, then calculating the A/T percentage for the sequence within each bin. The number of bins was chosen to reflect the fact that λ-DNA double tethered at 12 μm end-to-end distance spanned 45 pixels in the camera of this imaging platform. Thus, fluorescence intensity measured at any pixel along the entire filament should represent the amount of mNG-TnsC bound to the DNA within the window of 1078 bp.

Western blot. An N-terminal Flag epitope tag was cloned into TnsC expressed with a T7 or lac promoter. Cells were grown at the same conditions as the E. coli transposition assay in the presence of 0.1 mM IPTG. 2×10⁷ were incubated with 5 μL of 6×SDS loading dye and boiled for 10 min at 95° C. The samples were resolved on Mini-PROTEAN TGX Stain-Free Precast Gels and imaged to confirm equal loading. The gel was transferred using iBlot 2 Gel Transfer Device using the manufacturer's protocol. The transferred blots were washed twice with wash buffer (1% PBS, 0.1% Tween) for 5 min each and then washed with blocking buffer (1% PBS, 0.1% Tween, 5% BSA) for another 5 min. The blot was then incubated for 30 min in 70 mL blocking buffer at RT with gentle shaking. After 30 min, the blot was again washed with block buffer and incubated for 1 h with either Monoclonal ANTI-FLAG M2 (Sigma) (1:7500 dilution) in 15 mL volume or GAPDH Loading Control Monoclonal Antibody (Thermo Scientific) (1:2500 dilution) in 5 mL volume in blocking buffer with gentle shaking. The same wash protocol was repeated and incubated with a secondary HRP conjugated antibody, Goat Anti-Mouse IgG1 HRP (Abcam) (1:15000 dilution) in 15 mL volume and incubated for 2 h. The blot was washed twice in the wash buffer for 5 min each and imaged using SuperSignal West Dura Extended Duration Substrate (Thermo Scientific) after incubating for 2 min.

Statistics and reproducibility. All E. coli transposition assays and NGS were performed in two independent biological replicates. qPCR for the same was performed in two independent replicates. E. coli growth curves were measured in minimum of two independent replicates. Titrations for in vitro transposition were performed once for each concentration. Single molecule experiments were done in replicates (data not shown). Fluorescence polarization and ATP hydrolysis assays were performed in two technical replicates.

Data availability. Next-generation sequencing data will be made available in the National Center for Biotechnology Information (NCBI) Sequence Read Archive. Datasets generated and analyzed in the current study are available from the corresponding author upon reasonable request.

Code availability. Custom python scripts used for computational analysis of next generation sequencing data are available at GitHub (github.com/sternberglab/George_et_al_2023).

TABLE 1
Description and sequence of plasmids used in this study.

| Plasmid | | | Plasmid |
|---|---|---|---|
| ID | System | Plasmid description | SEQ ID NO |
| pSL0008 | Type V-K ShCAST | pCOLADuet-1 | 1 |
| pSL1225 | Type V-K ShCAST | pUC19, ShCAST_pHelper, NT-guide | 2 |
| pSL1226 | Type V-K ShCAST | pUC19, ShCAST_pDonor, wildtype | 3 |
| pSL1396 | Type V-K ShCAST | pUC19, ShCAST_pHelper, wildtype (sgRNA-1) | 4 |
| pSL1398 | Type V-K ShCAST | pUC19, ShCAST_pHelper, wildtype (sgRNA-5) | 5 |
| pSL3751 | Type V-K ShCAST | pUC19, ShCAST_pHelper, mNeonGreen-TnsC | 6 |
| | | (sgRNA-1) | |
| pSL4043 | Type V-K ShCAST | pUC19, pTarget (targeted by sgRNA-6) | 7 |
| pSL4141 | Type V-K ShCAST | pUC19, ShCAST_pHelper, TnsC R189E (sgRNA-1) | 8 |
| pSL4145 | Type V-K ShCAST | pUC19, ShCAST_pHelper, TnsC K103A (sgRNA-1) | 9 |
| pSL4228 | Type V-K ShCAST | pCOLADuet-1_T7_promoter_pTnsC | 10 |
| pSL4232 | Type V-K ShCAST | pUC19, ShCAST_pHelper, Cas12k-XTEN-GS-TnsC | 11 |
| | | fusion (sgRNA-1) | |
| pSL4246 | Type V-K ShCAST | pUC19, ShCAST_pHelper, ΔCas12k, ΔsgRNA | 12 |
| pSL4623 | Type V-K ShCAST | pUC19, ShCAST_pHelper, Cas12k-XTEN-GS-S15 | 13 |
| | | fusion (sgRNA-1) | |
| pSL4643 | Type V-K ShCAST | pUC19, ShCAST_pHelper, TniQ-XTEN-GS-S15 fusion | 14 |
| | | (sgRNA-1) | |
| pSL4644 | Type V-K ShCAST | pUC19, ShCAST_pHelper, S15-XTEN-GS-TniQ fusion | 15 |
| | | (sgRNA-1) | |
| pSL4787 | Type V-K ShCAST | pCOLADuet-1, ShCAST_pHelper, dCas9-XTEN-GS- | 16 |
| | | TnsC fusion (sgRNA-8) | |
| pSL4788 | Type V-K ShCAST | pCOLADuet-1, ShCAST_pHelper, TnsC-XTEN-GS- | 17 |
| | | dCas9 fusion (sgRNA-8) | |
| pSL4789 | Type V-K ShCAST | pUC19, ShCAST_pHelper, ΔCas12k, ΔsgRNA, ΔTniQ | 18 |
| pSL4843 | Type V-K ShCAST | pCOLADuet-1_Lac_promoter_pTnsC | 19 |
| pSL4853 | Type V-K ShCAST | pCOLADuet-1_Lac_promoter_pCas12k (sgRNA-1) | 20 |
| pSL4854 | Type V-K ShCAST | pCOLADuet-1_J23114_promoter_pCas12k (sgRNA-1) | 21 |
| pSL4855 | Type V-K ShCAST | pCOLADuet-1_J23105_promoter_pCas12k (sgRNA-1) | 22 |
| pSL4857 | Type V-K ShCAST | pCOLADuet-1_J23119_promoter_pCas12k (sgRNA-1) | 23 |
| pSL4858 | Type V-K ShCAST | pUC19, ShCAST_pHelper, ΔTnsC (sgRNA-1) | 24 |
| pSL4859 | Type V-K ShCAST | pUC19, ShCAST_pHelper, ΔTnsB (sgRNA-1) | 25 |
| pSL4963 | Type V-K ShCAST | pUC19, pTarget/pT-2 (targeted by sgRNA-6) | 26 |
| pSL4964 | Type V-K ShCAST | pUC19, pTarget/pT-3 (targeted by sgRNA-6) | 27 |
| pSL4965 | Type V-K ShCAST | pUC19, pTarget/pT-4 (targeted by sgRNA-6) | 28 |
| pSL4966 | Type V-K ShCAST | pUC19, pTarget/pT-5 (targeted by sgRNA-6) | 29 |
| pSL4967 | Type V-K ShCAST | pUC19, pTarget/pT-6 (targeted by sgRNA-6) | 30 |
| pSL4971 | Type V-K ShCAST | pUC19_T7_promoter_pTnsB | 31 |
| pSL5041 | Type V-K ShCAST | pUC19, ShCAST_pHelper, ΔTnsC (sgRNA-2) | 32 |
| pSL5043 | Type V-K ShCAST | pUC19, ShCAST_pHelper, wildtype (sgRNA-5) | 33 |
| pSL5044 | Type V-K ShCAST | pUC19, ShCAST_pHelper, ΔTnsC (sgRNA-3) | 34 |
| pSL5045 | Type V-K ShCAST | pUC19, ShCAST_pHelper, ΔTnsC (sgRNA-4) | 35 |
| pSL5060 | Type V-K ShCAST | pUC19, ShCAST_pHelper, wildtype (sgRNA-2) | 36 |
| pSL5062 | Type V-K ShCAST | pUC19, ShCAST_pHelper, wildtype (sgRNA-3) | 37 |
| pSL5063 | Type V-K ShCAST | pUC19, ShCAST_pHelper, wildtype (sgRNA-4) | 38 |
| pSL5097 | Type V-K ShCAST | pCOLADuet-1_T7_promoter_pTnsC R189E | 39 |
| pSL5098 | Type V-K ShCAST | pCOLADuet-1_T7_promoter_3XFlag-pTnsC | 40 |
| pSL5099 | Type V-K ShCAST | pCOLADuet-1_Lac_promoter_3XFlag-pTnsC | 41 |
| pSL4558 | Type V-K ShCAST | pCOLADuet-1_T7_promoter_ShCas12k_sgRNA | 42 |
| | | (sgRNA-1) | |
| pSL4262 | Type V-K ShCAST | pCOLADuet-1_T7_promoter_TnsB | 43 |
| pSL3752 | Type V-K ShCAST | p1S, His6-SUMO-tev-TnsC | 44 |
| pSL3491 | Type V-K ShCAST | p1S, His-SUMO-TEV-TnsB | 45 |
| pSL3492 | Type V-K ShCAST | p1S, His-SUMO-TEV-TniQ | 46 |
| pSL3914 | Type V-K ShCAST | p1S, His-SUMO-TEV-Cas12k | 47 |
| pSL3426 | Type V-K ShCAST | p1S, His6-SUMO-tev-mNeonGreen-TnsC | 48 |
| pSL4622 | Type V-K ShCAST | p1S, His6-SUMO-tev-S15 | 49 |

TABLE 2
Recombinantly Purified CAST Proteins

| Protein | | | Protein Sequence | Protein |
|---|---|---|---|---|
| Name | Strain | System | (after TEV cleavage) | length (aa) |
| Cas12k | Scytonema | Type V- | SNMSQITIQARLISFESNRQ | 641 |
| | hofmannii | K | QLWKLMADLNTPLINELLCQ | |
| | (UTEX | CAST | LGQHPDFEKWQQKGKLPSTV | |
| | B 2349) | | VSQLCQPLKTDPRFAGQPSR | |
| | | | LYMSAIHIVDYIYKSWLAIQ | |
| | | | KRLQQQLDGKTRWLEMLNSD | |
| | | | AELVELSGDTLEAIRVKAAE | |
| | | | ILAIAMPASESDSASPKGKK | |
| | | | GKKEKKPSSSSPKRSLSKTL | |
| | | | FDAYQETEDIKSRSAISYLL | |
| | | | KNGCKLTDKEEDSEKFAKRR | |
| | | | RQVEIQIQRLTEKLISRMPK | |
| | | | GRDLTNAKWLETLLTATTTV | |
| | | | AEDNAQAKRWQDILLTRSSS | |
| | | | LPFPLVFETNEDMVWSKNQK | |
| | | | GRLCVHFNGLSDLIFEVYCG | |
| | | | NRQLHWFQRFLEDQQTKRKS | |
| | | | KNQHSSGLFTLRNGHLVWLE | |
| | | | GEGKGEPWNLHHLTLYCCVD | |
| | | | NRLWTEEGTEIVRQEKADEI | |
| | | | TKFITNMKKKSDLSDTQQAL | |
| | | | IQRKQSTLTRINNSFERPSQ | |
| | | | PLYQGQSHILVGVSLGLEKP | |
| | | | ATVAVVDAIANKVLAYRSIK | |
| | | | QLLGDNYELLNRQRRQQQYL | |
| | | | SHERHKAQKNFSPNQFGASE | |
| | | | LGQHIDRLLAKAIVALARTY | |
| | | | KAGSIVLPKLGDMREVVQSE | |
| | | | IQAIAEQKFPGYIEGQQKYA | |
| | | | KQYRVNVHRWSYGRLIQSIQ | |
| | | | SKAAQTGIVIEEGKQPIRGS | |
| | | | PHDKAKELALSAYNLRLTRR | |
| | | | S (SEQ ID NO: 50) | |
| TnsB | Scytonema | Type V- | SNNSQQNPDLAVHPLAIPME | 585 |
| | hofmannii | K CAST | GLLGESATTLEKNVIATQLS | |
| | (UTEX | | EEAQVKLEVIQSLLEPCDRT | |
| | B 2349) | | TYGQKLREAAEKLNVSLRTV | |
| | | | QRLVKNWEQDGLVGLTQTSR | |
| | | | ADKGKHRIGEFWENFITKTY | |
| | | | KEGNKGSKRMTPKQVALRVE | |
| | | | AKARELKDSKPPNYKTVLRV | |
| | | | LAPILEKQQKAKSIRSPGWR | |
| | | | GTTLSVKTREGKDLSVDYSN | |
| | | | HVWQCDHTRVDVLLVDQHGE | |
| | | | ILSRPWLTTVIDTYSRCIMG | |
| | | | INLGFDAPSSGVVALALRHA | |
| | | | ILPKRYGSEYKLHCEWGTYG | |
| | | | KPEHFYTDGGKDFRSNHLSQ | |
| | | | IGAQLGFVCHLRDRPSEGGV | |
| | | | VERPFKTLNDQLFSTLPGYT | |
| | | | GSNVQERPEDAEKDARLTLR | |
| | | | ELEQLLVRYIVDRYNQSIDA | |
| | | | RMGDQTRFERWEAGLPTVPV | |
| | | | PIPERDLDICLMKQSRRTVQ | |
| | | | RGGCLQFQNLMYRGEYLAGY | |
| | | | AGETVNLRFDPRDITTILVY | |
| | | | RQENNQEVFLTRAHAQGLET | |
| | | | EQLALDEAEAASRRLRTAGK | |
| | | | TISNQSLLQEVVDRDALVAT | |
| | | | KKSRKERQKLEQTVLRSAAV | |
| | | | DESNRESLPSQIVEPDEVES | |
| | | | TETVHSQYEDIEVWDYEQLR | |
| | | | EEYGF (SEQ ID NO: 51) | |
| TnsC | Scytonema | Type V- | SNATEAQAIAKQLGGVKPDD | 278 |
| | hofmannii | K CAST | EWLQAEIARLKGKSIVPLQQ | |
| | (UTEX | | VKTLHDWLDGKRKARKSCRV | |
| | B 2349) | | VGESRTGKTVACDAYRYRHK | |
| | | | PQQEAGRPPTVPVVYIRPHQ | |
| | | | KCGPKDLFKKITEYLKYRVT | |
| | | | KGTVSDFRDRTIEVLKGCGV | |
| | | | EMLIIDEADRLKPETFADVR | |
| | | | DIAEDLGIAVVLVGTDRLDA | |
| | | | VIKRDEQVLERFRAHLRFGK | |
| | | | LSGEDFKNTVEMWEQMVLKL | |
| | | | PVSSNLKSKEMLRILTSATE | |
| | | | GYIGRLDEILREAAIRSLSR | |
| | | | GLKKIDKAVLQEVAKEYK | |
| | | | (SEQ ID NO: 52) | |
| TniQ | Scytonema | Type V- | SNIEAPDVKPWLFLIKPYEG | 168 |
| | hofmannii | K | ESLSHFLGRFRRANHLSASG | |
| | (UTEX | CAST | LGTLAGIGAIVARWERFHFN | |
| | B 2349) | | PRPSQQELEAIASVVEVDAQ | |
| | | | RLAQMLPPAGVGMQHEPIRL | |
| | | | CGACYAESPCHRIEWQYKSV | |
| | | | WKCDRHQLKILAKCPNCQAP | |
| | | | FKMPALWEDGCCHRCRMPFA | |
| | | | EMAKLQKV | |
| | | | (SEQ ID NO: 53) | |
| S15 | Scytonema | Type V- | SNMALTQERKQEIIVNYQVH | 91 |
| | hofmannii | K | ETDTGSADVQVAMLTERINR | |
| | (UTEX | CAST | LSLHLQANKKDHSSRRGLLK | |
| | B 2349) | | LIGQRKRLLAYIQKDSREKY | |
| | | | QALIGRLGIRG | |
| | | | (SEQ ID NO: 54) | |
| mNeonGreen- | Scytonema | Type V- | SNAMVSKGEEDNMASLPATH | 519 |
| TnsC | hofmannii | K | ELHIFGSINGVDFDMVGQGT | |
| | (UTEX | CAST | GNPNDGYEELNLKSTKGDLQ | |
| | B 2349) | | FSPWILVPHIGYGFHQYLPY | |
| | | | PDGMSPFQAAMVDGSGYQVH | |
| | | | RTMQFEDGASLTVNYRYTYE | |
| | | | GSHIKGEAQVKGTGFPADGP | |
| | | | VMTNSLTAADWCRSKKTYPN | |
| | | | DKTIISTFKWSYTTGNGKRY | |
| | | | RSTARTTYTFAKPMAANYLK | |
| | | | NQPMYVFRKTELKHSKTELN | |
| | | | FKEWQKAFTDVMGMDELYKG | |
| | | | GSGSTEAQAIAKQLGGVKPD | |
| | | | DEWLQAEIARLKGKSIVPLQ | |
| | | | QVKTLHDWLDGKRKARKSCR | |
| | | | VVGESRTGKTVACDAYRYRH | |
| | | | KPQQEAGRPPTVPVVYIRPH | |
| | | | QKCGPKDLFKKITEYLKYRV | |
| | | | TKGTVSDFRDRTIEVLKGCG | |
| | | | VEMLIIDEADRLKPETFADV | |
| | | | RDIAEDLGIAVVLVGTDRLD | |
| | | | AVIKRDEQVLERFRAHLRFG | |
| | | | KLSGEDFKNTVEMWEQMVLK | |
| | | | LPVSSNLKSKEMLRILTSAT | |
| | | | EGYIGRLDEILREAAIRSLS | |
| | | | RGLKKIDKAVLQEVAKEYK | |
| | | | (SEQID NO: 55) | |

TABLE 3
DNA Oligos used in this study for biochemical experiments, PCR, qPCR, nd NGS

| | | | |
|---|---|---|---|
| Oligo ID | Description | Sequence | SEQ ID NO |

DNA Oligos for Bulk Biochemical Experiments

| | | | |
|---|---|---|---|
| STC-1_LE1 | DNA oligo for BCQ strand | GTCACAATGACATTAATCTGTCACCGAC | 56 |
| | transfer complex | GACAGATAATTTGTCACTGTACAGTAGA | |
| | | ATATAGATGCGCATCTATATAGATGCAAA | |
| | | TTGAGTGGCCTTATTAAATGACTTCTCAA | |
| | | CCAGTCAGCACGCCCAGACCAGGGCAC | |
| STC-2_LE2 | DNA oligo for BCQ strand | GCGTGCTGACTGGTTCTCTTCAGTAAAT | 57 |
| | transfer complex | TATTGGCCACTCAATTTGCATCTATATAG | |
| | | ATGCGCATCTATAT | |
| STC-3_LE3 | DNA oligo for BCQ strand | TGTACAGTGACAAATTATCTGTCGTCGG | 58 |
| | transfer complex | TGACAGATTAATGTCATTGTGAC | |
| STC-4_RE1 | DNA oligo for BCQ strand | TTACTGATGACAATAATTTGTCACAACG | 59 |
| | transfer complex | ACATATAATTAGTCACTGTACATCTACGA | |
| | | TACGTAGCGGCCGACGCG | |
| STC-5_RE2 | DNA oligo for BCQ strand | TGTACAGTGACTAATTATATGTCGTTGTG | 60 |
| | transfer complex | ACAAATTATTGTCATCAGTAA | |
| STC-6_RE3 | DNA oligo for BCQ strand | CGCGTCGGCCGCTACGTATC | 61 |
| | transfer complex | | |
| oSL1542 | DNA oligo for ATP | ACCAACGTGACCTATCCCATTACGGTCA | 62 |
| | hydrolysis assay | ATCCGCCGTTTGTTCCCACGGAGAATC | |
| oSL1543 | Complementary DNA oligo | GATTCTCCGTGGGAACAAACGGCGGATT | 63 |
| | for ATP hydrolysis assay | GACCGTAATGGGATAGGTCACGTTGGT | |
| oSL7033 | 5′ FAM (/56-FAM/)-labeled | GAGGAGGGCTGTTTTTACAAAATCCGGT | 64 |
| | DNA oligo for fluorescence | AGTAACTTGCTAACCAATTCCTAGGCA | |
| | polarization | | |
| oSL7044 | Complementary DNA oligo | TGCCTAGGAATTGGTTAGCAAGTTACTA | 65 |
| | for fluorescence polarization | CCGGATTTTGTAAAAACAGCCCTCCTC | |
| IVT-T1 | DNA oligo used for sgRNA- | GCGTAATACGACTCACTATAGGATATTAA | 66 |
| | 6 transcription | TAGCGCCGCAATTCATGCTGCTTGCAGC | |
| | | CTCTGAATTTTGTTAAATGAGGGTTAGTT | |
| | | TGACTGTATAAATACAGTCTTGCTTTCTG | |
| | | ACCCTGGTAGCTGCTCACCCTGATGCTG | |
| | | CTGTCAATAGACAGGATAGGTGCGCTCC | |
| | | CAGCAATAAGGGCGCGGATGTACTGCTG | |
| | | TAGTGGCTACTGAATCACCCCCGATCAA | |
| | | GGGGGAACCCTCCAAAAGGTGGGTTGA | |
| | | AAGAGCAAGTTACTACCGGATTTTGT | |
| IVT-T2 | Complementary DNA oligo | ACAAAATCCGGTAGTAACTTGCTCTTTC | 67 |
| | used for sgRNA-6 | AACCCACCTTTTGGAGGGTTCCCCCTTG | |
| | transcription | ATCGGGGGTGATTCAGTAGCCACTACAG | |
| | | CAGTACATCCGCGCCCTTATTGCTGGGA | |
| | | GCGCACCTATCCTGTCTATTGACAGCAG | |
| | | CATCAGGGTGAGCAGCTACCAGGGTCA | |
| | | GAAAGCAAGACTGTATTTATACAGTCAA | |
| | | ACTAACCCTCATTTAACAAAATTCAGAG | |
| | | GCTGCAAGCAGCATGAATTGCGGCGCTA | |
| | | TTAATATCCTATAGTGAGTCGTATTACGC | |

PCR

| | | | |
|---|---|---|---|
| oSL7692 | ShCAST LE specific - F1 | GTATAATAATTGCAGAGCATATTATATTG | 68 |
| | | ATG | |
| oSL7693 | pTarget specific primer | GATAACAATTTGGTTAGCAAGTTACTAC | 69 |
| | (targeted by sgRNA-6) - R1 | | |

qPCR

| | | | |
|---|---|---|---|
| oSL2999 | ShCAST LE specific (for E. | CAATTAATTAAGCAACGCTGATG | 70 |
| | coli and pTarget integration) | | |
| | - F2-6/R8 | | |
| oSL2928 | Genome specific (targeted | CGATGAGCGTGGTGGTTATG | 71 |
| | by sgRNA-1) - R2 | | |
| oSL12856 | Genome specific (targeted | GTAATGACGATTTCCGCACTG | 72 |
| | by sgRNA-2) - R3 | | |
| oSL12858 | Genome specific (targeted | CACCTCTTTTAACCCTTGAAGTC | 73 |
| | by sgRNA-3) - R4 | | |
| oSL12860 | Genome specific (targeted | GGTGTTGGCCTGATGAGTTATAG | 74 |
| | by sgRNA-4) - R5 | | |
| oSL12862 | Genome specific (targeted | CAGCGATTTCCATGTTGC | 75 |
| | by sgRNA-5) - R6 | | |
| oSL12507 | Universal Taqman probe for | CATTGTGACTATTTAATTGTCGTCGTGAC | 76 |
| | on-target (genomic); 5′/56- | C | |
| | FAM/and 3′/3IABKFQ/ | | |
| | labeled;/ZEN/internal | | |
| | quencher at position 9 | | |
| oSL4256 | Control (rssA) - F7 | CATGCAGTATTCCAGGACTCA | 77 |
| oSL4257 | Control (rssA) - R7 | AAGGAGAGCAAATCTTGTTGC | 78 |
| oSL12508 | Taqman probe for rssA; 5′ | CAGTCGTTAACCCAATTCCTATTTCCCTC | 79 |
| | /5SUN/and 3′/3IABkFQ/ | A | |
| | labeled;/ZEN/internal | | |
| | quencher at position 9 | | |
| oSL8898 | pTarget specific primer | AGCAAGTTACTACCGGATTTTGT | 80 |
| | (targeted by sgRNA-6) - F8 | | |
| oSL8114 | Control pTarget backbone - | TACGGATGGCATGACAGTAAG | 81 |
| | F9 | | |
| oSL8115 | Control pTarget backbone - | TCAAGGCGAGTTACATGATCC | 82 |
| | R9 | | |

NGS

| | | | |
|---|---|---|---|
| oSL7915 | Amplicon Sequencing - i7 | CTGGAGTTCAGACGTGTGCTCTTCCGAT | 83 |
| | overhang (TrueSeq) for | CTCAATTTGGTTAGCAAGTTACTACC | |
| | pTarget specific primer | | |
| | (targeted by sgRNA-6) - | | |
| | F10 | | |
| oSL7855 | Amplicon Sequencing - i5 | CTCTTTCCCTACACGACGCTCTTCCGATC | 84 |
| | overhang (TrueSeq) for | TGACATTAATCTGTCACCGAC | |
| | ShCAST LE specific - R10 | | |
| n/a | Amplicon/TagTn | AATGATACGGCGACCACCGAGATCTACA | 85 |
| | Sequencing - TrueSeq | CXXXXXXXXACACTCTTTCCCTACACG | |
| | specific P5 overhang - | ACGC | |
| | F11/F13 | | |
| n/a | Amplicon Sequencing - | CAAGCAGAAGACGGCATACGAGATXXX | 86 |
| | TrueSeq specific P7 | XXXXXGTGACTGGAGTTCAGACGTGTG | |
| | overhang - R11 | C | |
| oSL8537 | Y3 mix - i5 overhang | CTCTTTCCCTACACGACGCTCTTCCGATC | 87 |
| | (TrueSeq) for ShCAST LE | TGACATTAATCTGTCACCGACGAC | |
| | specific - R12 | | |
| oSL8538 | Y3 mix - i5 overhang | CTCTTTCCCTACACGACGCTCTTCCGATC | 88 |
| | (TrueSeq) for ShCAST LE | TAGACATTAATCTGTCACCGACGAC | |
| | specific - R12 | | |
| oSL8539 | Y3 mix - i5 overhang | CTCTTTCCCTACACGACGCTCTTCCGATC | 89 |
| | (TrueSeq) for ShCAST LE | TATGACATTAATCTGTCACCGACGAC | |
| | specific - R12 | | |
| oSL8540 | i7 overhang (Nextera) - F12 | TAGTCTCGTGGGCTCGG | 90 |
| n/a | TagTn Sequencing - Nextera | CAAGCAGAAGACGGCATACGAGATXXX | 91 |
| | specific P7 overhang - R13 | XXXXXGTCTCGTGGGCTCGG | |

TABLE 4
Guide RNAS and genomic target sites used in this study

| | | | | | Spacer | | |
|---|---|---|---|---|---|---|---|
| Guide | Encoded | | Targeting | Full length | sequence | | Flanking |
| RNA ID | by | Description | system | sgRNA | (5′→3′) | | PAM |
| sgRNA-NT | pSL1225 | Non- | E. coli | AUAUUAAUAGCGCC | CGGUCU | 5′ | n/a |
| | | targeting | genome | GCAAUUCAUGCUGC | UCUCGA | | |
| | | sgRNA | | UUGCAGCCUCUGAA | CGAGAA | | |
| | | | | UUUUGUUAAAUGA | GACAC | | |
| | | | | GGGUUAGUUUGACU | (SEQ ID | | |
| | | | | GUAUAAAUACAGUC | NO: 101) | | |
| | | | | UUGCUUUCUGACCC | | | |
| | | | | UGGUAGCUGCUCAC | | | |
| | | | | CCUGAUGCUGCUGU | | | |
| | | | | CAAUAGACAGGAUA | | | |
| | | | | GGUGCGCUCCCAGC | | | |
| | | | | AAUAAGGGCGCGGA | | | |
| | | | | UGUACUGCUGUAGU | | | |
| | | | | GGCUACUGAAUCAC | | | |
| | | | | CCCCGAUCAAGGGG | | | |
| | | | | GAACCCUCCAAAAG | | | |
| | | | | GUGGGUUGAAAGCG | | | |
| | | | | GUCUUCUCGACGAG | | | |
| | | | | AAGACAC (SEQ ID | | | |
| | | | | NO: 92) | | | |
| sgRNA-1 | pSL1396, | sgRNA | E. coli | AUAUUAAUAGCGCC | AUGCCG | 5′ | GGTT |
| | pSL4858 | targeting | genome | GCAAUUCAUGCUGC | AUCGCG | | |
| | | lacZ | | UUGCAGCCUCUGAA | TCACAC | | |
| | | | | UUUUGUUAAAUGA | UACGU | | |
| | | | | GGGUUAGUUUGACU | (SEQ ID | | |
| | | | | GUAUAAAUACAGUC | NO: 102) | | |
| | | | | UUGCUUUCUGACCC | | | |
| | | | | UGGUAGCUGCUCAC | | | |
| | | | | CCUGAUGCUGCUGU | | | |
| | | | | CAAUAGACAGGAUA | | | |
| | | | | GGUGCGCUCCCAGC | | | |
| | | | | AAUAAGGGCGCGGA | | | |
| | | | | UGUACUGCUGUAGU | | | |
| | | | | GGCUACUGAAUCAC | | | |
| | | | | CCCCGAUCAAGGGG | | | |
| | | | | GAACCCUCCAAAAG | | | |
| | | | | GUGGGUUGAAAGau | | | |
| | | | | gccgaucgcgucac | | | |
| | | | | acuacgu | | | |
| | | | | (SEQ ID NO: 93) | | | |
| sgRNA-2 | pSL5041, | sgRNA | E. coli | AUAUUAAUAGCGCC | UUGUCA | 5′ | AGTT |
| | pSL5060 | targeting | genome | GCAAUUCAUGCUGC | GATAUU | | |
| | | upstream | | UUGCAGCCUCUGAA | ACGCCU | | |
| | | to | | UUUUGUUAAAUGA | GUGUG | | |
| | | yebK | | GGGUUAGUUUGACU | (SEQ ID | | |
| | | | | GUAUAAAUACAGUC | NO: 103) | | |
| | | | | UUGCUUUCUGACCC | | | |
| | | | | UGGUAGCUGCUCAC | | | |
| | | | | CCUGAUGCUGCUGU | | | |
| | | | | CAAUAGACAGGAUA | | | |
| | | | | GGUGCGCUCCCAGC | | | |
| | | | | AAUAAGGGCGCGGA | | | |
| | | | | UGUACUGCUGUAGU | | | |
| | | | | GGCUACUGAAUCAC | | | |
| | | | | CCCCGAUCAAGGGG | | | |
| | | | | GAACCCUCCAAAAG | | | |
| | | | | GUGGGUUGAAAGuu | | | |
| | | | | gucagauauuacgc | | | |
| | | | | cugugug | | | |
| | | | | (SEQ ID NO: 94) | | | |
| sgRNA-3 | pSL5044, | sgRNA | E. coli | AUAUUAAUAGCGCC | ACUGCC | 5′ | AGTC |
| | pSL5062 | targeting | genome | GCAAUUCAUGCUGC | CGUUUC | | |
| | | oxyS | | UUGCAGCCUCUGAA | GAGAGU | | |
| | | | | UUUUGUUAAAUGA | UUCUC | | |
| | | | | GGGUUAGUUUGACU | (SEQ ID | | |
| | | | | GUAUAAAUACAGUC | NO: 104) | | |
| | | | | UUGCUUUCUGACCC | | | |
| | | | | UGGUAGCUGCUCAC | | | |
| | | | | CCUGAUGCUGCUGU | | | |
| | | | | CAAUAGACAGGAUA | | | |
| | | | | GGUGCGCUCCCAGC | | | |
| | | | | AAUAAGGGCGCGGA | | | |
| | | | | UGUACUGCUGUAGU | | | |
| | | | | GGCUACUGAAUCAC | | | |
| | | | | CCCCGAUCAAGGGG | | | |
| | | | | GAACCCUCCAAAAG | | | |
| | | | | GUGGGUUGAAAGacu | | | |
| | | | | gcccguuucgagag | | | |
| | | | | uuucuc | | | |
| | | | | (SEQ ID NO: 95) | | | |
| sgRNA-4 | pSL5045, | sgRNA | E. coli | AUAUUAAUAGCGCC | AUAGCG | 5′ | AGTT |
| | pSL5063 | targeting | genome | GCAAUUCAUGCUGC | AUCCCU | | |
| | | upstream to | | UUGCAGCCUCUGAA | UGCUGA | | |
| | | yidQ | | UUUUGUUAAAUGA | AAAUA | | |
| | | | | GGGUUAGUUUGACU | (SEQ ID | | |
| | | | | GUAUAAAUACAGUC | NO: 105) | | |
| | | | | UUGCUUUCUGACCC | | | |
| | | | | UGGUAGCUGCUCAC | | | |
| | | | | CCUGAUGCUGCUGU | | | |
| | | | | CAAUAGACAGGAUA | | | |
| | | | | GGUGCGCUCCCAGC | | | |
| | | | | AAUAAGGGCGCGGA | | | |
| | | | | UGUACUGCUGUAGU | | | |
| | | | | GGCUACUGAAUCAC | | | |
| | | | | CCCCGAUCAAGGGG | | | |
| | | | | GAACCCUCCAAAAG | | | |
| | | | | GUGGGUUGAAAGaua | | | |
| | | | | gcgaucccuugcug | | | |
| | | | | aaaaua | | | |
| | | | | (SEQ ID NO: 96) | | | |
| sgRNA-5 | pSL1398, | sgRNA | E. coli | AUAUUAAUAGCGCC | GCCACU | 5′ | TGTT |
| | pSL5044 | targeting | genome | GCAAUUCAUGCUGC | CGCUUU | | |
| | | lacZ | | UUGCAGCCUCUGAA | AAUGAU | | |
| | | | | UUUUGUUAAAUGA | GAUUU | | |
| | | | | GGGUUAGUUUGACU | (SEQ ID | | |
| | | | | GUAUAAAUACAGUC | NO: 106) | | |
| | | | | UUGCUUUCUGACCC | | | |
| | | | | UGGUAGCUGCUCAC | | | |
| | | | | CCUGAUGCUGCUGU | | | |
| | | | | CAAUAGACAGGAUA | | | |
| | | | | GGUGCGCUCCCAGC | | | |
| | | | | AAUAAGGGCGCGGA | | | |
| | | | | UGUACUGCUGUAGU | | | |
| | | | | GGCUACUGAAUCAC | | | |
| | | | | CCCCGAUCAAGGGG | | | |
| | | | | GAACCCUCCAAAAG | | | |
| | | | | GUGGGUUGAAAGgcc | | | |
| | | | | acucgcuuuaauga | | | |
| | | | | ugauuu | | | |
| | | | | (SEQ ID NO: 97) | | | |
| sgRNA-6 | n/a | sgRNA | pTarget, | GGAUAUUAAUAGCG | AGCAAG | 5′ | GGTT |
| | | targeting | λ-DNA | CCGCAAUUCAUGCU | UUACUA | | |
| | | pTarget, λ- | | GCUUGCAGCCUCUG | CCGGAU | | |
| | | DNA | | AAUUUUGUUAAAU | UUUGU | | |
| | | | | GAGGGUUAGUUUG | (SEQ ID | | |
| | | | | ACUGUAUAAAUACA | NO: 107) | | |
| | | | | GUCUUGCUUUCUGA | | | |
| | | | | CCCUGGUAGCUGCU | | | |
| | | | | CACCCUGAUGCUGC | | | |
| | | | | UGUCAAUAGACAGG | | | |
| | | | | AUAGGUGCGCUCCC | | | |
| | | | | AGCAAUAAGGGCGC | | | |
| | | | | GGAUGUACUGCUGU | | | |
| | | | | AGUGGCUACUGAAU | | | |
| | | | | CACCCCCGAUCAAG | | | |
| | | | | GGGGAACCCUCCAA | | | |
| | | | | AAGGUGGGUUGAA | | | |
| | | | | AGAGCAAGUUACUA | | | |
| | | | | CCGGAUUUUGU | | | |
| | | | | (SEQ ID NO: 98) | | | |
| sgRNA-7 | n/a | sgRNA | E. coli | GUACUAAUAGCGCC | CAGCGC | 5′ | AGTA |
| | | targeting | genome | GCAGUUCAUGCUCU | GGCUGA | | |
| | | lacZ | | UUAAGAGUCUCUGU | AAUCAU | | |
| | | | | ACUGUGGAAAAUCU | CAUUA | | |
| | | | | GGGUUAGUUUGACG | (SEQ ID | | |
| | | | | GUUGGAAAACCGUU | NO: 108) | | |
| | | | | UUGCUUUCUGACCC | | | |
| | | | | UGGUAGCUGCCCGC | | | |
| | | | | UUCUCAUGCUCUGA | | | |
| | | | | CUUUUCACGUUAUG | | | |
| | | | | UGGAAAAAGUAACG | | | |
| | | | | UAAUUUCGUUAGUU | | | |
| | | | | AAGACUUACCGUAA | | | |
| | | | | AAAGUCAGUUCUGA | | | |
| | | | | UGCUGCUGUCGCAA | | | |
| | | | | GACAGGAUAGGUGC | | | |
| | | | | GCUCCCAGCAAAAG | | | |
| | | | | GAGUAUGUCUUGAA | | | |
| | | | | AAAGACUAGCCGUU | | | |
| | | | | CUAGUAACGGUGCG | | | |
| | | | | GAUUACCGCAGUGG | | | |
| | | | | UGGCUACUGAAUCA | | | |
| | | | | CCCCCUUCGUCGGG | | | |
| | | | | GGAACCCUCCAAAA | | | |
| | | | | GGUGGGUUGAAAGC | | | |
| | | | | AGCGCGGCUGAAAU | | | |
| | | | | CAUCAUUA (SEQ ID | | | |
| | | | | NO: 99) | | | |
| sgRNA-8 | pSL4787, | dCas9 | E. coli | UGAUUUCAGCCGCG | UGAUUU | 3′ | TGG |
| | pSL4788 | sgRNA | genome | CUGUACGUUUUAGA | CAGCCG | | |
| | | targeting | | GCUAGAAAUAGCAA | CGCUGU | | |
| | | lacZ | | GUUAAAAUAAGGCU | AC (SEQ | | |
| | | | | AGUCCGUUAUCAAC | ID NO: | | |
| | | | | UUGAAAAAGUGGCA | 109) | | |
| | | | | CCGAGUCGGUGC | | | |
| | | | | (SEQ ID NO: 100) | | | |

The scope of the present invention is not limited by what has been specifically shown and described hereinabove. Those skilled in the art will recognize that there are suitable alternatives to the depicted examples of materials, configurations, constructions, and dimensions. Variations, modifications, and other implementations of what is described herein will occur to those of ordinary skill in the art without departing from the spirit and scope of the invention.

Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety.

1. A system er kit for enhancing the specificity of CRISPR-associated transposon (CAST) system comprising:
an engineered Clustered Regularly Interspaced Short Palindromic Repeats (CRISPR)-associated transposon (CAST) system or one or more nucleic acids encoding the engineered CAST system, wherein the CAST system comprises:
one or more Cas proteins;
one or more transposon associated proteins comprising at least TnsC; and
optionally, at least one gRNA complementary to at least a portion of the target nucleic acid sequence,
wherein the system comprises less TnsC, or a nucleic acid encoding TnsC, compared to any or all remaining components of the CAST system or nucleic acids encoding thereof,
wherein TnsC is encoded by an individual nucleic acid under a promoter with lower copy number compared to the promoter utilized for expressing any or all remaining components of the CAST system, and/or
wherein the TnsC comprises:
one or more mutations to decrease affinity for DNA binding at non-specific sites and/or increase affinity for target site DNA binding;
one or more mutations to disrupt the formation of TnsC filaments; and/or
an N-terminal moiety.

2. The system of claim 1, wherein the CAST system is derived from a Type I CRISPR-Cas system or a Type V CRISPR-Cas system.

3. The system of claim 1, wherein the CAST system is derived from a type V-K, type I-F, type I-B, or type I-D CRISPR-Cas system.

4. The system of claim 1, wherein the one or more Cas proteins comprise one or more of Cas12, Cas5, Cas6, Cas7, and Cas8.

5. The system of claim 1, wherein the one or more transposon associated proteins further comprises one or both of TnsB and TniQ.

6. A method for modifying a target nucleic acid comprising contacting a target nucleic acid sequence with the system of claim 1.

7. The method of claim 6, wherein the method results in less off-target modification than a method using a non-engineered CAST system.

8. A method for enhancing the specificity of CRISPR-associated transposon (CAST) system, comprising decreasing levels of TnsC in the CAST system, perturbing TnsC N-terminus, and/or modulating the affinity of TnsC for DNA binding,
wherein the CAST system comprises:
one or more Cas proteins;
one or more transposon associated proteins comprising at least TnsC; and
optionally, at least one gRNA complementary to at least a portion of the target nucleic acid sequence; or
one or more nucleic acid encoding the one or more Cas proteins, one or more transposon associated proteins, and the at least one gRNA.

9. The method of claim 8, wherein the CAST system is derived from a Type I CRISPR-Cas system or a Type V CRISPR-Cas system.

10. The method of claim 8, wherein the one or more Cas proteins comprise one or more of Cas12, Cas5, Cas6, Cas7, and Cas8 and/or wherein the one or more transposon associate proteins further comprises one or both of TnsB and TniQ.

11. The method of claim 8, wherein modulating the affinity of TnsC for DNA binding comprises:
introducing mutations in TnsC to decrease TnsC affinity for DNA binding at non-specific sites and/or increase affinity for target site DNA binding;
introducing mutations in TnsC to disrupt the formation of TnsC filaments; and/or
adding an agent to disrupt or control formation of TnsC filaments.

12. The method of claim 8, wherein perturbing TnsC N-terminus comprises fusing a moiety to the TnsC N-terminus or introducing to the CAST system an N-terminal TnsC fusion protein.

13. The method of claim 12, wherein the moiety comprises an effector domain, one of the one or more Cas proteins or other components of a CAST system, or an exogenous protein or protein domain.

14. The method of claim 8, wherein the CAST system is in a cell and decreasing the levels of TnsC in the CAST system comprises decreasing TnsC expression levels in the cell.

15. The method of claim 14, wherein decreasing TnsC expression levels comprises expressing TnsC from a lower copy number promoter.
